SEQUENCE LISTING (47 sequences)

SEQ ID NO: 1 - DNA, 20 bp, Artificial Sequence
  Exemplary sequence encoding an sgRNA targeting sequence
tctacccacagggatcaatg

SEQ ID NO: 2 - DNA, 20 bp, Artificial Sequence
  Exemplary sequence encoding an sgRNA targeting sequence
gccttgcagattaagtacac

SEQ ID NO: 3 - DNA, 20 bp, Artificial Sequence
  Exemplary sequence encoding an sgRNA targeting sequence
taatgtgaaccggttcacag

SEQ ID NO: 4 - DNA, 20 bp, Artificial Sequence
  Exemplary sequence encoding an sgRNA targeting sequence
gcagctgcgaagatatcggg

SEQ ID NO: 5 - DNA, 20 bp, Artificial Sequence
  Exemplary sequence encoding an sgRNA targeting sequence
tgagtgcaaaactgagcgcg

SEQ ID NO: 6 - DNA, 20 bp, Artificial Sequence
  Exemplary sequence encoding an sgRNA targeting sequence
tcttctacctcgagatcatg

SEQ ID NO: 7 - DNA, 20 bp, Artificial Sequence
  Exemplary sequence encoding an sgRNA targeting sequence
gaagatacgccaaagcgtcg

SEQ ID NO: 8 - DNA, 20 bp, Artificial Sequence
  Exemplary sequence encoding an sgRNA targeting sequence
aaaacacttgcataacgatg

SEQ ID NO: 9 - DNA, 20 bp, Artificial Sequence
  Exemplary sequence encoding an sgRNA targeting sequence
acttgcagtaaattagctcg

SEQ ID NO: 10 - DNA, 20 bp, Artificial Sequence
  Exemplary sequence encoding an sgRNA targeting sequence
caggaggcaagctaacacgg

SEQ ID NO: 11 - DNA, 20 bp, Artificial Sequence
  Exemplary sequence encoding an sgRNA targeting sequence
gttgccgacagaccgaacga

SEQ ID NO: 12 - DNA, 20 bp, Artificial Sequence
  Exemplary sequence encoding an sgRNA targeting sequence
attaaccggtttctcccggg

SEQ ID NO: 13 - DNA, 20 bp, Artificial Sequence
  Exemplary sequence encoding an sgRNA targeting sequence
gttctgtgtaaaatcacact

SEQ ID NO: 14 - DNA, 20 bp, Artificial Sequence
  Exemplary sequence encoding an sgRNA targeting sequence
atggctttatgcaacacgtg

SEQ ID NO: 15 - DNA, 20 bp, Artificial Sequence
  Exemplary sequence encoding an sgRNA targeting sequence
caatccggctaagtcggacg

SEQ ID NO: 16 - DNA, 20 bp, Artificial Sequence
  Exemplary sequence encoding an sgRNA targeting sequence
agagctaaattaattgagtg

SEQ ID NO: 17 - DNA, 20 bp, Artificial Sequence
  Exemplary sequence encoding an sgRNA targeting sequence
gcagccgaatcgccaaccgt

SEQ ID NO: 18 - DNA, 20 bp, Artificial Sequence
  Exemplary sequence encoding an sgRNA targeting sequence
gccatcgacctgatgtacgg

SEQ ID NO: 19 - DNA, 20 bp, Artificial Sequence
  Exemplary sequence encoding an sgRNA targeting sequence
ccaggtttcccgatccagag

SEQ ID NO: 20 - DNA, 20 bp, Artificial Sequence
  Exemplary sequence encoding an sgRNA targeting sequence
gaaggtctgtccccgctaca

SEQ ID NO: 21 - DNA, 20 bp, Artificial Sequence
  Exemplary sequence encoding an sgRNA targeting sequence
tttccttctcgacacaactg

SEQ ID NO: 22 - DNA, 20 bp, Artificial Sequence
  Exemplary sequence encoding an sgRNA targeting sequence
tcatgaaacacaccaaacga

SEQ ID NO: 23 - DNA, 20 bp, Artificial Sequence
  Exemplary sequence encoding an sgRNA targeting sequence
ttagtagtcaatctgtaccc

SEQ ID NO: 24 - DNA, 20 bp, Artificial Sequence
  Exemplary sequence encoding an sgRNA targeting sequence
gatgaccgatgtactcaagg

SEQ ID NO: 25 - DNA, 20 bp, Artificial Sequence
  Exemplary sequence encoding a non-targeting sgRNA
aaaaagtccgcgattacgtc

SEQ ID NO: 26 - DNA, 35 bp, Artificial Sequence
  Primer sequence
ggctttatatatcttgtggaaaggacgaaacaccg

SEQ ID NO: 27 - DNA, 38 bp, Artificial Sequence
  Primer sequence
ctagccttattttaacttgctatttctagctctaaaac

SEQ ID NO: 28 - DNA, 22 bp, Artificial Sequence
  Primer sequence
cttccctcgacgaattcccaac

SEQ ID NO: 29 - DNA, 1794 bp, Homo sapiens
atgggcaagaagcacaagaagcacaaggccgagtggcgctcgtcctacgaggattatgccgacaagcccctggagaagcctctaaagctagtcctgaaggtcggaggaagtgaagtgactgaactctcaggatccggccacgactccagttactatgatgacaggtcagaccatgagcgagagaggcacaaagaaaagaaaaagaagaagaagaagaagtccgagaaggagaagcatctggacgatgaggaaagaaggaagcgaaaggaagagaagaagcggaagcgagagagggagcactgtgacacggagggagaggctgacgactttgatcctgggaagaaggtggaggtggagccgcccccagatcggccagtccgagcgtgccggacacagccagccgaaaatgagagcacacctattcagcaactcctggaacacttcctccgccagcttcagagaaaagatccccatggattttttgcttttcctgtcacggatgcaattgctcctggatattcaatgataataaaacatcccatggattttggcaccatgaaagacaaaattgtagctaatgaatacaagtcagttacggaatttaaggcagatttcaagctgatgtgtgataatgcaatgacatacaataggccagataccgtgtactacaagttggcgaagaagatccttcacgcaggctttaagatgatgagcaaacaggcagctcttttgggcaatgaagatacagctgttgaggaacctgtccctgaagttgtaccagtacaagtagaaactgccaagaaatccaaaaagccgagtagagaagttatcagctgcatgtttgagcctgaagggaatgcctgcagcttgacggacagtaccgcagaggagcacgtgctggcgctggtggagcacgcagctgacgaagctcgggacaggatcaaccggttcctcccaggcggcaagatgggctatctgaagaggaacggggacgggagcctgctctacagcgtggtcaacacggccgagccggacgctgatgaggaggagacccacccggtggacttgagctcgctctccagtaagctactcccaggcttcaccacgctgggcttcaaagacgagagaagaaacaaagtcacctttctctccagtgccactactgcgctttcgatgcagaataattcagtatttggcgacttgaagtcggacgagatggagctgctctactcagcctacggagatgagacaggcgtgcagtgtgcgctgagcctgcaggagtttgtgaaggatgctgggagctacagcaagaaagtggtggacgacctcctggaccagatcacaggcggagaccactctaggacgctcttccagctgaagcagagaagaaatgttcccatgaagcctccagatgaagccaaggttggggacaccctaggagacagcagcagctctgttctggagttcatgtcgatgaagtcctatcccgacgtttctgtggatatctccatgctcagctctctggggaaggtgaagaaggagctggaccctgacgacagccatttgaacttggatgagacgacgaagctcctgcaggacctgcacgaagcacaggcggagcgcggcggctctcggccgtcgtccaacctcagctccctgtccaacgcctccgagagggaccagcaccacctgggaagcccttctcgcctgagtgtcggggagcagccagacgtcacccacgacccctatgagtttcttcagtctccagagcctgcggcctctgccaagacctaa

SEQ ID NO: 30 - protein, 597 aa, Homo sapiens
MGKKHKKHKAEWRSSYEDYADKPLEKPLKLVLKVGGSEVTELSGSGHDSSYYDDRSDHERERHKEKKKKKKKKSEKEKHLDDEERRKRKEEKKRKREREHCDTEGEADDFDPGKKVEVEPPPDRPVRACRTQPAENESTPIQQLLEHFLRQLQRKDPHGFFAFPVTDAIAPGYSMIIKHPMDFGTMKDKIVANEYKSVTEFKADFKLMCDNAMTYNRPDTVYYKLAKKILHAGFKMMSKQAALLGNEDTAVEEPVPEVVPVQVETAKKSKKPSREVISCMFEPEGNACSLTDSTAEEHVLALVEHAADEARDRINRFLPGGKMGYLKRNGDGSLLYSVVNTAEPDADEEETHPVDLSSLSSKLLPGFTTLGFKDERRNKVTFLSSATTALSMQNNSVFGDLKSDEMELLYSAYGDETGVQCALSLQEFVKDAGSYSKKVVDDLLDQITGGDHSRTLFQLKQRRNVPMKPPDEAKVGDTLGDSSSSVLEFMSMKSYPDVSVDISMLSSLGKVKKELDPDDSHLNLDETTKLLQDLHEAQAERGGSRPSSNLSSLSNASERDQHHLGSPSRLSVGEQPDVTHDPYEFLQSPEPAASAKT

SEQ ID NO: 31 - DNA, 1959 bp, Homo sapiens
atgggcaagaagcacaagaagcacaagtcggacaaacacctctacgaggagtatgtagagaagcccttgaagctggtcctcaaagtaggagggaacgaagtcaccgaactctccacgggcagctcggggcacgactccagcctcttcgaagacaaaaacgatcatgacaaacacaaggacagaaagcggaaaaagagaaagaaaggagagaagcagattccaggggaagaaaaggggagaaaacggagaagagttaaggaggataaaaagaagcgagatcgagaccgggtggagaatgaggcagaaaaagatctccagtgtcacgcccctgtgagattagacttgcctcctgagaagcctctcacaagctctttagccaaacaagaagaagtagaacagacaccccttcaagaagctttgaatcaactgatgagacaattgcagagaaaagatccaagtgctttcttttcatttcctgtgactgattttattgctcctggctactccatgatcattaaacacccaatggattttagtaccatgaaagaaaagatcaagaacaatgactatcagtccatagaagaactaaaggataacttcaaactaatgtgtactaatgccatgatttacaataaaccagagaccatttattataaagctgcaaagaagctgttgcactcaggaatgaaaattcttagccaggaaagaattcagagcctgaagcagagcatagacttcatggctgacttgcagaaaactcgaaagcagaaagatggaacagacacctcacagagtggggaggacggaggctgctggcagagagagagagaggactctggagatgccgaagcacacgccttcaagagtcccagcaaagaaaataaaaagaaagacaaagatatgcttgaagataagtttaaaagcaataatttagagagagagcaggagcagcttgaccgcatcgtgaaggaatctggaggaaagctgaccaggcggcttgtgaacagtcagtgcgaatttgaaagaagaaaaccagatggaacaacgacgttgggacttctccatcctgtggatcccattgtaggagagccaggctactgccctgtgagactgggaatgacaactggaagacttcagtctggagtgaatactttgcaggggttcaaagaggataaaaggaacaaagtcactccagtgttatatttgaattatgggccctacagttcttatgcaccgcattatgactccacatttgcaaatatcagcaaggatgattctgatttaatctattcaacctatggggaagactctgatcttccaagtgatttcagcatccatgagtttttggccacgtgccaagattatccgtatgtcatggcagatagtttactggatgttttaacaaaaggagggcattccaggaccctacaagagatggagatgtcattgcctgaagatgaaggccatactaggacacttgacacagcaaaagaaatggagcagattacagaagtagagccaccagggcgtttggactccagtactcaagacaggctcatagcgctgaaagcagtaacaaattttggcgttccagttgaagtttttgactctgaagaagctgaaatattccagaagaaacttgatgagaccaccagattgctcagggaactccaggaagcccagaatgaacgtttgagcaccagaccccctccgaacatgatctgtctcttgggtccctcatacagagaaatgcatcttgctgaacaagtgaccaataatcttaaagaacttgcacagcaagtaactccaggtgatatcgtaagcacgtatggagttcgaaaagcaatggggatttccattccttcccccgtcatggaaaacaactttgtggatttgacagaagacactgaagaacctaaaaagacggatgttgctgagtgtggacctggtggaagttga

SEQ ID NO: 32 - protein, 652 aa, Homo sapiens
MGKKHKKHKSDKHLYEEYVEKPLKLVLKVGGNEVTELSTGSSGHDSSLFEDKNDHDKHKDRKRKKRKKGEKQIPGEEKGRKRRRVKEDKKKRDRDRVENEAEKDLQCHAPVRLDLPPEKPLTSSLAKQEEVEQTPLQEALNQLMRQLQRKDPSAFFSFPVTDFIAPGYSMIIKHPMDFSTMKEKIKNNDYQSIEELKDNFKLMCTNAMIYNKPETIYYKAAKKLLHSGMKILSQERIQSLKQSIDFMADLQKTRKQKDGTDTSQSGEDGGCWQREREDSGDAEAHAFKSPSKENKKKDKDMLEDKFKSNNLEREQEQLDRIVKESGGKLTRRLVNSQCEFERRKPDGTTTLGLLHPVDPIVGEPGYCPVRLGMTTGRLQSGVNTLQGFKEDKRNKVTPVLYLNYGPYSSYAPHYDSTFANISKDDSDLIYSTYGEDSDLPSDFSIHEFLATCQDYPYVMADSLLDVLTKGGHSRTLQEMEMSLPEDEGHTRTLDTAKEMEQITEVEPPGRLDSSTQDRLIALKAVTNFGVPVEVFDSEEAEIFQKKLDETTRLLRELQEAQNERLSTRPPPNMICLLGPSYREMHLAEQVTNNLKELAQQVTPGDIVSTYGVRKAMGISIPSPVMENNFVDLTEDTEEPKKTDVAECGPGGS

SEQ ID NO: 33 - DNA, 4749 bp, Homo sapiens
atgggttccaagagaagaagagctacctccccttccagcagtgtcagcggggactttgatgatgggcaccattctgtgtcaacaccaggcccaagcaggaaaaggaggagactttccaatcttccaactgtagatcctattgccgtgtgccatgaactctataataccatccgagactataaggatgaacagggcagacttctctgtgagctcttcattagggcaccaaagcgaagaaatcaaccagactattatgaagtggtttctcagcccattgacttgatgaaaatccaacagaaactaaaaatggaagagtatgatgatgttaatttgctgactgctgacttccagcttctttttaacaatgcaaagtcctattataagccagattctcctgaatataaagccgcttgcaaactctgggatttgtaccttcgaacaagaaatgagtttgttcagaaaggagaagcagatgacgaagatgatgatgaagatgggcaagacaatcagggcacagtgactgaaggatcttctccagcttacttgaaggagatcctggagcagcttcttgaagccatagttgtagctacaaatccatcaggacgtctcattagcgaactttttcagaaactgccttctaaagtgcaatatccagattattatgcaataattaaggagcctatagatctcaagaccattgcccagaggatacagaatggaagctacaaaagtattcatgcaatggccaaagatatagatctcctcgcaaaaaatgccaaaacttataatgagcctggctctcaagtattcaaggatgcaaattcaattaaaaaaatattttatatgaaaaaggctgaaattgaacatcatgaaatggctaagtcaagtcttcgaatgaggactccatccaacttggctgcagccagactgacaggtccttcacacagtaaaggcagccttggtgaagagagaaatcccactagcaagtattaccgtaataaaagagcagtacaaggaggtcgtttatcagcaattacaatggcacttcaatatggctcagaaagtgaagaagatgctgctttagctgctgcacgctatgaagagggagagtcagaagcagaaagcatcacttcctttatggatgtttcaaatcctttttatcagctttatgacacagttaggagttgtcggaataaccaagggcagctaatagctgaacctttttaccatttgccttcaaagaaaaaataccctgattattaccagcaaattaaaatgcccatatcactacaacagatccgaacaaaactgaagaatcaagaatatgaaactttagatcatttggagtgtgatctgaatttaatgtttgaaaatgccaaacgctataatgtgcccaattcagccatctacaagcgagttctaaaattgcagcaagttatgcaggcaaagaagaaagagcttgccaggagagacgatatcgaggacggagacagcatgatctcttcagccacctctgatactggtagtgccaaaagaaaaagtaaaaagaacataagaaagcagcgaatgaaaatcttattcaatgttgttcttgaagctcgagagccaggttcaggcagaagactttgtgacctatttatggttaaaccatccaaaaaggactatcctgattattataaaatcatcttggagccaatggacttgaaaataattgagcataacatccgcaatgacaaatatgctggtgaagagggaatgatagaagacatgaagctgatgttccggaatgccaggcactataatgaggagggctcccaggtttataatgatgcacatatcctggagaagttactcaaggagaaaaggaaagagctgggcccactgcctgatgatgatgacatggcttctcccaaactcaagctgagtaggaagagtggcatttctcctaaaaaatcaaaatacatgactccaatgcagcagaaactaaatgaggtctatgaagctgtaaagaactatactgataagaggggtcgccgcctcagtgccatatttctgaggcttccctctagatctgagttgcctgactactatctgactattaaaaagcccatggacatggaaaaaattcgaagtcacatgatggccaacaagtaccaagatattgactctatggttgaggactttgtcatgatgtttaataatgcctgtacatacaatgagccggagtctttgatctacaaagatgctcttgttctacacaaagtcctgcttgaaacacgcagagacctggagggagatgaggactctcatgtcccaaatgtgactttgctgattcaagagcttatccacaatctttttgtgtcagtcatgagtcatcaggatgatgagggaagatgctacagcgattctttagcagaaattcctgctgtggatcccaactttcctaacaaaccaccccttacatttgacataattaggaagaatgttgaaaataatcgctaccgtcggcttgatttatttcaagagcatatgtttgaagtattggaacgagcaagaaggatgaatcggacagattcagaaatatatgaagatgcagtagaacttcagcagttttttattaaaattcgtgatgaactctgcaaaaatggagagattcttctttcaccggcactcagctataccacaaaacatttgcataatgatgtggagaaagagagaaaggaaaaattgccaaaagaaatagaggaagataaactaaaacgagaagaagaaaaaagagaagctgaaaagagtgaagattcctctggtgctgcaggcctctcaggcttacatcgcacatacagccaggactgtagctttaaaaacagcatgtaccatgttggagattacgtctatgtggaacctgcagaggccaacctacaaccacatatcgtctgtattgaaagactgtgggaggattcagctgaaaaagaagtttttaagagtgactattacaacaaagttccagttagtaaaattctaggcaagtgtgtggtcatgtttgtcaaggaatactttaagttatgcccagaaaacttccgagatgaggatgtttttgtctgtgaatcacggtattctgccaaaaccaaatcttttaagaaaattaaactgtggaccatgcccatcagctcagtcaggtttgtccctcgggatgtgcctctgcctgtggttcgcgtggcctctgtatttgcaaatgcagataaaggtgatgatgagaagaatacagacaactcagaggacagtcgagctgaagacaattttaacttggaaaaggaaaaagaagatgtccctgtggaaatgtccaatggtgaaccaggttgccactactttgagcagctccattacaatgacatgtggctgaaggttggcgactgtgtcttcatcaagtcccatggcctggtgcgtcctcgtgtgggcagaattgaaaaagtatgggttcgagatggagctgcatatttttatggccccatcttcattcacccagaagaaacagagcatgagcccacaaaaatgttctacaaaaaagaagtatttctgagtaatctggaagaaacctgccccatgacatgtattctcggaaagtgtgctgtgttgtcattcaaggacttcctctcctgcaggccaactgaaataccagaaaatgacattctgctttgtgagagccgctacaatgagagcgacaagcagatgaagaaattcaaaggattgaagaggttttcactctctgctaaagtggtagatgatgaaatttactacttcagaaaaccaattgttcctcagaaggagccatcacctttgctggaaaagaagatccagttgctagaagctaaatttgccgagttagaaggtggagatgatgatattgaagagatgggagaagaagatagtgagtctaccccaaagtctgccaaaggcagtgcaaagaaggaaggctccaaacggaaaatcaacatgagtggctacatcctgttcagcagtgagatgagggctgtgattaaggcccaacacccagactactctttcggggagctcagccgcctggtggggacagaatggagaaatcttgagacagccaagaaagcagaatatgaaggcatgatgggtggctatccgccaggccttccacctttgcagggcccagttgatggccttgttagcatgggcagcatgcagccacttcaccctggggggcctccaccccaccatcttccgccaggtgtgcctggcctcccgggcatcccaccaccgggtgtgatgaaccaaggagtggcccctatggtagggactccagcaccaggtggaagtccatatggacaacaggtgggagttttggggcctccagggcagcaggcaccacctccatatcccggcccacatccagctggaccccctgtcatacagcagccaacaacacccatgtttgtagctcccccaccaaagacccagcggcttcttcactcagaggcctacctgaaatacattgaaggactcagtgcggagtccaacagcattagcaagtgggatcagacactggcagctcgaagacgcgacgtccatttgtcgaaagaacaggagagccgcctaccctctcactggctgaaaagcaaaggggcccacaccaccatggcagatgccctctggcgccttcgagatttgatgctccgggacaccctcaacattcgccaagcatacaacctagaaaatgtttaa

SEQ ID NO: 34 - protein, 1582 aa, Homo sapiens
MGSKRRRATSPSSSVSGDFDDGHHSVSTPGPSRKRRRLSNLPTVDPIAVCHELYNTIRDYKDEQGRLLCELFIRAPKRRNQPDYYEVVSQPIDLMKIQQKLKMEEYDDVNLLTADFQLLFNNAKSYYKPDSPEYKAACKLWDLYLRTRNEFVQKGEADDEDDDEDGQDNQGTVTEGSSPAYLKEILEQLLEAIVVATNPSGRLISELFQKLPSKVQYPDYYAIIKEPIDLKTIAQRIQNGSYKSIHAMAKDIDLLAKNAKTYNEPGSQVFKDANSIKKIFYMKKAEIEHHEMAKSSLRMRTPSNLAAARLTGPSHSKGSLGEERNPTSKYYRNKRAVQGGRLSAITMALQYGSESEEDAALAAARYEEGESEAESITSFMDVSNPFYQLYDTVRSCRNNQGQLIAEPFYHLPSKKKYPDYYQQIKMPISLQQIRTKLKNQEYETLDHLECDLNLMFENAKRYNVPNSAIYKRVLKLQQVMQAKKKELARRDDIEDGDSMISSATSDTGSAKRKSKKNIRKQRMKILFNVVLEAREPGSGRRLCDLFMVKPSKKDYPDYYKIILEPMDLKIIEHNIRNDKYAGEEGMIEDMKLMFRNARHYNEEGSQVYNDAHILEKLLKEKRKELGPLPDDDDMASPKLKLSRKSGISPKKSKYMTPMQQKLNEVYEAVKNYTDKRGRRLSAIFLRLPSRSELPDYYLTIKKPMDMEKIRSHMMANKYQDIDSMVEDFVMMFNNACTYNEPESLIYKDALVLHKVLLETRRDLEGDEDSHVPNVTLLIQELIHNLFVSVMSHQDDEGRCYSDSLAEIPAVDPNFPNKPPLTFDIIRKNVENNRYRRLDLFQEHMFEVLERARRMNRTDSEIYEDAVELQQFFIKIRDELCKNGEILLSPALSYTTKHLHNDVEKERKEKLPKEIEEDKLKREEEKREAEKSEDSSGAAGLSGLHRTYSQDCSFKNSMYHVGDYVYVEPAEANLQPHIVCIERLWEDSAEKEVFKSDYYNKVPVSKILGKCVVMFVKEYFKLCPENFRDEDVFVCESRYSAKTKSFKKIKLWTMPISSVRFVPRDVPLPVVRVASVFANADKGDDEKNTDNSEDSRAEDNFNLEKEKEDVPVEMSNGEPGCHYFEQLHYNDMWLKVGDCVFIKSHGLVRPRVGRIEKVWVRDGAAYFYGPIFIHPEETEHEPTKMFYKKEVFLSNLEETCPMTCILGKCAVLSFKDFLSCRPTEIPENDILLCESRYNESDKQMKKFKGLKRFSLSAKVVDDEIYYFRKPIVPQKEPSPLLEKKIQLLEAKFAELEGGDDDIEEMGEEDSESTPKSAKGSAKKEGSKRKINMSGYILFSSEMRAVIKAQHPDYSFGELSRLVGTEWRNLETAKKAEYEGMMGGYPPGLPPLQGPVDGLVSMGSMQPLHPGGPPPHHLPPGVPGLPGIPPPGVMNQGVAPMVGTPAPGGSPYGQQVGVLGPPGQQAPPPYPGPHPAGPPVIQQPTTPMFVAPPPKTQRLLHSEAYLKYIEGLSAESNSISKWDQTLAARRRDVHLSKEQESRLPSHWLKSKGAHTTMADALWRLRDLMLRDTLNIRQAYNLENV

SEQ ID NO: 35 - DNA, 1794 bp, Mus musculus
atgggcaaaaagcacaagaagcacaaggcggaatggcgctcgtcctacgaagattatacagacacgccactggagaagcctctgaagctggtgctcaaggtgggaggaagtgaagtgacagagctctcaggatctggccacgactccagctactacgacgatcgctcagaccacgaacgggagagacacagagaaaagaagaaaaagaagaagaaaaagtcagagaaggagaagcacctcgatgaggaggagaggaggaagcggaaggaagagaagaaacggaaacgggagaaggaacactgcgactcagagggggaggctgatgctttcgaccctggaaagaaggtggaggtggagccacccccagaccgaccagtgagagcctgccgaacacagccagctgagaacgagagcacacctatccagaggcttctggaacacttcctccgccagctacagagaaaagatcctcatggattttttgcttttcctgttacggatgcaattgctcctgggtattcaatgataataaaacatcctatggactttggcacgatgaaagacaagattgtagctaatgaatataaatcagtcacagaatttaaggcagatttcaaattaatgtgtgataatgcgatgacgtacaatagaccagacaccgtgtactacaaattagccaagaagatcctgcacgcgggctttaagatgatgagcaaacaggcagctctcttgggcagtgaagacccagcagctgaggaacctgttcccgaggttgtcccagtgcaagtagaaactaccaagaaatccaaaaagccgagtagagaagttatcagctgcatgtttgagcctgaagggaatgcctgcagcctgacagacagcacggcagaggagcatgtgctagccctggtagagcacgcagctgatgaggctcgggacaggattaaccggtttctcccgggtggcaagatggggtacctgaagaagcttggagatggaagtctgctctacagcgtggtgaacgcacctgagcctgatgctgatgaggaggagacacaccctgtggacctgagttcactgtctagcaagttgctcccaggttttacaacattgggtttcaaagatgaaagaagaaataaagtcacattcctctccagtgccagcactgcactttcaatgcagaacaactctgtgtttggggacctgaagtcagatgagatggagcttctgtattccgcctatggagatgagactggtgtgcagtgtgcactgagcctgcaggaattcgtgaaggatgctggaagctatagcaagaagatggtagatgacctcctggaccaaatcacaggtggtgatcactcaaggatgatcttccagctgaagcagaggaggagcatccccatgagacctgcagatgagatgaaggttggggatccactgggagagagtggtggccctgttctggacttcatgtcaatgaaacagtatcctgatgtctccctggatgtgtccatgctcagctctctcgggaaagtaaagaaggagctggaccatgaagatagccacttgaacttggatgagacagccaggctcctgcaggacttacacgaagcacaagcagagcgaggaggctctcggccatcctccaaccttagctctctgtccactgcctctgagagggagcatcctcctccaggaagtccttctcgccttagtgttggggagcagccggatgtcgcccacgacccttatgaattccttcagtctccagaacctgcagctcctgccaagaactaa

SEQ ID NO: 36 - protein, 597 aa, Mus musculus
MGKKHKKHKAEWRSSYEDYTDTPLEKPLKLVLKVGGSEVTELSGSGHDSSYYDDRSDHERERHREKKKKKKKKSEKEKHLDEEERRKRKEEKKRKREKEHCDSEGEADAFDPGKKVEVEPPPDRPVRACRTQPAENESTPIQRLLEHFLRQLQRKDPHGFFAFPVTDAIAPGYSMIIKHPMDFGTMKDKIVANEYKSVTEFKADFKLMCDNAMTYNRPDTVYYKLAKKILHAGFKMMSKQAALLGSEDPAAEEPVPEVVPVQVETTKKSKKPSREVISCMFEPEGNACSLTDSTAEEHVLALVEHAADEARDRINRFLPGGKMGYLKKLGDGSLLYSVVNAPEPDADEEETHPVDLSSLSSKLLPGFTTLGFKDERRNKVTFLSSASTALSMQNNSVFGDLKSDEMELLYSAYGDETGVQCALSLQEFVKDAGSYSKKMVDDLLDQITGGDHSRMIFQLKQRRSIPMRPADEMKVGDPLGESGGPVLDFMSMKQYPDVSLDVSMLSSLGKVKKELDHEDSHLNLDETARLLQDLHEAQAERGGSRPSSNLSSLSTASEREHPPPGSPSRLSVGEQPDVAHDPYEFLQSPEPAAPAKN

SEQ ID NO: 37 - DNA, 1956 bp, Mus musculus
atgggcaagaagcacaagaagcacaagtcggaccgccacttctacgaggagtacgtggagaagcccctgaagctggtcctcaaagtcggggggagcgaggtcaccgagctctccacgggcagctccgggcacgactccagcctcttcgaagacagaagcgaccatgacaaacacaaggacagaaaacggaaaaagaggaagaaaggcgagaagcaggctccgggggaagagaaggggagaaaacggagaagagtcaaggaggataaaaagaagcgggatcgagaccgtgcagagaatgaggtggacagagatctccagtgtcatgtccctataagattagacttacctcctgagaagcctcttacaagctcgttagccaaacaagaagaagtagaacagacaccccttcaggaagctttgaatcagctcatgagacaattgcaaagaaaagacccaagtgctttcttttcatttcctgtgacggattttattgcgcctggctactccatgattattaaacacccaatggattttagtaccatgaaagaaaagatcaagaataacgactaccagtccatagaagaactaaaggataacttcaagctaatgtgtactaatgcaatgatttacaataagccagagaccatttattataaagctgcaaagaagctgttgcactcagggatgaaaattctcagtcaggagagaattcagagcctgaagcagagtatagacttcatgtcagacttgcagaaaactcggaagcagaaagaacgaacagatgcctgtcagagtggggaggacagcggctgctggcagcgcgagagggaagactctggagatgctgaaacacaggccttcagaagccccgctaaggacaataaaaggaaagacaaagatgtgcttgaagacaaatggagaagcagcaactcagaaagggagcatgagcagattgagcgcgttgtccaggagtcaggaggcaagctaacacggcggctggcaaacagtcagtgtgaatttgaaagaagaaaaccagatgggacaacaacactggggcttctccatcctgtggatcccattgtgggagagccaggctactgccctgtgagattggggatgacaactggaagactgcagtctggagtgaacactctgcaggggttcaaagaggataaaaggaacagagtaaccccagtattatacttgaattatggaccctacagttcttatgccccacattatgactctacatttgccaatattagcaaagatgattctgatttaatctactcaacatatggggaagactctgaccttccaaacaatttcagcatctctgagtttttggccacatgccaagattacccgtatgttatggcagatagtttactggatgttctaacaaaaggaggacattccaggagcctgcaggacttggacatgtcatctcctgaagatgaaggccagaccagagcattggacacagcaaaagaagcagagattacacaaatagagccaacagggcgtttggagtccagcagtcaggacaggctcacagcactgcaagctgtaacaacctttggtgctccagctgaagtctttgactccgaagaggctgaggtgttccagaggaagcttgatgagacgacaagattgctcagggagctccaggaggcacagaatgagcgactgagcactaggcctcctcccaatatgatctgtctcctgggtccttcttacagagaaatgtaccttgctgaacaagtgaccaataacctcaaagaactcacacagcaagtgactccaggtgatgttgtaagcatacacggagtgcgaaaagcaatggggatttctgttccttcccccatcgtgggaaacagcttcgtagatttgacaggagagtgtgaagaacctaaggagaccagcactgctgagtgtgggcctgacgcgagctga

SEQ ID NO: 38 - protein, 651 aa, Mus musculus
MGKKHKKHKSDRHFYEEYVEKPLKLVLKVGGSEVTELSTGSSGHDSSLFEDRSDHDKHKDRKRKKRKKGEKQAPGEEKGRKRRRVKEDKKKRDRDRAENEVDRDLQCHVPIRLDLPPEKPLTSSLAKQEEVEQTPLQEALNQLMRQLQRKDPSAFFSFPVTDFIAPGYSMIIKHPMDFSTMKEKIKNNDYQSIEELKDNFKLMCTNAMIYNKPETIYYKAAKKLLHSGMKILSQERIQSLKQSIDFMSDLQKTRKQKERTDACQSGEDSGCWQREREDSGDAETQAFRSPAKDNKRKDKDVLEDKWRSSNSEREHEQIERVVQESGGKLTRRLANSQCEFERRKPDGTTTLGLLHPVDPIVGEPGYCPVRLGMTTGRLQSGVNTLQGFKEDKRNRVTPVLYLNYGPYSSYAPHYDSTFANISKDDSDLIYSTYGEDSDLPNNFSISEFLATCQDYPYVMADSLLDVLTKGGHSRSLQDLDMSSPEDEGQTRALDTAKEAEITQIEPTGRLESSSQDRLTALQAVTTFGAPAEVFDSEEAEVFQRKLDETTRLLRELQEAQNERLSTRPPPNMICLLGPSYREMYLAEQVTNNLKELTQQVTPGDVVSIHGVRKAMGISVPSPIVGNSFVDLTGECEEPKETSTAECGPDAS

SEQ ID NO: 39 - DNA, 5115 bp, Mus musculus
atgggttccaagagaagaagagccacctctccttccagcagtgtcagtggagactttgatgacgggcaccattctgtgcctacaccaggcccaagcaggaaaaggagaagactgtccaatcttccaactgtagatcctattgctgtgtgccatgaactctataacaccatccgagactataaggatgaacagggcagactcctctgtgagctgttcattagggctccaaagcggagaaatcaaccagactattatgaagtggtttctcagcccattgacttgatgaaaatccaacagaaacttaaaatggaagagtatgatgatgttaatctactgactgctgacttccagctgctttttaacaatgcaaaggcctactataagccagattcccctgagtataaagctgcttgtaaactctgggatttgtaccttcgaacaagaaatgagtttgttcagaaaggagaagcagacgatgaagatgatgacgaagatgggcaagacaatcaaggcacactggctgacggctcttctccaggttatctgaaggagatcctggagcagcttcttgaagccatagttgtagccacaaatccatcaggacggctcatcagtgaactttttcagaaactgccttccaaagtgcaatatccagactattatgcaataattaaggaacctatagatctcaagaccattgctcagaggatacagaatggaagctacaaaagtatacacgcaatggccaaagatatagatcttctagcaaaaaatgccaaaacatacaatgagcctgggtctcaagtattcaaggatgccaattcgattaaaaaaatattttatatgaaaaaggcagaaattgaacatcatgaaatgactaaatcaagtcttcgaataaggactgcatcaaatttggctgcagccaggctgacaggtccttcgcacaataaaagcagccttggtgaagaaagaaaccccactagcaagtattaccgtaataaaagagcagtccaagggggtcgcttgtcagcaattaccatggcacttcagtatggatcagagagtgaagaggacgctgctttagctgctgcacgctatgaagaaggggaatctgaagcagagagcatcacttccttcatggacgtttccaacccctttcatcagctttacgacacagttaggagctgtaggaatcaccaagggcagctcatagctgaacctttcttccatttgccttcaaagaaaaaatacccagattattatcagcaaattaaaatgcccatatcacttcaacagatcagaacaaagctaaagaaccaagaatatgaaactttagatcatttggagtgtgatctgaatttaatgtttgaaaatgccaaacgttataacgttcccaattcagccatctataagcgagttctaaaactgcagcaagtcatgcaggcaaagaagaaggagcttgcgaggagagatgacattgaggacggagacagcatgatctcctcagccacttctgacactggtagtgccaaaaggaaaaggaatactcatgacagtgagatgttgggtctcaggaggctatccagtaaaaagaacataagaaaacagcgaatgaaaattttattcaatgttgttcttgaagctcgagagccaggttcaggcagaagactttgcgatctatttatggttaagccatccaagaaggactatcctgattattataaaatcatcttagagccaatggacctgaaaataattgagcataacatccgaaatgacaaatatgcaggtgaagaaggaatgatggaagacatgaaactcatgttccgcaatgccaggcactacaatgaggagggctcccaggtatacaatgatgcccatatcctggagaagttactcaaagataaaaggaaagagctgggccctctgcctgatgatgatgacatggcttctcccaaacttaaattgagtaggaagagtggtgtttctcctaagaaatcaaagtacatgactccaatgcagcagaaactgaatgaagtgtatgaagctgtaaagaactatactgataagaggggtcgccgccttagtgctatatttctaagactcccctctagatcagagctgcctgactactacctgaccattaaaaagcccatggacatggaaaaaattcgaagtcacatgatggcaaacaagtaccaagacatagattctatggtagaggactttgtcatgatgtttaataatgcctgtacctacaatgaaccagagtctttgatctacaaagatgcccttgtactgcataaagtcctccttgagactcggagagacctggagggagatgaggattctcatgtccctaatgtgacgttgctgattcaagagctcatccataacctttttgtgtcagtcatgagtcatcaggatgacgaagggaggtgttacagcgactccttagcagaaattcctgctgtggatcccaactctcccaataaacctccccttacatttgacattatcaggaaaaatgttgaaagtaatcggtatcggcgacttgatttatttcaggagcatatgtttgaagtattggaacgggcaagaaggatgaaccggacagattccgaaatatatgaggatgctgtagaacttcagcagttttttattagaattcgtgatgaactctgcaaaaatggagagatccttctttctccagcactcagctataccacaaaacacttgcataacgatgtggaaaaagaaaaaaaggaaaaattgcctaaagaaatagaggaagataaactaaaacgcgaagaagaaaaaagagaagctgaaaaaagtgaagattcctcaggtactacaggcctctcaggcttacatcgtacatacagccaggactgcagctttaagaacagcatgtatcatgtcggagattatgtctatgttgaacctgcggaggccaatctacaaccacatatagtgtgtattgagagactgtgggaggattcagctggtgaaaaatggttgtacggctgttggttttatcggccaaatgaaacattccatttggctacacgaaaatttctagaaaaagaagtttttaagagtgactactacaataaagtacctgttagtaaaattctaggcaaatgtgtagtcatgtttgtcaaggaatactttaaattatgtccagaaaactttcgcgatgaggatgtttttgtctgtgaatcgaggtattctgccaaaaccaaatcttttaagaaaattaaactgtggaccatgcccatcagttcagttagatttgtccctcgggatgtgcctttgcctgtggtccgagtggcctctgtgtttgcaaatgcagataaaggggatgatgagaagaatacagacaactcagatgacaatagagctgaagacaattttaacttggaaaaggaaaaagaagatgttcctgtggagatgtccaatggtgagccaggttgccactactttgagcagcttcggtacaatgacatgtggctgaaggttggtgattgtgtcttcatcaaatcccacggcttggtgcgccctcgtgtgggcagaattgagaaagtatgggtccgagatggagctgcatatttttatggccctatcttcattcatccagaagaaacagaacatgagcccacaaaaatgttctacaaaaaagaagtgtttctgagtaatctggaagagacctgccctatgagttgtattctggggaaatgtgcagtgctgtcattcaaggacttcctctcctgcaggccaactgaaataccagaaaatgacattctgctttgtgagagccgctataatgagagtgacaagcagatgaagaagttcaagggtttgaagaggttttcactctctgctaaagttgtagatgatgaaatctactacttcagaaaaccaatcattcctcagaaggaaccctcacctttgttagaaaagaagatacaattgctagaagctaaatttgcagagttagaaggaggagatgatgatattgaggagatgggagaagaggatagtgaagtcattgaagctccatctctacctcaactgcagacacccctggccaatgagttggacctcatgccctatacacccccacagtctaccccaaagtctgccaaaggcagtgcaaagaaggaaagttctaaacgaaaaatcaacatgagtggctacattttgttcagcagtgaaatgagagctgtgattaaagcccagcacccagactactcttttggggagctcagcagactggtggggacagaatggagaaaccttgaaacagccaagaaagcagaatatgaagagcgggcagctaaagttgctgagcagcaggagagagagcgagcagcacagcaacagcagccgagtgcttctccccgagcaggcacccctgtgggggctctcatgggggtggtgccaccaccaacaccaatggggatgctcaatcagcagttgacacctgttgcaggcatgatgggtggctatccgccaggccttccacctttgcagggcccagttgatggccttgttagcatgggcagcatgcagccacttcaccctggggggcctccacctcaccatcttccgccaggtgtgcctggcctcccaggcatcccaccaccgggtgtgatgaatcaaggagtagcccccatggtagggactccagcaccaggtggaagtccgtatggacaacaggtaggagttttgggacctccagggcagcaggcaccacctccatatcctggtcctcatccagctggcccccctgtcatacagcagccaacaacgcccatgtttgtggctcccccaccaaagacccaaaggcttctccactcagaggcctacctgaaatacattgaaggactcagtgctgaatccaacagcattagcaagtgggaccaaactttggcagctcgaagacgggatgtccatttgtccaaagaacaggagagccgcctaccttctcactggctcaaaagtaaaggggcacacaccaccatggcagatgccctctggcgcctacgggatttaatgcttcgagacactctcaacatccgacaggcatacaacctagaaaatgtttaa

SEQ ID NO: 40 - protein, 1704 aa, Mus musculus
MGSKRRRATSPSSSVSGDFDDGHHSVPTPGPSRKRRRLSNLPTVDPIAVCHELYNTIRDYKDEQGRLLCELFIRAPKRRNQPDYYEVVSQPIDLMKIQQKLKMEEYDDVNLLTADFQLLFNNAKAYYKPDSPEYKAACKLWDLYLRTRNEFVQKGEADDEDDDEDGQDNQGTLADGSSPGYLKEILEQLLEAIVVATNPSGRLISELFQKLPSKVQYPDYYAIIKEPIDLKTIAQRIQNGSYKSIHAMAKDIDLLAKNAKTYNEPGSQVFKDANSIKKIFYMKKAEIEHHEMTKSSLRIRTASNLAAARLTGPSHNKSSLGEERNPTSKYYRNKRAVQGGRLSAITMALQYGSESEEDAALAAARYEEGESEAESITSFMDVSNPFHQLYDTVRSCRNHQGQLIAEPFFHLPSKKKYPDYYQQIKMPISLQQIRTKLKNQEYETLDHLECDLNLMFENAKRYNVPNSAIYKRVLKLQQVMQAKKKELARRDDIEDGDSMISSATSDTGSAKRKRNTHDSEMLGLRRLSSKKNIRKQRMKILFNVVLEAREPGSGRRLCDLFMVKPSKKDYPDYYKIILEPMDLKIIEHNIRNDKYAGEEGMMEDMKLMFRNARHYNEEGSQVYNDAHILEKLLKDKRKELGPLPDDDDMASPKLKLSRKSGVSPKKSKYMTPMQQKLNEVYEAVKNYTDKRGRRLSAIFLRLPSRSELPDYYLTIKKPMDMEKIRSHMMANKYQDIDSMVEDFVMMFNNACTYNEPESLIYKDALVLHKVLLETRRDLEGDEDSHVPNVTLLIQELIHNLFVSVMSHQDDEGRCYSDSLAEIPAVDPNSPNKPPLTFDIIRKNVESNRYRRLDLFQEHMFEVLERARRMNRTDSEIYEDAVELQQFFIRIRDELCKNGEILLSPALSYTTKHLHNDVEKEKKEKLPKEIEEDKLKREEEKREAEKSEDSSGTTGLSGLHRTYSQDCSFKNSMYHVGDYVYVEPAEANLQPHIVCIERLWEDSAGEKWLYGCWFYRPNETFHLATRKFLEKEVFKSDYYNKVPVSKILGKCVVMFVKEYFKLCPENFRDEDVFVCESRYSAKTKSFKKIKLWTMPISSVRFVPRDVPLPVVRVASVFANADKGDDEKNTDNSDDNRAEDNFNLEKEKEDVPVEMSNGEPGCHYFEQLRYNDMWLKVGDCVFIKSHGLVRPRVGRIEKVWVRDGAAYFYGPIFIHPEETEHEPTKMFYKKEVFLSNLEETCPMSCILGKCAVLSFKDFLSCRPTEIPENDILLCESRYNESDKQMKKFKGLKRFSLSAKVVDDEIYYFRKPIIPQKEPSPLLEKKIQLLEAKFAELEGGDDDIEEMGEEDSEVIEAPSLPQLQTPLANELDLMPYTPPQSTPKSAKGSAKKESSKRKINMSGYILFSSEMRAVIKAQHPDYSFGELSRLVGTEWRNLETAKKAEYEERAAKVAEQQERERAAQQQQPSASPRAGTPVGALMGVVPPPTPMGMLNQQLTPVAGMMGGYPPGLPPLQGPVDGLVSMGSMQPLHPGGPPPHHLPPGVPGLPGIPPPGVMNQGVAPMVGTPAPGGSPYGQQVGVLGPPGQQAPPPYPGPHPAGPPVIQQPTTPMFVAPPPKTQRLLHSEAYLKYIEGLSAESNSISKWDQTLAARRRDVHLSKEQESRLPSHWLKSKGAHTTMADALWRLRDLMLRDTLNIRQAYNLENV

SEQ ID NO: 41 - DNA, 23 bp, Artificial Sequence
  Exemplary cloning site in pSIRG-NGFR
  misc_feature (11)..(13): “n” is any nucleotide and any number of nucleotides.
aaacaccgaannngtcgttttag

SEQ ID NO: 42 - DNA, 21 bp, Artificial Sequence
  Exemplary siRNA
gaaggaacactgcgactcaga

SEQ ID NO: 43 - DNA, 21 bp, Artificial Sequence
  Exemplary siRNA
gcctggctactccatgattat

SEQ ID NO: 44 - DNA, 21 bp, Artificial Sequence
  Exemplary siRNA
ggcacttcagtatggatcaga

SEQ ID NO: 45 - DNA, 23 bp, Artificial Sequence
  Exemplary gRNA
tgctcatcatcttaaagcccgcg

SEQ ID NO: 46 - DNA, 23 bp, Artificial Sequence
  Exemplary gRNA
tttaataatcatggagtagccag

SEQ ID NO: 47 - DNA, 23 bp, Artificial Sequence
  Exemplary gRNA
ttatagagttcatggcacacagc

(19) United States
(12) Patent Application Publication
(10) Pub. No.: US 2024/0150755 A1
(43) Pub. Date: May 9, 2024

(54) MODULATING REGULATORY T CELL FUNCTION IN AUTOIMMUNE DISEASE AND CANCER

(71) Applicant: Salk Institute for Biological Studies, La Jolla, CA (US)

(72) Inventors: Ye Zheng, La Jolla, CA (US); Diana Hargreaves, La Jolla, CA (US); Chin-San Loo, La Jolla, CA (US); Jovylyn Gatchalian, La Jolla, CA (US)

(73) Assignee: Salk Institute for Biological Studies, La Jolla, CA (US)

(21) Appl. No.: 18/278,824

(22) PCT Filed: Feb. 25, 2022
(86) PCT No.: PCT/US2022/018006

§ 371 Date: Aug. 24, 2023

Related U.S. Application Data
(60) Provisional application No. 63/154,612, filed on Feb. 26, 2021.

(51) Int. Cl. C12N 15/113; A61K 38/46; A61P 35/00

(52) U.S. Cl. CPC C12N 15/113; A61K 38/465; A61P 35/00; C12N 2310/14

(57) ABSTRACT

Methods of modulating regulatory T (Treg) suppressor activity are provided. Also provided are methods of treating autoimmune diseases and methods of treating cancer. The methods include increasing or reducing the expression or activity of bromodomain-containing 9 (Brd9), bromodomain-containing 7 (Brd7), and/or polybromo 1 (Pbrm1) in a Treg cell or in a subject.

### CROSS-REFERENCE TO RELATED APPLICATION

This claims the benefit of U.S. Provisional Application No. 63/154,612, filed Feb. 26, 2021, herein incorporated by reference in its entirety.

### ACKNOWLEDGMENT OF GOVERNMENT SUPPORT

This invention was made with government support under grant numbers AI107027 and GM128943 awarded by the National Institutes of Health. The government has certain rights in the invention.

### FIELD

This disclosure relates to methods of modulating regulatory T (Treg) cell activity and includes methods of treating autoimmune diseases and cancer.

### BACKGROUND

Autoimmune diseases are estimated to affect between 3-5% of individuals in western societies. While each autoimmune disorder is unique, they all are caused by a breakdown of tolerance to endogenous proteins. This leads to auto-inflammatory events that ultimately result in the destruction of tissues and organs. Regulatory T (Treg) cells play an important role in suppressing auto-reactive T cells and maintaining immune homeostasis. Treg cells are capable of suppressing auto-inflammatory events, for example, by secreting anti-inflammatory cytokines (such as TGF-β, IL-10, and IL-35), but are often poorly functioning in patients with an autoimmune disease (Arelleno et al. Discov Med. 22(119): 73-80, 2016). Thus, increasing Treg suppressor function could have beneficial role in treating or preventing autoimmune diseases or disorders.

Conversely, the immunosuppressive function of Treg cells can create a barrier in the treatment of cancer. The immunosuppressive activity of Treg cells can suppress natural anti-tumor responses, thereby allowing cancers to grow and spread. Thus, decreasing Treg suppressor activity could be useful in the treatment of various cancers, or to enhance existing cancer immunotherapies.

### SUMMARY

Disclosed herein are methods of treating an autoimmune disease or disorder, for example multiple sclerosis, in a subject. In some embodiments, a therapeutically effective amount of an agent that reduces expression or activity of Brd7 and/or Pbrm1 is administered to the subject. In other or additional embodiments, a therapeutically effective amount of an agent that increases expression or activity of Brd9 is administered to the subject. In some examples, Brd9, Brd7, and/or Pbrm1 expression is increased or reduced in a regulatory T cell (Treg) in the subject.

Also provided are methods of treating cancer, for example treating glioblastoma, in a subject. In some embodiments, a therapeutically effective amount of an agent that reduces expression or activity of Brd9 is administered to the subject. In other or additional embodiments, a therapeutically effective amount of an agent that increases expression or activity of Brd7 or Pbrm1 is administered to the subject. In some examples, Brd9, Brd7, and/or Pbrm1 expression is increased or reduced in a regulatory T cell (Treg) in the subject. In some examples, the agent is administered with an additional immunotherapy and the effective amount is an amount that enhances the additional immunotherapy.

Also provided are methods of increasing Treg suppressor activity, for example by reducing expression or activity of Brd7 and/or Pbrm1, or increasing expression or activity of Brd9 in a Treg cell. Methods of reducing Treg suppressor activity are also provided, for example by increasing expression or activity of Brd7 and/or Pbrm1, or reducing expression or activity of Brd9 in a Treg cell.

The expression or activity of Brd9, Brd7, or Pbrm1, can be increased, for example through techniques such as contacting the cell with an activator of Brd9, Brd7, or Pbrm1, respectively, or an expression vector encoding Brd9, Brd7, or Pbrm1, respectively. The expression or activity of Brd9, Brd7, or Pbrm1, can be reduced, for example through techniques such as genome editing, RNAi, or contacting the cell with a small molecule inhibitor of Brd9, Brd7, or Pbrm1, respectively. In some examples, the Treg cell is in a subject, the method is performed in vivo, and the subject is administered a small molecule inhibitor, an RNAi, an activator, or an expression vector encoding Brd9, Brd7, and/or Pbrm1.

The foregoing and other objects and features of the disclosure will become more apparent from the following detailed description, which proceeds with reference to the accompanying figures.

### BRIEF DESCRIPTION OF THE DRAWINGS

FIGS. 1A-1B: FIG. 1A shows workflow of the CRISPR screen in Treg cells. FIG. 1B shows FACS plots showing Foxp3 expression in Treg cells after sgRNA targeting of Foxp3 (sgFoxp3), positive regulator Cbfb (sgCbfb), and negative regulator Dnmt1 (sgDnmt1). Red and green gates were set based on Foxp3 low 20% and high 20% in sgNT Treg, respectively.

FIGS. 2A-2B: FIG. 2A shows mean fluorescence intensity (MFI) of Foxp3. FIG. 2B shows relative Log₂FC of cell count comparing Foxp3^{lo} to Foxp3^{hi} after deletion of the indicated target gene (n=3 per group).

FIGS. 3A-3B: A scatter plot of the Treg screen result showing positive regulators (FIG. 3A) and negative regulators (FIG. 3B). Genes that have met cutoff criteria (P-value<0.01, and Log₂FC>±0.5) are shown as dark grey dots.

FIGS. 4A-4B: FIG. 4A shows the distribution of sgRNA Log₂FC comparing Foxp3^{lo} to Foxp3^{hi}. Stripes for Foxp3, Cbfb, Runx3, and Usp7 represent sgRNAs from positive Foxp3 regulators, whereas stripes for Dnmt1 and Stub1 represent sgRNAs from negative Foxp3 regulators. FIG. 4B shows a Venn diagram showing the overlap of Foxp3 regulators with genes involved in cell contraction or expansion.

FIGS. 5A-5B: Gene Ontology analysis of positive Foxp3 regulators (FIG. 5A) and negative Foxp3 regulators (FIG. 5B).

FIG. 6: On the left are diagrams showing three different variants of SWI/SNF complexes: BAF, ncBAF, and PBAF. BAF-specific subunits include Arid1a, Dpf1-3, ncBAF-specific subunits include Brd9, Smarcd1, Gltscr11, Gltscr1, and PBAF-specific subunits include Pbrm1, Arid2, Brd7, Phf10. On the right is an immunoprecipitation assay of Arid1a, Brd9, Phf10, and Smarca4 in Treg cells. The co-precipitated proteins were probed for shared subunits (Smarca4, Smarcc1, Smarcb1), BAF-specific Arid1a, ncBAF-specific Brd9, and PBAF-specific Pbrm1.

FIG. 7: FACS histogram of Foxp3 expression in Treg cells after sgRNA targeting of the indicated SWI/SNF subunits.

FIG. 8: Mean fluorescence intensity (MFI) of Foxp3 after sgRNA targeting of the indicated SWI/SNF subunits. Data represents mean and standard deviation of biological replicates (n=3-21). Statistical analyses were performed using unpaired two-tailed Student's t test (ns: p≥0.05, *p<0.05, **p<0.01, ***p<0.001, ****p<0.0001).

FIGS. 9A-9B: FIG. 9A shows Principal component analysis of RNA-seq data collected from Treg cells transduced with guides against the indicated SWI/SNF subunits. In cases where two independent guides were used to target a gene, the second guide for targeting gene indicated as “−2.” FIG. 9B shows MFI of Foxp3 expression in Treg cells after treatment with either DMSO or 0.16-10 μM dBRD9 for 4 days. Data represent mean±s.d. Statistical analyses were performed using unpaired two-tailed Student's t test (ns: *p≥0.05, **p<0.01, ***p<0.001, **** p<0.0001).

FIG. 10: Brd9 deletion reduces Foxp3 binding at CNS0 and CNS2 enhancers. Genome browser tracks of Smarca4, Brd9, Phf10 with ChIP-seq and ATAC-seq signal, as well as Foxp3 ChIP-seq in sgNT, sgFoxp3, sgBrd9 and sgPbrm1 Treg cells and Foxp3 in DMSO and dBRD9 treated Treg cells (2.5 μM dBRD9 for 4 days). Foxp3 locus is shown with CNS0 and CNS2 enhancers indicated in gray ovals.

FIGS. 11A-11B: FIG. 11A shows a heat map of Foxp3, Brd9, Smarca4, and Phf10, ChIP-seq and ATAC-seq signal±3 kb centered on Foxp3-bound sites in Treg, ranked according to Foxp3 read density. FIG. 11B shows a Venn diagram of the overlap between ChIP-seq peaks in Treg for Brd9, Foxp3, and Phf10 (hypergeometric p value of Brd9:Foxp3 overlap=e⁻²⁷⁶⁶⁵, hypergeometric p value of PHF10:Foxp3 overlap=e⁻¹⁷¹⁸⁵, hypergeometric p value of Brd9:PHF10 overlap=e⁻¹⁴²¹⁷).

FIG. 12A-12B: FIG. 12A shows a heat map of Foxp3 ChIP-seq signal in sgNT, sgFoxp3, sgBrd9 and sgPbrm1 Treg cells±3 kilobases (kb) centered on Foxp3-bound sites in sgNT, ranked according to read density. FIG. 12B shows Foxp3 ChIP-seq signal in DMSO- and dBRD9 treated Treg cells at all Foxp3-bound sites in DMSO.

FIGS. 13A-13C: FIG. 13A shows a Venn diagram of the overlap (hypergeometric p value=e^{-11,653}) between sites that significantly lose Foxp3 binding (FC1.5, Poisson p value<0.0001) in sgFoxp3 and sgBrd9, overlaid on all Foxp3-bound sites in sgNT (in gray). FIG. 13B is the same as FIG. 13A, except it shows the overlap between sites that lose H3K27ac (FC1.5, Poisson p value<0.0001, hypergeometric p value of overlap=e^{-7,938}). FIG. 13C shows sites that significantly lose Foxp3 binding in dBRD9 treated Treg cells versus DMSO (FC1.5, Poisson p value<0.0001).

FIG. 14A-14D: FIG. 14A shows a histogram of Foxp3 ChIP read density±1 kb surrounding the peak center of sites that significantly lose Foxp3 binding in both sgFoxp3 and sgBrd9 (n=1,699) in sgNT, sgFoxp3, sgBrd9 and sgPbrm1. FIG. 14B shows H3K27ac ChIP read density. FIG. 14C shows a histogram of Foxp3 ChIP read density for DMSO and dBRD9 treated cells. FIG. 14D shows a histogram of Foxp3 ChIP read density for Treg cells transduced with either sgNT or sgBrd9, with ectopic expression of either MIGR vector control or Foxp3.

FIGS. 15A-15C: FIG. 15A shows a volcano plot of log₂ fold change RNA expression in sgFoxp3/sgNT Treg cells versus adjusted p value (Benjamin-Hochberg). The number of down and up genes are indicated, on the left or right, respectively. FIG. 15B shows significance of enrichment of Foxp3-dependent genes in each gene ontology. FIG. 15C shows a pie chart of Foxp3 and Brd9 binding by ChIP-seq for Foxp3-dependent genes.

FIG. 16: Gene set enrichment analysis (GSEA) enrichment plot for up and down genes in sgBrd9/sgNT compared with RNA-seq data of genes that significantly change in sgFoxp3/sgNT Treg cells. ES: Enrichment Score, NES: Normalized Enrichment Score, FWER: Familywise Error Rate.

FIG. 17: GSEA analysis like FIG. 16, but shows up and down genes in dBRD9/DMSO Treg cells.

FIG. 18: GSEA of the sgFoxp3/sgNT RNA-seq data; plot shows the familywise error rate (FWER) p value versus the normalized enrichment score (NES).

FIG. 19: In vitro suppression assay of Treg cells with sgRNA targeting of Brd9, Smarcd1, Pbrm1, and Phf10. sgNT was used as non-targeting control. Statistical analyses were performed using unpaired two-tailed Student's t test (ns: p≥0.05, *p<0.05, **p<0.01, ***p<0.001).

FIGS. 20A-20B: FIG. 20A shows in vitro suppression assay of sgBrd9 or sgNT with ectopic expression of Foxp3 or control vector MIGR1. (n=3 per group, data represent ±s.d.). FIG. 20B shows experimental design for in vivo T cell transfer induced colitis assay. Statistical analyses were performed using unpaired two-tailed Student's t test (ns: p≥0.05, *p<0.05, **p<0.01,***p<0.001).

FIG. 21: Body weight loss of Rag1^{-/-} mice colitis model after transfer of CD45.1⁺CD4⁺CD25⁻CD45RB^{hi} effector T cell (Teff) only, or co-transfer with Teff along with CD45.2⁺ Treg cells transduced with sgBrd9, or control sgNT. Statistical analyses were performed using unpaired two-tailed Student's t test (ns: p≥0.05, *p<0.05, **p<0.01, ***p<0.001).

FIG. 22: Colitis scores (left) and Colon histology (right) seven weeks after transfer of CD45.1⁺CD4⁺CD25⁻CD45RB^{hi} effector T cell (Teff) only, or co-transfer with Teff along with CD45.2⁺ Treg cells transduced with sgBrd9, or control sgNT, in Rag1^{-/-} colitis model. Statistical analyses were performed using unpaired two-tailed Student's t test (ns: p≥0.05, *p<0.05, **p<0.01,***p<0.001).

FIG. 23: The percentage of Foxp3⁺ cells in transferred CD45.2⁺CD4⁺ Treg population at the end point of the induced colitis model (n=4-6 per group; data represent mean±s.e.m.). Statistical analyses were performed using unpaired two-tailed Student's t test (ns: p≥0.05, *p<0.05,**p<0.01, ***p<0.001).

FIGS. 24A-24C: FIG. 24A shows the experiment procedure to measure function of sgNT or sgBrd9 Treg cells relative to no Treg cells in MC38 tumor model. FIG. 24B shows the tumor growth curve. FIG. 24C shows tumor weight at the end point. Statistical analyses were performed using unpaired two-tailed Student's t test (ns: p≥0.05, *p<0.05, **p<0.01, ***p<0.001).

FIGS. 25A-25B: Bar graphs of total CD4 T cell (FIG. 25A) and total CD8 T cell (FIG. 25B) percentage in the CD45⁺ immune cell population. Statistical analyses were performed using unpaired two-tailed Student's t test (ns: p≥0.05, *p<0.05, **p<0.01, ***p<0.001).

FIGS. 26A-26B: Bar graphs of IFN-γ⁺ cell percentage in CD4 T cells (FIG. 26A) and in CD8 T cells (FIG. 26B). Statistical analyses were performed using unpaired two-tailed Student's t test (ns: p≥0.05, *p<0.05, **p<0.01, ***p<0.001).

FIGS. 27A-27B: FIG. 27A shows a bar graph of the CD4⁺GFP⁺Foxp3⁺ donor cell percentage in CD4 T cells. FIG. 27B shows the ratio of CD8/Treg cells. Statistical analyses were performed using unpaired two-tailed Student's t test (ns: p≥0.05, *p<0.05, **p<0.01, ***p<0.001).

FIGS. 28A-28B: FIG. 28A shows a bar graph of Foxp3⁻ ex-Treg cell percentage in the transferred Treg population marked by the GFP reporter. FIG. 28B shows a bar graph of Foxp3⁻IFN-γ⁺ cell percentage in the transferred Treg population (n=5-7 per group; data represent mean±s.e.m.). Statistical analyses were performed using unpaired two-tailed Student's t test (ns: p≥0.05,*p<0.05, **p<0.01, ***p<0.001).

FIG. 29: Schematic of ncBAF, BAF, and PBAF complexes.

FIG. 30: Experimental design and procedure for the autoimmune encephalomyelitis (EAE) model.

FIG. 31: EAE score (limb paralysis) over time for WT and Pbrm1 conditional knock-out mice.

FIG. 32: Histopathological analysis of spinal cord sections. Left figures show representative samples. The top row contains brightfield microscope images. The lower row contains samples stained with Luxol Blue to measure demyelination. Bar graphs on the right row scoring distribution.

FIGS. 33A-33C: FACS analysis measuring infiltration of CD4⁺IFN-γ+(FIG. 33A), CD4⁺IL17A⁺ (FIG. 33B), and CD8⁺IFN-γ+(FIG. 33C) T cells infiltrating the CNS tissue or spleen of the Pbrm1 cKO mice at the endpoint (day 16).

FIG. 34: Percent survival by day of control and Brd9 conditional knock out mice in glioblastoma model.

FIG. 35: A bar graph showing brain weight of the control and Brd9 conditional knock-out. On the right is a photo of representative brains for the control and Brd9 conditional knock-out at the end point.

FIG. 36: FACS analysis of brain samples showing that intra-tumor CD4⁺ T cells were significantly higher in the Brd9 cKO mice as compared to WT control mice.

FIGS. 37A-37E: Construction of a retroviral sgRNA CRISPR library (pSIRG-NGFR-Brie). FIG. 37A shows the map of pSIRG-NGFR, a self-inactivating retroviral vector containing a sgRNA expressing cassette and a truncated human NGFR surface marker. FIG. 37B shows an overview of the process for cloning a sgRNA into pSIRG-NGFR. A pair of annealed sgRNA oligomers can be directly cloned into BbsI-digested pSIRG-NGFR by T4 ligation (sequence shown is SEQ ID NO: 41). FIG. 37C shows the validation of the transduction and knockout efficiency of pSIRG-NGFR. Cas9-expressing naïve CD4 T cells were transduced with either non-targeting control virus (sgNT) or Foxp3 targeting virus (sgFoxp3) in the presence of TGF-β and IL-2 for Foxp3 induction. NGFR and Foxp3 expression were measured by FACS 3 days post infection. FIG. 37D shows correlation of sgRNA representation comparing lentiCRISPRv2-Brie library to pSIRG-NGFR-Brie library (left). Read distribution of sgRNAs and genes in pSIRG-NGFR-Brie (right). FIG. 37E shows statistics of sgRNAs and genes represented in lentiCRISPRv2-Brie and pSIRG-NGFR-Brie. Quantification of sgRNAs and genes was computed by PinAPL-Py program.

FIGS. 38A-38L: Quality control analysis of samples comparing between Foxp3Lo and Foxp3Hi populations (38A-38F) or between Day 6 and Day 3 NGFR+ transduced populations (38G-38L). FIGS. 38A and 38G show mapped (dark blue) and unmapped (light blue) reads for each sample. The percentage of unmapped reads is labeled on each bar. FIGS. 38B and 38H show the number of missed gRNAs with zero mapped reads. FIGS. 38C and 38I show Gini Index for each sample measuring inequality between read counts. FIGS. 38D and 38J show the distribution of normalized read counts for each sample. FIGS. 38E and 38K show the cumulative distribution function of normalized read counts for each sample. FIGS. 38F and 38L show correlation between normalized log 10 read counts of samples.

FIGS. 39A-39B: Identification of genes that regulate cell proliferation and survival from the screen in Treg cells. Scatter plots showing genes enriched in the cell contraction pool (39A) or cell expansion pool (39B) by comparing NGFR+ transduced cells on day 6 to NGFR+ transduced cells on day 3, from the screen in Treg cells. Cutoff was set for contraction is P-value<0.002 and LFC>1 (dark grey dots), whereas cutoff for expansion was set P value<0.002 and LFC>0.5 (dark grey dots).

FIGS. 40A-40D: The SAGA complex regulates Foxp3 expression and Treg suppressor activity. FIG. 40A shows distribution of sgRNA Log₂FC comparing Foxp3Lo to Foxp3Hi. Stripes represent sgRNAs from positive Foxp3 regulators. Genes with a P-value of less than 0.01 are marked with an asterisk. FIG. 40B shows a FACS plot of Foxp3 expression in Treg cells transduced with sgRNAs against Ccdc101, Tada3, (HAT module), Eny2, Atxn713 and Usp22 (DUB module), and Tada1, Taf61, Supt20, Supt5 (structural subunits) of SAGA complex (n=3 per group). FIG. 40C shows the mean fluorescent intensity (MFI) of Foxp3 in Treg cells transduced with sgRNAs against SAGA subunits. FIG. 40D shows in vitro suppression assay of Treg cells transduced with sgUsp22. sgNT is non-targeting control. n=3 per group. Data represent mean±s.d. Statistical analyses were performed using unpaired two-tailed Student's t test (***p<0.001).

FIGS. 41A-41C: Brd9 degrader dBRD9 reduces Foxp3 expression without affecting cell viability and proliferation. FIG. 41A shows an immunoblotting analysis of Brd9, Foxp3, and TATA-binding protein (Tbp) in nuclear lysates from Treg cells treated with either DMSO or 2.5 μM dBRD9 for four days. Normalized protein levels are indicated. FIGS. 41B and C show Foxp3 expression, cell viability by Ghost Dye™, and cell division determined by CellTrace™ dilution in Treg cells after treatment of dBRD9 in increasing concentrations for 4 days (n=3 per group). On 41B, grey shade: DMSO, black line: dBRD9. Data represents mean±sd. Statistical analyses were performed using unpaired two-tailed Student's t-test. (*p<0.05, **p<0.01, ***p<0.001).

FIGS. 42A-42J: FIG. 42A shows a stacked bar graph of sites bound by Foxp3, Brd9, and Phf10 that localize to the indicated genomic elements. FIG. 42B shows a bar graph showing the top five de novo motifs enriched at Foxp3 (left) and Brd9 (right) ChIP-seq peaks, the percentage of sites that contain the motif, and the negative log of P value (Binomial distribution against random genomic background). FIG. 42C shows a heatmap of Foxp3 ChIP-seq signal in sgNT, <0.0001). BRD9 ChIP-seq signal is also shown in DMSO- and dBRD9-treated Treg cells. Signal is plotted ±3 kb centered on Foxp3 peaks. FIG. 42D shows a Scatterplot of Foxp3 ChIP-seq tags in sgNT and sgBrd9 (left) and sgNT+MIGR and sgBrd9+MIGR (right) at all Foxp3-bound sites. Numerical values represent the number of sites that are significantly up and down by 1.5-fold (Benjamin Hochberg FDR<0.05)) in sgBrd9 vs sgNT. Black dashed line represents y=x. FIG. 42E shows a heatmap of Foxp3 ChIP-seq density at the union of sites that significantly lose Foxp3 in sgBrd9 vs sgNT in the two experiments shown in D. FIG. 42F shows a metaplot of Foxp3 ChIP read density surrounding the peak center of sites in E. FIG. 42G shows a scatterplot of Log₂ ATAC-seq mean tags of duplicates in sgNT versus sgBrd9 Treg cells. FIG. 42H shows a heatmap of k-means clusters based on Log₂FC Foxp3 ChIP-seq signal in sgBrd9+MIGR vs sgNT+MIGR and sgBrd9+Foxp3 vs sgNT+MIGR at sites that significantly lose Foxp3 binding in sgBrd9+MIGR vs sgNT+MIGR. FIG. 42I shows a bar graph showing Foxp3 ChIPseq signal at select genomic regions. FIG. 42J shows Log₂FC RNA in sgBrd9/sgNT, sgSmarcd1/sgNT, and sgPbrm1/sgNT of genes that are annotated to sites that are most and least affected by Brd9 dependent Foxp3 change in binding. See Example 1 (Materials and Methods) for details of analysis.

FIGS. 43A-43D: sgRNA targeting of ncBAF or PBAF subunits or chemical degradation Brd9 alters Treg lineage stability and suppressor function. FIG. 43A shows in vitro suppression assay of Treg cells transduced with sgBrd9, sgSmarcd1, sgPbrm1, and sghf10. sgNT was used as non-targeting control. FIG. 43B shows in vitro suppression assay using Treg cells treated with dBRD9 or vehicle DMSO. Representative histograms of effector T cell divisions in different Treg:Teff ratios. FIG. 43C shows in vitro suppression assay of Treg cells transduced with sgNT or sgBrd9, with ectopic expression of Foxp3 or empty vector MIGR. Representative histogram of effector T cells divisions in Treg:Teff mixed in 1:8 ratio. (n=3 per group, data represent mean±s.d.). FIG. 43D shows FACS analysis of Foxp3 and IFN-γ expression in donor Treg cell population (CD4⁺GFP⁺) in MC38 tumor and spleen at the end point. Statistical analyses were performed using unpaired two-tailed Student's t test (ns: p>=0.05, *p<0.05, **p<0.01, ***p<0.001).

### SEQUENCE LISTING

Any nucleic acid and amino acid sequences listed herein are shown using standard letter abbreviations for nucleotide bases and amino acids, as defined in 37 C.F.R. § 1.822. In at least some cases, only one strand of each nucleic acid sequence is shown, but the complementary strand is understood as included by any reference to the displayed strand. The Sequence Listing is submitted as an ASCII text file (Sequence_listing.txt), created on Feb. 24, 2022, 81,920 bytes, which is incorporated by reference herein. In the accompanying sequence listing:

SEQ ID NO: 1 is an exemplary sequence encoding
an sgRNA targeting sequence for
Foxp3. TCTACCCACAGGGATCAATG
SEQ ID NO: 2 is an exemplary sequence encoding
an sgRNA targeting sequence for Cbfb.
GCCTTGCAGATTAAGTACAC
SEQ ID NO: 3 is an exemplary sequence encoding
an sgRNA targeting sequence for
Dnmt1. TAATGTGAACCGGTTCACAG
SEQ ID NO: 4 is an exemplary sequence encoding
an sgRNA targeting sequence for
Aridla. GCAGCTGCGAAGATATCGGG
SEQ ID NO: 5 is an exemplary sequence encoding
an sgRNA targeting sequence for
Arid1b. TGAGTGCAAAACTGAGCGCG
SEQ ID NO: 6 is an exemplary sequence encoding
an sgRNA targeting sequence for Dpf1.
TCTTCTACCTCGAGATCATG
SEQ ID NO: 7 is an exemplary sequence encoding
an sgRNA targeting sequence for Dpf2.
GAAGATACGCCAAAGCGTCG
SEQ ID NO: 8 is an exemplary sequence encoding
an sgRNA targeting sequence for Pbrm1.
AAAACACTTGCATAACGATG
SEQ ID NO: 9 is an exemplary sequence encoding
an sgRNA targeting sequence for Arid2.
ACTTGCAGTAAATTAGCTCG
SEQ ID NO: 10 is an exemplary sequence encoding
an sgRNA targeting sequence for Brd7.
CAGGAGGCAAGCTAACACGG
SEQ ID NO: 11 is an exemplary sequence encoding
an sgRNA targeting sequence for Phf10.
GTTGCCGACAGACCGAACGA
SEQ ID NO: 12 is an exemplary sequence encoding
an sgRNA targeting sequence for Brd9.
ATTAACCGGTTTCTCCCGGG
SEQ ID NO: 13 is an exemplary sequence encoding
an sgRNA targeting sequence for Gltscr1.
GTTCTGTGTAAAATCACACT
SEQ ID NO: 14 is an exemplary sequence encoding
an sgRNA targeting sequence for
Gltscr11. ATGGCTTTATGCAACACGTG
SEQ ID NO: 15 is an exemplary sequence encoding
an sgRNA targeting sequence for
Smarcd1. CAATCCGGCTAAGTCGGACG
SEQ ID NO: 16 is an exemplary sequence encoding
an sgRNA targeting sequence for Eny2.
AGAGCTAAATTAATTGAGTG
SEQ ID NO: 17 is an exemplary sequence encoding
an sgRNA targeting sequence for Atxn713.
GCAGCCGAATCGCCAACCGT
SEQ ID NO: 18 is an exemplary sequence encoding
an sgRNA targeting sequence for Usp22.
GCCATCGACCTGATGTACGG
SEQ ID NO: 19 is an exemplary sequence encoding
an sgRNA targeting sequence for Ccdc101/Sgf29.
CCAGGTTTCCCGATCCAGAG
SEQ ID NO: 20 is an exemplary sequence encoding
an sgRNA targeting sequence for Tada3.
GAAGGTCTGTCCCCGCTACA
SEQ ID NO: 21 is an exemplary sequence encoding
an sgRNA targeting sequence for Tada1.
TTTCCTTCTCGACACAACTG
SEQ ID NO: 22 is an exemplary sequence encoding
an sgRNA targeting sequence for Taf6l.
TCATGAAACACACCAAACGA
SEQ ID NO: 23 is an exemplary sequence encoding
an sgRNA targeting sequence for Supt20.
TTAGTAGTCAATCTGTACCC
SEQ ID NO: 24 is an exemplary sequence encoding
an sgRNA targeting sequence for Supt5.
GATGACCGATGTACTCAAGG
SEQ ID NO: 25 is an exemplary sequence encoding
a non-targeting sgRNA (sgNT).
AAAAAGTCCGCGATTACGTC
SEQ ID NO: 26 a nucleic acid sequence of a primer.
GGCTTTATATATCTTGTGGAAAGGACGAAACACCG
SEQ ID NO: 27 a nucleic acid sequence of a primer.
CTAGCCTTATTTTAACTTGCTATTTCTAGCTCTAAAAC
SEQ ID NO: 28 a nucleic acid sequence of a primer.
CTTCCCTCGACGAATTCCCAAC
SEQ ID NO: 29 is an exemplary nucleic acid
sequence encoding human Brd9.
ATGGGCAAGAAGCACAAGAAGCACAAGGCCGAGTGGCGCTCGTCC
TACGAGGATTATGCCGACAAGCCCCTGGAGAAGCCTCTAAAGCTA
GTCCTGAAGGTCGGAGGAAGTGAAGTGACTGAACTCTCAGGATCC
GGCCACGACTCCAGTTACTATGATGACAGGTCAGACCATGAGCGA
GAGAGGCACAAAGAAAAGAAAAAGAAGAAGAAGAAGAAGTCCGAG
AAGGAGAAGCATCTGGACGATGAGGAAAGAAGGAAGCGAAAGGAA
GAGAAGAAGCGGAAGCGAGAGAGGGAGCACTGTGACACGGAGGGA
GAGGCTGACGACTTTGATCCTGGGAAGAAGGTGGAGGTGGAGCCG
CCCCCAGATCGGCCAGTCCGAGCGTGCCGGACACAGCCAGCCGAA
AATGAGAGCACACCTATTCAGCAACTCCTGGAACACTTCCTCCGC
CAGCTTCAGAGAAAAGATCCCCATGGATTTTTTGCTTTTCCTGTC
ACGGATGCAATTGCTCCTGGATATTCAATGATAATAAAACATCCC
ATGGATTTTGGCACCATGAAAGACAAAATTGTAGCTAATGAATAC
AAGTCAGTTACGGAATTTAAGGCAGATTTCAAGCTGATGTGTGAT
AATGCAATGACATACAATAGGCCAGATACCGTGTACTACAAGTTG
GCGAAGAAGATCCTTCACGCAGGCTTTAAGATGATGAGCAAACAG
GCAGCTCTTTTGGGCAATGAAGATACAGCTGTTGAGGAACCTGTC
CCTGAAGTTGTACCAGTACAAGTAGAAACTGCCAAGAAATCCAAA
AAGCCGAGTAGAGAAGTTATCAGCTGCATGTTTGAGCCTGAAGGG
AATGCCTGCAGCTTGACGGACAGTACCGCAGAGGAGCACGTGCTG
GCGCTGGTGGAGCACGCAGCTGACGAAGCTCGGGACAGGATCAAC
CGGTTCCTCCCAGGCGGCAAGATGGGCTATCTGAAGAGGAACGGG
GACGGGAGCCTGCTCTACAGCGTGGTCAACACGGCCGAGCCGGAC
GCTGATGAGGAGGAGACCCACCCGGTGGACTTGAGCTCGCTCTCC
AGTAAGCTACTCCCAGGCTTCACCACGCTGGGCTTCAAAGACGAG
AGAAGAAACAAAGTCACCTTTCTCTCCAGTGCCACTACTGCGCTT
TCGATGCAGAATAATTCAGTATTTGGCGACTTGAAGTCGGACGAG
ATGGAGCTGCTCTACTCAGCCTACGGAGATGAGACAGGCGTGCAG
TGTGCGCTGAGCCTGCAGGAGTTTGTGAAGGATGCTGGGAGCTAC
AGCAAGAAAGTGGTGGACGACCTCCTGGACCAGATCACAGGCGGA
GACCACTCTAGGACGCTCTTCCAGCTGAAGCAGAGAAGAAATGTT
CCCATGAAGCCTCCAGATGAAGCCAAGGTTGGGGACACCCTAGGA
GACAGCAGCAGCTCTGTTCTGGAGTTCATGTCGATGAAGTCCTAT
CCCGACGTTTCTGTGGATATCTCCATGCTCAGCTCTCTGGGGAAG
GTGAAGAAGGAGCTGGACCCTGACGACAGCCATTTGAACTTGGAT
GAGACGACGAAGCTCCTGCAGGACCTGCACGAAGCACAGGCGGAG
CGCGGCGGCTCTCGGCCGTCGTCCAACCTCAGCTCCCTGTCCAAC
GCCTCCGAGAGGGACCAGCACCACCTGGGAAGCCCTTCTCGCCTG
AGTGTCGGGGAGCAGCCAGACGTCACCCACGACCCCTATGAGTTT
CTTCAGTCTCCAGAGCCTGCGGCCTCTGCCAAGACCTAA
SEQ ID NO: 30 is an exemplary human Brd
9 amino acid sequence.
MGKKHKKHKAEWRSSYEDYADKPLEKPLKLVLKVGGSEVTELSGS
GHDSSYYDDRSDHERERHKEKKKKKKKKSEKEKHLDDEERRKRKE
EKKRKREREHCDTEGEADDFDPGKKVEVEPPPDRPVRACRTQPAE
NESTPIQQLLEHFLRQLQRKDPHGFFAFPVTDAIAPGYSMIIKHP
MDFGTMKDKIVANEYKSVTEFKADFKLMCDNAMTYNRPDTVYYKL
AKKILHAGFKMMSKQAALLGNEDTAVEEPVPEVVPVQVETAKKSK
KPSREVISCMFEPEGNACSLTDSTAEEHVLALVEHAADEARDRIN
RFLPGGKMGYLKRNGDGSLLYSVVNTAEPDADEEETHPVDLSSLS
SKLLPGFTTLGFKDERRNKVTFLSSATTALSMQNNSVFGDLKSDE
MELLYSAYGDETGVQCALSLQEFVKDAGSYSKKVVDDLLDQITGG
DHSRTLFQLKQRRNVPMKPPDEAKVGDTLGDSSSSVLEFMSMKSY
PDVSVDISMLSSLGKVKKELDPDDSHLNLDETTKLLQDLHEAQAE
RGGSRPSSNLSSLSNASERDQHHLGSPSRLSVGEQPDVTHDPYEF
LQSPEPAASAKT
SEQ ID NO: 31 is an exemplary nucleic acid
sequence encoding human Brd7.
ATGGGCAAGAAGCACAAGAAGCACAAGTCGGACAAACACCTCTAC
GAGGAGTATGTAGAGAAGCCCTTGAAGCTGGTCCTCAAAGTAGGA
GGGAACGAAGTCACCGAACTCTCCACGGGCAGCTCGGGGCACGAC
TCCAGCCTCTTCGAAGACAAAAACGATCATGACAAACACAAGGAC
AGAAAGCGGAAAAAGAGAAAGAAAGGAGAGAAGCAGATTCCAGGG
GAAGAAAAGGGGAGAAAACGGAGAAGAGTTAAGGAGGATAAAAAG
AAGCGAGATCGAGACCGGGTGGAGAATGAGGCAGAAAAAGATCTC
CAGTGTCACGCCCCTGTGAGATTAGACTTGCCTCCTGAGAAGCCT
CTCACAAGCTCTTTAGCCAAACAAGAAGAAGTAGAACAGACACCC
CTTCAAGAAGCTTTGAATCAACTGATGAGACAATTGCAGAGAAAA
GATCCAAGTGCTTTCTTTTCATTTCCTGTGACTGATTTTATTGCT
CCTGGCTACTCCATGATCATTAAACACCCAATGGATTTTAGTACC
ATGAAAGAAAAGATCAAGAACAATGACTATCAGTCCATAGAAGAA
CTAAAGGATAACTTCAAACTAATGTGTACTAATGCCATGATTTAC
AATAAACCAGAGACCATTTATTATAAAGCTGCAAAGAAGCTGTTG
CACTCAGGAATGAAAATTCTTAGCCAGGAAAGAATTCAGAGCCTG
AAGCAGAGCATAGACTTCATGGCTGACTTGCAGAAAACTCGAAAG
CAGAAAGATGGAACAGACACCTCACAGAGTGGGGAGGACGGAGGC
TGCTGGCAGAGAGAGAGAGAGGACTCTGGAGATGCCGAAGCACAC
GCCTTCAAGAGTCCCAGCAAAGAAAATAAAAAGAAAGACAAAGAT
ATGCTTGAAGATAAGTTTAAAAGCAATAATTTAGAGAGAGAGCAG
GAGCAGCTTGACCGCATCGTGAAGGAATCTGGAGGAAAGCTGACC
AGGCGGCTTGTGAACAGTCAGTGCGAATTTGAAAGAAGAAAACCA
GATGGAACAACGACGTTGGGACTTCTCCATCCTGTGGATCCCATT
GTAGGAGAGCCAGGCTACTGCCCTGTGAGACTGGGAATGACAACT
GGAAGACTTCAGTCTGGAGTGAATACTTTGCAGGGGTTCAAAGAG
GATAAAAGGAACAAAGTCACTCCAGTGTTATATTTGAATTATGGG
CCCTACAGTTCTTATGCACCGCATTATGACTCCACATTTGCAAAT
ATCAGCAAGGATGATTCTGATTTAATCTATTCAACCTATGGGGAA
GACTCTGATCTTCCAAGTGATTTCAGCATCCATGAGTTTTTGGCC
ACGTGCCAAGATTATCCGTATGTCATGGCAGATAGTTTACTGGAT
GTTTTAACAAAAGGAGGGCATTCCAGGACCCTACAAGAGATGGAG
ATGTCATTGCCTGAAGATGAAGGCCATACTAGGACACTTGACACA
GCAAAAGAAATGGAGCAGATTACAGAAGTAGAGCCACCAGGGCGT
TTGGACTCCAGTACTCAAGACAGGCTCATAGCGCTGAAAGCAGTA
ACAAATTTTGGCGTTCCAGTTGAAGTTTTTGACTCTGAAGAAGCT
GAAATATTCCAGAAGAAACTTGATGAGACCACCAGATTGCTCAGG
GAACTCCAGGAAGCCCAGAATGAACGTTTGAGCACCAGACCCCCT
CCGAACATGATCTGTCTCTTGGGTCCCTCATACAGAGAAATGCAT
CTTGCTGAACAAGTGACCAATAATCTTAAAGAACTTGCACAGCAA
GTAACTCCAGGTGATATCGTAAGCACGTATGGAGTTCGAAAAGCA
ATGGGGATTTCCATTCCTTCCCCCGTCATGGAAAACAACTTTGTG
GATTTGACAGAAGACACTGAAGAACCTAAAAAGACGGATGTTGCT
GAGTGTGGACCTGGTGGAAGTTGA
SEQ ID NO: 32 is an exemplary human Brd7
amino acid sequence.
MGKKHKKHKSDKHLYEEYVEKPLKLVLKVGGNEVTELSTGSSGHD
SSLFEDKNDHDKHKDRKRKKRKKGEKQIPGEEKGRKRRRVKEDKK
KRDRDRVENEAEKDLQCHAPVRLDLPPEKPLTSSLAKQEEVEQTP
LQEALNQLMRQLQRKDPSAFFSFPVTDFIAPGYSMIIKHPMDFST
MKEKIKNNDYQSIEELKDNFKLMCTNAMIYNKPETIYYKAAKKLL
HSGMKILSQERIQSLKQSIDFMADLQKTRKQKDGTDTSQSGEDGG
CWQREREDSGDAEAHAFKSPSKENKKKDKDMLEDKFKSNNLEREQ
EQLDRIVKESGGKLTRRLVNSQCEFERRKPDGTTTLGLLHPVDPI
VGEPGYCPVRLGMTTGRLQSGVNTLQGFKEDKRNKVTPVLYLNYG
PYSSYAPHYDSTFANISKDDSDLIYSTYGEDSDLPSDFSIHEFLA
TCQDYPYVMADSLLDVLTKGGHSRTLQEMEMSLPEDEGHTRTLDT
AKEMEQITEVEPPGRLDSSTQDRLIALKAVTNFGVPVEVFDSEEA
EIFQKKLDETTRLLRELQEAQNERLSTRPPPNMICLLGPSYREMH
LAEQVTNNLKELAQQVTPGDIVSTYGVRKAMGISIPSPVMENNFV
DLTEDTEEPKKTDVAECGPGGS
SEQ ID NO: 33 is an exemplary nucleic acid
sequence encoding human Pbrm1.
ATGGGTTCCAAGAGAAGAAGAGCTACCTCCCCTTCCAGCAGTGTC
AGCGGGGACTTTGATGATGGGCACCATTCTGTGTCAACACCAGGC
CCAAGCAGGAAAAGGAGGAGACTTTCCAATCTTCCAACTGTAGAT
CCTATTGCCGTGTGCCATGAACTCTATAATACCATCCGAGACTAT
AAGGATGAACAGGGCAGACTTCTCTGTGAGCTCTTCATTAGGGCA
CCAAAGCGAAGAAATCAACCAGACTATTATGAAGTGGTTTCTCAG
CCCATTGACTTGATGAAAATCCAACAGAAACTAAAAATGGAAGAG
TATGATGATGTTAATTTGCTGACTGCTGACTTCCAGCTTCTTTTT
AACAATGCAAAGTCCTATTATAAGCCAGATTCTCCTGAATATAAA
GCCGCTTGCAAACTCTGGGATTTGTACCTTCGAACAAGAAATGAG
TTTGTTCAGAAAGGAGAAGCAGATGACGAAGATGATGATGAAGAT
GGGCAAGACAATCAGGGCACAGTGACTGAAGGATCTTCTCCAGCT
TACTTGAAGGAGATCCTGGAGCAGCTTCTTGAAGCCATAGTTGTA
GCTACAAATCCATCAGGACGTCTCATTAGCGAACTTTTTCAGAAA
CTGCCTTCTAAAGTGCAATATCCAGATTATTATGCAATAATTAAG
GAGCCTATAGATCTCAAGACCATTGCCCAGAGGATACAGAATGGA
AGCTACAAAAGTATTCATGCAATGGCCAAAGATATAGATCTCCTC
GCAAAAAATGCCAAAACTTATAATGAGCCTGGCTCTCAAGTATTC
AAGGATGCAAATTCAATTAAAAAAATATTTTATATGAAAAAGGCT
GAAATTGAACATCATGAAATGGCTAAGTCAAGTCTTCGAATGAGG
ACTCCATCCAACTTGGCTGCAGCCAGACTGACAGGTCCTTCACAC
AGTAAAGGCAGCCTTGGTGAAGAGAGAAATCCCACTAGCAAGTAT
TACCGTAATAAAAGAGCAGTACAAGGAGGTCGTTTATCAGCAATT
ACAATGGCACTTCAATATGGCTCAGAAAGTGAAGAAGATGCTGCT
TTAGCTGCTGCACGCTATGAAGAGGGAGAGTCAGAAGCAGAAAGC
ATCACTTCCTTTATGGATGTTTCAAATCCTTTTTATCAGCTTTAT
GACACAGTTAGGAGTTGTCGGAATAACCAAGGGCAGCTAATAGCT
GAACCTTTTTACCATTTGCCTTCAAAGAAAAAATACCCTGATTAT
TACCAGCAAATTAAAATGCCCATATCACTACAACAGATCCGAACA
AAACTGAAGAATCAAGAATATGAAACTTTAGATCATTTGGAGTGT
GATCTGAATTTAATGTTTGAAAATGCCAAACGCTATAATGTGCCC
AATTCAGCCATCTACAAGCGAGTTCTAAAATTGCAGCAAGTTATG
CAGGCAAAGAAGAAAGAGCTTGCCAGGAGAGACGATATCGAGGAC
GGAGACAGCATGATCTCTTCAGCCACCTCTGATACTGGTAGTGCC
AAAAGAAAAAGTAAAAAGAACATAAGAAAGCAGCGAATGAAAATC
TTATTCAATGTTGTTCTTGAAGCTCGAGAGCCAGGTTCAGGCAGA
AGACTTTGTGACCTATTTATGGTTAAACCATCCAAAAAGGACTAT
CCTGATTATTATAAAATCATCTTGGAGCCAATGGACTTGAAAATA
ATTGAGCATAACATCCGCAATGACAAATATGCTGGTGAAGAGGGA
ATGATAGAAGACATGAAGCTGATGTTCCGGAATGCCAGGCACTAT
AATGAGGAGGGCTCCCAGGTTTATAATGATGCACATATCCTGGAG
AAGTTACTCAAGGAGAAAAGGAAAGAGCTGGGCCCACTGCCTGAT
GATGATGACATGGCTTCTCCCAAACTCAAGCTGAGTAGGAAGAGT
GGCATTTCTCCTAAAAAATCAAAATACATGACTCCAATGCAGCAG
AAACTAAATGAGGTCTATGAAGCTGTAAAGAACTATACTGATAAG
AGGGGTCGCCGCCTCAGTGCCATATTTCTGAGGCTTCCCTCTAGA
TCTGAGTTGCCTGACTACTATCTGACTATTAAAAAGCCCATGGAC
ATGGAAAAAATTCGAAGTCACATGATGGCCAACAAGTACCAAGAT
ATTGACTCTATGGTTGAGGACTTTGTCATGATGTTTAATAATGCC
TGTACATACAATGAGCCGGAGTCTTTGATCTACAAAGATGCTCTT
GTTCTACACAAAGTCCTGCTTGAAACACGCAGAGACCTGGAGGGA
GATGAGGACTCTCATGTCCCAAATGTGACTTTGCTGATTCAAGAG
CTTATCCACAATCTTTTTGTGTCAGTCATGAGTCATCAGGATGAT
GAGGGAAGATGCTACAGCGATTCTTTAGCAGAAATTCCTGCTGTG
GATCCCAACTTTCCTAACAAACCACCCCTTACATTTGACATAATT
AGGAAGAATGTTGAAAATAATCGCTACCGTCGGCTTGATTTATTT
CAAGAGCATATGTTTGAAGTATTGGAACGAGCAAGAAGGATGAAT
CGGACAGATTCAGAAATATATGAAGATGCAGTAGAACTTCAGCAG
TTTTTTATTAAAATTCGTGATGAACTCTGCAAAAATGGAGAGATT
CTTCTTTCACCGGCACTCAGCTATACCACAAAACATTTGCATAAT
GATGTGGAGAAAGAGAGAAAGGAAAAATTGCCAAAAGAAATAGAG
GAAGATAAACTAAAACGAGAAGAAGAAAAAAGAGAAGCTGAAAAG
AGTGAAGATTCCTCTGGTGCTGCAGGCCTCTCAGGCTTACATCGC
ACATACAGCCAGGACTGTAGCTTTAAAAACAGCATGTACCATGTT
GGAGATTACGTCTATGTGGAACCTGCAGAGGCCAACCTACAACCA
CATATCGTCTGTATTGAAAGACTGTGGGAGGATTCAGCTGAAAAA
GAAGTTTTTAAGAGTGACTATTACAACAAAGTTCCAGTTAGTAAA
ATTCTAGGCAAGTGTGTGGTCATGTTTGTCAAGGAATACTTTAAG
TTATGCCCAGAAAACTTCCGAGATGAGGATGTTTTTGTCTGTGAA
TCACGGTATTCTGCCAAAACCAAATCTTTTAAGAAAATTAAACTG
TGGACCATGCCCATCAGCTCAGTCAGGTTTGTCCCTCGGGATGTG
CCTCTGCCTGTGGTTCGCGTGGCCTCTGTATTTGCAAATGCAGAT
AAAGGTGATGATGAGAAGAATACAGACAACTCAGAGGACAGTCGA
GCTGAAGACAATTTTAACTTGGAAAAGGAAAAAGAAGATGTCCCT
GTGGAAATGTCCAATGGTGAACCAGGTTGCCACTACTTTGAGCAG
CTCCATTACAATGACATGTGGCTGAAGGTTGGCGACTGTGTCTTC
ATCAAGTCCCATGGCCTGGTGCGTCCTCGTGTGGGCAGAATTGAA
AAAGTATGGGTTCGAGATGGAGCTGCATATTTTTATGGCCCCATC
TTCATTCACCCAGAAGAAACAGAGCATGAGCCCACAAAAATGTTC
TACAAAAAAGAAGTATTTCTGAGTAATCTGGAAGAAACCTGCCCC
ATGACATGTATTCTCGGAAAGTGTGCTGTGTTGTCATTCAAGGAC
TTCCTCTCCTGCAGGCCAACTGAAATACCAGAAAATGACATTCTG
CTTTGTGAGAGCCGCTACAATGAGAGCGACAAGCAGATGAAGAAA
TTCAAAGGATTGAAGAGGTTTTCACTCTCTGCTAAAGTGGTAGAT
GATGAAATTTACTACTTCAGAAAACCAATTGTTCCTCAGAAGGAG
CCATCACCTTTGCTGGAAAAGAAGATCCAGTTGCTAGAAGCTAAA
TTTGCCGAGTTAGAAGGTGGAGATGATGATATTGAAGAGATGGGA
GAAGAAGATAGTGAGTCTACCCCAAAGTCTGCCAAAGGCAGTGCA
AAGAAGGAAGGCTCCAAACGGAAAATCAACATGAGTGGCTACATC
CTGTTCAGCAGTGAGATGAGGGCTGTGATTAAGGCCCAACACCCA
GACTACTCTTTCGGGGAGCTCAGCCGCCTGGTGGGGACAGAATGG
AGAAATCTTGAGACAGCCAAGAAAGCAGAATATGAAGGCATGATG
GGTGGCTATCCGCCAGGCCTTCCACCTTTGCAGGGCCCAGTTGAT
GGCCTTGTTAGCATGGGCAGCATGCAGCCACTTCACCCTGGGGGG
CCTCCACCCCACCATCTTCCGCCAGGTGTGCCTGGCCTCCCGGGC
ATCCCACCACCGGGTGTGATGAACCAAGGAGTGGCCCCTATGGTA
GGGACTCCAGCACCAGGTGGAAGTCCATATGGACAACAGGTGGGA
GTTTTGGGGCCTCCAGGGCAGCAGGCACCACCTCCATATCCCGGC
CCACATCCAGCTGGACCCCCTGTCATACAGCAGCCAACAACACCC
ATGTTTGTAGCTCCCCCACCAAAGACCCAGCGGCTTCTTCACTCA
GAGGCCTACCTGAAATACATTGAAGGACTCAGTGCGGAGTCCAAC
AGCATTAGCAAGTGGGATCAGACACTGGCAGCTCGAAGACGCGAC
GTCCATTTGTCGAAAGAACAGGAGAGCCGCCTACCCTCTCACTGG
CTGAAAAGCAAAGGGGCCCACACCACCATGGCAGATGCCCTCTGG
CGCCTTCGAGATTTGATGCTCCGGGACACCCTCAACATTCGCCAA
GCATACAACCTAGAAAATGTTTAA
SEQ ID NO: 34 is an exemplary human Pbrm1
amino acid sequence.
MGSKRRRATSPSSSVSGDFDDGHHSVSTPGPSRKRRRLSNLPTVD
PIAVCHELYNTIRDYKDEQGRLLCELFIRAPKRRNQPDYYEVVSQ
PIDLMKIQQKLKMEEYDDVNLLTADFQLLFNNAKSYYKPDSPEYK
AACKLWDLYLRTRNEFVQKGEADDEDDDEDGQDNQGTVTEGSSPA
YLKEILEQLLEAIVVATNPSGRLISELFQKLPSKVQYPDYYAIIK
EPIDLKTIAQRIQNGSYKSIHAMAKDIDLLAKNAKTYNEPGSQVF
KDANSIKKIFYMKKAEIEHHEMAKSSLRMRTPSNLAAARLTGPSH
SKGSLGEERNPTSKYYRNKRAVQGGRLSAITMALQYGSESEEDAA
LAAARYEEGESEAESITSFMDVSNPFYQLYDTVRSCRNNQGQLIA
EPFYHLPSKKKYPDYYQQIKMPISLQQIRTKLKNQEYETLDHLEC
DLNLMFENAKRYNVPNSAIYKRVLKLQQVMQAKKKELARRDDIED
GDSMISSATSDTGSAKRKSKKNIRKQRMKILFNVVLEAREPGSGR
RLCDLFMVKPSKKDYPDYYKIILEPMDLKIIEHNIRNDKYAGEEG
MIEDMKLMFRNARHYNEEGSQVYNDAHILEKLLKEKRKELGPLPD
DDDMASPKLKLSRKSGISPKKSKYMTPMQQKLNEVYEAVKNYTDK
RGRRLSAIFLRLPSRSELPDYYLTIKKPMDMEKIRSHMMANKYQD
IDSMVEDFVMMFNNACTYNEPESLIYKDALVLHKVLLETRRDLEG
DEDSHVPNVTLLIQELIHNLFVSVMSHQDDEGRCYSDSLAEIPAV
DPNFPNKPPLTFDIIRKNVENNRYRRLDLFQEHMFEVLERARRMN
RTDSEIYEDAVELQQFFIKIRDELCKNGEILLSPALSYTTKHLHN
DVEKERKEKLPKEIEEDKLKREEEKREAEKSEDSSGAAGLSGLHR
TYSQDCSFKNSMYHVGDYVYVEPAEANLQPHIVCIERLWEDSAEK
EVFKSDYYNKVPVSKILGKCVVMFVKEYFKLCPENFRDEDVFVCE
SRYSAKTKSFKKIKLWTMPISSVRFVPRDVPLPVVRVASVFANAD
KGDDEKNTDNSEDSRAEDNFNLEKEKEDVPVEMSNGEPGCHYFEQ
LHYNDMWLKVGDCVFIKSHGLVRPRVGRIEKVWVRDGAAYFYGPI
FIHPEETEHEPTKMFYKKEVFLSNLEETCPMTCILGKCAVLSFKD
FLSCRPTEIPENDILLCESRYNESDKQMKKFKGLKRFSLSAKVVD
DEIYYFRKPIVPQKEPSPLLEKKIQLLEAKFAELEGGDDDIEEMG
EEDSESTPKSAKGSAKKEGSKRKINMSGYILFSSEMRAVIKAQHP
DYSFGELSRLVGTEWRNLETAKKAEYEGMMGGYPPGLPPLQGPVD
GLVSMGSMQPLHPGGPPPHHLPPGVPGLPGIPPPGVMNQGVAPMV
GTPAPGGSPYGQQVGVLGPPGQQAPPPYPGPHPAGPPVIQQPTTP
MFVAPPPKTQRLLHSEAYLKYIEGLSAESNSISKWDQTLAARRRD
VHLSKEQESRLPSHWLKSKGAHTTMADALWRLRDLMLRDTLNIRQ
AYNLENV
SEQ ID NO: 35 is an exemplary nucleic acid
sequence encoding mouse Brd9.
ATGGGCAAAAAGCACAAGAAGCACAAGGCGGAATGGCGCTCGTCC
TACGAAGATTATACAGACACGCCACTGGAGAAGCCTCTGAAGCTG
GTGCTCAAGGTGGGAGGAAGTGAAGTGACAGAGCTCTCAGGATCT
GGCCACGACTCCAGCTACTACGACGATCGCTCAGACCACGAACGG
GAGAGACACAGAGAAAAGAAGAAAAAGAAGAAGAAAAAGTCAGAG
AAGGAGAAGCACCTCGATGAGGAGGAGAGGAGGAAGCGGAAGGAA
GAGAAGAAACGGAAACGGGAGAAGGAACACTGCGACTCAGAGGGG
GAGGCTGATGCTTTCGACCCTGGAAAGAAGGTGGAGGTGGAGCCA
CCCCCAGACCGACCAGTGAGAGCCTGCCGAACACAGCCAGCTGAG
AACGAGAGCACACCTATCCAGAGGCTTCTGGAACACTTCCTCCGC
CAGCTACAGAGAAAAGATCCTCATGGATTTTTTGCTTTTCCTGTT
ACGGATGCAATTGCTCCTGGGTATTCAATGATAATAAAACATCCT
ATGGACTTTGGCACGATGAAAGACAAGATTGTAGCTAATGAATAT
AAATCAGTCACAGAATTTAAGGCAGATTTCAAATTAATGTGTGAT
AATGCGATGACGTACAATAGACCAGACACCGTGTACTACAAATTA
GCCAAGAAGATCCTGCACGCGGGCTTTAAGATGATGAGCAAACAG
GCAGCTCTCTTGGGCAGTGAAGACCCAGCAGCTGAGGAACCTGTT
CCCGAGGTTGTCCCAGTGCAAGTAGAAACTACCAAGAAATCCAAA
AAGCCGAGTAGAGAAGTTATCAGCTGCATGTTTGAGCCTGAAGGG
AATGCCTGCAGCCTGACAGACAGCACGGCAGAGGAGCATGTGCTA
GCCCTGGTAGAGCACGCAGCTGATGAGGCTCGGGACAGGATTAAC
CGGTTTCTCCCGGGTGGCAAGATGGGGTACCTGAAGAAGCTTGGA
GATGGAAGTCTGCTCTACAGCGTGGTGAACGCACCTGAGCCTGAT
GCTGATGAGGAGGAGACACACCCTGTGGACCTGAGTTCACTGTCT
AGCAAGTTGCTCCCAGGTTTTACAACATTGGGTTTCAAAGATGAA
AGAAGAAATAAAGTCACATTCCTCTCCAGTGCCAGCACTGCACTT
TCAATGCAGAACAACTCTGTGTTTGGGGACCTGAAGTCAGATGAG
ATGGAGCTTCTGTATTCCGCCTATGGAGATGAGACTGGTGTGCAG
TGTGCACTGAGCCTGCAGGAATTCGTGAAGGATGCTGGAAGCTAT
AGCAAGAAGATGGTAGATGACCTCCTGGACCAAATCACAGGTGGT
GATCACTCAAGGATGATCTTCCAGCTGAAGCAGAGGAGGAGCATC
CCCATGAGACCTGCAGATGAGATGAAGGTTGGGGATCCACTGGGA
GAGAGTGGTGGCCCTGTTCTGGACTTCATGTCAATGAAACAGTAT
CCTGATGTCTCCCTGGATGTGTCCATGCTCAGCTCTCTCGGGAAA
GTAAAGAAGGAGCTGGACCATGAAGATAGCCACTTGAACTTGGAT
GAGACAGCCAGGCTCCTGCAGGACTTACACGAAGCACAAGCAGAG
CGAGGAGGCTCTCGGCCATCCTCCAACCTTAGCTCTCTGTCCACT
GCCTCTGAGAGGGAGCATCCTCCTCCAGGAAGTCCTTCTCGCCTT
AGTGTTGGGGAGCAGCCGGATGTCGCCCACGACCCTTATGAATTC
CTTCAGTCTCCAGAACCTGCAGCTCCTGCCAAGAACTAA
SEQ ID NO: 36 is an exemplary mouse Brd9
amino acid sequence.
MGKKHKKHKAEWRSSYEDYTDTPLEKPLKLVLKVGGSEVTELSGS
GHDSSYYDDRSDHERERHREKKKKKKKKSEKEKHLDEEERRKRKE
EKKRKREKEHCDSEGEADAFDPGKKVEVEPPPDRPVRACRTQPAE
NESTPIQRLLEHFLRQLQRKDPHGFFAFPVTDAIAPGYSMIIKHP
MDFGTMKDKIVANEYKSVTEFKADFKLMCDNAMTYNRPDTVYYKL
AKKILHAGFKMMSKQAALLGSEDPAAEEPVPEVVPVQVETTKKSK
KPSREVISCMFEPEGNACSLTDSTAEEHVLALVEHAADEARDRIN
RFLPGGKMGYLKKLGDGSLLYSVVNAPEPDADEEETHPVDLSSLS
SKLLPGFTTLGFKDERRNKVTFLSSASTALSMQNNSVFGDLKSDE
MELLYSAYGDETGVQCALSLQEFVKDAGSYSKKMVDDLLDQITGG
DHSRMIFQLKQRRSIPMRPADEMKVGDPLGESGGPVLDFMSMKQY
PDVSLDVSMLSSLGKVKKELDHEDSHLNLDETARLLQDLHEAQAE
RGGSRPSSNLSSLSTASEREHPPPGSPSRLSVGEQPDVAHDPYEF
LQSPEPAAPAKN
SEQ ID NO: 37 is an exemplary nucleic
acid sequence encoding mouse Brd7.
ATGGGCAAGAAGCACAAGAAGCACAAGTCGGACCGCCACTTCTAC
GAGGAGTACGTGGAGAAGCCCCTGAAGCTGGTCCTCAAAGTCGGG
GGGAGCGAGGTCACCGAGCTCTCCACGGGCAGCTCCGGGCACGAC
TCCAGCCTCTTCGAAGACAGAAGCGACCATGACAAACACAAGGAC
AGAAAACGGAAAAAGAGGAAGAAAGGCGAGAAGCAGGCTCCGGGG
GAAGAGAAGGGGAGAAAACGGAGAAGAGTCAAGGAGGATAAAAAG
AAGCGGGATCGAGACCGTGCAGAGAATGAGGTGGACAGAGATCTC
CAGTGTCATGTCCCTATAAGATTAGACTTACCTCCTGAGAAGCCT
CTTACAAGCTCGTTAGCCAAACAAGAAGAAGTAGAACAGACACCC
CTTCAGGAAGCTTTGAATCAGCTCATGAGACAATTGCAAAGAAAA
GACCCAAGTGCTTTCTTTTCATTTCCTGTGACGGATTTTATTGCG
CCTGGCTACTCCATGATTATTAAACACCCAATGGATTTTAGTACC
ATGAAAGAAAAGATCAAGAATAACGACTACCAGTCCATAGAAGAA
CTAAAGGATAACTTCAAGCTAATGTGTACTAATGCAATGATTTAC
AATAAGCCAGAGACCATTTATTATAAAGCTGCAAAGAAGCTGTTG
CACTCAGGGATGAAAATTCTCAGTCAGGAGAGAATTCAGAGCCTG
AAGCAGAGTATAGACTTCATGTCAGACTTGCAGAAAACTCGGAAG
CAGAAAGAACGAACAGATGCCTGTCAGAGTGGGGAGGACAGCGGC
TGCTGGCAGCGCGAGAGGGAAGACTCTGGAGATGCTGAAACACAG
GCCTTCAGAAGCCCCGCTAAGGACAATAAAAGGAAAGACAAAGAT
GTGCTTGAAGACAAATGGAGAAGCAGCAACTCAGAAAGGGAGCAT
GAGCAGATTGAGCGCGTTGTCCAGGAGTCAGGAGGCAAGCTAACA
CGGCGGCTGGCAAACAGTCAGTGTGAATTTGAAAGAAGAAAACCA
GATGGGACAACAACACTGGGGCTTCTCCATCCTGTGGATCCCATT
GTGGGAGAGCCAGGCTACTGCCCTGTGAGATTGGGGATGACAACT
GGAAGACTGCAGTCTGGAGTGAACACTCTGCAGGGGTTCAAAGAG
GATAAAAGGAACAGAGTAACCCCAGTATTATACTTGAATTATGGA
CCCTACAGTTCTTATGCCCCACATTATGACTCTACATTTGCCAAT
ATTAGCAAAGATGATTCTGATTTAATCTACTCAACATATGGGGAA
GACTCTGACCTTCCAAACAATTTCAGCATCTCTGAGTTTTTGGCC
ACATGCCAAGATTACCCGTATGTTATGGCAGATAGTTTACTGGAT
GTTCTAACAAAAGGAGGACATTCCAGGAGCCTGCAGGACTTGGAC
ATGTCATCTCCTGAAGATGAAGGCCAGACCAGAGCATTGGACACA
GCAAAAGAAGCAGAGATTACACAAATAGAGCCAACAGGGCGTTTG
GAGTCCAGCAGTCAGGACAGGCTCACAGCACTGCAAGCTGTAACA
ACCTTTGGTGCTCCAGCTGAAGTCTTTGACTCCGAAGAGGCTGAG
GTGTTCCAGAGGAAGCTTGATGAGACGACAAGATTGCTCAGGGAG
CTCCAGGAGGCACAGAATGAGCGACTGAGCACTAGGCCTCCTCCC
AATATGATCTGTCTCCTGGGTCCTTCTTACAGAGAAATGTACCTT
GCTGAACAAGTGACCAATAACCTCAAAGAACTCACACAGCAAGTG
ACTCCAGGTGATGTTGTAAGCATACACGGAGTGCGAAAAGCAATG
GGGATTTCTGTTCCTTCCCCCATCGTGGGAAACAGCTTCGTAGAT
TTGACAGGAGAGTGTGAAGAACCTAAGGAGACCAGCACTGCTGAG
TGTGGGCCTGACGCGAGCTGA
SEQ ID NO: 38 is an exemplary mouse
Brd7 amino acid sequence.
MGKKHKKHKSDRHFYEEYVEKPLKLVLKVGGSEVTELSTGSSGHD
SSLFEDRSDHDKHKDRKRKKRKKGEKQAPGEEKGRKRRRVKEDKK
KRDRDRAENEVDRDLQCHVPIRLDLPPEKPLTSSLAKQEEVEQTP
LQEALNQLMRQLQRKDPSAFFSFPVTDFIAPGYSMIIKHPMDFST
MKEKIKNNDYQSIEELKDNFKLMCTNAMIYNKPETIYYKAAKKLL
HSGMKILSQERIQSLKQSIDFMSDLQKTRKQKERTDACQSGEDSG
CWQREREDSGDAETQAFRSPAKDNKRKDKDVLEDKWRSSNSEREH
EQIERVVQESGGKLTRRLANSQCEFERRKPDGTTTLGLLHPVDPI
VGEPGYCPVRLGMTTGRLQSGVNTLQGFKEDKRNRVTPVLYLNYG
PYSSYAPHYDSTFANISKDDSDLIYSTYGEDSDLPNNFSISEFLA
TCQDYPYVMADSLLDVLTKGGHSRSLQDLDMSSPEDEGQTRALDT
AKEAEITQIEPTGRLESSSQDRLTALQAVTTFGAPAEVFDSEEAE
VFQRKLDETTRLLRELQEAQNERLSTRPPPNMICLLGPSYREMYL
AEQVTNNLKELTQQVTPGDVVSIHGVRKAMGISVPSPIVGNSFVD
LTGECEEPKETSTAECGPDAS
SEQ ID NO: 39 is an exemplary nucleic
acid sequence encoding mouse Pbrm1.
ATGGGTTCCAAGAGAAGAAGAGCCACCTCTCCTTCCAGCAGTGTC
AGTGGAGACTTTGATGACGGGCACCATTCTGTGCCTACACCAGGC
CCAAGCAGGAAAAGGAGAAGACTGTCCAATCTTCCAACTGTAGAT
CCTATTGCTGTGTGCCATGAACTCTATAACACCATCCGAGACTAT
AAGGATGAACAGGGCAGACTCCTCTGTGAGCTGTTCATTAGGGCT
CCAAAGCGGAGAAATCAACCAGACTATTATGAAGTGGTTTCTCAG
CCCATTGACTTGATGAAAATCCAACAGAAACTTAAAATGGAAGAG
TATGATGATGTTAATCTACTGACTGCTGACTTCCAGCTGCTTTTT
AACAATGCAAAGGCCTACTATAAGCCAGATTCCCCTGAGTATAAA
GCTGCTTGTAAACTCTGGGATTTGTACCTTCGAACAAGAAATGAG
TTTGTTCAGAAAGGAGAAGCAGACGATGAAGATGATGACGAAGAT
GGGCAAGACAATCAAGGCACACTGGCTGACGGCTCTTCTCCAGGT
TATCTGAAGGAGATCCTGGAGCAGCTTCTTGAAGCCATAGTTGTA
GCCACAAATCCATCAGGACGGCTCATCAGTGAACTTTTTCAGAAA
CTGCCTTCCAAAGTGCAATATCCAGACTATTATGCAATAATTAAG
GAACCTATAGATCTCAAGACCATTGCTCAGAGGATACAGAATGGA
AGCTACAAAAGTATACACGCAATGGCCAAAGATATAGATCTTCTA
GCAAAAAATGCCAAAACATACAATGAGCCTGGGTCTCAAGTATTC
AAGGATGCCAATTCGATTAAAAAAATATTTTATATGAAAAAGGCA
GAAATTGAACATCATGAAATGACTAAATCAAGTCTTCGAATAAGG
ACTGCATCAAATTTGGCTGCAGCCAGGCTGACAGGTCCTTCGCAC
AATAAAAGCAGCCTTGGTGAAGAAAGAAACCCCACTAGCAAGTAT
TACCGTAATAAAAGAGCAGTCCAAGGGGGTCGCTTGTCAGCAATT
ACCATGGCACTTCAGTATGGATCAGAGAGTGAAGAGGACGCTGCT
TTAGCTGCTGCACGCTATGAAGAAGGGGAATCTGAAGCAGAGAGC
ATCACTTCCTTCATGGACGTTTCCAACCCCTTTCATCAGCTTTAC
GACACAGTTAGGAGCTGTAGGAATCACCAAGGGCAGCTCATAGCT
GAACCTTTCTTCCATTTGCCTTCAAAGAAAAAATACCCAGATTAT
TATCAGCAAATTAAAATGCCCATATCACTTCAACAGATCAGAACA
AAGCTAAAGAACCAAGAATATGAAACTTTAGATCATTTGGAGTGT
GATCTGAATTTAATGTTTGAAAATGCCAAACGTTATAACGTTCCC
AATTCAGCCATCTATAAGCGAGTTCTAAAACTGCAGCAAGTCATG
CAGGCAAAGAAGAAGGAGCTTGCGAGGAGAGATGACATTGAGGAC
GGAGACAGCATGATCTCCTCAGCCACTTCTGACACTGGTAGTGCC
AAAAGGAAAAGGAATACTCATGACAGTGAGATGTTGGGTCTCAGG
AGGCTATCCAGTAAAAAGAACATAAGAAAACAGCGAATGAAAATT
TTATTCAATGTTGTTCTTGAAGCTCGAGAGCCAGGTTCAGGCAGA
AGACTTTGCGATCTATTTATGGTTAAGCCATCCAAGAAGGACTAT
CCTGATTATTATAAAATCATCTTAGAGCCAATGGACCTGAAAATA
ATTGAGCATAACATCCGAAATGACAAATATGCAGGTGAAGAAGGA
ATGATGGAAGACATGAAACTCATGTTCCGCAATGCCAGGCACTAC
AATGAGGAGGGCTCCCAGGTATACAATGATGCCCATATCCTGGAG
AAGTTACTCAAAGATAAAAGGAAAGAGCTGGGCCCTCTGCCTGAT
GATGATGACATGGCTTCTCCCAAACTTAAATTGAGTAGGAAGAGT
GGTGTTTCTCCTAAGAAATCAAAGTACATGACTCCAATGCAGCAG
AAACTGAATGAAGTGTATGAAGCTGTAAAGAACTATACTGATAAG
AGGGGTCGCCGCCTTAGTGCTATATTTCTAAGACTCCCCTCTAGA
TCAGAGCTGCCTGACTACTACCTGACCATTAAAAAGCCCATGGAC
ATGGAAAAAATTCGAAGTCACATGATGGCAAACAAGTACCAAGAC
ATAGATTCTATGGTAGAGGACTTTGTCATGATGTTTAATAATGCC
TGTACCTACAATGAACCAGAGTCTTTGATCTACAAAGATGCCCTT
GTACTGCATAAAGTCCTCCTTGAGACTCGGAGAGACCTGGAGGGA
GATGAGGATTCTCATGTCCCTAATGTGACGTTGCTGATTCAAGAG
CTCATCCATAACCTTTTTGTGTCAGTCATGAGTCATCAGGATGAC
GAAGGGAGGTGTTACAGCGACTCCTTAGCAGAAATTCCTGCTGTG
GATCCCAACTCTCCCAATAAACCTCCCCTTACATTTGACATTATC
AGGAAAAATGTTGAAAGTAATCGGTATCGGCGACTTGATTTATTT
CAGGAGCATATGTTTGAAGTATTGGAACGGGCAAGAAGGATGAAC
CGGACAGATTCCGAAATATATGAGGATGCTGTAGAACTTCAGCAG
TTTTTTATTAGAATTCGTGATGAACTCTGCAAAAATGGAGAGATC
CTTCTTTCTCCAGCACTCAGCTATACCACAAAACACTTGCATAAC
GATGTGGAAAAAGAAAAAAAGGAAAAATTGCCTAAAGAAATAGAG
GAAGATAAACTAAAACGCGAAGAAGAAAAAAGAGAAGCTGAAAAA
AGTGAAGATTCCTCAGGTACTACAGGCCTCTCAGGCTTACATCGT
ACATACAGCCAGGACTGCAGCTTTAAGAACAGCATGTATCATGTC
GGAGATTATGTCTATGTTGAACCTGCGGAGGCCAATCTACAACCA
CATATAGTGTGTATTGAGAGACTGTGGGAGGATTCAGCTGGTGAA
AAATGGTTGTACGGCTGTTGGTTTTATCGGCCAAATGAAACATTC
CATTTGGCTACACGAAAATTTCTAGAAAAAGAAGTTTTTAAGAGT
GACTACTACAATAAAGTACCTGTTAGTAAAATTCTAGGCAAATGT
GTAGTCATGTTTGTCAAGGAATACTTTAAATTATGTCCAGAAAAC
TTTCGCGATGAGGATGTTTTTGTCTGTGAATCGAGGTATTCTGCC
AAAACCAAATCTTTTAAGAAAATTAAACTGTGGACCATGCCCATC
AGTTCAGTTAGATTTGTCCCTCGGGATGTGCCTTTGCCTGTGGTC
CGAGTGGCCTCTGTGTTTGCAAATGCAGATAAAGGGGATGATGAG
AAGAATACAGACAACTCAGATGACAATAGAGCTGAAGACAATTTT
AACTTGGAAAAGGAAAAAGAAGATGTTCCTGTGGAGATGTCCAAT
GGTGAGCCAGGTTGCCACTACTTTGAGCAGCTTCGGTACAATGAC
ATGTGGCTGAAGGTTGGTGATTGTGTCTTCATCAAATCCCACGGC
TTGGTGCGCCCTCGTGTGGGCAGAATTGAGAAAGTATGGGTCCGA
GATGGAGCTGCATATTTTTATGGCCCTATCTTCATTCATCCAGAA
GAAACAGAACATGAGCCCACAAAAATGTTCTACAAAAAAGAAGTG
TTTCTGAGTAATCTGGAAGAGACCTGCCCTATGAGTTGTATTCTG
GGGAAATGTGCAGTGCTGTCATTCAAGGACTTCCTCTCCTGCAGG
CCAACTGAAATACCAGAAAATGACATTCTGCTTTGTGAGAGCCGC
TATAATGAGAGTGACAAGCAGATGAAGAAGTTCAAGGGTTTGAAG
AGGTTTTCACTCTCTGCTAAAGTTGTAGATGATGAAATCTACTAC
TTCAGAAAACCAATCATTCCTCAGAAGGAACCCTCACCTTTGTTA
GAAAAGAAGATACAATTGCTAGAAGCTAAATTTGCAGAGTTAGAA
GGAGGAGATGATGATATTGAGGAGATGGGAGAAGAGGATAGTGAA
GTCATTGAAGCTCCATCTCTACCTCAACTGCAGACACCCCTGGCC
AATGAGTTGGACCTCATGCCCTATACACCCCCACAGTCTACCCCA
AAGTCTGCCAAAGGCAGTGCAAAGAAGGAAAGTTCTAAACGAAAA
ATCAACATGAGTGGCTACATTTTGTTCAGCAGTGAAATGAGAGCT
GTGATTAAAGCCCAGCACCCAGACTACTCTTTTGGGGAGCTCAGC
AGACTGGTGGGGACAGAATGGAGAAACCTTGAAACAGCCAAGAAA
GCAGAATATGAAGAGCGGGCAGCTAAAGTTGCTGAGCAGCAGGAG
AGAGAGCGAGCAGCACAGCAACAGCAGCCGAGTGCTTCTCCCCGA
GCAGGCACCCCTGTGGGGGCTCTCATGGGGGTGGTGCCACCACCA
ACACCAATGGGGATGCTCAATCAGCAGTTGACACCTGTTGCAGGC
ATGATGGGTGGCTATCCGCCAGGCCTTCCACCTTTGCAGGGCCCA
GTTGATGGCCTTGTTAGCATGGGCAGCATGCAGCCACTTCACCCT
GGGGGGCCTCCACCTCACCATCTTCCGCCAGGTGTGCCTGGCCTC
CCAGGCATCCCACCACCGGGTGTGATGAATCAAGGAGTAGCCCCC
ATGGTAGGGACTCCAGCACCAGGTGGAAGTCCGTATGGACAACAG
GTAGGAGTTTTGGGACCTCCAGGGCAGCAGGCACCACCTCCATAT
CCTGGTCCTCATCCAGCTGGCCCCCCTGTCATACAGCAGCCAACA
ACGCCCATGTTTGTGGCTCCCCCACCAAAGACCCAAAGGCTTCTC
CACTCAGAGGCCTACCTGAAATACATTGAAGGACTCAGTGCTGAA
TCCAACAGCATTAGCAAGTGGGACCAAACTTTGGCAGCTCGAAGA
CGGGATGTCCATTTGTCCAAAGAACAGGAGAGCCGCCTACCTTCT
CACTGGCTCAAAAGTAAAGGGGCACACACCACCATGGCAGATGCC
CTCTGGCGCCTACGGGATTTAATGCTTCGAGACACTCTCAACATC
CGACAGGCATACAACCTAGAAAATGTTTAA
SEQ ID NO: 40 is an exemplary amino acid
sequenceofmousePbrm1.MGSKRRRATSPSSSVSGDFDDGHH
SVPTPGPSRKRRRLSNLPTVDPIAVCHELYNTIRDYKDEQGRLLC
ELFIRAPKRRNQPDYYEVVSQPIDLMKIQQKLKMEEYDDVNLLTA
DFQLLFNNAKAYYKPDSPEYKAACKLWDLYLRTRNEFVQKGEADD
EDDDEDGQDNQGTLADGSSPGYLKEILEQLLEAIVVATNPSGRLI
SELFQKLPSKVQYPDYYAIIKEPIDLKTIAQRIQNGSYKSIHAMA
KDIDLLAKNAKTYNEPGSQVFKDANSIKKIFYMKKAEIEHHEMTK
SSLRIRTASNLAAARLTGPSHNKSSLGEERNPTSKYYRNKRAVQG
GRLSAITMALQYGSESEEDAALAAARYEEGESEAESITSFMDVSN
PFHQLYDTVRSCRNHQGQLIAEPFFHLPSKKKYPDYYQQIKMPIS
LQQIRTKLKNQEYETLDHLECDLNLMFENAKRYNVPNSAIYKRVL
KLQQVMQAKKKELARRDDIEDGDSMISSATSDTGSAKRKRNTHDS
EMLGLRRLSSKKNIRKQRMKILFNVVLEAREPGSGRRLCDLFMVK
PSKKDYPDYYKIILEPMDLKIIEHNIRNDKYAGEEGMMEDMKLMF
RNARHYNEEGSQVYNDAHILEKLLKDKRKELGPLPDDDDMASPKL
KLSRKSGVSPKKSKYMTPMQQKLNEVYEAVKNYTDKRGRRLSAIF
LRLPSRSELPDYYLTIKKPMDMEKIRSHMMANKYQDIDSMVEDFV
MMFNNACTYNEPESLIYKDALVLHKVLLETRRDLEGDEDSHVPNV
TLLIQELIHNLFVSVMSHQDDEGRCYSDSLAEIPAVDPNSPNKPP
LTFDIIRKNVESNRYRRLDLFQEHMFEVLERARRMNRTDSEIYED
AVELQQFFIRIRDELCKNGEILLSPALSYTTKHLHNDVEKEKKEK
LPKEIEEDKLKREEEKREAEKSEDSSGTTGLSGLHRTYSQDCSFK
NSMYHVGDYVYVEPAEANLQPHIVCIERLWEDSAGEKWLYGCWFY
RPNETFHLATRKFLEKEVFKSDYYNKVPVSKILGKCVVMFVKEYF
KLCPENFRDEDVFVCESRYSAKTKSFKKIKLWTMPISSVRFVPRD
VPLPVVRVASVFANADKGDDEKNTDNSDDNRAEDNFNLEKEKEDV
PVEMSNGEPGCHYFEQLRYNDMWLKVGDCVFIKSHGLVRPRVGRI
EKVWVRDGAAYFYGPIFIHPEETEHEPTKMFYKKEVFLSNLEETC
PMSCILGKCAVLSFKDFLSCRPTEIPENDILLCESRYNESDKQMK
KFKGLKRFSLSAKVVDDEIYYFRKPIIPQKEPSPLLEKKIQLLEA
KFAELEGGDDDIEEMGEEDSEVIEAPSLPQLQTPLANELDLMPYT
PPQSTPKSAKGSAKKESSKRKINMSGYILFSSEMRAVIKAQHPDY
SFGELSRLVGTEWRNLETAKKAEYEERAAKVAEQQERERAAQQQQ
PSASPRAGTPVGALMGVVPPPTPMGMLNQQLTPVAGMMGGYPPGL
PPLQGPVDGLVSMGSMQPLHPGGPPPHHLPPGVPGLPGIPPPGVM
NQGVAPMVGTPAPGGSPYGQQVGVLGPPGQQAPPPYPGPHPAGPP
VIQQPTTPMFVAPPPKTQRLLHSEAYLKYIEGLSAESNSISKWDQ
TLAARRRDVHLSKEQESRLPSHWLKSKGAHTTMADALWRLRDLML
RDTLNIRQAYNLENV
SEQ ID NO: 41 is exemplary flanking regions
when an sgRNA is ligated into BbsI
digested pSIRG-NGFR. AAACACCGAANNNGTCGTTTTAG
SEQ ID NO: 42 is an exemplary siRNA
targeting Brd9.
GAAGGAACACTGCGACTCAGA
SEQ ID NO: 43 is an exemplary siRNA
targeting Brd7.
GCCTGGCTACTCCATGATTAT
SEQ ID NO: 44 is an exemplary siRNA
targeting PBRMI.
GGCACTTCAGTATGGATCAGA
SEQ ID NO: 45 is an exemplary gRNA
targeting Brd9 for the Cas13 system.
TGCTCATCATCTTAAAGCCCGCG
SEQ ID NO: 46 is an exemplary gRNA
targeting Brd7 for the Cas13 system.
TTTAATAATCATGGAGTAGCCAG
SEQ ID NO: 47 is an exemplary gRNA
targeting PBRM1 for the Cas13 system.
TTATAGAGTTCATGGCACACAGC

### DETAILED DESCRIPTION

### I. Abbreviations

ARID2 AT-rich interactive domain-containing protein 2
Brd7 bromodomain-containing 7
Brd9 bromodomain-containing 9
CAR chimeric antigen receptor
CRISPR clustered regularly interspaced short palindromic repeats
Foxp3 forkhead box P3
IL-10 interleukin 10
IL-35 interleukin 35
IFN-γ interferon gamma
Gltscr1 glioma tumor suppressor candidate region 1
Gltscr11 glioma tumor suppressor candidate region 1-like
ncBAF non-canonical brahma-associated factor
Phf10 PHD Finger Protein 10
PBAF polybromo-associated brahma-associated factor
Pbrm1 polybromo 1
RNAi RNA interference
Smarca4 SWI/SNF related, matrix associated, actin dependent regulator of chromatin, subfamily A, member 4
SWI/SNF SWItch/Sucrose Non-Fermentable
TALEN transcription activator-like effector nucleases
TGF-β transforming growth factor beta
Treg T regulatory cell

### II. Terms

Unless otherwise noted, technical terms are used according to conventional usage. Definitions of common terms in molecular biology may be found in Lewin's Genes X, ed. Krebs et al., Jones and Bartlett Publishers, 2009 (ISBN 0763766321); Kendrew et al. (eds.), The Encyclopedia of Molecular Biology, published by Blackwell Publishers, 1994 (ISBN 0632021829); Robert A. Meyers (ed.), Molecular Biology and Biotechnology: a Comprehensive Desk Reference, published by Wiley, John & Sons, Inc., 1995 (ISBN 0471186341); and George P. Rédei, Encyclopedic Dictionary of Genetics, Genomics, Proteomics and Informatics, 3rd Edition, Springer, (ISBN: 1402067534), and other similar references.

Unless otherwise explained, all technical and scientific terms used herein have the same meaning as commonly understood by one of ordinary skill in the art to which this disclosure belongs. The singular terms “a,” “an,” and “the” include plural referents unless the context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description.

Although methods and materials similar or equivalent to those described herein can be used in the practice or testing of the present disclosure, suitable methods and materials are described below. In case of conflict, the present specification, including explanations of terms, will control. In addition, the materials, methods, and examples are illustrative only and not intended to be limiting.

In order to facilitate review of the various embodiments of the disclosure, the following explanations of specific terms are provided:
Activator: A composition that increases expression of a gene or increases activity of a gene product. For example, an activator targeting Brd7, Brd9, or Pbrm1 can increase transcription of a Brd7, Brd9, or Pbrm1 gene, respectively, increase translation of Brd7, Brd9, or Pbrm1 mRNA, respectively, or decrease degradation of Brd7, Brd9, or Pbrm1 mRNA or protein, respectively, thereby increasing the level of Brd7, Brd9, or Pbrm1 protein in the subject or target cell, such as a Treg cell. In some embodiments, accumulation or levels of a gene product (such as a Brd7, Brd9, or Pbrm1 gene product) is increased at least 10%, at least 25%, at least 50%, at least 75%, at least 90%, or at least 95% relative to a control, such as untreated control cells. In other examples, an activator targeting Brd7, Brd9, or Pbrm1 increases activity of Brd7, Brd9, or Pbrm1 protein, respectively. In some embodiments, Brd7, Brd9, or Pbrm1 activity is increased at least 10%, at least 25%, at least 50%, at least 75%, at least 90%, or at least 95% relative to a control, such as untreated control cells.

Administration: The introduction of a composition, such as a small molecule inhibitor, RNAi, or gRNA, into a subject by a chosen route. Administration can be local or systemic. Exemplary routes of administration include, but are not limited to, oral, injection (such as subcutaneous, intramuscular, intradermal, intraperitoneal, intratumoral, intravenous), sublingual, rectal, transdermal (for example, topical), intranasal, vaginal, and inhalation routes.

Agent: Any substance, compound or drug that is useful for achieving a particular outcome. For example, the agent can be a substance, compound, or drug capable of modulating expression or activity of one or more components of the ncBAF or PBAF complex. In some embodiments, the agent is a compound that modulates expression or activity of Brd9, Brd7, or Pbrm1. In some embodiments, the agent is a therapeutic agent, such as a therapeutic agent for the treatment of an autoimmune disease or cancer, or a therapeutic agent that enhances cancer immunotherapy.

Autoimmune Disease or Disorder: A disease or condition in which the immune system responds to self-antigens (autoreactive immune cells) resulting in self-destruction of healthy tissue.

Examples of autoimmune disease or disorders include rheumatoid arthritis, systemic lupus erythematosus, type 1 diabetes, multiple sclerosis, Sjögren's syndrome, Graves' disease, myasthenia gravis, ulcerative colitis, Hashimoto's thyroiditis, celiac disease, Crohn's disease, arthritis, inflammatory bowel disease, or scleroderma.

Bromodomain-containing 7 (Brd7): A component of the PBAF nucleosome remodeling complex (Loo et al., Immunity 53, 143-157, 2020). Sequence information for human Pbrm1 can be found, for example, on the Consensus CDS Protein Set Database as CCDS54007.1 (ncbi.nlm.nih.gov/CCDS/CcdsBrowse.cgi), herein incorporated by reference in its entirety. Similarly, sequence information for mouse Pbrm1 can also be found on the Consensus CDS Protein Set Database as CCDS22510.1 (ncbi.nlm.nih.gov/CCDS/CcdsBrowse.cgi), herein incorporated by reference in its entirety. Exemplary Brd7 sequences that can be targeted or used with the disclosed methods are provided in SEQ ID NOS: 31, 32, 37 and 38.

Bromodomain-containing 9 (Brd9): A component of the ncBAF nucleosome remodeling complex (Loo et al., Immunity 53, 143-157, 2020). Sequence information for human Pbrm1 can be found, for example, on the Consensus CDS Protein Set Database as CCDS34127.2 (ncbi.nlm.nih.gov/CCDS/CcdsBrowse.cgi), herein incorporated by reference in its entirety. Similarly, sequence information for mouse Pbrm1 can also be found on the Consensus CDS Protein Set Database as CCDS36728.1 (ncbi.nlm.nih.gov/CCDS/CcdsBrowse.cgi), herein incorporated by reference in its entirety. Exemplary Brd9 sequences that can be targeted or used with the disclosed methods are provided in SEQ ID NOS: 29, 30, 35 and 36.

Cancer: A malignant tumor characterized by abnormal or uncontrolled cell growth. Other features often associated with cancer include metastasis, interference with the normal functioning of neighboring cells, release of cytokines or other secretory products at abnormal levels, suppression or aggravation of inflammatory or immunological response, invasion of surrounding or distant tissues or organs, such as lymph nodes, etc. “Metastatic disease” refers to cancer cells that have left the original tumor site and migrated to other parts of the body, for example via the bloodstream or lymph system.

Checkpoint Inhibitor: Cell cycle checkpoints refer to safeguard mechanisms that ensure a cell correctly completes each cell cycle phase during mitotic division. Checkpoint inhibitors can sensitize cancer cells to DNA damaging drugs by causing cells with DNA damage to bypass the S and G2/M arrest and enter mitosis, leading to cell death by mitotic catastrophe. Cell cycle checkpoint inhibitors are described in more detail, for example, by Visconti et al., J Exp Clin Cancer Res. 35(1): 153, 2016.

Exemplary checkpoint inhibitors include ipilimumab (Yervoy®), nivolumab (Opdivo®), pembrolizumab (Keytruda®), atezolizumab (Tencentriq®), avelumab (Bavencio®), durvalumab (Imfinzi®), cemiplimab (Libtayo®), palbociclib (Ibrance®), ribociclib (Kisquali®), and abemaciclib (Verzenio®). Further examples are provided in Qiu et al., Journal of the European Society for Therapeutic Radiology and Oncology, 126(3):450-464, 2018; Visconti et al., J Exp Clin Cancer Res. 35(1): 153, 2016; and Mills et al. Cancer Res. 77(23): 6489-6498, 2017.

A checkpoint inhibitor may also include a spindle assembly checkpoint inhibitor. For example, spindle assembly checkpoint inhibitors include MK-1775 (AZD1775), taxanes, or vinca alkaloids (see Zhou and Giannakakou. Curr Med Chem Anticancer Agents. 5:65-71, 2005; and Visconti et al., J Exp Clin Cancer Res. 35(1): 153, 2016).

Chemotherapeutic agents: Any chemical agent with therapeutic usefulness in the treatment of diseases characterized by abnormal cell growth. Such diseases include tumors, neoplasms, and cancer as well as diseases characterized by hyperplastic growth, such as psoriasis. In some cases, a chemotherapeutic agent is a radioactive compound. In some cases, a chemotherapeutic agent is a biologic, such as a therapeutic monoclonal antibody. One can readily identify a chemotherapeutic agent of use (e.g., see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2^{nd} ed., 2000 Churchill Livingstone, Inc; Baltzer, L., Berkery, R. (eds): Oncology Pocket Guide to Chemotherapy, 2nd ed. St. Louis, Mosby-Year Book, 1995; Fischer, D. S., Knobf, M. F., Durivage, H. J. (eds): The Cancer Chemotherapy Handbook, 4th ed. St. Louis, Mosby-Year Book, 1993). Combination chemotherapy is the administration of more than one agent to treat cancer.

Chimeric antigen receptor (CAR): A chimeric molecule that includes an antigen-binding portion (such as a single domain antibody or scFv) and a signaling domain, such as a signaling domain from a T cell receptor (e.g., CD3). Typically, CARs include an antigen-binding portion, a transmembrane domain, and an intracellular domain. The intracellular domain typically includes a signaling chain having an immunoreceptor tyrosine-based activation motif (ITAM), such as CD3ζ or FcεRIγ. In some instances, the intracellular domain also includes the intracellular portion of at least one additional co-stimulatory domain, such as CD28, 4-1BB (CD137), ICOS, OX40 (CD134), CD27 and/or DAP10. In the context of cancer immunotherapy, the antigen-binding portion typically targets and binds cancer antigens.

Control: A reference standard. In some examples, the control may be a subject not receiving treatment with an agent or receiving an alternative treatment, or a baseline reading of the subject prior to treatment. Similarly, in other examples the control can be an untreated subject or Treg cell. In still other embodiments, the control is a historical control or standard reference value or range of values (such as a previously tested control sample, such as a group of patients diagnosed with a disease or condition, for example cancer or an autoimmune disease, that have a known prognosis or outcome, or a group of samples that represent baseline or normal values).

A difference between a test sample and a control can be an increase or conversely a decrease (reduction). The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

CRISPR/Cas editing: a widely used system for targeted DNA or RNA editing. CRISPR/Cas systems can be categorized into two classes (class I, class II), which are further subdivided into six types (type I-VI). Class I includes type I, III, and IV, and class II includes type II, V, and VI. Type I, II, and V systems recognize and cleave DNA, type VI can edit RNA, and type III edits both DNA and RNA. The CRISPR/Cas9 system (type II) specifically cleaves double-stranded DNA (dsDNA) in vitro and leads to double-strand breaks (DSBs), which is useful for genome editing. The CRISPR/Cas13 system (type VI) specifically cleaves RNA, which is useful for targeted knockdown of target transcripts. A guide RNA (gRNA) facilitates Cas nuclease targeting of a target sequence.

Forkhead box P3 (Foxp3): A transcription factor that regulates and orchestrates the molecular processes involved in Treg differentiation and function (Zheng and Rudensky, Nat. Immunol. 8:457-462, 2007). Treg cells are a type of T cell that have an important role in maintaining immune system homeostasis by suppressing over-reactive immune responses (Josefowicz et al. Annu. Rev. Immunol. 30, 531-564, 2012). Defects in Treg cells can lead to autoimmune disorders and immunopathology. Conversely, certain tumors are enriched with Treg cells that suppress anti-tumor immune responses (Tanaka and Sakaguchi, Cell Res. 27, 109-118, 2017). Increased Foxp3 activity enhances Treg suppressor function, whereas decreased Foxp3 activity suppresses Treg suppressor function (Loo et al., Immunity 53, 143-157, 2020).

Glioblastoma (or glioblastoma multiforme): The most common and most malignant of the glial tumors. While glioblastomas almost exclusively occur in the brain, they can also appear in the brain stem, cerebellum, and spinal cord. Standard therapy includes concurrent surgical resection with radiation and chemotherapy (temozolomide). However, treatment is often not curative, and prognosis remains poor with a median survival of only 15 months (Davis, Clin J Oncol Nurs. 20(5): S2-S8, 2016).

Guide RNA (gRNA): a RNA or RNA hybrid structure that functions as a guide for RNA- or DNA-targeting enzymes, such as Cas nucleases.

Increasing expression or activity: As used herein, an agent that increases expression or activity of a gene, gene product, or complex is a compound that increases the level of the mRNA or protein product encoded by the gene in a cell or tissue, or increases one or more activities of the gene product or complex. Some non-limiting examples include increasing transcription of Brd7, Brd9, or Pbrm1 genes, increasing translation of Brd7, Brd9, or Pbrm1 mRNA, or decreasing degradation of Brd7, Brd9, or Pbrm1 protein thereby increasing the level of Brd7, Brd9, or Pbrm1 protein in a subject or a cell (such as a Treg cell) as compared to a suitable control.

In some embodiments, accumulation or levels of a gene product (such as a Brd7, Brd9, or Pbrm1 gene product) is increased by at least 10%, for example, at least 25%, at least 50%, at least 75%, at least 90%, at least 100%, at least 200%, at least 300%, at least 400%, or more relative to a control. As an example, an expression vector encoding Brd7, Brd9, or Pbrm1 may increase activity of the Brd7, Brd9, or Pbrm1 protein by increasing expression of the Brd7, Brd9, or Pbrm1 gene. In some embodiments, activity of the gene product (such as Brd7, Brd9, or Pbrm1) is increased by at least 10%, for example, at least 25%, at least 50%, at least 75%, at least 90%, at least 100%, at least 200%, at least 300%, at least 400%, or more relative to an untreated control. An agent that increases the expression of Brd7, Brd9, or Pbrm1, or the activity of respective protein products, can increase the activity of an associated complex, such as increasing the activity of ncBAF or PBAF. In some embodiments, the activity of the complex (such as ncBAF or PBAF) is increased at by at least 10%, for example, at least 25%, at least 50%, at least 75%, at least 90%, at least 100%, at least 200%, at least 300%, at least 400%, or more relative to a suitable control.

Isolated: An “isolated” biological component (e.g. nucleic acid, protein, or cell) has been substantially separated or purified away from other biological components in the environment (such as a cell or tissue) in which the component occurs, e.g., other chromosomal and extra-chromosomal DNA and RNA, proteins and cells. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Modulating expression or activity: As used herein, modulating expression or activity refers to modifying the expression of a gene, including gene transcription or translation of a respective mRNA product, or modifying the activity of a gene product, such as increasing stability or levels (quantity) of the gene product in a subject or cell (such as a Treg cell), or decreasing stability or levels (quantity) of the gene product in a subject or cell (such as a Treg cell).

Multiple Sclerosis: An autoimmune disease of the brain and spinal cord caused by an autoimmune response to myelin, a substance that insulates nerve fibers. The cause of MS is not known, but genetic susceptibility, abnormalities in the immune system, and environmental factors may all be contributing to development of the disease. Diagnosis can be made by brain and spinal cord magnetic resonance imaging (MRI) analysis of the patient. Serial MRI studies can be used to indicate disease progression.

Non-canonical brahma-associated factor (ncBAF): A SWItch/Sucrose Non-Fermentable (SWI/SNF) nucleosome remodeling complex. The ncBAF complex contains multiple protein subunits, but uniquely incorporates Brd9, and Gltscr1 or the paralog Gltscr11. The ncBAF complex is related to PBAF, but lacks the PBAF-specific subunits Pbrm1, Brd7, and ARID2. The ncBAF complex promotes transcription of Foxp3, thus, deletion of ncBAF constituent Brd9 in Treg cells reduces Treg cell suppressor activity (Loo et al., Immunity 53, 143-157, 2020).

Pharmaceutically Acceptable Carrier: Includes any and all solvents, dispersion media, coatings, antibacterial and antifungal agents, isotonic and absorption delaying agents, and the like, compatible with pharmaceutical administration, for example administration of small molecules, cells, nucleic acid molecules, or proteins (see, e.g., Remington: The Science and Practice of Pharmacy, The University of the Sciences in Philadelphia, Editor, Lippincott, Williams, & Wilkins, Philadelphia, PA, 21^{st} Edition, 2005). Examples of such carriers or diluents include, but are not limited to, water, saline, Ringer's solutions, dextrose solution, balanced salt solutions, and 5% human serum albumin Liposomes and non-aqueous vehicles such as fixed oils may also be used. Supplementary active compounds can also be incorporated into the compositions. Actual methods for preparing administrable compositions include those provided in Remington: The Science and Practice of Pharmacy, The University of the Sciences in Philadelphia, Editor, Lippincott, Williams, & Wilkins, Philadelphia, PA, 21^{st} Edition (2005).

Polybromo-associated brahma-associated factor (PBAF): A SWI/SNF family nucleosome remodeling complex. The PBAF complex contains multiple protein subunits, and uniquely incorporates Pbrm1, Brd7, and ARID2. PBAF is in the same family as the ncBAF complex, but lacks ncBAF-specific subunits Brd9, Gltscr1 or the paralog Gltscr11. The PBAF complex represses transcription of Foxp3, thus deletion of PBAF constituent Pbrm1 or Brd7 in Treg cells increases Treg cell suppressor activity (Loo et al., Immunity 53, 143-157, 2020).

Polybromo 1 (Pbrm1): A component of the PBAF nucleosome remodeling complex (Loo et al., Immunity 53, 143-157, 2020). Sequence information for human Pbrm1 can be found, for example, on the Consensus CDS Protein Set Database as CCDS43099.1(ncbi.nlm.nih.gov/CCDS/CcdsBrowse.cgi?REQUEST=CCDS&GO=MainBrowse& DATA=CCDS43099.1), herein incorporated by reference in its entirety. Similarly, sequence information for mouse Pbrm1 can be found on the Consensus CDS Protein Set Database as CCDS36851.1 (ncbi.nlm.nih.gov/CCDS/CcdsBrowse.cgi?REQUEST=CCDS&GO=MainBrowse&DATA=CCDS43099.1), herein incorporated by reference in its entirety. Exemplary Pbrm1 sequences that can be targeted or used with the disclosed methods are provided in SEQ ID NOS: 33, 34, 39 and 40.

Preventing, treating or ameliorating a disease: “Preventing” a disease refers to inhibiting the full development of a disease. “Treating” refers to a therapeutic intervention that ameliorates a sign or symptom of a disease or pathological condition (such as cancer or an autoimmune disease or disorder) after it has begun to develop. “Ameliorating” refers to the reduction in the number or severity of signs or symptoms of a disease.

Promoter: An array of nucleic acid control sequences which direct transcription of a nucleic acid, such as a coding sequence or gRNA. A promoter includes necessary nucleic acid sequences near the start site of transcription. A promoter also optionally includes distal enhancer or repressor elements. A “constitutive promoter” is a promoter that is continuously active and is not subject to regulation by external signals or molecules. In contrast, the activity of an “inducible promoter” is regulated by an external signal or molecule (for example, a transcription factor).

Purified: The term purified does not require absolute purity; rather, it is intended as a relative term. Thus, for example, a purified protein, nucleic acid, or cell preparation is one in which the protein, nucleic acid, or cell is more enriched than the protein, nucleic acid, or cell is in its initial environment. In one embodiment, a preparation is purified such that the protein, nucleic acid, or cell represents at least 50% of the total content of the preparation. A substantially purified protein or nucleic acid is at least 60%, 70%, 80%, 90%, 95% or 98% pure. Thus, in one specific, non-limiting example, a substantially purified protein or nucleic acid is 90% free of other components.

Recombinant: A nucleic acid or protein that has a sequence that is not naturally occurring or has a sequence that is made by an artificial combination of two otherwise separated segments of sequence (e.g., a “chimeric” sequence). This artificial combination can be accomplished by chemical synthesis or by the artificial manipulation of isolated segments of nucleic acids, for example, by genetic engineering techniques.

Reducing (decreasing) expression or activity: As used herein, an agent that reduces (decreases) expression or activity of a gene, gene product, or complex is a compound that reduces the level of the mRNA or product encoded by the gene in a cell or tissue, or reduces (including eliminates or inhibits) one or more activities of the gene product or complex. Some non-limiting examples include an RNAi or gRNA (e.g., sgRNA) molecule targeting Brd7, Brd9, or Pbrm1, or a small molecule inhibitor of Brd7, Brd9, or Pbrm1.

In some embodiments, expression of a gene product (such as a Brd7, Brd9, or Pbrm1 gene product) is reduced by at least 10%, for example at least 20%, at least 25%, at least 30%, at least 40%, at least 50%, at least 60%, at least 70%, at least 75%, at least 80%, at least 90%, at least 95%, at least 98%, or even 100% relative to a control, such as an untreated subject or cells (such as Treg cells). As an example, an antibody or small molecule that specifically binds or targets Brd7, Brd9, or Pbrm1 may reduce activity of the Brd7, Brd9, or Pbrm1 protein by preventing the Brd7, Brd9, or Pbrm1 protein from interacting with another protein (such as other proteins in the ncBAF or PBAF complex) or by reducing activity or function of the protein. In some embodiments, activity of the gene product (such as Brd7, Brd9, or Pbrm1) is reduced by at least 10%, for example at least 20%, at least 25%, at least 30%, at least 40%, at least 50%, at least 60%, at least 70%, at least 75%, at least 80%, at least 90%, at least 95%, at least 98%, or even 100% relative to an untreated control. As another example, an agent that reduces the expression of Brd7, Brd9, or Pbrm1, or the activity of respective protein products, reduces the activity of an associated complex, such as reducing the activity of ncBAF or PBAF. In some embodiments, activity of the complex (such as ncBAF or PBAF) is reduced by at least 10%, for example at least 20%, at least 25%, at least 30%, at least 40%, at least 50%, at least 60%, at least 70%, at least 75%, at least 80%, at least 90%, at least 95%, at least 98%, or even 100% relative to a suitable control. In some examples, the agent that reduces expression or activity of Brd7, Brd9, or Pbrm1 is a small molecule inhibitor, siRNA, or gRNA, targeting Brd7, Brd9, or Pbrm1, respectively.

RNA interference (RNAi): A cellular process that inhibits expression of genes, including cellular and viral genes. RNAi is a form of antisense-mediated gene silencing involving the introduction of double stranded RNA-like oligonucleotides leading to the sequence-specific reduction of RNA transcripts. RNA molecules that inhibit gene expression through the RNAi pathway include siRNAs, miRNAs, and shRNAs.

Sequence identity/similarity: The similarity between amino acid (or nucleotide) sequences is expressed in terms of the similarity between the sequences, otherwise referred to as sequence identity. Sequence identity is frequently measured in terms of percentage identity (or similarity or homology); the higher the percentage, the more similar the two sequences are. When a nucleic acid molecule is in RNA form, “T” is understood to be “U.” Thus, “T” and “U” are interchangeable for purposes of determining sequence identity.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Variants of protein and nucleic acid sequences known in the art and disclosed herein are typically characterized by possession of at least about 80%, at least 90%, at least 95%, at least 96%, at least 97%, at least 98% or at least 99% sequence identity counted over the full length alignment with the amino acid sequence using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or at least 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Single guide RNA (sgRNA): A synthetic guide RNA (gRNA) used to recognize a target DNA sequence and direct a Cas nuclease (such as Cas9) to a target sequence. sgRNAs typically include a targeting sequence and guide RNA scaffold (binding scaffold) for the Cas nuclease. In some examples, the sgRNAs are generated from subcloning an optimized mouse genome-wide lentiviral CRISPR sgRNA library, such as lentiCRISPRv2-Brie (Doench et al., Nat Biotechnol 34:184-191, 2016, herein incorporated by reference in its entirety). In some examples, a sgRNA expression cassette further comprises a U6 promoter and/or a guide RNA scaffold.

Short hairpin RNA (shRNA): A sequence of RNA that makes a tight hairpin turn and can be used to silence gene expression via the RNAi pathway. The shRNA hairpin structure is cleaved by cellular machinery into siRNA.

Small interfering RNA (siRNA): A double-stranded nucleic acid molecule that modulates gene expression through the RNAi pathway. siRNA molecules are generally 15 to 40 nucleotides in length, such as 20-30 or 20-25 nucleotides in length, with 0 to 5 (such as 2)-nucleotide overhangs on each 3′ end. However, siRNAs can also be blunt ended. Generally, one strand of a siRNA molecule is at least partially complementary to a target nucleic acid, such as a target mRNA. siRNAs are also referred to as “small inhibitory RNAs.” Exemplary sequences encoding siRNA targeting Brd9, Brd7, and Pbrm1 are provided as SEQ ID NO: 42, SEQ ID NO: 43, and SEQ ID NO: 44, respectively.

Small molecule inhibitor: A molecule, typically with a molecular weight less than about Daltons, or in some embodiments, less than about 500 Daltons, wherein the molecule is capable of modulating, to some measurable extent, an activity of a target molecule (such as stability or activity of a Brd7, Brd9, or Pbrm1 protein).

Stability (of a protein): The activity of a protein can be modulated by modifying protein stability. In the context of the present disclosure, the stability of a protein refers to the rate of turnover (e.g., degradation) of the protein in a cell, such as a Treg or CAR T cell. The half-life of a protein is directly correlated with stability of the protein—the greater the half-life of a protein the greater the stability of the protein. Stability of a protein can be effected by several factors, including mutations in the protein and external factors, such as the presence of proteases or elevated temperatures. Thus, an agent that “promotes stability” is an agent that inhibits degradation or the rate of degradation of a protein. In some examples, an agent that promotes stability of a protein is an agent that inhibits degradation of a protein by at least 10%, at least 20%, at least 30%, at least 40%, at least 50%, at least 60%, at least 70%, at least 80%, at least 90%, at least 100% or more compared to degradation of the protein in the absence of the agent. In other examples, an agent that promotes stability of a protein is an agent that increases half-life of the protein by at least 10%, at least 20%, at least 30%, at least 40%, at least 50%, at least 60%, at least 70%, at least 80%, at least 90%, at least 100% or more compared to half-life of the protein in the absence of the agent. In yet other examples, an agent that promotes stability of a protein is an agent leads to an increase in levels of the protein in a cell, such as an increase of at least 10%, at least 20%, at least 30%, at least 40%, at least 50%, at least 60%, at least 70%, at least 80%, at least 90%, at least 100% or more compared to levels of the protein in the absence of the agent.

Subject: Living multi-cellular vertebrate organisms, a category that includes human and non-human mammals, including but not limited to non-human primates, rodents, and the like. In specific examples disclosed herein, the subject is human.

T cell: A white blood cell (lymphocyte) that is an important mediator of the immune response. T cells include, but are not limited to, CD4⁺ T cells and CD8⁺ T cells. A CD4⁺ T cell is an immune cell that carries a marker on its surface known as “cluster of differentiation 4” (CD4). These cells, also known as helper T cells, help orchestrate the immune response, including antibody responses as well as killer T cell responses. CD8⁺ T cells carry the “cluster of differentiation 8” (CD8) marker.

Activated T cells can be detected by an increase in cell proliferation and/or expression of or secretion of one or more cytokines (such as IL-2, IL-4, IL-6, IFN-γ, or TNFα). Activation of CD8⁺ T cells can also be detected by an increase in cytolytic activity in response to an antigen.

A regulatory T (Treg) cell is a class of T cell that has a role in maintaining immune system homeostasis by suppressing over-reactive immune responses (Josefowicz et al. Annu. Rev. Immunol. 30, 531-564, 2012). Defects in Treg cells lead to autoimmune disorders and immunopathology, whereas certain tumors are enriched with Treg cells that suppress anti-tumor immune responses (Tanaka and Sakaguchi, Cell Res. 27, 109-118, 2017).

In some examples, a “modified T cell” is a T cell transduced or transformed with a heterologous nucleic acid (such as one or more of the nucleic acids or vectors disclosed herein) or expressing one or more heterologous proteins. The terms “modified T cell” and “transduced T cell” are used interchangeably in some examples herein. Similarly, a “modified Treg cell” is a Treg cell transduced or transformed with a heterologous nucleic acid (such as one or more of the nucleic acids or vectors disclosed herein) or expressing one or more heterologous proteins.

Therapeutically effective amount: The quantity of an agent (e.g., small molecule inhibitors, activators, gRNAs (e.g., sgRNAs), RNAi compositions, or expression vectors), that is sufficient to treat, reduce, and/or ameliorate the symptoms and/or underlying cause of a disease or pathological condition, such as cancer or an autoimmune disorder in a subject. In a specific non-limiting example, an effective amount is an amount sufficient to inhibit or reduce tumor growth in the subject. In another specific non-limiting example, an effective amount is an amount sufficient to inhibit or reduce inflammation in the subject.

In some examples, a therapeutically effective amount is the amount necessary to increase activity or expression of Brd7, Brd9, or Pbrm1 at least 10%, at least 20%, at least 30%, at least 40%, at least 50%, at least 60%, at least 70%, at least 80%, at least 90%, at least 100% or more compared to the activity or expression of a suitable control. In some examples, the therapeutically effective amount is the amount necessary to increase the amount of Brd7, Brd9, or Pbrm1 protein in a cell by at least 10%, at least 20%, at least 30%, at least 40%, at least 50%, at least 60%, at least 70%, at least 80%, at least 90%, at least 100% or more compared to a suitable control.

In some examples, a therapeutically effective amount is the amount necessary to reduce activity or expression of Brd7, Brd9, or Pbrm1 at least 10%, at least 20%, at least 30%, at least 40%, at least 50%, at least 60%, at least 70%, at least 80%, at least 90%, at least 100% or more compared to a suitable control. In some examples, the therapeutically effective amount is the amount necessary to reduce the amount of Brd7, Brd9, or Pbrm1 protein in a cell by at least 10%, at least 20%, at least 30%, at least 40%, at least 50%, at least 60%, at least 70%, at least 80%, at least 90%, at least 100% or more compared to a suitable control.

Transduced or Transformed: A transformed cell is a cell into which a nucleic acid molecule has been introduced by molecular biology techniques. As used herein, the terms transduction and transformation encompass all techniques by which a nucleic acid molecule might be introduced into such a cell, including transduction or transfection with viral vectors, the use of plasmid vectors, and introduction of DNA by electroporation, lipofection, and particle gun acceleration.

Tumor, neoplasia, malignancy or cancer: A neoplasm is an abnormal growth of tissue or cells which results from excessive cell division. Neoplastic growth can produce a tumor. The amount of a tumor in an individual is the “tumor burden” which can be measured as the number, volume, or weight of the tumor. A tumor that does not metastasize is referred to as “benign.” A tumor that invades the surrounding tissue and/or can metastasize is referred to as “malignant.” A “non-cancerous tissue” is a tissue from the same organ wherein the malignant neoplasm formed, but does not have the characteristic pathology of the neoplasm. Generally, noncancerous tissue appears histologically normal. A “normal tissue” is tissue from an organ, wherein the organ is not affected by cancer or another disease or disorder of that organ. A “cancer-free” subject has not been diagnosed with a cancer of that organ and does not have detectable cancer.

Exemplary tumors, such as cancers, that can be treated with the disclosed methods include solid tumors, such as breast carcinomas (e.g. lobular and duct carcinomas), sarcomas, carcinomas of the lung (e.g., non-small cell carcinoma, large cell carcinoma, squamous carcinoma, and adenocarcinoma), mesothelioma of the lung, colorectal adenocarcinoma, head and neck cancers, stomach carcinoma, prostatic adenocarcinoma, ovarian carcinoma (such as serous cystadenocarcinoma and mucinous cystadenocarcinoma), ovarian germ cell tumors, testicular carcinomas and germ cell tumors, pancreatic adenocarcinoma, biliary adenocarcinoma, hepatocellular carcinoma, bladder carcinoma (including, for instance, transitional cell carcinoma, adenocarcinoma, and squamous carcinoma), renal cell adenocarcinoma, endometrial carcinomas (including, e.g., adenocarcinomas and mixed Mullerian tumors (carcinosarcomas)), carcinomas of the endocervix, ectocervix, and vagina (such as adenocarcinoma and squamous carcinoma of each of same), tumors of the skin (e.g., squamous cell carcinoma, basal cell carcinoma, malignant melanoma, skin appendage tumors, Kaposi sarcoma, cutaneous lymphoma, skin adnexal tumors and various types of sarcomas and Merkel cell carcinoma), esophageal carcinoma, carcinomas of the nasopharynx and oropharynx (including squamous carcinoma and adenocarcinomas of same), salivary gland carcinomas, brain and central nervous system tumors (including, for example, tumors of glial, neuronal, and meningeal origin), tumors of peripheral nerve, soft tissue sarcomas and sarcomas of bone and cartilage, and lymphatic tumors (including B-cell and T-cell malignant lymphoma). In one example, the tumor is an adenocarcinoma. In one example the tumor is a glioblastoma.

The methods can also be used to treat liquid tumors, such as a lymphatic, white blood cell, or other type of leukemia. In a specific example, the tumor treated is a tumor of the blood, such as a leukemia (for example acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia (T-PLL), large granular lymphocytic leukemia, and adult T-cell leukemia), lymphomas (such as Hodgkin's lymphoma and non-Hodgkin's lymphoma), and myelomas).

Vector: A nucleic acid molecule that can be introduced into a host cell (for example, by transfection or transduction), thereby producing a transformed host cell. Recombinant DNA vectors are vectors having recombinant DNA. A vector can include nucleic acid sequences that permit it to replicate in a host cell, such as an origin of replication. A vector can also include one or more selectable marker genes and other known genetic elements or selectable markers (such as an antibiotic, such as puromycin, hygromycin, or a detectable marker such as GFP or other fluorophore).

One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). A replication deficient viral vector is a vector that requires complementation of one or more regions of the viral genome required for replication due to a deficiency in at least one replication-essential gene function.

In some embodiments, the vector is a lentivirus (such as an integration-deficient lentiviral vector) or adeno-associated viral (AAV) vector. Other exemplary viral vectors that can be used include polyoma, SV40, vaccinia virus, herpes viruses including HSV and EBV, Sindbis viruses, alphaviruses and retroviruses of avian, murine, and human origin, baculovirus (Autographa californica multinuclear polyhedrosis virus; AcMNPV) vectors, retrovirus vectors, orthopox vectors, avipox vectors, fowlpox vectors, capripox vectors, suipox vectors, adenoviral vectors, herpes virus vectors, alpha virus vectors, baculovirus vectors, Sindbis virus vectors, vaccinia virus vectors and poliovirus vectors. Specific exemplary vectors are poxvirus vectors such as vaccinia virus, fowlpox virus and a highly attenuated vaccinia virus (MVA), adenovirus, baculovirus and the like. Pox viruses of use include orthopox, suipox, avipox, and capripox virus. Orthopox include vaccinia, ectromelia, and raccoon pox. One example of an orthopox of use is vaccinia. Avipox includes fowlpox, canary pox and pigeon pox. Capripox include goatpox and sheeppox. In one example, the suipox is swinepox. Specific viral vectors that can be used include other DNA viruses such as herpes simplex virus and adenoviruses, and RNA viruses such as retroviruses and polio.

Certain vectors are capable of directing the expression of genes to which they are operatively-linked. Such vectors are referred to herein as “expression vectors.” Recombinant expression vectors can comprise a nucleic acid provided herein (such as a guide RNA or nucleic acid coding sequence) in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). A vector can be introduced into host cells to produce transcripts, proteins encoded by nucleic acids as described herein (e.g., nuclease, Brd7, Brd9, or Pbrm1).

### III. Overview of Several Embodiments

Disclosed herein are methods of treating an autoimmune disease or disorder, for example multiple sclerosis, in a subject. In some embodiments, a therapeutically effective amount of an agent that reduces expression or activity of Brd7 or Pbrm1 is administered to the subject. In other or additional embodiments, a therapeutically effective amount of an agent that increases expression or activity of Brd9 is administered to the subject. In some embodiments, Brd9, Brd7, and/or Pbrm1 expression is increased or reduced in a regulatory T cell (Treg) in the subject.

Also provided are methods of treating cancer, for example treating glioblastoma, in a subject. In some embodiments, a therapeutically effective amount of an agent that reduces expression or activity of Brd9 is administered to the subject. In other or additional embodiments, a therapeutically effective amount of an agent that increases expression or activity of Brd7 or Pbrm1 is administered to the subject. In some examples, Brd9, Brd7, and/or Pbrm1 expression is increased or reduced in a regulatory T cell (Treg) in the subject. In some embodiments, the agent is administered with an additional immunotherapy and the effective amount of the agent is an amount that enhances the additional immunotherapy.

Also provided are methods of increasing Treg suppressor activity, for example by reducing expression or activity of Brd7 and/or Pbrm1, or increasing expression or activity of Brd9 in a Treg cell. Methods of reducing Treg suppressor activity are also provided, for example by increasing expression or activity of Brd7 and/or Pbrm1, or reducing expression or activity of Brd9 in a Treg cell. In some examples, the Treg is in a subject, the method is performed in vivo, and the subject is administered a small molecule inhibitor, an RNAi, an activator, or an expression vector encoding Brd9, Brd7, or Pbrm1, respectively.

In any of the provided methods, the expression or activity of Brd9, Brd7, or Pbrm1, can be increased, for example through techniques such as contacting the cell with an activator or expression vector encoding Brd9, Brd7, or Pbrm1, respectively. The expression or activity of Brd9, Brd7, or Pbrm1, can also be reduced, for example through techniques such as genome editing, RNAi, or contacting the cell with a small molecule inhibitor of Brd9, Brd7, or Pbrm1, respectively.

### I. Methods of Treating an Autoimmune Disease or Disorder

Methods are provided herein for the treatment of subjects that have an autoimmune disease or disorder, such as multiple sclerosis. Although the treatment of multiple sclerosis is exemplified herein, any type of autoimmune disorder can be treated using the disclosed compositions and methods, e.g. rheumatoid arthritis, systemic lupus erythematosus, type 1 diabetes, multiple sclerosis, Sjögren's syndrome, Graves' disease, myasthenia gravis, ulcerative colitis, Hashimoto's thyroiditis, celiac disease, Crohn's disease, arthritis, inflammatory bowel disease, psoriasis, or scleroderma. In some examples the method reduces one or more symptoms of an autoimmune disease by at least 10%, at least 20%, at least 30%, at least 40%, at least 50%, at least 60%, at least 70%, at least 80%, at least 90%, or even 100%.

Disclosed herein are methods of treating a subject with an autoimmune disease or disorder, comprising administering a therapeutically effective amount of an agent. In some examples, the agent increases Foxp3 expression or activity in the subject. In some examples, the agent reduces activity of a PBAF complex or increases activity of a ncBAF complex in the subject. In some examples, the agent reduces expression or activity of Brd7, reduces expression or activity of Pbrm1, or increases expression or activity of Brd9, or combinations thereof, in the subject. The agent is not limited to any particular mode of action, and may modulate the expression or activity of Brd7, Pbrm1, or Brd9 by targeting a gene, mRNA, protein, or other target of which the result is an impact on the expression or activity of a Brd7, Pbrm1, or Brd9 gene, mRNA, or gene product (e.g., protein).

In some examples, the agent that reduces expression or activity of Brd7 or Pbrm1 is a small molecule inhibitor. Non-limiting examples of small molecule inhibitors of Brd7 include LP99, BI-7273, VZ-185. In some examples, the small molecule inhibitor is a degrader of Brd7, for example, VZ-185. Brd7 inhibitors are described, for example in Karim et al., J. Med. Chem. 2020, 63, 6, 3227-3237 and Hügle et al. J. Med. Chem. 2020, 63, 24, 15603, herein incorporated by reference in their entireties. Non-limiting examples of small molecule inhibitors of Pbrm1 include ACBI1, AU-15330, BRM014, and PFI-3. In some examples, the small molecule inhibitor is a degrader of Pbrm1, for example, ACBI1, AU-15330, BRM014, and PFI-3 (see, e.g., Xiao et al., (2022) Nature, 601: 434-439; and Papillon et al. (2018) Med. Chem. 61(22): 10155-10172). In some examples, administering the small molecule inhibitor reduces activity of Brd7 or Prbm1 by at least 10%, at least 20%, at least 30%, at least 40%, at least 50%, at least 60%, at least 70%, at least 80%, at least 90%, at least 100% or more relative to a suitable control (e.g., an untreated subject or a baseline reading of the same subject prior to treatment). In some examples, administering the small molecule inhibitor reduces protein levels of Brd7 or Prbm1 by at least 10%, at least 20%, at least 30%, at least 40%, at least 50%, at least 60%, at least 70%, at least 80%, at least 90%, at least 100% or more relative to a suitable control (e.g., an untreated subject or a baseline reading of the same subject prior to treatment). In some examples, administering the small molecule inhibitor reduces expression of Brd7 or Prbm1 by at least 10%, at least 20%, at least 30%, at least 40%, at least 50%, at least 60%, at least 70%, at least 80%, at least 90%, at least 100% or more relative to a suitable control (e.g., an untreated subject or a baseline reading of the same subject prior to treatment).

In some examples, the agent that reduces expression or activity of a Brd7 or Pbrm1 is an RNAi molecule targeting Brd7 or Pbrm1. RNAi generically refers to a cellular process that inhibits expression of genes. Molecules that inhibit gene expression through the RNAi pathway include siRNAs, miRNAs, and shRNAs. Further information regarding RNAi-based therapeutics can be found, for example, in Setten, et al. Nat Rev Drug Discov 18, 421-446 (2019). In other examples, the agent is a gRNA (e.g., sgRNA) and targets Brd7 or Pbrm1. In some examples the RNAi or gRNA target a sequence comprising at least 70%, at least 80%, at least 90%, at least 95%, or 100% sequence identity to a contiguous portion of SEQ ID NO: 31 or SEQ ID NO: 33, respectively. In some examples, the contiguous portion is 10-30 nucleotides in length, for example, 10-25 nucleotides, 10-20 nucleotides, 10-15 nucleotides, 15-30 nucleotides, 20-30 nucleotides, 25-30 nucleotides, 17-24 nucleotides, 18-15 nucleotides, or 20-25 nucleotides. In a specific, non-limiting example, the contiguous portion is 19-21 nucleotides.

In some examples, the siRNA targets Brd7 or Pbrm1, and has at least 70%, 80%, 90%, or 95% sequence identity to SEQ ID NO: 43 or SEQ ID NO: 44, respectively. In some examples, the siRNA targets Brd7 or Pbrm1, and comprises or consists of SEQ ID NO: 43 or SEQ ID NO: 44, respectively. In some examples, the gRNA targets Brd7 or Pbrm1, and has at least 70%, 80%, 90%, or 95% sequence identity to SEQ ID NO: 10 or SEQ ID NO: 8, respectively. In some examples, the gRNA consists of or comprises SEQ ID NO: 10 or SEQ ID NO: 8, respectively. In other examples, the gRNA targets Brd7 or Pbrm1, and has at least 70%, 80%, 90%, or 95% sequence identity to SEQ ID NO: 46 or SEQ ID NO: 47, respectively. In some examples, the gRNA consists of or comprises SEQ ID NO: 46 or SEQ ID NO: 47, respectively. In further examples, the gRNA targeting Brd7 or Pbrm1 is a sgRNA that has at least 70%, 80%, 90%, or 95% sequence identity to SEQ ID NO: 10, SEQ ID NO: 8, SEQ ID NO: 46, or SEQ ID NO: 47. In some examples, the sgRNA targeting Brd7 or Pbrm1 comprises SEQ ID NO: 10, SEQ ID NO: 8, SEQ ID NO: 46, or SEQ ID NO: 47.

In some examples, a vector comprises the RNAi or gRNA molecule targeting Brd7 or Pbrm1, which in some examples is operably linked to a promoter. The vector may facilitate transient expression of the RNAi or gRNA molecule in the subject or may facilitate chromosomal integration of the RNAi or gRNA molecule or expression cassette comprising the RNAi or gRNA for stable expression in the subject. In some embodiments, a target cell (e.g., Treg) expresses a Cas nuclease or is contacted with a Cas nuclease (e.g., Cas9, Cas13). In some examples, the vector further encodes a Cas nuclease. In other examples, an additional vector encodes a Cas nuclease.

In some examples, administering the RNAi or gRNA (e.g., sgRNA) molecule reduces expression of Brd7 or Prbm1 by at least 10%, at least 20%, at least 30%, at least 40%, at least 50%, at least 60%, at least 70%, at least 80%, at least 90%, at least 100% or more relative to a suitable control (e.g., an untreated subject, untreated cell, or a baseline reading of the same subject or cell prior to treatment). In some examples, administering the RNAi or gRNA molecule reduces protein levels of Brd7 or Prbm1 by at least 10%, at least 20%, at least 30%, at least 40%, at least 50%, at least 60%, at least 70%, at least 80%, at least 90%, at least 100% or more relative to a suitable control (e.g., an untreated subject, untreated cell, or a baseline reading of the same subject or cell prior to treatment).

In some examples, the agent that reduces expression or activity of a Brd7 or Pbrm1 is an agent that deletes all or a portion of a Brd7 or Pbrm1 gene. In some examples, the agent that deletes all or a portion of the Brd7 or Pbrm1 gene facilitates genome editing in the subject. For example, CRISPR and/or TALEN can be used for targeted genome editing. Methods of genome editing and targeted therapy for the treatment of human diseases is described, for example in Li et al., Sig Transduct Target Ther, 5, 1, 2020. In some examples, the agent that deletes all or a portion of a Brd7 or Pbrm1 comprises a gRNA (e.g., sgRNA) targeting a Brd7 or Pbrm1 gene, such as a sequence comprising at least 70%, at least 80%, at least 90%, at least 95%, or 100% sequence identity to a contiguous portion of SEQ ID NO: 31 or SEQ ID NO: 33, respectively. In some examples, the contiguous portion is 10-30 nucleotides in length, for example, 10-25 nucleotides, 10-20 nucleotides, 10-15 nucleotides, 15-30 nucleotides, 20-30 nucleotides, 25-30 nucleotides, 17-nucleotides, 18-15 nucleotides, or 20-25 nucleotides. In a specific, non-limiting example, the contiguous portion is 19-21 nucleotides. In some examples, the gRNA targets Brd7 or Pbrm1, and has at least 70%, 80%, 90%, or 95% sequence identity to SEQ ID NO: 10 or SEQ ID NO: 8, respectively. In some examples, the gRNA consists of or comprises SEQ ID NO: 10 or SEQ ID NO: 8, respectively. In some embodiments, a target cell (e.g., Treg) expresses a Cas nuclease or is contacted with a Cas nuclease (e.g., Cas9, Cas13).

In some examples, administering the agent that deletes all or a portion of Brd7 or Pbrm1 reduces expression of Brd7 or Prbm1, for example by at least 10%, at least 20%, at least 30%, at least 40%, at least 50%, at least 60%, at least 70%, at least 80%, at least 90%, at least 100% or more relative to a suitable control (e.g., an untreated subject, untreated cell, or a baseline reading of the same subject or cell prior to treatment). In some examples, administering the agent that deletes all or a portion of Brd7 or Pbrm1 reduces functional protein levels in the subject, for example reducing functional Brd7 or Prbm1 protein by at least 10%, at least 20%, at least 30%, at least 40%, at least 50%, at least 60%, at least 70%, at least 80%, at least 90%, at least 100% or more relative to a suitable control (e.g., an untreated subject, untreated cell, or a baseline reading of the same subject or cell prior to treatment).

In some examples, the agent that increases expression or activity of Brd9 is an activator. In some examples, the activator targeting Brd9 increases transcription of a Brd9 gene. For example, the Brd9 activator may increase transcription of Brd9 by at least 10%, at least 20%, at least 30%, at least 40%, at least 50%, at least 60%, at least 70%, at least 80%, at least 90%, at least 100%, or more relative to a control (e.g., an untreated subject, untreated cell, or a baseline reading of the same subject or cell prior to treatment). In some examples, the activator targeting Brd9 increases translation of Brd9 mRNA, thereby increasing levels of Brd9 gene product in the subject. For example, the activator may increase levels of Brd9 protein by at least 10%, at least 20%, at least 30%, at least 40%, at least 50%, at least 60%, at least 70%, at least 80%, at least 90%, at least 100%, or more relative to a suitable control (e.g., an untreated subject, untreated cell, or a baseline reading of the same subject or cell prior to treatment).

In further examples, the activator decreases degradation or increases protein stability of Brd9 mRNA or protein, thereby increasing the level of Brd9 gene product in the subject. For example, the activator may increase levels of a Brd9 gene product in the subject by at least 10%, at least 20%, at least 30%, at least 40%, at least 50%, at least 60%, at least 70%, at least 80%, at least 90%, at least 100%, or more relative to a suitable control (e.g., an untreated subject, untreated cell, or a baseline reading of the same subject or cell prior to treatment). In other examples, an activator targeting Brd9 increases activity or a function of Brd9 protein in the subject. In some examples, Brd9 activity is increased by at least 10%, at least 20%, at least 30%, at least 40%, at least 50%, at least 60%, at least 70%, at least 80%, at least 90%, at least 100%, or more relative to a suitable control (e.g., an untreated subject, untreated cell, or a baseline reading of the same subject or cell prior to treatment).

In some examples, administering the activator of Brd9 increases expression of Brd9 in the subject, for example by at least 10%, at least 20%, at least 30%, at least 40%, at least 50%, at least 60%, at least 70%, at least 80%, at least 90%, at least 100% or more relative to a suitable control (e.g., an untreated subject, untreated cell, or a baseline reading of the same subject or cell prior to treatment). In some examples, administering the agent that activates Brd9 increases protein levels in the subject, for example increasing Brd9 protein by at least 10%, at least 20%, at least 30%, at least 40%, at least 50%, at least 60%, at least 70%, at least 80%, at least 90%, at least 100% or more relative to a suitable control (e.g., an untreated subject, untreated cell, or a baseline reading of the same subject or cell prior to treatment).

In some examples, the agent that increases expression or activity of Brd9 is an expression vector encoding a Brd9 gene product (e.g., a vector encoding SEQ ID NO: 30 or an amino acid having at least 90%, or at least 95% identity to SEQ ID NO: 30). The vector may facilitate transient expression of the Brd9 gene product in the subject or may facilitate chromosomal integration of a nucleic acid molecule or expression cassette comprising a nucleic acid molecule encoding the Brd9 gene product for stable expression in the subject.

In some examples, administering the expression vector encoding Brd9 increases expression of Brd9, for example by at least 10%, at least 20%, at least 30%, at least 40%, at least 50%, at least 60%, at least 70%, at least 80%, at least 90%, at least 100% or more relative to a suitable control (e.g., an untreated subject, untreated cell, or a baseline reading of the same subject or cell prior to treatment). In some examples, administering the expression vector encoding Brd9 increases a Brd9 protein level in the subject, for example increasing Brd9 protein by at least 10%, at least 20%, at least 30%, at least 40%, at least 50%, at least 60%, at least 70%, at least 80%, at least 90%, at least 100% or more relative to a suitable control (e.g., an untreated subject, untreated cell, or a baseline reading of the same subject or cell prior to treatment). In some examples a coding sequence comprising at least 70%, at least 80%, at least 90%, at least 95%, or 100% sequence identity to SEQ ID NO: 29 is administered, which can be part of a vector, and may be operably linked to a promoter (such as a constitutive, inducible, or tissue specific promoter).

In other examples, the methods include administering to the subject the agent and a pharmaceutically acceptable carrier, such as buffered saline. A “pharmaceutically acceptable carrier” includes any and all solvents, dispersion media, coatings, antibacterial and antifungal agents, isotonic and absorption delaying agents, and the like, compatible with pharmaceutical administration (see, e.g., Remington: The Science and Practice of Pharmacy, The University of the Sciences in Philadelphia, Editor, Lippincott, Williams, & Wilkins, Philadelphia, PA, 21^{st} Edition, 2005). Examples of such carriers or diluents include, but are not limited to, water, saline, Ringer's solutions, dextrose solution, balanced salt solutions, and 5% human serum albumin. Liposomes and non-aqueous vehicles such as fixed oils may also be used. Supplementary active compounds can also be incorporated into the compositions. Actual methods for preparing administrable compositions include those provided in Remington: The Science and Practice of Pharmacy, The University of the Sciences in Philadelphia, Editor, Lippincott, Williams, & Wilkins, Philadelphia, PA, 21^{st} Edition (2005).

Administration of the agent can be local or systemic. Exemplary routes of administration include, but are not limited to, oral, injection (such as subcutaneous, intramuscular, intradermal, intraperitoneal, intravenous, intracranial, intracerebral, intrathecal, intraspinal), sublingual, rectal, transdermal (for example, topical), intranasal, vaginal, and inhalation routes. In some examples, the agent is injected or infused into an afflicted area (local administration). Appropriate routes of administration can be determined by a skilled clinician based on factors such as the subject, the condition being treated, and other factors.

Multiple doses of the agent can be administered to a subject. For example, the agent can be administered daily, every other day, twice per week, weekly, every other week, every three weeks, monthly, or less frequently. A skilled clinician can select an administration schedule based on the subject, the condition being treated, the previous treatment history, and other factors.

In some examples, the effective amount of agent, is an amount sufficient to prevent, treat, reduce, and/or ameliorate one or more signs or symptoms of an autoimmune disease or disorder in the subject. In a specific, non-limiting example, the effective amount is an amount sufficient to reduce inflammation in the subject. For example, reducing inflammation in the subject by at least 10%, at least 20%, at least 30%, at least 40%, at least 50%, at least 60%, at least 70%, at least 80%, at least 90%, at least 100%, or more as compared to a suitable control (e.g., an untreated subject or a baseline reading of the same subject prior to treatment).

In some examples, the autoimmune disease is multiple sclerosis and an effective amount of the agent is an amount that slows disease progression, such an amount that slows the rate of demyelination in the subject as compared to a suitable control (e.g., a baseline measurement from the same subject or comparison to a different subject not receiving the agent). In other examples, the effective amount of the agent is an amount that reduces a number of lesions detected by a magnetic resonance imaging (MRI) scan in the subject. For example, MRI detected lesions are reduced by at least about 10%, 20%, 30%, 40%, 50%, 60%, 70%, 80%, 90%, or by more than 95%, as compared to a baseline measurement for the same subject, or as compared to a suitable control (e.g., a subject receiving a placebo treatment or not receiving the agent). Similarly, in some examples, treatment with the agent, either alone or in combination with other additional treatments, reduces the average number of multiple sclerosis exacerbations per subject in a given period (e.g., 6, 12, 18 or 24 months) by at least about 10%, 20%, 30%, 40%, 50%, 60%, 70%, 80%, 90%, or by more than 95%. The control subjects can be untreated subject, or subjects not receiving the agent (e.g., subjects receiving other agents or alternative therapies). Treatment with the agent, alone or in combination with other agents, can also reduce the average rate of increase in the subject's disability score over some period (e.g., 6, 12, 18 or 24 months), for example, as measured by an Expanded Disability Status Scale (EDSS) score, by at least about 10% or about 20%, such as by at least about 30%, 40%, or 50%. In one embodiment, the reduction in the average rate of increase in the EDSS score is at least about 60%, at least about 75%, or at least about 90%, or can even lead to actual improvement in the disability score compared to control subjects, such as untreated subjects or subjects not receiving the agent, but possibly receiving other therapeutics.

In some examples, the effective amount reduces expression or activity of Brd7 or Pbrm1. For example, reducing gene expression of Brd7 or Pbrm1 at least 10%, at least 20%, at least 30%, at least 40%, at least 50%, at least 60%, at least 70%, at least 80%, at least 90%, or 100%, as compared to the activity or expression of Brd7 or Pbrm1, respectively, in a suitable control. Reducing gene expression is typically measured by mRNA levels, thus modes of action that target transcriptional regulation as well as post-transcriptional regulation (e.g., decreasing mRNA transcript stability) may be used to reduce the expression of a particular gene. In specific examples, the therapeutically effective amount is the amount necessary to reduce the amount of Brd7 or Pbrm1 protein by at least 10%, at least 20%, at least 30%, at least 40%, at least 50%, at least 60%, at least 70%, at least 80%, at least 90%, at least 100% or more as compared to the amount of Brd7 or Pbrm1 in a suitable control (e.g., an untreated subject, untreated cell, or a baseline reading of the same subject or cell prior to treatment).

In some examples, the effective amount increases expression or activity of Brd9. For example, increasing gene expression of Brd9 at least 10%, at least 20%, at least 30%, at least 40%, at least 50%, at least 60%, at least 70%, at least 80%, at least 90%, at least 100% or more compared as compared the activity or expression of Brd9 in a suitable control. Increasing gene expression is typically measured by mRNA levels, thus modes of action that target transcriptional regulation as well as post-transcriptional regulation (e.g., increasing mRNA transcript stability) may be used to increase the expression of a particular gene. In other examples, the therapeutically effective amount is the amount necessary to increase the amount of Brd9 protein by at least 10%, at least 20%, at least 30%, at least 40%, at least 50%, at least 60%, at least 70%, at least 80%, at least 90%, at least 100% or more compared to the amount of protein in a suitable control (e.g., an untreated subject, untreated cell, or a baseline reading of the same subject or cell prior to treatment).

In some examples, the agent reduces expression or activity of Brd7, reduces expression or activity of Pbrm1, increases expression or activity of Brd9, or combinations thereof, in Treg cells in the subject. In some examples, reducing the expression or activity of Brd7, reducing the expression or activity of Pbrm1, increasing the expression or activity of Brd9, or combinations thereof, in Treg cells increases Treg immunosuppressive activity, thereby reducing autoimmune responses in the subject.

In some examples, the subject receives an additional treatment, such as one or more of an anti-inflammatory, humanized monoclonal antibody (e.g., ocrelizumab), beta interferon (e.g., Avonex (interferon beta 1a), Rebif (interferon beta 1a), Plegridy (peginterferon beta 1a), Betaferon (interferon beta 1b), Extavia (interferon beta 1b)), I1-17 inhibitors (e.g. Secukinumab, Ixekizumab, Brodalumab) or cell migration inhibitors (e.g., Natalizumab, Fingolimod). In specific non-limiting examples, the additional treatment is a corticosteroid (e.g., prednisone or methylprednisolone), Glatiramer acetate, Fingolimod, Dimethyl fumarate, Diroximel fumarate, Teriflunomide, Siponimod, Cladribine, Ocrelizumab, Natalizumab, and/or Alemtuzumab. Such additional treatments can be administered before, after, or concurrently with the agent that reduces expression or activity of Brd7, the agent that reduces expression or activity of Pbrm1, the agent that increases expression or activity of Brd9 (or combinations of such agents).

### II. Methods of Treating Cancer or Enhancing Cancer Immunotherapy

Cancers, including glioblastoma, secrete numerous regulatory T cell (Treg)-inducing cytokines that promote tumor proliferation and immune escape. Thus, the strategic modulation of Treg activity in glioblastoma patients (as well patients with other types of cancer) present opportunity for more effective immunotherapy. Tumors of the central nervous system often affect “immunologically privileged” tissue, underscoring a need to develop new therapies to augment host immune responses to such tumors, including malignant glioblastoma. In some examples the method reduces one or more symptoms of a tumor (such as the size of a tumor, volume of a tumor, and/or a number of tumors) by at least 10%, at least 20%, at least 30%, at least 40%, at least 50%, at least 60%, at least 70%, at least 80%, at least 90%, or even 100%, for example relative to an amount before treatment with the methods provided herein. In some examples the method reduces the size of a metastasis, volume of a metastasis, and/or a number of metastasis by at least 10%, at least 20%, at least 30%, at least 40%, at least 50%, at least 60%, at least 70%, at least 80%, at least 90%, or even 100%, for example relative to an amount before treatment with the methods provided herein. In some examples, combinations of these effects are achieved.

Glioblastoma multiforme is the most common and aggressive type of primary brain tumor. Other common malignant gliomas include anaplastic gliomas, including anaplastic astrocytomas. Patients with glioblastoma have a median survival of approximately 15 months. In addition, low-grade gliomas often progress to more malignant gliomas when they recur. No current treatment is curative because these tumors tend to grow aggressively and invasively in sensitive areas of the brain. The current treatment standard is chemotherapy with temozolomide (TMZ) combined with radiotherapy, which has demonstrated limited prolongation of survival. In some examples, the methods provided herein are used to treat anaplastic glioma, such as anaplastic astrocytoma.

Although the treatment of glioma is exemplified herein, any type of cancer can be treated using the disclosed compositions and methods. Both hematological and solid cancers can be treated. Thus, in some embodiments, the hematological (or hematogenous) cancer treated with the methods provided herein is a leukemia, such as lymphoma, Hodgkin's disease, non-Hodgkin's lymphoma (indolent or high grade forms), multiple myeloma, Waldenstrom's macroglobulinemia, heavy chain disease, myelodysplastic syndrome, hairy cell leukemia or myelodysplasia. In some cases, lymphomas are considered solid tumors.

In some embodiments, the cancer treated with the methods provided herein is a solid tumor. Solid tumors can be benign or malignant. Examples of solid tumors, such as sarcomas and carcinomas, that can be treated with the methods provided herein include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, head and neck cancers, neuroblastoma, retinoblastoma and brain metastasis.

Disclosed herein are methods of treating a subject with cancer which include administering a therapeutically effective amount of an agent. In some examples, the agent reduces Foxp3 expression or activity in the subject. In some examples, the agent reduces activity of an ncBAF complex or increases activity of a PBAF complex in the subject. In some examples, the subject is administered a therapeutically effective amount of an agent that reduces expression or activity of Brd9 in the subject, a therapeutically effective amount of an agent that increases expression or activity of Brd7, a therapeutically effective amount of an agent that increases expression or activity of Pbrm1, or combinations thereof, in the subject. The agent is not limited to any particular mode of action and may modulate the expression or activity of Brd7, Pbrm1, or Brd9 by targeting a gene, mRNA, protein, or other target of which the result is an impact on the expression or activity of a Brd7, Pbrm1, or Brd9 gene, mRNA, or gene product (e.g., protein).

In some examples, the agent that reduces expression or activity of a Brd9 is a small molecule inhibitor, for example, I-BRD9, LP99, BI-7273, BI-9564, VZ-185, dBRD9, dBRD9-A (see, e.g., Martin et al., (2020) Med. Chem., 63(6): 3227-3237). In a non-limiting example, the agent that reduces expression or activity of a Brd9 is the small molecule inhibitor dBRD9 or dBRD9-A.

In some examples, administering the small molecule inhibitor reduces activity of Brd9 by at least 10%, at least 20%, at least 30%, at least 40%, at least 50%, at least 60%, at least 70%, at least 80%, at least 90%, at least 100% or more relative to a suitable control (e.g., an untreated subject, untreated cell, or a baseline reading of the same subject or cell prior to treatment). In some examples, administering the small molecule inhibitor reduces protein levels of Brd9 by at least 10%, at least 20%, at least 30%, at least 40%, at least 50%, at least 60%, at least 70%, at least 80%, at least 90%, at least 100% or more relative to a suitable control (e.g., an untreated subject, untreated cell, or a baseline reading of the same subject or cell prior to treatment). In some examples, administering the small molecule inhibitor reduces expression of Brd9 by at least 10%, at least 20%, at least 30%, at least 40%, at least 50%, at least 60%, at least 70%, at least 80%, at least 90%, at least 100% or more relative to a suitable control (e.g., an untreated subject, untreated cell, or a baseline reading of the same subject or cell prior to treatment).

In some examples, the agent that reduces expression or activity of a Brd9 is an RNAi molecule targeting Brd9. RNAi generically refers to a cellular process that inhibits expression of genes. Molecules that inhibit gene expression through the RNAi pathway include siRNAs, miRNAs, and shRNAs. Further information regarding RNAi-based therapeutics can be found, for example, in Setten, et al. Nat Rev Drug Discov 18, 421-446 (2019). In other examples, the agent is a gRNA (e.g., sgRNA) that targets Brd9. In some examples the RNAi or gRNA target a sequence comprising at least 70%, at least 80%, at least 90%, at least 95%, or 100% sequence identity to a contiguous portion of SEQ ID NO: 29. In some examples, the contiguous portion is 10-30 nucleotides in length, for example, 10-25 nucleotides, 10-20 nucleotides, 10-15 nucleotides, 15-30 nucleotides, 20-30 nucleotides, 25-30 nucleotides, 17-24 nucleotides, 18-15 nucleotides, or 20-25 nucleotides. In a specific, non-limiting example, the contiguous portion is 19-21 nucleotides. In some examples, the gRNA targets a sequence comprising at least 70%, at least 80%, at least 90%, at least 95%, or 100% sequence identity to a contiguous portion of SEQ ID NO: 29.

In some examples, the siRNA targets Brd9 and has at least 70%, 80%, 90%, or 95% sequence identity to SEQ ID NO: 42. In some examples, the siRNA targets Brd9 and comprises or consists of SEQ ID NO: 42. In some examples, the gRNA targets Brd9 and has at least 70%, 80%, 90%, or 95% sequence identity to SEQ ID NO: 12. In some examples, the gRNA comprises or consists of SEQ ID NO: 12. In other examples, the gRNA targets Brd9 and has at least 70%, 80%, 90%, or 95% sequence identity to SEQ ID NO: 45. In some examples, the gRNA consists of or comprises SEQ ID NO: 45. In further examples, the gRNA targeting Brd9 is a sgRNA that has at least 70%, 80%, 90%, or 95% sequence identity to SEQ ID NO: 12 or SEQ ID NO: 45. In some examples, the sgRNA targeting Brd9 comprises SEQ ID NO: 12 or SEQ ID NO: 45.

In some examples, a vector includes the RNAi or gRNA molecule targeting Brd9, which may be operably linked to a promoter (such as a constitutive, inducible, or tissue-specific promoter). The vector may facilitate transient expression of the RNAi or gRNA molecule in the subject and/or may facilitate chromosomal integration of the RNAi or gRNA molecule or expression cassette comprising the RNAi or gRNA for stable expression in the subject. In some embodiments, a target cell (e.g., Treg) expresses a Cas nuclease or is contacted with a Cas nuclease (e.g., Cas9, Cas13). In some examples, the vector further encodes a Cas nuclease. In other examples, an additional vector encodes a Cas nuclease.

In some examples, administering the RNAi or gRNA molecule reduces expression of Brd9 by at least 10%, at least 20%, at least 30%, at least 40%, at least 50%, at least 60%, at least 70%, at least 80%, at least 90%, at least 100% or more relative to a suitable control (e.g., an untreated subject, untreated cell, or a baseline reading of the same subject or cell prior to treatment). In some examples, administering the RNAi or gRNA molecule reduces protein levels or accumulation Brd9 by at least 10%, at least 20%, at least 30%, at least 40%, at least 50%, at least 60%, at least 70%, at least 80%, at least 90%, at least 100% or more relative to a suitable control (e.g., an untreated subject, untreated cell, or a baseline reading of the same subject or cell prior to treatment).

In some examples, the agent that reduces expression or activity of a Brd9 is an agent that deletes all or a portion of a Brd9 gene. In some examples, the agent that deletes all or a portion of the Brd9 gene facilitates genome editing in the subject. For example, CRISPR and/or TALEN can be used for targeted genome editing. Methods of genome editing and targeted therapy of human diseases is described, for example in Li et al., Sig Transduct Target Ther, 5, 1, 2020. For example, the agent may include a gRNA molecule targeting a Brd9 gene, for example targets a sequence comprising at least 70%, at least 80%, at least 90%, at least 95%, or 100% sequence identity to a contiguous portion of SEQ ID NO: 29. In some examples, the contiguous portion is 10-30 nucleotides in length, for example, 10-25 nucleotides, 10-20 nucleotides, 10-15 nucleotides, 15-30 nucleotides, 20-30 nucleotides, 25-30 nucleotides, 17-24 nucleotides, 18-15 nucleotides, or 20-25 nucleotides. In a specific, non-limiting example, the contiguous portion is 19-21 nucleotides. In some examples, the gRNA targets Brd9 and has at least 70%, 80%, 90%, or 95% sequence identity to SEQ ID NO: 12. In some examples, the gRNA consists of or comprises SEQ ID NO: 12. In some embodiments, a target cell (e.g., Treg) expresses a Cas nuclease or is contacted with a Cas nuclease (e.g., Cas9, Cas13).

In some examples, administering the agent that deletes all or a portion of Brd9 reduces expression of Brd9, for example by at least 10%, at least 20%, at least 30%, at least 40%, at least 50%, at least 60%, at least 70%, at least 80%, at least 90%, at least 100% or more relative to a suitable control (e.g., an untreated subject, untreated cell, or a baseline reading of the same subject or cell prior to treatment t). In some examples, administering the agent that deletes all or a portion of Brd9 reduces functional protein levels in the subject, for example reducing functional Brd9 protein by at least 10%, at least 20%, at least 30%, at least 40%, at least 50%, at least 60%, at least 70%, at least 80%, at least 90%, at least 100% or more relative to a suitable control (e.g., an untreated subject, untreated cell, or a baseline reading of the same subject or cell prior to treatment).

In some examples, the agent that increases expression or activity of Brd7 or the agent that increases expression or activity of Pbrm1 gene is an activator. In some examples, the activator targeting Brd7 or Pbrm1 increases transcription of a Brd7 or Pbrm1 gene, respectively. For example, the activator can increase transcription of Brd7 or Pbrm1 gene by at least 10%, at least 20%, at least 30%, at least 40%, at least 50%, at least 60%, at least 70%, at least 80%, at least 90%, at least 100%, or more relative to a control (e.g., an untreated subject, untreated cell, or a baseline reading of the same subject or cell prior to treatment). In some examples, the activator targeting Brd7 or Pbrm1 increases translation of Brd7 or Pbrm1 mRNA, respectively, thereby increasing levels of a respective protein product in the subject. For example, the activator can increase levels of a gene product (such as a Brd7 or Pbrm1 gene product) by at least 10%, at least 20%, at least 30%, at least 40%, at least 50%, at least 60%, at least 70%, at least 80%, at least 90%, at least 100%, or more relative to a suitable control (e.g., an untreated subject, untreated cell, or a baseline reading of the same subject or cell prior to treatment).

In further examples, the activator decreases degradation or increases protein stability of Brd7 or Pbrm1 mRNA or protein, thereby increasing the level of Brd7 or Pbrm1 protein, respectively. For example, the activator can increase levels of a gene product (such as a Brd7 or Pbrm1 gene product) by at least 10%, at least 20%, at least 30%, at least 40%, at least 50%, at least 60%, at least 70%, at least 80%, at least 90%, at least 100%, or more relative to a suitable control (e.g., an untreated subject, untreated cell, or a baseline reading of the same subject or cell prior to treatment). In other examples, an activator targeting Brd7 or Pbrm1 increases activity or a function of Brd7 or Pbrm1 protein, respectively. In some embodiments, Brd7 or Pbrm1 activity is increased by at least 10%, at least 20%, at least 30%, at least 40%, at least 50%, at least 60%, at least 70%, at least 80%, at least 90%, at least 100%, or more relative to a suitable control (e.g., an untreated subject, untreated cell, or a baseline reading of the same subject or cell prior to treatment).

In some examples, the activator of Brd7 or Pbrm1 increases expression of Brd7 or Prbm1, for example by at least 10%, at least 20%, at least 30%, at least 40%, at least 50%, at least 60%, at least 70%, at least 80%, at least 90%, at least 100% or more relative to a suitable control (e.g., an untreated subject, untreated cell, or a baseline reading of the same subject or cell prior to treatment). In some examples, the activator of Brd7 or Pbrm1 increases protein levels in the subject, for example increasing Brd7 or Prbm1 protein by at least 10%, at least 20%, at least 30%, at least 40%, at least 50%, at least 60%, at least 70%, at least 80%, at least 90%, at least 100% or more relative to a suitable control (e.g., an untreated subject, untreated cell, or a baseline reading of the same subject or cell prior to treatment).

In some examples, the agent that increases expression or activity of Brd7 or Pbrm1 gene is an expression vector encoding a Brd7 or Pbrm1 gene product (e.g., a vector encoding SEQ ID NOs: or 34, or an amino acid sequence having at least 90% or at least 95% identity to SEQ ID NOs: 32 or 34). The vector may facilitate transient expression of a Brd7 or Pbrm1 gene product in the subject or may facilitate chromosomal integration of a nucleic acid molecule or expression cassette comprising a nucleic acid molecule encoding the Brd7 or Pbrm1 gene product for stable expression in the subject. In some examples a coding sequence comprising at least 70%, at least 80%, at least 90%, at least 95%, or 100% sequence identity to SEQ ID NO: 31 or SEQ ID NO: 33 is administered, which can be part of a vector, and which can be operably linked to a promoter (such as a constitutive, inducible, or tissue-specific promoter).

In some examples, administering the expression vector encoding Brd7 or Pbrm1 increases expression of Brd7 or Pbrm1, respectively, for example by at least 10%, at least 20%, at least 30%, at least 40%, at least 50%, at least 60%, at least 70%, at least 80%, at least 90%, at least 100% or more relative to a suitable control (e.g., an untreated subject, untreated cell, or a baseline reading of the same subject or cell prior to treatment). In some examples, administering the expression vector encoding Brd7 or Pbrm1 increases a Brd7 or Pbrm1 protein level, respectively, in the subject, for example increasing Brd7 or Pbrm1 protein by at least 10%, at least 20%, at least 30%, at least 40%, at least 50%, at least 60%, at least 70%, at least 80%, at least 90%, at least 100% or more relative to a suitable control (e.g., an untreated subject, untreated cell, or a baseline reading of the same subject or cell prior to treatment).

In other examples, the methods include administering to the subject the agent and a pharmaceutically acceptable carrier, such as buffered saline.

Administration of the agent can be local or systemic. Exemplary routes of administration include, but are not limited to, oral, injection (such as subcutaneous, intramuscular, intradermal, intraperitoneal, intravenous, intracranial, intracerebral, intrathecal, intraspinal), sublingual, rectal, transdermal (for example, topical), intranasal, vaginal, and inhalation routes. In some examples, the agent is injected or infused into a tumor, or close to a tumor (local administration), or administered to the peritoneal cavity. Appropriate routes of administration can be determined by a skilled clinician based on factors such as the subject, the condition being treated, and other factors.

Multiple doses of the agent can be administered to a subject. For example, the agent can be administered daily, every other day, twice per week, weekly, every other week, every three weeks, monthly, or less frequently. A skilled clinician can select an administration schedule based on the subject, the condition being treated, the previous treatment history, and other factors.

In some examples, the effective amount of agent, is an amount sufficient to prevent, treat, reduce, and/or ameliorate one or more signs or symptoms of cancer in the subject. For example, an amount sufficient to reduce tumor size or tumor load in the subject by at least 10%, at least 20%, at least 30%, at least 40%, at least 50%, at least 60%, at least 70%, at least 80%, at least 90%, at least 100%, or more, as compared to a baseline measurement for the same subject, or a suitable control. In some examples, the effective amount is an amount sufficient to inhibit or slow metastasis in the subject. For example, by decreasing tumor spread in the subject by at least 10%, at least 20%, at least 30%, at least 40%, at least 50%, at least 60%, at least 70%, at least 80%, at least 90%, at least 100%, or more, as compared to a suitable control (e.g., an untreated subject or a baseline reading of the same subject prior to treatment). In some examples, the effective amount is an amount that increases life expectancy of the subject, for example, by at least 10%, at least 20%, at least 30%, at least 40%, at least 50%, at least 60%, at least 70%, at least 80%, at least 90%, at least 100%, at least 200%, or more. The control subjects can be untreated subject, subjects not receiving the agent (e.g., subjects receiving other agents or alternative therapies).

In some examples, the effective amount reduces expression or activity of Brd9. For example, reducing gene expression of Brd9 by at least 10%, at least 20%, at least 30%, at least 40%, at least 50%, at least 60%, at least 70%, at least 80%, at least 90%, or 100%, as compared to the activity or expression of Brd9 in a suitable control (e.g., a subject not receiving treatment, or not receiving the agent but receiving an alternative therapeutic). Reducing gene expression is typically measured by mRNA levels, thus modes of action that target transcriptional regulation as well as post-transcriptional regulation (e.g. decreasing mRNA transcript stability) may be used to reduce the expression of a particular gene. In specific examples, the therapeutically effective amount is the amount necessary to reduce the amount of Brd9 protein by at least 10%, at least 20%, at least 30%, at least 40%, at least 50%, at least 60%, at least 70%, at least 80%, at least 90%, at least 100% or more as compared to the amount of Brd9 protein in a suitable control (e.g. an untreated subject or a baseline reading of the same subject prior to treatment).

In other examples, the therapeutically effective amount of the agent that increases the expression or activity of Brd7 or the therapeutically effective amount of the agent that increases the expression or activity of Pbrm1. For example, increasing gene expression of Brd7 and/or Pbrm1 at least 10%, at least 20%, at least 30%, at least 40%, at least 50%, at least 60%, at least 70%, at least 80%, at least 90%, at least 100% or more compared as compared the activity or expression of Brd7 and/or Pbrm1 in a suitable control (e.g., an untreated subject, untreated cell, or a baseline reading of the same subject or cell prior to treatment. Increasing gene expression can be measured by mRNA levels, thus modes of action that target transcriptional regulation as well as post-transcriptional regulation (e.g., increasing mRNA transcript stability) may be used to increase the expression of a particular gene. In other examples, the therapeutically effective amount is the amount necessary to increase the amount of Brd7 or Pbrm1 protein by at least 10%, at least 20%, at least 30%, at least 40%, at least 50%, at least 60%, at least 70%, at least 80%, at least 90%, at least 100% or more compared to the amount of protein in a suitable control (e.g., an untreated subject, untreated cell, or a baseline reading of the same subject or cell prior to treatment).

In some examples, the effective amount is an amount that enhances an additional therapy, such as an additional immunotherapy (e.g., monoclonal antibody, a chimeric antigen receptor (CAR)-expressing T cell, an immunotoxin, or an anti-tumor vaccine). For example, an amount sufficient that when administered with an additional immunotherapy, reduces tumor size or tumor load in the subject by at least 10%, at least 20%, at least 30%, at least 40%, at least 50%, at least 60%, at least 70%, at least 80%, at least 90%, at least 100%, or more, as compared to a suitable control (e.g., a subject not receiving the combination treatment). In some examples, the effective amount to enhance immunotherapy is an amount sufficient to inhibit or slow metastasis in the subject. For example, by decreasing tumor spread in the subject by at least 10%, at least 20%, at least 30%, at least 40%, at least 50%, at least 60%, at least 70%, at least 80%, at least 90%, at least 100%, or more, as compared to a suitable control. In some examples, the effective amount to enhance immunotherapy is an amount that increases life expectancy of the subject, for example, by at least 10%, at least 20%, at least 30%, at least 40%, at least 50%, at least 60%, at least 70%, at least 80%, at least 90%, at least 100%, at least 200%, or more. The control subjects can be untreated subject, or subjects not receiving the agent (e.g., subjects receiving other agents or alternative therapies), or subjects not receiving a combination treatment included the agent.

In further examples, the agent reduces expression or activity of Brd9, increases expression or activity of Brd7, increases expression or activity of Pbrm1, or combinations thereof, in Treg cells in the subject. In some examples, reducing the expression or activity of Brd9, increasing the expression or activity of Brd7 or increasing expression or activity of Pbrm1, or combinations thereof in Treg cells decreases Treg mediated immunosuppressive activity.

In some examples, the effective amount is an amount of agent that reduces expression or activity of Brd9, increases expression or activity of Brd7, increases expression or activity of Pbrm1, or combinations thereof, relative to a suitable control (e.g., an untreated subject, untreated cell, or a baseline reading of the same subject or cell prior to treatment). In specific, non-limiting examples, the effective amount is an amount that reduces expression or activity of Brd9, increases the expression or activity of Brd7, increases the expression or activity of Pbrm1, or combinations thereof, in a T regulatory cell (Treg) in the subject as compared to a suitable control (e.g., an untreated subject, untreated cell, or a baseline reading of the same subject or cell prior to treatment). In some examples, the subject has a cancer that secretes Treg-inducing cytokines, e.g. TGF-β and/or IL-10. In a specific non-limiting example, the subject has glioblastoma. In a specific non-limiting example, the subject has melanoma. In a specific non-limiting example, the subject has =non-small cell lung cancer.

In some examples, the subject receives an additional treatment, such as one or more of surgery, radiation, chemotherapy, immunotherapy, or other therapeutic. Exemplary chemotherapeutic agents include (but are not limited to) alkylating agents, such as nitrogen mustards (such as mechlorethamine, cyclophosphamide, melphalan, uracil mustard or chlorambucil), alkyl sulfonates (such as busulfan), nitrosoureas (such as carmustine, lomustine, semustine, streptozocin, or dacarbazine); antimetabolites such as folic acid analogs (such as methotrexate), pyrimidine analogs (such as 5-FU or cytarabine), and purine analogs, such as mercaptopurine or thioguanine; or natural products, for example vinca alkaloids (such as vinblastine, vincristine, or vindesine), epipodophyllotoxins (such as etoposide or teniposide), antibiotics (such as dactinomycin, daunorubicin, doxorubicin, bleomycin, plicamycin, or mitocycin C), and enzymes (such as L-asparaginase). Additional agents include platinum coordination complexes (such as cis-diamine-dichloroplatinum II, also known as cisplatin), substituted ureas (such as hydroxyurea), methyl hydrazine derivatives (such as procarbazine), and adrenocrotical suppressants (such as mitotane and aminoglutethimide); hormones and antagonists, such as adrenocorticosteroids (such as prednisone), progestins (such as hydroxyprogesterone caproate, medroxyprogesterone acetate, and magestrol acetate), estrogens (such as diethylstilbestrol and ethinyl estradiol), antiestrogens (such as tamoxifen), and androgens (such as testosterone proprionate and fluoxymesterone). Examples of the most commonly used chemotherapy drugs include adriamycin, melphalan (Alkeran®) Ara-C (cytarabine), carmustine, busulfan, lomustine, carboplatinum, cisplatinum, cyclophosphamide (Cytoxan®), daunorubicin, dacarbazine, 5-fluorouracil, fludarabine, hydroxyurea, idarubicin, ifosfamide, methotrexate, mithramycin, mitomycin, mitoxantrone, nitrogen mustard, paclitaxel (or other taxanes, such as docetaxel), vinblastine, vincristine, VP-16, while newer drugs include gemcitabine (Gemzar®), trastuzumab (Herceptin®), irinotecan (CPT-11), leustatin, navelbine, rituximab (Rituxan®) imatinib (STI-571), Topotecan (Hycamtin®), capecitabine, ibritumomab (Zevalin®), and calcitriol. A skilled clinician can select appropriate additional therapies (from those listed here or other current therapies) for the subject, depending on factors such as the subject, the cancer being treated, treatment history, and other factors.

In some examples, the additional therapeutic is a cell cycle or checkpoint inhibitor. In some examples, the checkpoint inhibitor targets PD-1, PD-L1, CTLA-4, CDK4, and/or CDK6. Exemplary inhibitors include ipilimumab, nivolumab, pembrolizumab, atezolizumab, avelumab, durvalumab, cemiplimab, palbociclib, ribociclib, and abemaciclib.

In some examples, the additional treatment is immunotherapy and comprises administering to the subject a monoclonal antibody, a chimeric antigen receptor (CAR)-expressing T cell, an immunotoxin, or an anti-tumor vaccine. In some examples, the subject is administered an effective amount of the agent and an additional immunotherapy, and the effective amount of the agent is an amount that enhances the additional immunotherapy (e.g., synergistic).

Such additional treatments can be administered before, after, or concurrently with the agent that increases expression or activity of Brd7, the agent that increases expression or activity of Pbrm1, the agent that decreases expression or activity of Brd9 (or combinations of such agents).

### III. Methods of Modulating Treg Activity

### Methods of Increasing Treg Suppressor Activity

Disclosed herein are methods of increasing Treg suppressor activity, for example, by at least 10%, at least 20%, at least 30%, at least 40%, at least 50%, at least 60%, at least 70%, at least 80%, at least 90%, at least 100%, at least 200%, or more, for example relative to an amount of such activity prior to treatment with the disclosed methods. In some examples, the method includes increasing Foxp3 expression in a Treg cell. In some examples, Foxp3 expression or activity is increased by increasing activity of an ncBAF complex or reducing activity of a PBAF complex in a Treg cell. In some examples, Treg suppressor activity is increased by increasing expression or activity of Brd9, reducing expression or activity of Brd7, reducing expression or activity of Pbrm1, or combinations thereof, in a Treg cell. The expression or activity of Brd9, Brd7, or Pbrm1 may refer to a Brd9, Brd7, or Pbrm1 gene, mRNA, or gene product (e.g., protein), respectively.

In some examples, Brd7 expression or activity, Pbrm1 expression or activity, or both, is reduced in a Treg cell by contacting the Treg cell with a small molecule inhibitor targeting Brd7, a small molecule inhibitor targeting Pbrm1, or both, respectively. Non-limiting examples of Brd7 inhibitors include LP99, BI-7273, VZ-185. In some examples, the small molecule inhibitor is a degrader of Brd7, for example, VZ-185. Brd7 inhibitors have been previously described, for example in Karim et al., J. Med. Chem. 2020, 63, 6, 3227-3237 and Hügle et al. J. Med. Chem. 2020, 63, 24, 15603, herein incorporated by reference in their entirety. Non-limiting examples of Pbrm1 inhibitors include ACBI1, AU-15330, BRM014, and PFI-3. In some examples, the small molecule inhibitor is a degrader of Pbrm1, for example, ACBI1, AU-15330, BRM014, and PFI-3 (see, e.g., Xiao et al., (2022) Nature, 601: 434-439; and Papillon et al. (2018) Med. Chem. 61(22): 10155-10172).

In some examples, Brd7 or Pbrm1 expression or activity is reduced in a Treg cell by silencing expression of Brd7 or Pbrm1 in the Treg cell, respectively. For example, by delivering an RNAi molecule targeting Brd7 or Pbrm1 to the Treg cell. RNAi generically refers to a cellular process that inhibits expression of genes. Molecules that inhibit gene expression through the RNAi pathway include siRNAs, miRNAs, and shRNAs. In another example, Brd7 and/or Pbrm1 expression is silenced by delivering a gRNA (e.g., sgRNA) molecule targeting Brd7 or Pbrm1 (respectively) to the Treg cell, for example by transforming the cell. In some examples the RNAi or gRNA targets a sequence comprising at least 70%, at least 80%, at least 90%, at least 95%, or 100% sequence identity to a contiguous portion of SEQ ID NO: 31 or SEQ ID NO: 33. The contiguous portion can be 10-30 nucleotides in length, for example, 10-25 nucleotides, 10-20 nucleotides, 10-15 nucleotides, 15-30 nucleotides, 20-30 nucleotides, 25-30 nucleotides, 17-24 nucleotides, 18-15 nucleotides, or 20-25 nucleotides.

In some examples, the siRNA targets Brd7 or Pbrm1, and has at least 70%, 80%, 90%, or 95% sequence identity to SEQ ID NO: 43 or SEQ ID NO: 44, respectively. In some examples, the siRNA targets Brd7 or Pbrm1, and comprises or consists of SEQ ID NO: 43 or SEQ ID NO: 44, respectively. The gRNA targeting Brd7 or Pbrm1 can have at least 70%, at least 80%, at least 90%, or at least 95% sequence identity to SEQ ID NO: 10 or SEQ ID NO: 8, respectively. In some examples, the gRNA targets Brd7 or Pbrm1, and comprises or consists of SEQ ID NO: 10 or SEQ ID NO: 8, respectively. In further examples, the gRNA targets Brd7 or Pbrm1, and has at least 70%, 80%, 90%, or 95% sequence identity to SEQ ID NO: 46 or SEQ ID NO: 47, respectively. In some examples, the gRNA consists of or comprises SEQ ID NO: 46 or SEQ ID NO: 47, respectively. In further examples, the gRNA targeting Brd7 or Pbrm1 is a sgRNA that has at least 70%, 80%, 90%, or 95% sequence identity to SEQ ID NO: 10, SEQ ID NO: 8, SEQ ID NO: 46, or SEQ ID NO: 47. In some examples, the sgRNA targeting Brd7 or Pbrm1 comprises SEQ ID NO: 10, SEQ ID NO: 8, SEQ ID NO: 46, or SEQ ID NO: 47. In some embodiments, the Treg cell expresses a Cas nuclease or is contacted with a Cas nuclease (e.g., Cas9, Cas13) before, after, or substantially at the same time as the gRNA.

In some examples, a vector comprises the RNAi or gRNA (e.g., sgRNA) molecule targeting Brd7 or Pbrm1, which may be operably linked to a promoter (such as a constitutive, inducible, or tissue-specific promoter). The vector may facilitate transient expression of the RNAi or gRNA molecule in the Treg cell and/or may facilitate chromosomal integration of the RNAi or gRNA molecule or expression cassette comprising the RNAi or gRNA for stable expression in the Treg cell.

In some examples, administering the RNAi or gRNA (e.g., sgRNA) molecule reduces expression of Brd7 or Prbm1 by at least 10%, at least 20%, at least 30%, at least 40%, at least 50%, at least 60%, at least 70%, at least 80%, at least 90%, at least 100% or more relative to a suitable control (e.g., an unmodified Treg cell). In some examples, administering the RNAi or gRNA molecule reduces protein levels or accumulation of Brd7 or Prbm1 by at least 10%, at least 20%, at least 30%, at least 40%, at least 50%, at least 60%, at least 70%, at least 80%, at least 90%, at least 100% or more relative to a suitable control (e.g., an unmodified Treg cell).

In some examples, Brd7 or Pbrm1 expression or activity is reduced in a Treg cell by deleting all or a portion of a Brd7 or Pbrm1 gene, respectively, in the Treg cell. In some examples, all or a portion of the Brd7 or Pbrm1 gene is deleted using genome editing techniques, for example, CRISPR and/or TALEN genome editing (Li et al., Sig Transduct Target Ther, 5, 1, 2020).

In some examples, deleting all or a portion of Brd7 or Pbrm1 gene results in reduced expression of Brd7 or Pbrm1, respectively, in the Treg cell, for example by at least 10%, at least 20%, at least 30%, at least 40%, at least 50%, at least 60%, at least 70%, at least 80%, at least 90%, at least 100% or more relative to a suitable control (e g, unmodified Treg cell). In some examples, deleting all or a portion of Brd7 or Pbrm1 gene results in reduced levels of functional protein in the Treg cell, for example reducing functional Brd7 or Pbrm1 protein by at least 10%, at least 20%, at least 30%, at least 40%, at least 50%, at least 60%, at least 70%, at least 80%, at least 90%, at least 100% or more relative to a suitable control (e.g., unmodified Treg cell).

In some examples, Brd9 expression or activity is increased in the Treg cell by contacting the Treg cell with a Brd9 activator. In some examples, the activator targeting Brd9 increases transcription of a Brd9 gene in the Treg cell. For example, the Brd9 activator may increase transcription of Brd9 by at least 10%, at least 20%, at least 30%, at least 40%, at least 50%, at least 60%, at least 70%, at least 80%, at least 90%, at least 100%, or more relative to a control, such as an untreated Treg cell. In some examples, the activator targeting Brd9 increases translation of Brd9 mRNA, thereby increasing levels of Brd9 gene product in the Treg cell. For example, the activator increases levels of Brd9 protein by at least 10%, at least 20%, at least 30%, at least 40%, at least 50%, at least 60%, at least 70%, at least 80%, at least 90%, at least 100%, or more relative to a suitable control, such as an untreated Treg cell.

In further examples, the activator decreases degradation or increases protein stability of Brd9 mRNA or protein, thereby increasing the level of Brd9 protein in the Treg cell. For example, the activator increases levels of a Brd9 gene product in the Treg cell by at least 10%, at least 20%, at least 30%, at least 40%, at least 50%, at least 60%, at least 70%, at least 80%, at least 90%, at least 100%, or more relative to a suitable control, such as an untreated Treg cell. In other examples, an activator targeting Brd9 increases activity or a function of Brd9 protein in the Treg cell. In some embodiments, Brd9 activity is increased by at least 10%, at least 20%, at least 30%, at least 40%, at least 50%, at least 60%, at least 70%, at least 80%, at least 90%, at least 100%, or more relative to a suitable control, such as an untreated Treg cell.

In some examples, the Brd9 activator increases expression of Brd9, for example by at least 10%, at least 20%, at least 30%, at least 40%, at least 50%, at least 60%, at least 70%, at least 80%, at least 90%, at least 100% or more relative to a suitable control. In some examples, the Brd9 activator increases protein levels in the Treg cell, for example increasing Brd9 protein by at least 10%, at least 20%, at least 30%, at least 40%, at least 50%, at least 60%, at least 70%, at least 80%, at least 90%, at least 100% or more relative to a suitable control.

In some examples, Brd9 expression or activity is increased in the Treg cell by introducing (e.g., transforming) an expression vector encoding a Brd9 gene product into the Treg cell (e.g., a vector encoding SEQ ID NO: 30 or an amino acid sequence having at least 90% or at least 95% identity to SEQ ID NO: 30). The vector may facilitate transient expression of a Brd9 gene product in the Treg cell or may facilitate chromosomal integration of a nucleic acid molecule or expression cassette comprising a nucleic acid molecule encoding the Brd9 gene product for stable expression in the Treg cell. In some examples, a sequence comprising at least 70%, at least 80%, at least 90%, at least 95%, or 100% sequence identity to SEQ ID NO: 29 is introduced into the cell, for example as part of a vector, which may be operably linked to a promoter (such as a constitutive, inducible, or tissue-specific promoter).

In some examples, introducing the expression vector encoding Brd9 into the Treg cell increases expression of Brd9 in the Treg cell, for example by at least 10%, at least 20%, at least 30%, at least 40%, at least 50%, at least 60%, at least 70%, at least 80%, at least 90%, at least 100% or more relative to a suitable control (e.g., an untransformed Treg cell, or transformed with empty vector). In some examples, introducing the expression vector encoding Brd9 into the Treg cell increases a Brd9 protein level in the Treg cell, respectively, for example increasing Brd9 protein by at least 10%, at least 20%, at least 30%, at least 40%, at least 50%, at least 60%, at least 70%, at least 80%, at least 90%, at least 100% or more relative to a suitable control (e.g., an untransformed Treg cell, or transformed with empty vector).

### Methods of Reducing Treg Suppressor Activity

Disclosed herein are methods of reducing Treg suppressor activity. In some examples, the method includes reducing Foxp3 expression in a Treg cell. In some examples, Foxp3 expression or activity is reduced by reducing activity of an ncBAF complex or increasing activity of a PBAF complex in a Treg cell. In some examples, Treg suppressor activity is reduced by reducing expression or activity of Brd9, increasing expression or activity of Brd7, increasing expression or activity of Pbrm1, or combinations thereof, in a Treg cell. The expression or activity of Brd9, Brd7, or Pbrm1 may refer to a Brd9, Brd7, or Pbrm1 gene, mRNA, or protein, respectively.

In some examples, Brd9 expression or activity is reduced in a Treg cell by contacting the Treg cell with a small molecule inhibitor targeting Brd9, for example, I-BRD9, LP99, BI-7273, BI-9564, VZ-185, dBRD9, dBRD9-A. In specific, non-limiting examples, the small molecule inhibitor is dBRD9 or dBRD9-A.

In some examples, Brd9 expression or activity is reduced in a Treg cell by silencing expression of Brd9 in the Treg cell. For example, by delivering an RNAi molecule targeting Brd9 to the Treg cell, for example an RNAi that targets a sequence comprising at least 70%, at least 80%, at least 90%, at least 95%, or 100% sequence identity to a contiguous portion of SEQ ID NO: 29. RNAi generically refers to a cellular process that inhibits expression of genes. Molecules that inhibit gene expression through the RNAi pathway include siRNAs, miRNAs, and shRNAs. In another example, Brd9 expression is silenced by delivering a gRNA (e.g., sgRNA) targeting Brd9 to the Treg cell for example by transforming the cell. In some examples the RNAi or gRNA target a sequence comprising at least 70%, at least 80%, at least 90%, at least 95%, or 100% sequence identity to a contiguous portion of SEQ ID NO: 29. In some examples, the contiguous portion is 10-30 nucleotides in length, for example, 10-25 nucleotides, 10-20 nucleotides, 10-15 nucleotides, 15-30 nucleotides, 20-30 nucleotides, 25-30 nucleotides, 17-24 nucleotides, 18-15 nucleotides, or 20-25 nucleotides. In a specific, non-limiting example, the contiguous portion is 19-21 nucleotides.

In some examples, the siRNA targets Brd9 and has at least 70%, 80%, 90%, or 95% sequence identity to SEQ ID NO: 42. In some examples, the siRNA targets Brd9 and comprises or consists of SEQ ID NO: 42. In some examples, the gRNA targeting Brd9 has at least 70%, 80%, 90%, or 95% sequence identity to SEQ ID NO: 12. In some examples, the gRNA targets Brd9, and comprises or consists of SEQ ID NO: 12. In other examples, the gRNA targets Brd9, and has at least 70%, 80%, 90%, or 95% sequence identity to SEQ ID NO: 45. In some examples, the gRNA consists of or comprises SEQ ID NO: 45. In further examples, the gRNA targeting Brd9 is a sgRNA that has at least 70%, 80%, 90%, or 95% sequence identity to SEQ ID NO: 12 or SEQ ID NO: 45. In some examples, the sgRNA targeting Brd9 comprises SEQ ID NO: 12 or SEQ ID NO: 45. In some embodiments, the Treg cell expresses a Cas nuclease or is contacted with a Cas nuclease (e.g., Cas9, Cas13) before, after, or substantially at the same time as the gRNA.

In some examples, a vector comprises the RNAi or gRNA (e.g., sgRNA) molecule targeting Brd9, which may be operably linked to a promoter (such as a constitutive, inducible, or tissue-specific promoter). The vector may facilitate transient expression of the RNAi or gRNA molecule in the Treg cell and/or may facilitate chromosomal integration of the RNAi or gRNA molecule or expression cassette comprising the RNAi or gRNA for stable expression in the Treg cell.

In some examples, administering the RNAi or gRNA (e.g., sgRNA) molecule reduces expression of Brd9 by at least 10%, at least 20%, at least 30%, at least 40%, at least 50%, at least 60%, at least 70%, at least 80%, at least 90%, at least 100% or more relative to a suitable control (e.g., an unmodified Treg cell). In some examples, administering the RNAi or gRNA molecule reduces protein levels of Brd9 by at least 10%, at least 20%, at least 30%, at least 40%, at least 50%, at least 60%, at least 70%, at least 80%, at least 90%, at least 100% or more relative to a suitable control (e.g., an unmodified Treg cell).

In some examples, Brd9 expression or activity is reduced in a Treg cell by deleting all or a portion of a Brd9 gene in the Treg cell. In some examples, all or a portion of the Brd9 gene is deleted using genome editing techniques, for example, CRISPR and/or TALEN genome editing (Li et al., Sig Transduct Target Ther, 5, 1, 2020).

In some examples, deleting all or a portion of a Brd9 gene or coding sequence results in reduced expression of Brd9 in the Treg cell, for example by at least 10%, at least 20%, at least 30%, at least 40%, at least 50%, at least 60%, at least 70%, at least 80%, at least 90%, at least 100% or more relative to a suitable control (e.g., an unmodified Treg cell). In some examples, deleting all or a portion of a Brd9 gene or coding sequence results in reduced levels of functional protein in the Treg cell, for example reducing functional Brd9 protein by at least 10%, at least 20%, at least 30%, at least 40%, at least 50%, at least 60%, at least 70%, at least 80%, at least 90%, at least 100% or more relative to a suitable control (e.g., an unmodified Treg cell).

In some examples, Brd7 or Pbrm1 expression or activity is increased in the Treg cell by contacting the Treg cell with a Brd7 or Pbrm1 activator, respectively. In some examples, the activator targeting Brd7 or Pbrm1 increases transcription of a Brd7 or Pbrm1 gene, respectively, in the Treg cell, respectively. For example, the activator can increase transcription of Brd7 or Pbrm1 gene by at least 10%, at least 20%, at least 30%, at least 40%, at least 50%, at least 60%, at least 70%, at least 80%, at least 90%, at least 100%, or more relative to a control, such as an untreated Treg cell. In some examples, the activator targeting Brd7 or Pbrm1 increases translation of Brd7 or Pbrm1 mRNA, respectively, thereby increasing levels of a respective protein product in the Treg cell. For example, the activator can increase levels or accumulation of a gene product (such as a Brd7 or Pbrm1 gene product) by at least 10%, at least 20%, at least 30%, at least 40%, at least 50%, at least 60%, at least 70%, at least 80%, at least 90%, at least 100%, or more relative to a suitable control, such as an untreated Treg cell.

In further examples, the activator decreases degradation or increases protein stability of Brd7 or Pbrm1 mRNA or protein, thereby increasing the level of Brd7 or Pbrm1 protein, respectively, in the Treg cell. For example, the activator can increase levels of a gene product (such as a Brd7 or Pbrm1 gene product) by at least 10%, at least 20%, at least 30%, at least 40%, at least 50%, at least 60%, at least 70%, at least 80%, at least 90%, at least 100%, or more relative to a suitable control, such as an untreated Treg cell. In other examples, the activator targeting Brd7 or Pbrm1 increases activity or a function of Brd7 or Pbrm1 protein, respectively. In some embodiments, Brd7 or Pbrm1 activity is increased by at least 10%, at least 20%, at least 30%, at least 40%, at least 50%, at least 60%, at least 70%, at least 80%, at least 90%, at least 100%, or more relative to a suitable control.

In some examples, the Brd7 or Pbrm1 activator increases expression of Brd7 or Prbm1 in the Treg cell, for example by at least 10%, at least 20%, at least 30%, at least 40%, at least 50%, at least 60%, at least 70%, at least 80%, at least 90%, at least 100% or more relative to a suitable control. In some examples, the Brd7 or Pbrm1 activator increases protein levels in the Treg cell, for example increasing Brd7 or Prbm1 protein by at least 10%, at least 20%, at least 30%, at least 40%, at least 50%, at least 60%, at least 70%, at least 80%, at least 90%, at least 100% or more relative to a suitable control.

In some examples, Brd7 or Pbrm1 expression or activity is increased in the Treg cell by introducing an expression vector encoding a Brd7 or Pbrm1 gene product (e.g., a vector encoding SEQ ID NO: 32 or 34, or an amino acid sequence having at least 95% identity to SEQ ID NO: 32 or 34), which may be operably linked to a promoter (such as a constitutive, inducible, or tissue-specific promoter, into the Treg cell (e.g., transforming the cell with the vector). The vector may facilitate transient expression of a Brd7 or Pbrm1 gene product in the Treg cell or may facilitate chromosomal integration of a nucleic acid molecule or expression cassette comprising a nucleic acid molecule encoding the Brd7 or Pbrm1 gene product for stable expression in the Treg cell. In some examples a sequence comprising at least 70%, at least 80%, at least 90%, at least 95%, or 100% sequence identity to SEQ ID NO: 31 or SEQ ID NO: 33 is introduced into the cell, for example as part of a vector.

In some examples, introducing an expression vector encoding Brd7 or Pbrm1 into the Treg cell increases expression of Brd7 or Pbrm1 in the Treg cell, respectively, for example by at least 10%, at least 20%, at least 30%, at least 40%, at least 50%, at least 60%, at least 70%, at least 80%, at least 90%, at least 100% or more relative to a suitable control (e.g., an untransformed Treg cell, or transformed with empty vector). In some examples, introducing the expression vector encoding Brd7 or Pbrm1 into the Treg cell increases a Brd7 or Pbrm1 protein level in the Treg cell, respectively, for example increasing Brd7 or Pbrm1 protein by at least 10%, at least 20%, at least 30%, at least 40%, at least 50%, at least 60%, at least 70%, at least 80%, at least 90%, at least 100% or more relative to a suitable control (e.g., an untransformed Treg cell, or transformed with empty vector).

### IV. Modified Cells

Also provided herein are modified cells (e.g., Treg cells) that include a heterologous nucleic acid molecule. In some examples, the cells are mammalian cells, such as human cells, dog cells, or mouse cells. In some embodiments, the heterologous nucleic acid molecule encodes a Brd9, Brd7, or Pbrm1 protein, or combinations thereof. In some examples, the heterologous nucleic acid molecule encodes Brd9. In further examples, the heterologous nucleic acid molecule encodes Brd7 and/or Pbrm1. In some examples, the Brd9, Brd7, or Pbrm1 is mammalian, for example, human or mouse Brd9, Brd7, or Pbrm1. In some embodiments, the heterologous nucleic acid molecule encodes an amino acid sequence having at least 80%, at least 85%, at least 90%, or at least 95% sequence identity to SEQ ID NO: 30, SEQ ID NO: 32, or SEQ ID NO: 34. In some examples, the heterologous nucleic acid molecule encodes an amino acid sequence comprising or consisting of SEQ ID NO: 30, SEQ ID NO: 32, or SEQ ID NO: 34. In further examples, the heterologous nucleic acid molecule comprises or consists of a sequence having at least 80%, at least 85%, at least 90%, or at least 95% sequence identity to SEQ ID NO: 29, SEQ ID NO: 31, or SEQ ID NO: 33. In some examples, the heterologous nucleic acid molecule comprises or consists of SEQ ID NO: 29, SEQ ID NO: 31, or SEQ ID NO: 33. The heterologous nucleic acid molecule can be operably linked to a promoter, such as a native or non-native promoter. In some examples, the promoter is constitutive. In some examples the promoter is inducible.

In some embodiments, the heterologous nucleic acid molecule encodes an siRNA or gRNA (e.g., sgRNA) targeting Brd9, Brd7, Pbrm1, or combinations thereof. In some examples, the RNAi or gRNA targets a sequence comprising at least 70%, at least 80%, at least 90%, at least 95%, or 100% sequence identity to a contiguous portion of SEQ ID NO: 29, SEQ ID NO: 31, or SEQ ID NO: 33. In some examples, the contiguous portion is 10-30 nucleotides in length, for example, 10-nucleotides, 10-20 nucleotides, 10-15 nucleotides, 15-30 nucleotides, 20-30 nucleotides, 25-30 nucleotides, 17-24 nucleotides, 18-15 nucleotides, or 20-25 nucleotides. In a specific, non-limiting example, the contiguous portion is 19-21 nucleotides.

In some examples, the siRNA targets Brd9 and has at least 70%, 80%, 90%, or 95% sequence identity to SEQ ID NO: 42. In some examples, the siRNA targets Brd9 and comprises or consists of SEQ ID NO: 42. In some examples, the siRNA targets Brd7 or Pbrm1, and has at least 70%, 80%, 90%, or 95% sequence identity to SEQ ID NO: 43 or SEQ ID NO: 44, respectively. In some examples, the siRNA targets Brd7 or Pbrm1, and comprises or consists of SEQ ID NO: 43 or SEQ ID NO: 44, respectively.

In some examples, the heterologous nucleic acid molecule encodes an gRNA targeting Brd9. The gRNA targeting Brd9 can have at least 70%, 80%, 90%, or 95% sequence identity to SEQ ID NO: 12 or SEQ ID NO: 45. In some examples, the gRNA targets Brd9, and comprises or consists of SEQ ID NO: 12 or SEQ ID NO: 45. In further examples, the gRNA targeting Brd9 is a sgRNA that has at least 70%, 80%, 90%, or 95% sequence identity to SEQ ID NO: 12 or SEQ ID NO: 45. In some examples, the sgRNA targeting Brd9 comprises SEQ ID NO: 12 or SEQ ID NO: 45.

In some examples, the heterologous nucleic acid molecule encodes an gRNA targeting Brd7 and/or Pbrm1. The gRNA targeting Brd7 can have at least 70%, 80%, 90%, or 95% sequence identity to SEQ ID NO: 10 or SEQ ID NO: 46. In some examples, the gRNA targets Brd7 and comprises or consists of SEQ ID NO: 10 or SEQ ID NO: 46. In further examples, the gRNA targeting Brd7 is a sgRNA that has at least 70%, 80%, 90%, or 95% sequence identity to SEQ ID NO: 10 or SEQ ID NO: 46. In some examples, the sgRNA targeting Brd7 comprises SEQ ID NO: 10 or SEQ ID NO: 46. The gRNA targeting Pbrm1 can have at least 70%, 80%, 90%, or 95% sequence identity to SEQ ID NO: 8 or SEQ ID NO: 47. In some examples, the gRNA targets Pbrm1 and comprises or consists of SEQ ID NO: 8 or SEQ ID NO: 47. In further examples, the gRNA targeting Pbrm1 is a sgRNA that has at least 70%, 80%, 90%, or 95% sequence identity to SEQ ID NO: 8 or SEQ ID NO: 47. In some examples, the sgRNA targeting Pbrm1 comprises SEQ ID NO: 8 or SEQ ID NO: 47. In some embodiments, the Treg cell expresses a Cas nuclease or is contacted with a Cas nuclease (e.g., Cas9, Cas13) before, after, or substantially at the same time as the gRNA.

In some embodiments, the modified Treg cells are transduced or transformed with the heterologous nucleic acid molecule, or an expression vector encoding the heterologous nucleic acid. In some embodiments, the expression vector also encodes a Cas nuclease, such as Cas9 or Cas13. Any suitable technique for transducing or transforming Treg cells can be used, non-limiting examples include electroporation, lipofection, polyfection, viral transduction (e.g., with retroviral or lentiviral vectors), or particle bombardment.

In some embodiments, the heterologous nucleic acid molecule is encoded on a vector. The nucleic acid molecule may be operably linked to a promoter (such as a constitutive, inducible, or tissue-specific promoter). The vector may facilitate transient expression of the heterologous nucleic acid molecule in the modified Treg cell and/or may facilitate chromosomal integration of the heterologous nucleic acid molecule or expression cassette comprising the heterologous nucleic acid molecule for stable expression in the modified Treg cell.

Suitable vectors are described herein, for example, plasmid or viral vectors. A plasmid refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). A replication deficient viral vector is a vector that requires complementation of one or more regions of the viral genome required for replication due to a deficiency in at least one replication-essential gene function.

In some embodiments, the vector is a lentivirus (such as an integration-deficient lentiviral vector) or adeno-associated viral (AAV) vector. Other exemplary viral vectors that can be used include polyoma, SV40, vaccinia virus, herpes viruses including HSV and EBV, Sindbis viruses, alphaviruses and retroviruses of avian, murine, and human origin, baculovirus (Autographa californica multinuclear polyhedrosis virus; AcMNPV) vectors, retrovirus vectors, orthopox vectors, avipox vectors, fowlpox vectors, capripox vectors, suipox vectors, adenoviral vectors, herpes virus vectors, alpha virus vectors, baculovirus vectors, Sindbis virus vectors, vaccinia virus vectors and poliovirus vectors. Specific exemplary vectors are poxvirus vectors such as vaccinia virus, fowlpox virus and a highly attenuated vaccinia virus (MVA), adenovirus, baculovirus and the like. Pox viruses of use include orthopox, suipox, avipox, and capripox virus. Orthopox include vaccinia, ectromelia, and raccoon pox. One example of an orthopox of use is vaccinia. Avipox includes fowlpox, canary pox and pigeon pox. Capripox include goatpox and sheeppox. In one example, the suipox is swinepox. Specific viral vectors that can be used include other DNA viruses such as herpes simplex virus and adenoviruses, and RNA viruses such as retroviruses and polio. In some examples, the vector is a retroviral vector, such as pSIRG-NGFR.

Also described herein is a modified Treg cell including a small molecule inhibitor targeting Brd9, Brd7, or Pbrm1, or an activator targeting Brd9, Brd7, or Pbrm1, or combinations thereof. In some embodiments, the modified Treg cell includes a small molecule inhibitor, for example, ACBI1, AU-15330, BRM014, PFI-3, LP99, BI-7273, VZ-185, I-BRD9, BI-9564, dBRD9, dBRD9-A, or combinations thereof. In a specific, non-limiting example, the modified Treg cell includes dBRD9.

Also provided are nucleic acid molecules encoding a RNAi or gRNA targeting Brd7, Brd9 or Pbrm1, as disclosed herein, and vectors comprising the nucleic acid molecules, as disclosed herein.

The following examples are provided to illustrate certain features and/or embodiments. These examples should not be construed to limit the disclosure to the particular features or embodiments described.

### Examples

Examples 1-7 as well as their accompanying figures are described in Loo et al., Immunity 53, 143-157, 2020, which is herein incorporated by reference in its entirety.

### Example 1: Materials and Methods

### List of Antibodies

| | | Application |
|---|---|---|
| Target protein | Antibody source | (dilution) |
| CD4-Alexa fluor 700 | eBioscience 56-0042-82 | Flow (1:400) |
| Foxp3-eFluor 450 | eBioscience 48-5773-82 | Flow (1:400) |
| NGFR-PE | Biolegend 345106 | Flow (1:400) |
| NGFR-APC | Biolegend 345108 | Flow (1:400) |
| Thy1.1-PE | eBioscience 12-0900-83 | Flow (1:400) |
| Ghost Viability Dye | TONBO 13-0865-T100 | Flow (1:800) |
| Foxp3 | In-house | WB (1:2000); |
| | | ChIP (1:100) |
| BRG1/SMARCA4 | Abcam 110641 | WB (1:2000); |
| | | IP, ChIP (1:100) |
| BAF155/SMARCC1 | Santa Cruz sc-10756 | WB (1:1000) |
| BAF47/SMARCB1 | Santa Cruz sc-166165 | WB (1:1000) |
| BRD9 | Active Motif 61537 | WB (1:2000); |
| | | IP, ChIP (1:100) |
| PBRM1 | Bethyl A301-591A | WB (1:2000) |
| PHF10 | Thermo Fisher PA5-30678 | IP, ChIP (1:100) |
| ARID1A | Santa Cruz sc-32761 | WB (1:1000) |
| Histone H3K27ac | Abcam ab4729 | ChIP (1:100) |
| IgG | Cell Signaling 2729S | IP (1:100) |
| anti-mouse secondary | Thermo Fisher A21058 | WB (1:20,000) |
| anti-rabbit secondary | Thermo Fisher SA535571 | WB (1:20,000) |

List of sgRNA Targeting Sequences

| | | sgRNA | |
|---|---|---|---|
| | Target | targeting | SEQ ID |
| Plasmid name | gene | sequence | NO: |
| pSIRG-NGFR-sgFoxp3 | Foxp3 | TCTACCCACAGGGATCAATG | 1 |
| pSIRG-NGFR-sgCbfb | Cbfb | GCCTTGCAGATTAAGTACAC | 2 |
| pSIRG-NGFR-sgDnmt1 | Dnmt1 | TAATGTGAACCGGTTCACAG | 3 |
| pSIRG-NGFR-sgArid1a | Arid1a | GCAGCTGCGAAGATATCGGG | 4 |
| pSIRG-NGFR-sgArid1b | Arid1b | TGAGTGCAAAACTGAGCGCG | 5 |
| pSIRG-NGFR-sgDpf1 | Dpf1 | TCTTCTACCTCGAGATCATG | 6 |
| pSIRG-NGFR-sgDpf2 | Dpf2 | GAAGATACGCCAAAGCGTCG | 7 |
| pSIRG-NGFR-sgPbrm1 | Pbrm1 | AAAACACTTGCATAACGATG | 8 |
| pSIRG-NGFR-sgArid2 | Arid2 | ACTTGCAGTAAATTAGCTCG | 9 |
| pSIRG-NGFR-sgBrd7 | Brd7 | CAGGAGGCAAGCTAACACGG | 10 |
| pSIRG-NGFR-sgPhf10 | Phf10 | GTTGCCGACAGACCGAACGA | 11 |
| pSIRG-NGFR-sgBrd9 | Brd9 | ATTAACCGGTTTCTCCCGGG | 12 |
| pSIRG-NGFR-sgGltscr1 | Gltscr1 | GTTCTGTGTAAAATCACACT | 13 |
| pSIRG-NGFR-sgGltscr1l | Gltscr1l | ATGGCTTTATGCAACACGTG | 14 |
| pSIRG-NGFR-sgSmarcd1 | Smarcd1 | CAATCCGGCTAAGTCGGACG | 15 |
| pSIRG-NGFR-sgEny2 | Eny2 | AGAGCTAAATTAATTGAGTG | 16 |
| pSIRG-NGFR-sgAtxn713 | Atxn713 | GCAGCCGAATCGCCAACCGT | 17 |
| pSIRG-NGFR-sgUsp22 | Usp22 | GCCATCGACCTGATGTACGG | 18 |
| pSIRG-NGFR-sgCcdc101 | Ccdc101/ | CCAGGTTTCCCGATCCAGAG | 19 |
| | Sgf29 | | |
| pSIRG-NGFR-sgTada3 | Tada3 | GAAGGTCTGTCCCCGCTACA | 20 |
| pSIRG-NGFR-sgTada1 | Tada1 | TTTCCTTCTCGACACAACTG | 21 |
| pSIRG-NGFR-sgTaf61 | Taf61 | TCATGAAACACACCAAACGA | 22 |
| pSIRG-NGFR-sgSupt20 | Supt20 | TTAGTAGTCAATCTGTACCC | 23 |
| pSIRG-NGFR-sgSupt5 | Supt5 | GATGACCGATGTACTCAAGG | 24 |
| pSIRG-NGFR-sgNT | Non- | AAAAAGTCCGCGATTACGTC | 25 |
| | targeting | | |

### Mice

C57BL/6 Rosa-Cas9/Foxp3^{Thy1.1} mice were generated by crossing Rosa26-LSL-Cas9 mice (The Jackson Laboratory #024857) with Foxp3^{Thy1.1} reporter mice (Liston et al., PNAS, 105:11903-11908, 2008). Male Cas9/Foxp3^{Thy1.1} mice at 8-12 weeks age were used to isolate Treg cells for the CRISPR screen, and no gender preference was given for other experiments. C57BL.6 Ly5.1+ congenic mice and Rag1^{-/-} mice purchased from the Jackson Laboratory were used for Treg suppression assay and adoptive T cell transfer in colitis and tumor models. All mice were bred and housed in the pathogen-free facilities and were conducted under the regulation of the Institutional Animal Care and Use Committee (IACUC) and institutional guidelines.

Retroviral Vectors and sgRNA Library Construction

Self-inactivating retroviral vector pSIRG-NGFR was generated by modifying pSIR-dsRed-Express2 (Addgene #51135), which enables cloning sgRNA as efficient as lentiCRISPRv2, to enrich transduced cells via magnetic beads isolation, and to perform intracellular staining without losing transduced reporter marker. All BbsI sites were mutated in pSIR-dsRed-Express2, then a sgRNA expressing cassette containing the U6 promoter, guide RNA scaffold and a 500 bp filler was inserted at BbsI cloning site. The dsRed cassette was replaced by cDNA sequence of a modified human nerve growth factor receptor (NGFR) with a truncated intracellular domain A pSIRG vector with GFP (pSIRG-GFP) was also generated for the purpose of T cell transfer in tumor studies, to minimizing potential immune rejection. The pSIRG-GFP was generated by cutting pSIRG-NGFR with XcmI restriction enzyme to remove the NGFR cassette and replace it with GFP cDNA by Gibson® cloning. For cloning single guide RNA (sgRNA) into the pSIRG vector, annealed sgRNA oligos were directly inserted into BbsI-digested pSIRG-NGFR by T4 ligation, similar to the cloning method utilized by lentiCRISPRv2 (Sanjana et al., Nat Methods 11:783-784, 2014). To create a pooled sgRNA library in pSIRG-NGFR, sgRNA sequences were amplified from an optimized mouse CRISPR sgRNA library lentiCRISPRv2-Brie (Addgene #73632). A total of eight 50 μL PCR reactions were performed to maximize coverage of sgRNA complexity. Each 50 μL PCR reaction contained Q5® High-Fidelity DNA polymerase and buffer (NEB #M0491), 15 ng of lentiCRISPRv2-Brie, and targeted primers (Forward: GGCTTTATATATCTTGTGGAAAGGACGAAACACCG (SEQ ID NO: 26), Reverse: CTAGCCTTATTTTAACTTGCTATTTCTAGCTCTAAAAC (SEQ ID NO: 27)). PCR was performed at 98° C. denature, 67° C. annealing, 72° C. extension for 12 cycles. The sgRNA library amplicons were then combined and separated using a 2% agarose gel, and purified by the QIAquick® Gel Extraction Kit (Qiagen #28704). The purified sgRNA amplicons was inserted into the BbsI-digested pSIRG-NGFR by NEBuilder® HIFI assembly (NEB #E2621S). The sgRNA representative of the retroviral CRISPR library (pSIRG-NGFR-Brie) was validated by deep sequencing and comparing to the original lentiCRISPRvs-Brie. The coverage of the new pSIRG-NGFR sgRNA library was evaluated by the PinAPL-Py program (Spahn et al., Sci Rep 7: 15854, 2017).

### T Cell Isolation and Culture

For large scale Treg culture, Tregs were first expanded in Rosa-Cas9/Foxp3^{Thy1.1} mice by injecting IL-2:IL-2 antibody immune complex according protocol described in Webster et. al (J Exp Med 206, 751-760, 2009). Spleen and lymph node Treg cells were labeled with PE-conjugated Thy1.1 antibody and isolated by magnetic selection using anti-PE microbeads (Mitenyl #130-048-801). All isolated Treg cells were activated by plate bound anti-CD3 and anti-CD28 antibodies and cultured with X-VIVO® 20 media (LONZA #04-448Q) supplemented by 1× Pen/Strep, 1× Sodium pyruvate, 1× HEPES, 1× GlutaMax™, 55 μM beta-mercaptoethanol in the presence of IL-2 at 500 units/mL. For experiments with Brd9 degradation, Treg cells were treated at day 0 with 2.5 μM dBRD9 (Tocris #6606) and cultured for four days for RNA- and ChIP-seq and 0.16-10 μM treated at day 0 and cultured dBRD9 for four days for Foxp3 MFI, cell viability and cell proliferation assays. Live cells were enriched by Ficoll-Paque® 1.084 (GE Health 17-5446-02) for RNA-seq and ChIP-seq.

### Retroviral Production and T Cell Transduction

HEK293T cells were seeded in 6-wells plate at 0.5 million cells per 2 mL DMEM media supplemented by 10% FBS, 1% Pen/Strep, 1× GlutaMax®, 1× Sodium Pyruvate, 1× HEPES, and 55 μM beta-mercaptoethanol. One day later, cells from each well were transfected with 1.2 μg of targeting vector pSIRG-NGFR and 0.8 μg of packaging vector pCL-Eco (Addgene, #12371) by using 4 μL of FuGENE® HD transfection reagent (Promega #E2311) according manufactured protocol. Cell culture media was replaced by 3 mL fresh DMEM complete media at 24 hours and hours after transfection. The retroviral supernatant was collected at 48 and 72 hours post transfection for T cell infection. For experiments with CRISPR sgRNA targeting, Cas9+ Treg cells were first seeded in 24-wells plate coated with CD3 and CD28 antibodies. At 24 hour post-activation, 70% of Treg media from each well was replaced by retroviral supernatant, supplemented with 4 μg/mL Polybrene™ (Milipore #TR-1003-G), and spun in a benchtop centrifuge at 1,258×g for 90 minutes at 32° C. After centrifugation, Treg media was replaced with fresh media supplemented with IL-2 and cultured for another three days. Transduced cells were analyzed for Foxp3 and cytokine expression in eBioscience® Fix/Perm buffer (eBioscience #00-5523-00) using flow cytometry. Transduced NGFR+ cells were FACS-sorted for subsequent RNA- and ChIP-seq experiments.

### Genome-Wide CRISPR Screen in Treg

Approximately 360 million Treg cells were isolated from Rosa-Cas9/Foxp3^{Thy1.1} mice and used for the Treg screen. On day 0, Treg cells were seeded at 1×10⁶ cells/mL into 24-wells plate coated with anti-CD3/28 and cultured with X-VIVO® complete media with IL-2 (500 U/ml). On day 1, sgRNA retroviral library transduction was performed with a MOI<0.2. On day 3, approximately 4 million (˜50×coverage) NGFR+ transduced cells were collected in three replicates as the starting state sgRNA input. Treg cells reached confluence on day 4. NGFR+ transduced cells were isolated via magnetic selection by anti-PE beads (Mitenyl #130-048-801), and then plated onto new 24-wells plates coated with anti-CD3/CD28, and cultured in X-VIVO® complete media with IL-2 (500 U/ml). On day 6, approximately 4 million NGFR+ transduced cells were collected in three replicates as the ending state sgRNA output. The remaining cells were fixed, permeabilized, and stained for intracellular Foxp3. Approximately 2 million Foxp3^{hi} (top 20%) and 2 million Foxp3^{lo} (bottom 20%) cell populations were sorted in three replicates by a FACSAria™ cell sorter for genomic DNA extraction and library construction.

Preparation of sgRNA Amplicons for Next-Generation Sequencing

To extract genomic DNA, cells were lysed with homemade digestion buffer (100 mM NaCl, 10 mM Tris, 25 mM EDTA, 0.5% SDS, 0.1 mg/mL Proteinase K) overnight in 50° C. On the following day, the lysed sample was mixed with phenol:chloroform:isoamyl alcohol (25:24:1, v/v) in 1:1 ratio, and spun at 6000 rpm for 15 min at room temperature. The supernatant containing genomic DNA was transferred into a new tube and mixed with twice volume of 100% ethanol, then spun at 12,500 rpm for 5 min in room temperature to precipitate DNA. Supernatant was removed, and the precipitated DNA was dissolved in ddH₂O. DNA concentration was measured by NanoDrop®. To generate sgRNA amplicons from extracted genomic DNA, a two-step PCR protocol was adopted from the protocol published by Shalem et al. was used (Science, 343:84-87, 2014). Eight 50 μL PCR reactions containing 2 μg genomic DNA, NEB Q5 polymerase, and buffer, and targeted primers (Forward: GGCTTTATATATCTTGTGGAAAGGACGAAACACCG (SEQ ID NO: 26), Reverse: CTAGCCTTATTTTAACTTGCTATTTCTAGCTCTAAAAC (SEQ ID NO:27)) were performed. PCR was performed at 98° C. denature, 70° C. annealing, 15s extension for 20 cycles. The products from the first PCR were pooled together, and purified by AMPure® XP SPRI™ beads according to manufacturer's protocol, and quantified by Qubit® dsDNA HS assay. For the second round PCR, eight 50 μL PCR reactions were performed containing 2 ng purified 1^{st} round PCR product, barcoded primer (see primer set from Shalem et al., Science, 343:84-87, 2014), priming site of reverse primer was changed to CTTCCCTCGACGAATTCCCAAC (SEQ ID NO: 28)), NEB® Q50 polymerase, and buffer. PCR was performed at 98° C. denature, 70° C. annealing, 15 second extension for 12 cycles. The 2^{nd} round PCR products were pooled, purified by AMPure® XP SPRIG beads, quantified by Qubit® dsDNA HS assay, and sequenced by NEXTSeq® sequencer at single end 75 bp.

### In Vitro Treg Suppression Assay

Treg cells were transduced by retrovirus expressing sgRNA targeting gene of interest and cultured in X-VIVO® complete media supplemented with IL-2 (500 U/ml). Four days after transduction, transduced cells were sorted and mixed with a fluorescence-activated cell sorter (FACS). CD45.1+ naive CD4 T cells (CD4⁺CD25⁻CD44^{lo}CD62^{hi}) were labeled with CellTrace™ Violet (Thermo Fisher Scientific #C34571) in different ratio in the presence of irradiated T cell depleted spleen cells as antigen-presenting cells (APC). Three days later, Treg suppression function was measured by the percentage of non-dividing cells within the CD45.1⁺ effector T cell population. For dBRD9 treatment experiment, dBRD9 was first dissolved in DMSO (10 mM stock) and added into Treg:Teff:APC mixture at 2.5 μM. For Foxp3 overexpression rescue experiment, Treg cells were first transduced with sgNT or sgBrd9 at 24 hour post-activation, and then transduced with MIGR empty vector or MIGR-Foxp3 at 48 hour post-activation. Double transduced Treg cells were FACS sorted on day 4 based on NGFR+ and GFP+ markers and then mixed with CellTrace™ labeled effector T cells in the presence of APC. Treg suppression readout was measured after three days of co-culture.

### Adoptive T Cells Transfer-Induced Colitis Model

Treg cells were transduced by retrovirus expressing sgRNA targeting gene of interest, and cultured in X-VIVO® complete media and IL-2 (500 U/ml). Four days after transduction, the NGFR⁺ transduced Treg cells were FACS sorted before transferred into recipient mice. To induce colitis, 2 million effector T cells (CD45.1⁺CD4⁺CD25⁻CD45RB^{hi}) and 1 million sgRNA transduced Treg cells (CD45.2⁺ CD4⁺ Thy1.1⁺ NGFR⁺) were mixed together and transferred into Rag1^{-/-} recipient mice. The body weight of recipient mice was monitored weekly for signs of wasting symptoms. Mice were harvested 7 weeks after T cell transfer. Spleens were used for profiling immune cell populations by FACS. Colons were collected for histopathological analysis.

### Adoptive T Cells Transfer and MC38 Tumor Model

Similar to the “Adoptive T cells transfer-induced colitis model,” Treg cells were activated in vitro and transduced with pSIRG-GFP expressing sgNT or sgBrd9. Four days after transduction, the GFP+ transduced Treg were FACS sorted. Concurrently, Treg depleted CD4 and CD8 T cells isolated from Rosa-Cas9/Foxp3^{Thy1.1} mice were used as effector T cells. A total of 1 million pSIRG-sgRNA transduced GFP+ Treg cells, 1 million effector CD8 T cells, and 2 million Treg-depleted CD4 T cells were mixed and transferred into Rag1^{-/-} recipient mice. On the following day, mice were implanted with 0.5 million MC38 cells by subcutaneous injection on the flank of mouse. When palpable tumor appeared, tumor size was measured every two day by electronic calipers. At the end point, spleen and tumor were collected for immune profiling. For tumor processing, tumor tissues were minced into small pieces and digested with 0.5 mg/mL Collagenase IV (Sigma #C5138) and DNAase I (Roche #4716728001) for 20 minutes and passed through 0.75 μm cell strainer to collect single cell suspension. Isolated cells were stimulated with PMA/Ionomycin and GolgiPlug™ for 5 hours, and then were subjected to Foxp3 and cytokines staining with eBioscience® Fix/Perm buffer (eBioscience #00-5523-00).

### Nuclear Protein Extraction

Nuclear lysates were collected from Treg cells following a revised Dignam protocol (Andrews and Faller, Nucleic Acids Res 19, 2499, 1991). After cellular swelling in Buffer A (10 mM Hepes pH 7.9, 1.5 mM MgCl₂, 10 mM KCl) supplemented with 1 mM DTT, 1 mM PMSF, 1 μM pepstatin, 10 μM leupeptin and 10 μM chymostatin, cells were lysed by homogenization using a 21-gauge needle with six to eight strokes. If lysis remained incomplete, cells were treated with 0.025-0.05% Igepal-630 for ten minutes on ice prior to nuclei collection. Nuclei were spun down at 700×g for five minutes then resuspended in Buffer C (20 mM Hepes pH 7.9, 20% glycerol, 420 mM NaCl, 1.5 mM MgCl₂, 0.2 mM EDTA) supplemented with 1 mM DTT, 1 mM PMSF, 1 μM pepstatin, 10 μM leupeptin and 10 μM chymostatin. After thirty minutes of end-to-end rotation at 4° C., the sample was clarified at 21,100×g for ten minutes. Supernatant was collected, flash frozen in liquid nitrogen and stored in the −80° C. freezer.

### Co-Immunoprecipitation

Nuclear lysates were thawed on ice then diluted with two-thirds of original volume of 50 mM Tris-HCl pH 8, 0.3% NP-40, EDTA, MgCl₂ to bring down the NaCl concentration. Proteins were quantified using Biorad® DC™ Protein Assay (Cat #5000112) according to manufacturer's instructions. For the co-IP reaction, 200-300 μg of proteins were incubated with antibody against normal IgG, Smarca4, Brd9, Arid1a or Phf10 overnight at 4° C., with end-to-end rotation. Precipitated proteins were bound to 50:50 Protein A:Protein G Dynabeads™ (Invitrogen) for one to two hours and washed extensively with IP wash buffer (50 mM Tris pH 8, 150 mM NaCl, 1 mM EDTA, 10% glycerol, 0.5% Triton X100). Proteins were eluted in SDS-PAGE loading solution with boiling for five minutes and analyzed by western blotting.

### Western Blot

Protein samples were run on 4-12% Bis-Tris gels (Life Technologies). After primary antibody incubation which is typically done overnight at 4° C., blots were probed with 1:20,000 dilution of fluorescently-labeled secondary antibodies in 2% BSA in PBST (1× Phospho-buffered saline with 0.1% Tween-20) for an hour at room temperature (RT). Fluorescent images were developed using Odyssey® and analyzed using Image Studio 2™. Protein quantitation was performed by first normalizing the measured fluorescence values of the proteins of interest against the loading control (TBP) then normalizing against the control sample (vehicle treated).

### RNA-Seq Sample Preparation

RNA from 1-3×10 6 cells was extracted and purified with TRIzol™ reagent (Thermo Fisher) according to manufacturer's instructions. RNA-seq libraries were prepared using Illumina® TruSeq® Stranded mRNA kit following manufacturer's instructions with 5 μg of input RNA.

### ChIP-Seq Sample Preparation

Treg cells were collected and cross-linked first in 3 mM disuccinimidyl glutarate (DSG) in 1×PBS for thirty minutes then in 1% formaldehyde for another ten minutes, both at RT, for chromatin binding protein ChIP or in 1% formaldehyde only for histone modification ChIP. After quenching the excess cross-linker with a final concentration of 125 mM glycine, the cells were washed in 1× PBS, pelleted, flash-frozen in liquid nitrogen, and stored at −80° C. Cell pellets were thawed on ice and incubated in lysis solution (50 mM HEPES-KOH pH 8, 140 mM NaCl, 1 mM EDTA, 10% glycerol, 0.5% NP40, 0.25% Triton X-100) for ten minutes. The isolated nuclei were washed with wash solution (10 mM Tris-HCl pH 8, 1 mM EDTA, 0.5 mM EGTA, 200 mM NaCl) and shearing buffer (0.1% SDS, 1 mM EDTA, 10 mM Tris-HCl pH 8) then sheared in a Covaris® E229 sonicator for ten minutes to generate DNA fragments between ˜200-1000 base pairs (bp). After clarification of insoluble material by centrifugation, the chromatin was immunoprecipitated overnight at 4° C. with antibodies against Foxp3, Smarca4, Brd9, Phf10 or H3K27ac. The next day, the antibody bound DNA was incubated with Protein A+G Dynabeads™ (Invitrogen) in ChIP buffer (50 mM HEPES-KOH pH 7.5, 300 mM NaCl, 1 mM EDTA, 1% Triton X-100, 0.1% DOC, 0.1% SDS), washed and treated with Proteinase K and RNase A. Cross-linking was reversed by incubation at 55° C. for two and a half hours. Purified ChIP DNA was used for library generation (NuGen Ovation® Ultralow Library System V2) according to manufacturer's instructions for subsequent sequencing.

### ATAC-Seq Sample Preparation

ATAC-seq was performed according to previously published protocol (Corces et al., Nat Methods 14:959-962, 2017). Briefly, Tregs transduced with either sgNT or sgBrd9 were subjected to Ficoll™ gradient purification to remove dead cells and ensure capture of cells that were 99% viable. 50,000 Treg cells were collected in duplicates per genotype and washed first with cold 1×PBS then with Resuspension buffer (RSB; 10 mM Tris-HCl pH 7.4, 10 mM NaCl, 3 mM MgCl₂). Cells were lysed in 50 μL of RSB supplemented with 0.1% NP40, 0.01% Digitonin and 0.1% Tween 20 for 3 minutes on ice then diluted with 1 mL of RSB with 0.1% Tween 20. Nuclei were isolated by centrifugation at 500×g for ten minutes then resuspended in 50 μL of transposition mix (25 μL 2× Illumina® Transposase buffer, 2.5 μL Illumina® Tn5 Transposase, 16.5 μL PBS, 0.5 μL 1% digitonin, 0.5 μL 10% Tween® 20, 5 μL water) for 30 minutes at 37° C. in a thermomixer with shaking at 1,000 rpm. Reactions were cleaned up with Qiagen® MinElute® columns. ATAC-seq libraries were prepared as described previously (Buenrostro et al., Nat Methods 10:1213-1218, 2013). Briefly, purified DNA was ligated with adapters and amplified to a target concentration of μL at 4 nM. Libraries were size selected using AMPure® XP beads (Beckman) and sequenced using NextSeq® for paired end 42 bp (PE42) sequencing.

### Data Analysis of Pooled CRISPR Screen

The screening hit identification and quality control was performed by MAGeCK-VISPR program (Li et al., Genome Biol 16, 281, 2015; Li et al., Genome Biol 15, 554, 2014a). The abundance of sgRNA from a sample fastq file was first quantified by MAGeCK “Count” module to generate a read count table. For hit calling, MAGeCK “test” module was used to generate a gene-ranking table that reporting RRA gene ranking score, p-value, and log 2 fold change. The size factor for normalization was adjusted according to 1000 non-targeting control assigned in the screen library. All sgRNAs that are zero read were removed from RRA analysis. The log 2 fold change of a gene was calculated from a mean of 4 sgRNA targeting per gene. The scatter plots showing the screen results were generated by using the R script Enhanced Volcano (github.com/kevinblighe/EnhancedVolcano). The R script that generated the sgRNA distribution histogram was provided by E. Shifrut and A. Marson (UCSF) (Shifrut et al., Cell 175:1958-1971, 2018). A gene list from Foxp3 regulators (either positive or negative) without affecting cell proliferation was subjected to Gene Ontology analysis using Metascape (Zhou et al., Nature Communications 10:1523, 2019). Genes were analyzed for enrichment for Functional Set, Pathway, and Structural Complex.

### Colon Histopathological Analysis

Histopathological analysis was performed in a blinded manner and scored using the following criteria: eight parameters were used, including (i) the degree of inflammatory infiltrate in the LP (0-3); (ii) Goblet cell loss (0-2); (iii) reactive epithelial hyperplasia/atypia with nuclear changes (0-3); (iv) the number of IELs in the epithelial crypts (0-3); (v) abnormal crypt architecture (distortion, branching, atrophy, crypt loss) (0-3); (vi) number of crypt abscesses (0-2); (vii) mucosal erosion to frank ulcerations (0-2) and (viii) submucosal spread to transmural involvement (0-2). The severity of lesion was scored independently in 3 regions (proximal, middle and distal colon) over a maximal score of 20. The overall colitis score was based as the average of each regional score (maximal score of 20).

### RNA-Seq Analysis

Single-end 50 bp reads were aligned to the mouse genome mm10 using STAR alignment tool (V2.5) (Dobin et al., Bioinformatics 29:15-21, 2013). RNA expression was quantified as raw integer counts using analyzeRepeats.pl in HOMER (Heinz et al., Mol Cell 38:576-589, 2010) using the following parameters: -strand both -count exons -condenseGenes -noadj. To identify differentially expressed genes, getDiffExpression.pl in HOMER was used, which uses the DESeq2R package to calculate the biological variation within replicates. Cut-offs were set at log 2 FC=0.585 and FDR at 0.05 (Benjamin-Hochberg). Principal Component Analysis (PCA) was performed with the mean of transcript per million (TPM) values using Cluster 3.0 with the following filter parameters: at least one observation with absolute value equal or greater than two and gene vector of four. TPM values were log transformed then centered on the mean.

### Gene Set Enrichment Analysis

GSEA software (Mootha et al., Nat Genet 34:267-273, 2003; Subramanian et al., PNAS 102:15545-15550, 2005) was used to perform the analyses with the following parameters: number of permutations=1000; enrichment statistic=weighted; and metric for ranking of genes=difference of classes (Input RNA-seq data was log-transformed). For GSEA analysis, input RNA-seq data contained the normalized log-transformed reads of the 1,325 differentially expressed genes (DEGs) in sgFoxp3/sgNT Treg cells. The compiled gene list included GSEA Gene Ontology, Immunological Signature, Curated Gene, and the up and down DEGs in sgBrd9/sgNT Treg cells.

The resulting normalized enrichment scores and FWER p values were combined to generate the graph.

### ChIP-Seq Analysis

Single-end 50 bp or paired-end 42 bp reads were aligned to mouse genome mm10 using STAR alignment tool (V2.5) (Dobin et al., Bioinformatics 29:15-21, 2013). ChIP-Seq peaks were called using findPeaks within HOMER using parameters for histone (-style histone) or transcription factor (-style factor) (homer.ucsd.edu/homer/index.html). Peaks were called when enriched >two-fold over input and >four-fold over local tag counts, with FDR 0.001. For histone ChIP, peaks within a 1000 bp range were stitched together to form regions. Differential ChIP peaks were found by merging peaks from control and experiment groups and called using getDiffExpression.pl with fold change ≥1.5 or ≤−1.5, Poisson p value<0.0001.

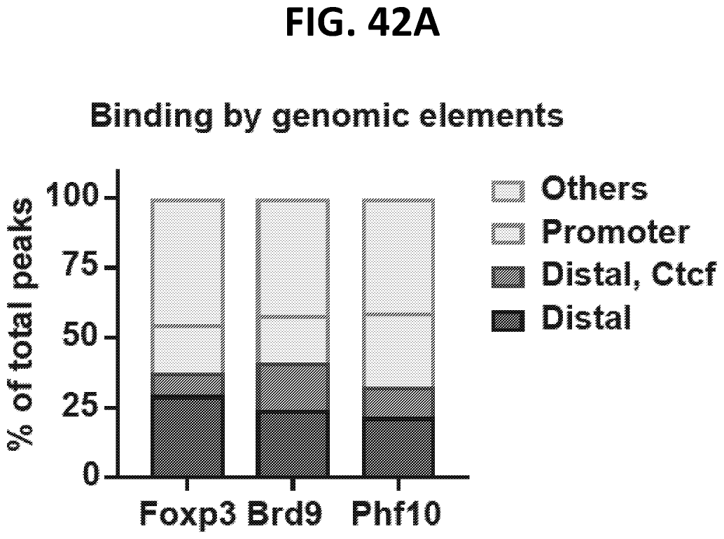

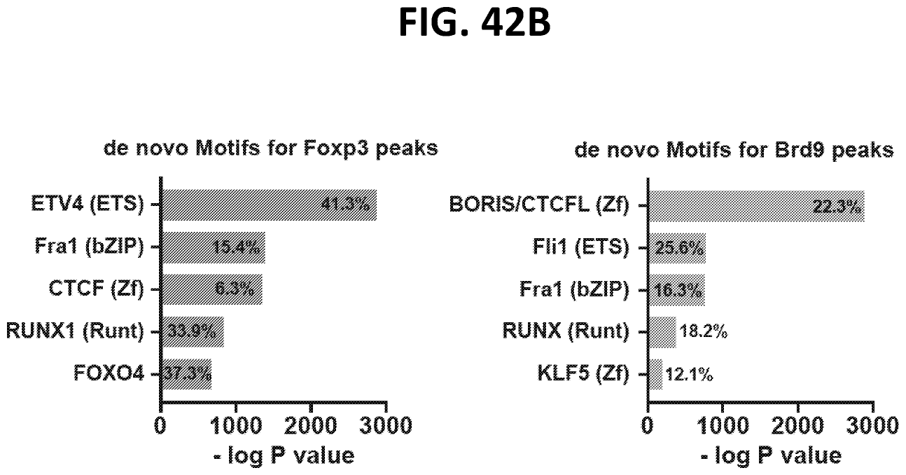

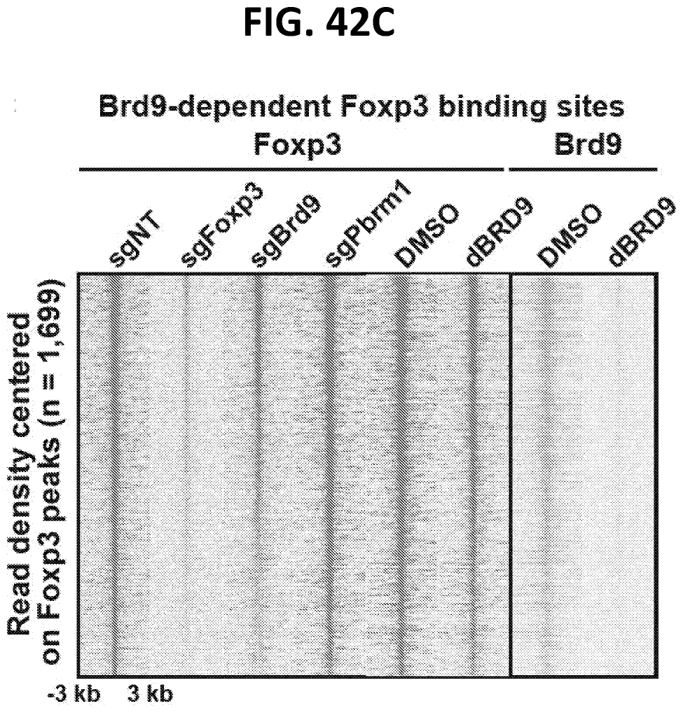

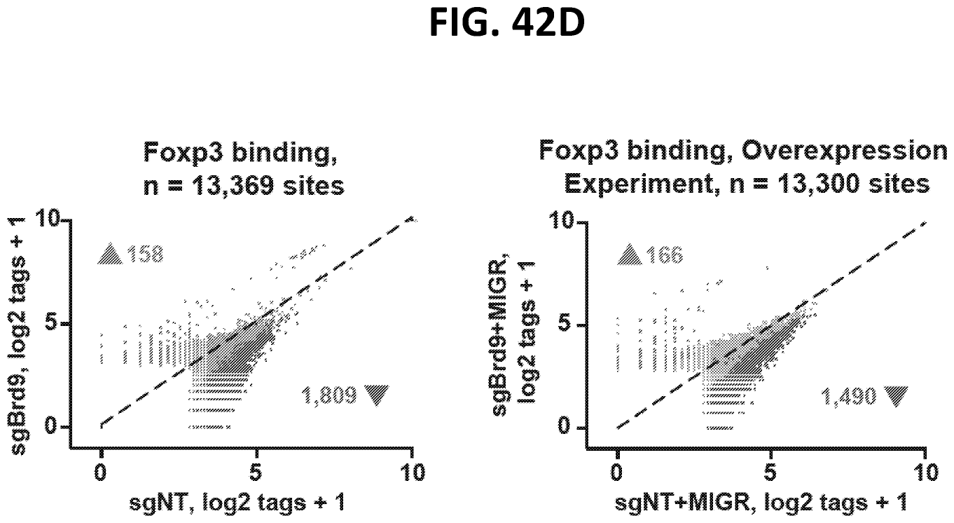

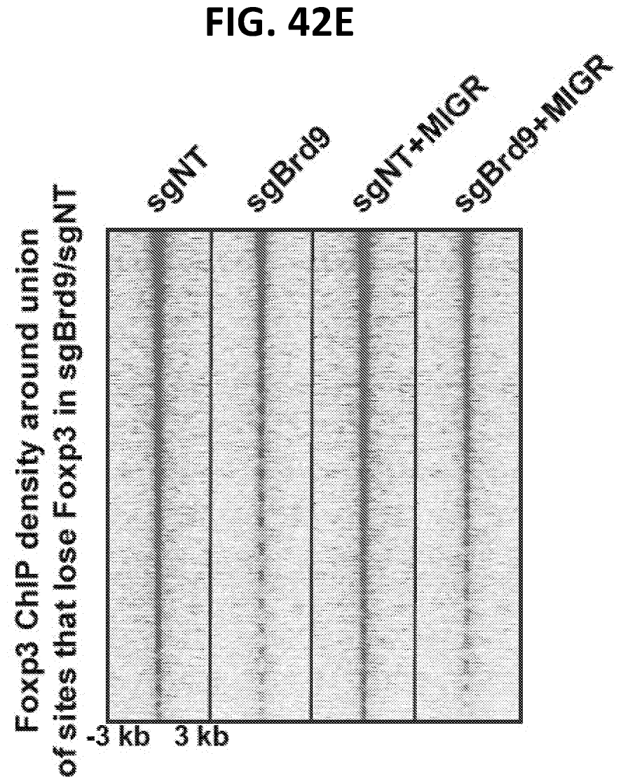

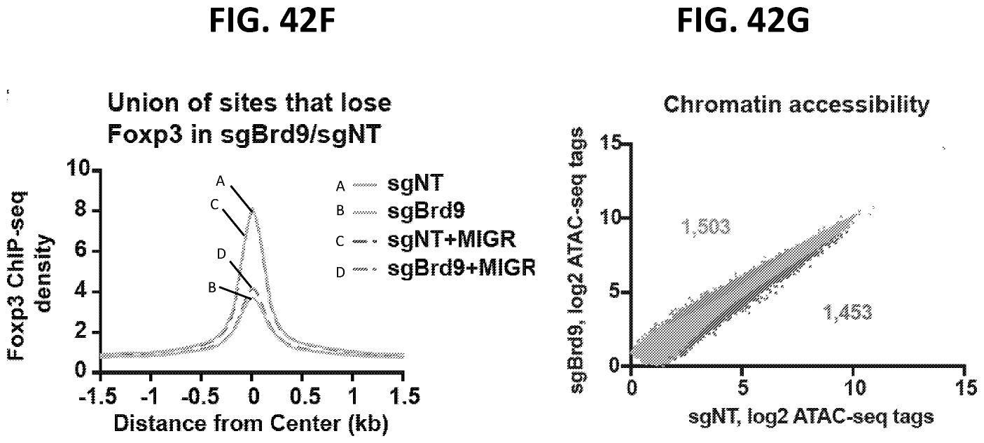

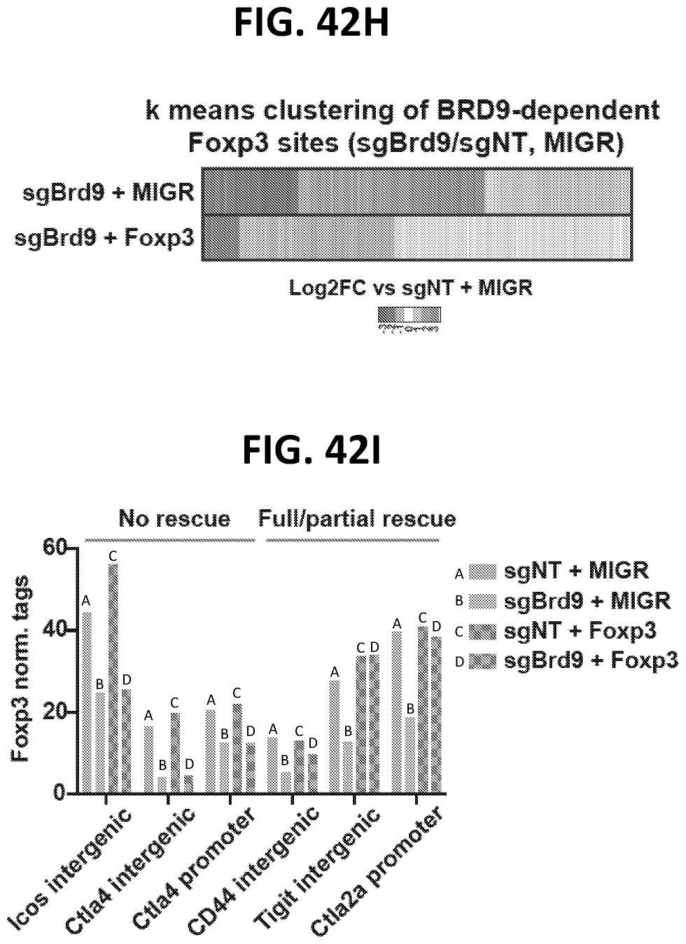

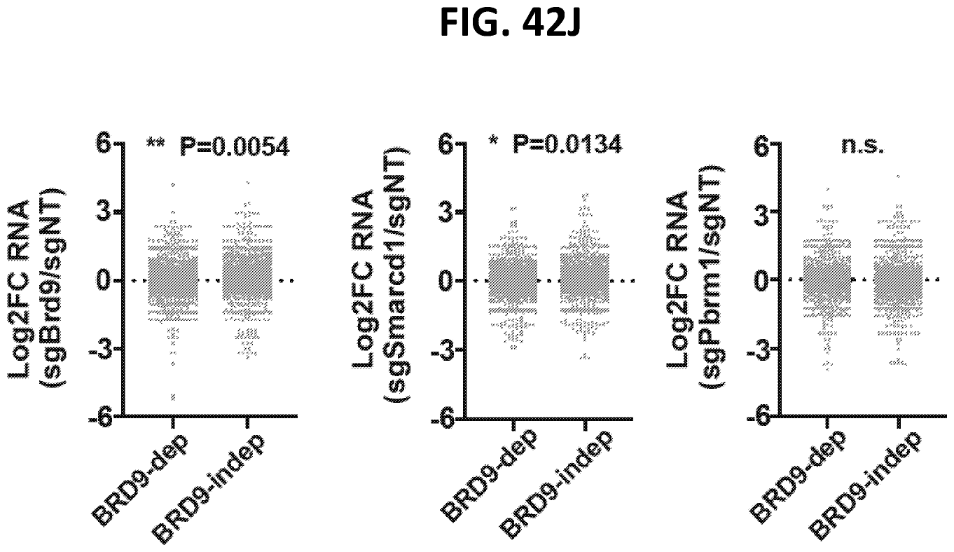

For k-means clustering analysis in FIG. 42D, Foxp3 ChIP-seq tags were quantified at the sites that significantly lose Foxp3 binding in sgBrd9, MIGR compared to sgNT, MIGR using the annotatePeaks.pl command in HOMER with -size given. Log₂FC values were calculated for sgBrd9, MIGR/sgNT, MIGR and sgBrd9, Foxp3/sgNT, MIGR. k-means clustering was performed using Gene Cluster 3.0 and visualized using Java TreeView.

For gene expression analysis in FIG. 42F, Foxp3 ChIP-seq tags were quantified at the union of sites bound by Foxp3 in sgNT and sgBrd9 using the annotatePeaks.pl command in HOMER with size-given and each site was annotated to a gene by mapping to the nearest TSS. Sites were ranked from least to largest Foxp3 ChIP-seq Log₂FC in sgBrd9 vs sgNT and divided into quartiles. Gene expression for the genes in the top and bottom quartiles (Brd9-dependent and -independent, respectively) was then plotted using RNA-seq data from Treg cells transduced with sgBrd9, sgSmarcd1, or sgPbrm1 compared to sgNT. Statistical analyses were performed using unpaired two-tailed Student's t test (ns: p≥0.05, *p<0.05, **p<0.01) in Graphpad Prism™.

### Motif Analysis

Sequences within 200 bp of peak centers were compared to motifs in the HOMER database using the findMotifsGenome.pl command using default fragment size and motif length parameters. Random GC content-matched genomic regions were used as background. Enriched motifs are statistically significant motifs in input over background by a p-value of less than 0.05. P-values were calculated using cumulative binomial distribution.

### ATAC-Seq Analysis

ATAC-seq data analysis used the following tools and versions: cutadapt (v2.4), samtools (v1.9), Picard (v1.7.1), BWA (v0.7.12), macs2 (v2.1.2), and HOMER (v4.11). Paired end 42 bp reads were trimmed using cutadapt to remove Nextera™ adapter sequences then aligned to the reference mouse genome mm10 using BWA. The following were filtered out using Picard and samtools: duplicate reads, mitochondrial reads, low quality reads (Q<20), and improperly paired or unpaired reads. Quality was assessed by calculating Fraction of Reads In Peaks (TRIP Score) which were >40% for all samples. TSS enrichment was determined using mm10 Refseq TSSs. Broad and narrow peaks were called using macs2 using the following parameters: --slocal 1000-qvalue 0.05-f BAMPE. Differentially accessible sites were determined using getDifferentialPeaksReplicates.pl command in HOMER using the union of peaks in sgNT and sgBrd9 with the following parameters: edgeR, fold change cutoff 1.5, adjusted p value<0.05.

### Data Availability

RNA-seq, ChIP-seq, and ATAC-seq data that support the findings of this study have been deposited in the Gene Expression Omnibus under the accession code GSE129846 (ncbi.nlm.nih.gov/geo/query/acc.cgi?acc=GSE129846) and is herein incorporated by reference in its entirety.

### Experimental Autoimmune Encephalomyelitis (EAE) Model

Mice were immunized with 200 ng of MOG peptide in CFA by subcutaneous injection on day 0 and received 200 ng of Pertussis toxin intraperitoneally on day 0 and day 2. Mice were monitored daily once mice started showing clinical symptoms. Clinical scores were determined based on guideline published by Stromnes and Goverman (Nat Protoc 1(4):1810-19, 2006). At the end of the experiment, brain, spinal cord, and spleen were harvested for histology and immune profiling. For characterizing immune cells in brain and spinal cord (CNS), the CNS tissues were minced and digested by collagenase IV and DNAase I for 30 minutes. Digested cells were passed through 75 um strainer to remove debris and followed by Percoll® isolation to enrich immune population. Cells were fixed and stained using eBioscience® Fix/Perm buffer.

Histopathological analysis of spinal cord was performed in a blinded manner. When examining inflammation in H & E stained sections, the following parameters were used: no evidence of inflammation (0), rare scattered small foci of cell inflammation (1), multiple isolated foci of cellular infiltration (2), multiple confluent foci of inflammation (3), foci of necrosis and/or neutrophilic infiltration. For examining demyelination in Luxol Blue stained sections, parameters were used following criteria: normal (0), minimal of few scattered degenerative neurons (1), moderate multifocal groups of degenerative neurons (2), marked of large multifocal degenerative neurons (3), severe or coalescing groups of degenerative neurons (4). The overall inflammation and demyelination scores were based on the average of each regional score (maximal score of 4).

### Glioblastoma Multiforme (GBM) Tumor Model

Mice were implanted with 0.1×10⁶ GL261 glioblastoma cells by stereotaxic injection into the brain. The site of injection was approximately halfway between the eye and the ear, just off the midline, in the medial posterior region of the top of the skull. First a small incision was made. Then using a sterile, disposable 27½G needle, mice were pierced directly through the cranium to a depth of 3 mm to deliver a 5-10 ul injection volume into the lateral ventricle. Needles were threaded through a safety sleeve that prevented insertion to depths greater than 3 mm. The incision was sealed with one drop of VetBond™. Mice were returned to cage and monitored post-op until fully alert and righting reflexes were evident. Mice were monitored daily post intracranial injection for 72 hours, and then at least twice a week. Mice were checked for gait disturbance, infection, appetite loss, poor hydration and any sign of discomfort. Mice showing signs of any of the above were monitored daily. At the end point, mouse brains were collected and digested by collagenase IV. Digested cells were passed through 75 um strainer to remove debris and followed by Percoll® isolation to enrich immune population Immune cell composition was determined by FACS analysis.

### Example 2

### Identification of Foxp3 Regulators

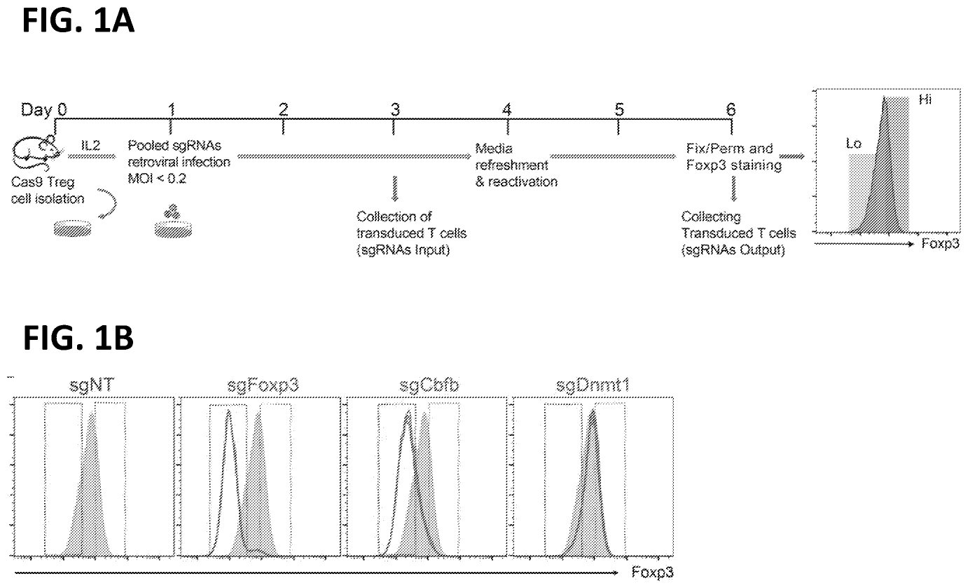

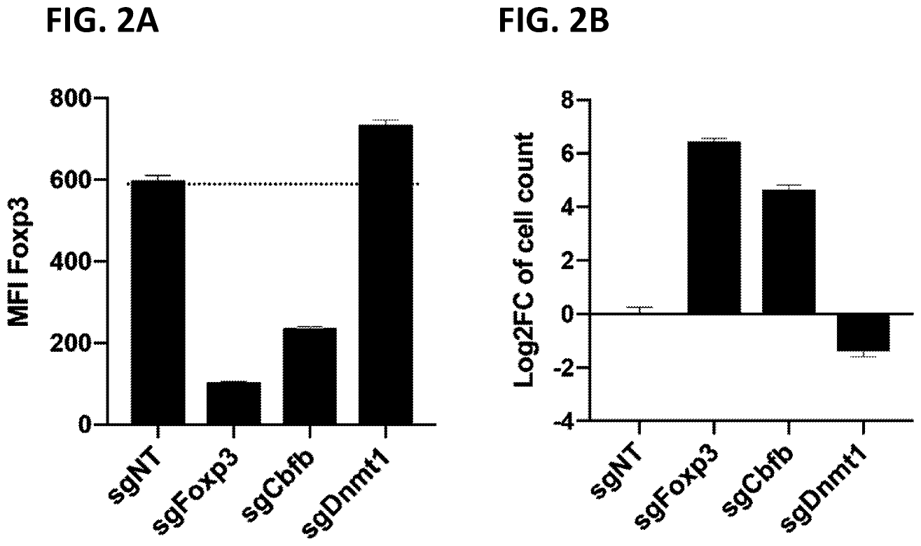

To screen for genes that regulate Foxp3 expression, a pooled retroviral CRISPR sgRNA library was developed by subcloning an optimized mouse genome-wide lentiviral CRISPR sgRNA library (lentiCRISPRv2-Brie) (Doench et al., Nat Biotechnol 34:184-191, 2016) into a newly engineered retroviral vector pSIRG-NGFR, which allowed us to efficiently transduce mouse primary T cells and to perform intracellular staining of Foxp3 without losing the transduction marker NGFR after cell permeabilization. Using this library, a CRISPR loss-of-function screen was performed on Treg cells to identify genes that regulate Foxp3 expression. CD4⁺Foxp3⁺ Treg cells isolated from Rosa-Cas9/Foxp3^{Thy1.1} reporter mice were activated with CD3 and CD28 antibodies and IL-2 (FIG. 1A). Treg cells were transduced 24 hours post-activation with the pooled retroviral sgRNA library at multiplicity of infection of less than 0.2 to ensure only one sgRNA was transduced per cell. NGFR transduced Treg cells were collected on day 3 and day 6 to identify genes that are essential for cell proliferation and survival. In addition, the bottom quintile (NGPR⁺Foxp3^{lo}) and top quintile (NGFR⁺Foxp3^{hi}) populations were collected on day 6 to identify genes that regulate Foxp3 expression. Screen conditions were validated by transducing Treg cells with sgRNAs targeting Foxp3 itself, as well as previously reported positive (Cbfb) (Rudra et al., Nat Immunol 10:1170-1177, 2009) and negative (Dnmt1) (Lal et al., Journal of Immunology 182:259-273, 2009) regulators of Foxp3 (FIGS. 1B and 2A-2B). Guide RNA sequences integrated within the genomic DNA of sorted cells were recovered by PCR amplification, constructed into amplicon libraries, and sequenced with a NextSeq® sequencer.

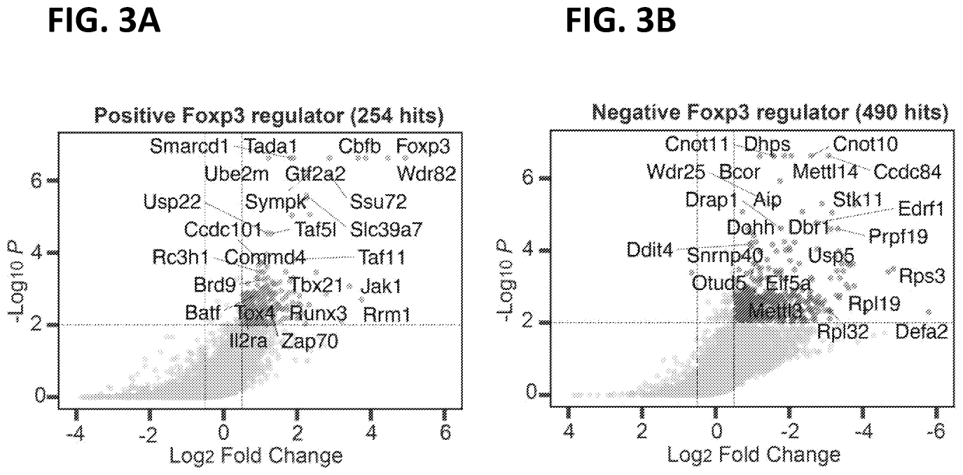

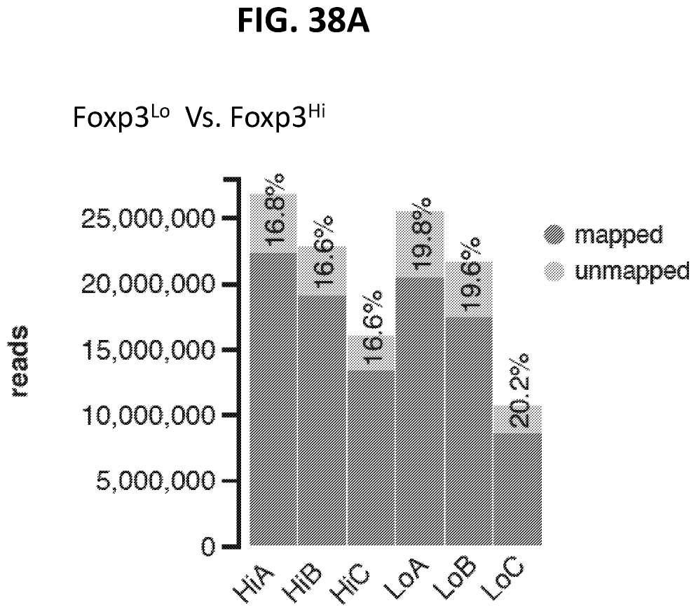

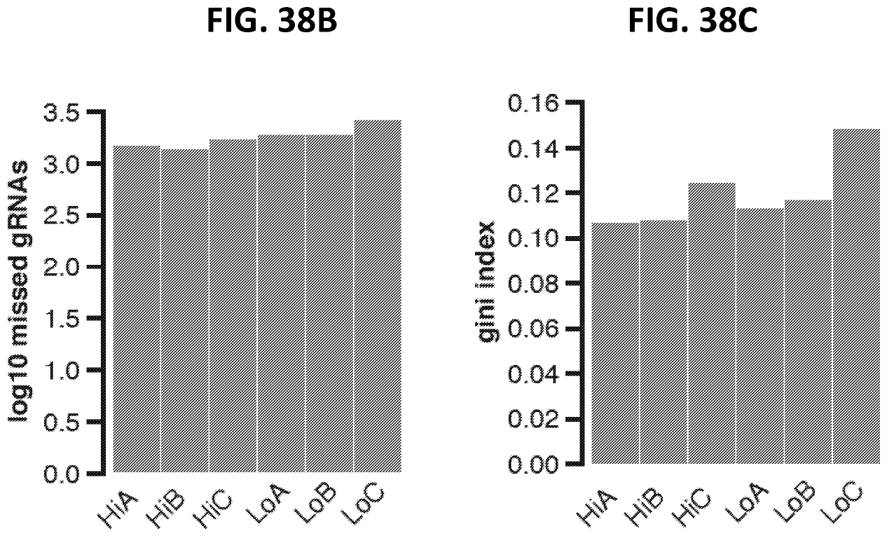

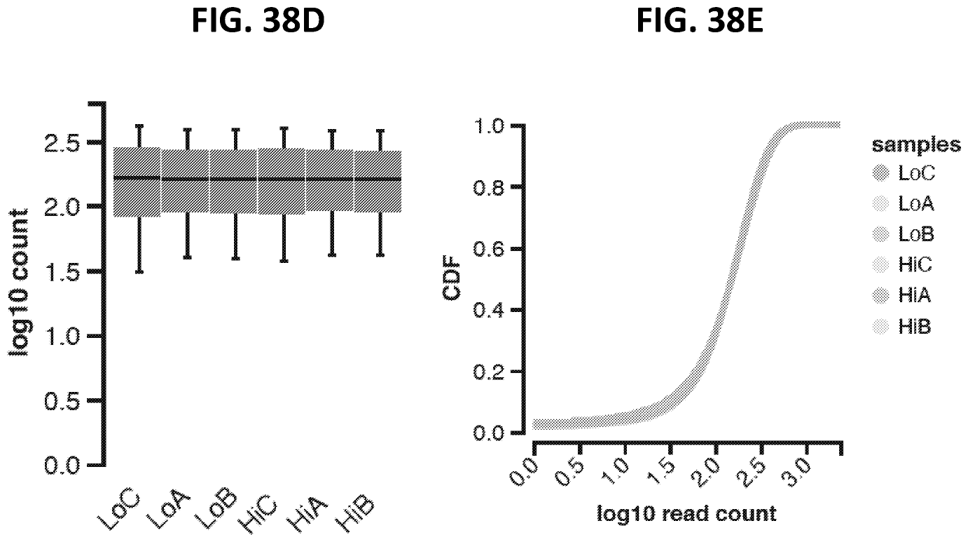

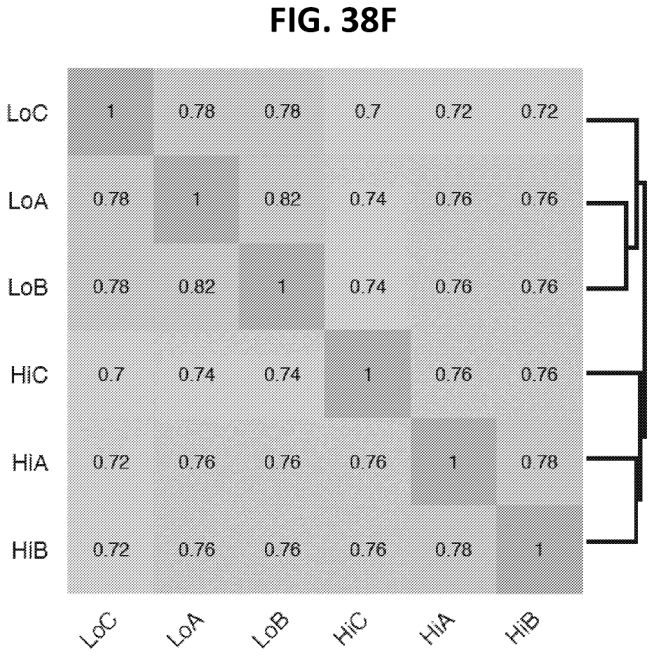

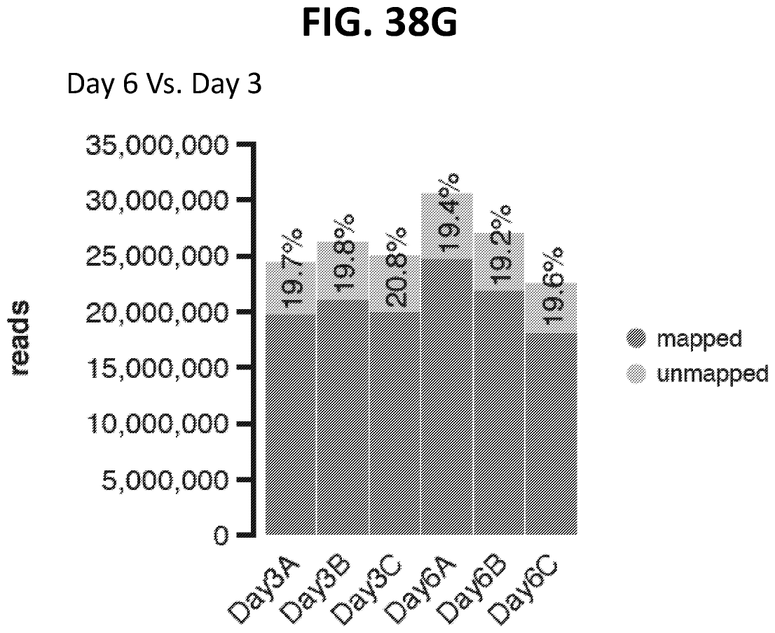

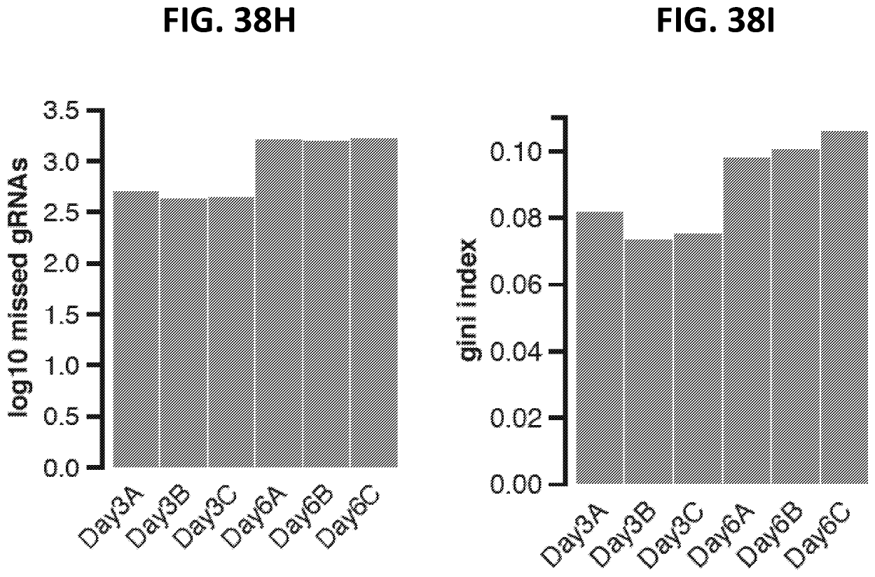

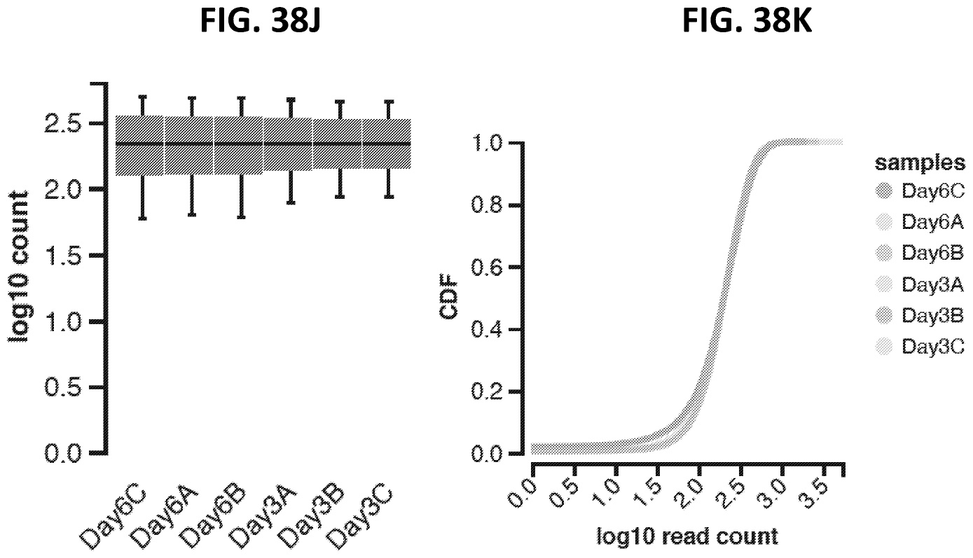

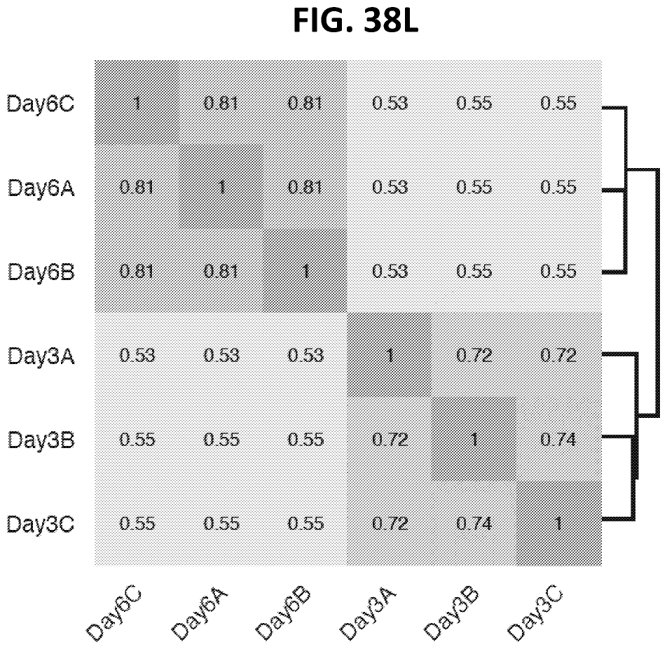

The relative enrichment of sgRNAs between samples and hit identification were computed by MAGeCK, which generates a normalized sgRNA read count table for each sample, calculates the fold change of sgRNA read counts between two cell populations, and further aggregates information of four sgRNAs targeting each gene to generate a ranked gene list (Li et al., Genome Biol 15:554, 2014). Prior to hit calling, the quality of screen samples determined by measuring the percentage of mapped reads to the sgRNA library and total read coverage, which showed a high mapping rate (79.8-83.4%) with an average of 236× coverage and a low number of missing sgRNAs (0.625-2.5%) (FIG. 38). With the cutoff criteria of log 2 fold change (LFC)>±0.5 and p-value less than 0.01, 254 potential positive Foxp3 regulators enriched in the Foxp3^{lo} population were identified and 490 potential negative Foxp3 regulators enriched in the Foxp3^{hi} population were identified (FIGS. 3A, 3B).

TABLE 1
Genes that positively regulate Foxp3

| | | |
|---|---|---|
| Foxp3 | Ncoa2 | Defb26 |
| Smarcd1 | Tada3 | Ubald2 |
| Tada1 | Olfr3 | Pam |
| Gtf2a2 | Uqcr11 | Setd5 |
| Ube2m | Stap1 | Cetn3 |
| Ssu72 | Ubtd2 | Gprc5b |
| Supt20 | Diras1 | Serpinb3c |
| Gtf2a1 | Bpifb4 | Mmp17 |
| Usp22 | Cyp2c65 | Elf1 |
| Taf5l | Zc3h4 | Timp1 |
| Taf11 | Runx3 | Spin2d |
| Noxred1 | Ttc21b | Prpf39 |
| Gnb2 | Bmp3 | Cd55 |
| Ccdc101 | Dok1 | Osbpl6 |
| Tada2b | Nudt12 | Mina |
| Taf6l | Cpne2 | Cldn10 |
| Olfr347 | Gkap1 | Fam209 |
| Commd4 | Dhx36 | Tmem5 |
| Impa1 | Gm2663 | Scaf4 |
| Ngb | Slc47a1 | Oxr1 |
| BC094916 | Ccdc169 | Zfp623 |
| Brd9 | Nkiras2 | Ugt2b5 |
| Tbx21 | Naif1 | Selk |
| Cdh6 | Obp1a | Ankrd53 |
| Cela1 | Krit1 | Prl2c3 |
| Serpinb9d | Galnt14 | Rnf145 |
| Rnmt | Rp9 | Ubald1 |
| Gpr174 | Prdx2 | Tmem175 |
| Rtl1 | Slc35b4 | Usp7 |
| Med29 | Obfc1 | Actn4 |
| Wdr48 | Smim22 | Rnf183 |
| Sertad1 | Nabp1 | Tmem154 |
| Vmn1r151 | Med27 | Dynlrb2 |
| Pcyox1l | Olfr74 | Mmp20 |
| Chd2 | Slc22a7 | Tnni3k |
| Traf3 | 4930502E18Rik | Cox20 |
| Rhebl1 | Enoph1 | Ddx50 |
| Sp140 | Zfp281 | Zbtb41 |
| 4930444G20Rik | Eepd1 | Fxyd5 |
| Traf6 | Kctd4 | Irs2 |
| Slc25a30 | Erp27 | Ggct |
| Rhag | Wnt7b | Pcdh8 |
| Timd2 | Magea6 | Xndc1 |
| Tox4 | Spo11 | Zfp280d |
| 0610009O20Rik | Sec22a | Prrc2c |
| Alg6 | Wdr46 | Tmco5 |
| Zfp790 | Brs3 | Psenen |
| Hmbs | Cd3d | Gfm2 |
| Cep57 | Ccdc43 | Trpm5 |
| Tmem176b | Ppig | Hint1 |
| Ube2cbp | Gast | Hoga1 |
| Cpsf41 | Rusc1 | Lrrc48 |
| Zmiz1 | Olfr59 | Ube2dnl2 |
| Gp1bb | Prep | Serpina1a |
| Rab3ip | Defb2 | Hmgn3 |
| Batf | Zdhhc13 | Rps6ka5 |
| Jakmip1 | Olfr902 | Gale |
| Zfp772 | Scgb1a1 | Kcna5 |
| Prrg1 | Tceb3 | Zfp704 |
| Vmn2r30 | Odc1 | Nsmf |
| Tmem121 | Inhba | Micalcl |
| Phf11a | Asnsd1 | Pdcd1lg2 |
| Olfr1122 | Man1c1 | Uqcrc2 |
| Bcl2l15 | Wdr24 | Pdzd9 |
| Ntn3 | Ccne2 | Ccpg1 |
| Arid1a | Surf1 | |

TABLE 2
Genes that negatively regulate Foxp3

| | | | | |
|---|---|---|---|---|
| Cnot11 | Anapc13 | Acsl3 | Sde2 | Alox12 |
| Bcor | Pex10 | Nlrp6 | Bbox1 | Egr1 |
| Dhps | Prss43 | Ccl24 | St6galnac1 | Ccdc130 |
| Cnot10 | Lamtor1 | Cers4 | Gpr137 | Gps1 |
| Atox1 | Tmem30a | Pou2f1 | Acacb | Frk |
| Mettl14 | Klhl30 | Krtap24-1 | Atcay | Mtx2 |
| Aip | Add2 | Zfp445 | Pdap1 | Gpx6 |
| Wdr25 | Fibcd1 | Abcb9 | Hpdl | Tnfsf13 |
| Slc13a3 | Rab8b | Tmem100 | Rasl10b | Sim2 |
| Edrf1 | Kcns1 | Tbx10 | Pramef17 | Lce3d |
| Dgkd | Acaa1a | Gsk3b | Shb | Upf3b |
| Drap1 | Ceacam18 | Ift43 | Olfr71 | Arf5 |
| Flcn | Gse1 | Zfp933 | Sesn2 | Parp14 |
| Ccdc88c | Gm6406 | Cxxc1 | Vmn1r175 | Serpinb9g |
| Dohh | 2510039O18Rik | Skint8 | Rarres1 | Pramef25 |
| Atp2a2 | Prr15 | Dr1 | Vmn1r13 | Olfr1111 |
| Ddit4 | Xkr4 | Ube3c | Tnks | Acin1 |
| Skiv2l | Adad2 | Slbp | Gca | Tubgcp5 |
| Usp9x | Cdh9 | Sptlc1 | Olfr1176 | 9130011E15Rik |
| Gnl3 | Sp1 | Celsr3 | Mad2l1bp | Upk3b |
| Tmem230 | Phyhipl | Kcna4 | 3110082I17Rik | Stub1 |
| Nrbp1 | Rbms3 | Galr2 | Tbl1x | Dlx3 |
| Pbld1 | Ttc29 | Cdkn2d | 2700094K13Rik | Setd1b |
| Otud5 | Dsg1a | Ankrd66 | Zc4h2 | Galnt11 |
| Bod1 | Insig1 | Tmie | C1ra | Bdh2 |
| Lsm10 | Nprl2 | Kiss1 | Cd4 | Slc16a7 |
| Pigf | Herc4 | Ebf1 | Lrrk2 | Pih1d2 |
| Bcl2a1b | Dido1 | Lrrc71 | Whsc1 | Gfra4 |
| Atp2b1 | Mrgpra1 | Slc4a1ap | Lyar | Bik |
| Atp7a | Edf1 | Olfr1034 | Rrs1 | Pacsin1 |
| Ror2 | Olfr39 | Gabarapl2 | BC005561 | Vmn2r38 |
| Pelo | Calm3 | Cacna1d | Neurl1a | Aagab |
| 2410127L17Rik | Ddx6 | Hira | Strada | Mccc1 |
| Abcb1a | Fam71b | Defb22 | Hc | Cxcl9 |
| Gigyf2 | Rtel1 | Brk1 | 1700055N04Rik | Hnrnpa3 |
| Clca3b | Cd2bp2 | Gm17689 | Sun1 | Nudt8 |
| Tex22 | Ndufv2 | Vac14 | Fibp | Casq2 |
| Oprm1 | Diap1 | Slc25a15 | Ptpro | Ang |
| Zmym5 | Sp3 | Acp1 | Gsk3a | Rel |
| Sfxn3 | Gm10324 | Gm7257 | E330017A01Rik | Grin2a |
| Gmip | P2ry6 | Slc19a1 | Olfr186 | Inha |
| Eif5a | Aldh1l2 | Zfy1 | BC005624 | Lsm3 |
| Btaf1 | Max | Zfpm2 | Slc22a18 | Gpr179 |
| Trmt5 | Dynlrb1 | Spryd3 | 3110079O15Rik | Teddm1a |
| C330007P06Rik | Dbf4 | Olfr1513 | Nup43 | Fam133b |
| Wfdc21 | 1300017J02Rik | Elac2 | Gm8720 | Zfp804b |
| Fbrs | Lsm11 | Dscaml1 | Gm15315 | Zrsr2 |
| E130309D02Rik | Cldn7 | Olfr1197 | BC048502 | Cwc25 |
| Fam117b | Nos3 | Idh3b | Ccnc | Sorl1 |
| Cml5 | Stk32b | Vmn1r212 | Tm9sf2 | Rilp |
| Polh | Arl2 | Olfr998 | Sytl4 | Fpr-rs6 |
| Mlycd | Tsc2 | Lta4h | Fbxw16 | Pgrmc2 |
| Mettl3 | Nup214 | Smg9 | Olfr360 | Ncapg2 |
| Rgs8 | Hrk | Bod1l | Ephb1 | Slc6a20a |
| Ddx3x | Thap11 | Arrb1 | Vmn1r59 | Arsk |
| Bricd5 | Klhl18 | Cnot1 | Otop3 | H2afz |
| Esrp2 | Cenpt | Ube2e3 | Wdr11 | Leprotl1 |
| Eml5 | Spty2d1 | Nr5a2 | Tas2r130 | Irgm2 |
| Trip4 | Zzz3 | Wfdc12 | Dbndd1 | Med24 |
| Rprd2 | Unc5c | Mrps17 | Ncor1 | Olfr122 |
| Wtap | Gm815 | Rpl28 | Adcy2 | Chd1 |
| Tmem131 | Unc45a | Nudt4 | Alkbh5 | Fkbpl |
| Ube2e1 | Hes1 | Tubb6 | Scd4 | Defa2 |
| Fanci | Pdgfrb | 1700102P08Rik | Trim35 | |
| Reg3g | Trpm7 | Ppp1r15b | Ube2c | |
| Ankrd24 | Dcaf5 | S1pr4 | Olfr1201 | |

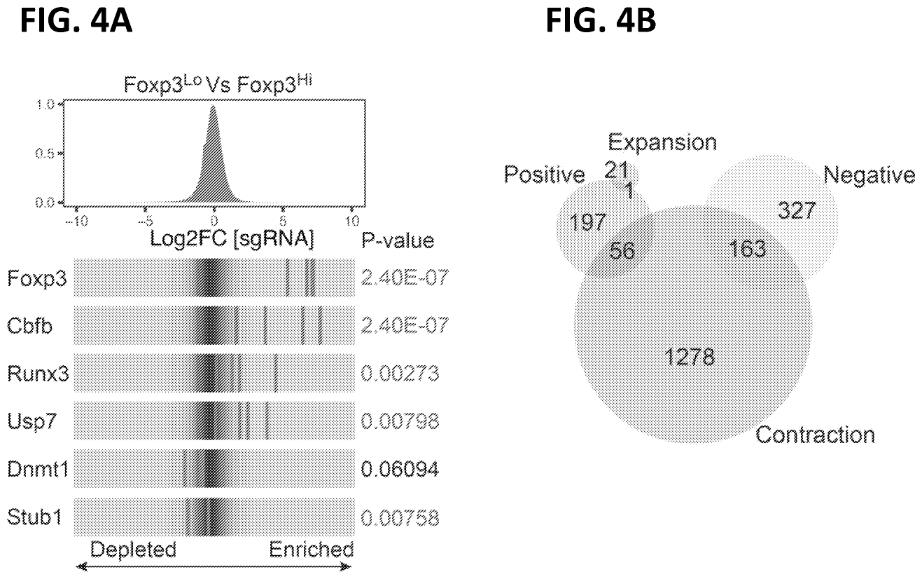

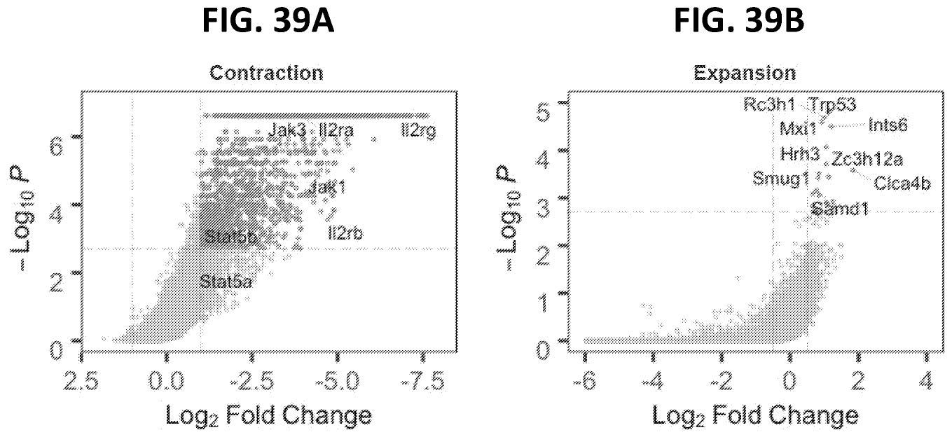

(see also Table S1 of Loo et al., Immunity 53, 143-157, 2020). In a parallel analysis, 22 and 1497 genes that affect cell expansion and contraction, respectively, were also identified (p-value<0.002, LFC>1, (FIGS. 39A-B) (see also Table S2 of Loo et al., Immunity 53, 143-157, 2020). As expected, genes belonging to pathways known to regulate Foxp3 expression both transcriptionally (Cbfb, Runx3) (Rudra et al., Nat Immunol 10:1170-1177, 2009) and post-transcriptionally through the regulation of Foxp3 protein stability (Usp7, Stub1) were identified (Chen et al., Immunity 39, 272-285, 2013; van Loosdregt et al., Trends Immunol 35, 368-378, 2013) (FIG. 4A).

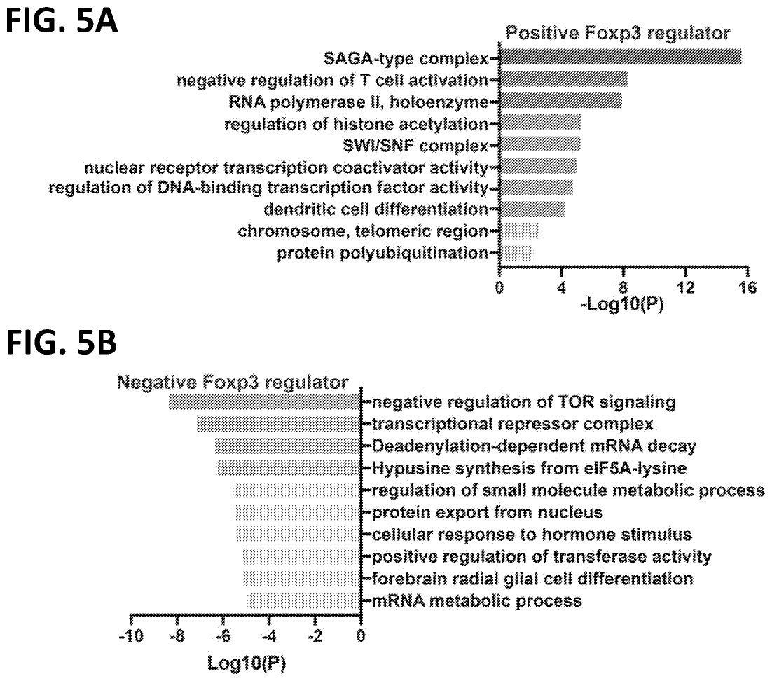

The potential positive and negative regulators were next compared with genes involved in cell contraction and expansion to exclude hits that might affect Foxp3 expression indirectly by affecting cellular fitness in general, leaving 197 positive Foxp3 regulators and 327 negative Foxp3 regulators (FIG. 4B) (see also Table S3 of Loo et al., Immunity 53, 143-157, 2020). Gene ontology analysis of positive Foxp3 regulators revealed a number of notable functional clusters including SAGA-type complex, negative regulation of T cell activation, RNA Polymerase II holoenzyme, positive regulation of histone modification, and SWI/SNF complex (FIG. 5A) (see also, Table S4 of Loo et al., Immunity 53, 143-157, 2020). Among negative Foxp3 regulators, genes are highly enriched in clusters related to negative regulation of TOR signaling, transcriptional repressor complex, mRNA decay and metabolism, and hypusine synthesis from eIF5A-lysine (FIG. 5B) (see also Table S4 of Loo et al., Immunity 53, 143-157, 2020). Several of these pathways, including mTOR signaling, Foxp3 ubiquitination/deubiquitination, and transcriptional regulation, have been implicated in Foxp3 regulation previously, suggesting that the screen is robust for the validation of known pathways and the discovery of additional regulators of Foxp3. Specifically, many genes encoding subunits of the SAGA (Ccdc101, Tada2b, Tada3, Usp22, Tada1, Taf61, Supt5, Supt20) and SWI/SNF (Arid1a, Brd9, Smarcd1) complexes were identified, strongly suggesting that these complexes could have indispensable roles for Foxp3 expression. Thus, further validation and characterization of the SAGA and SWI/SNF related complexes was performed.

### Example 3

### The SAGA Complex as a Regulator of Foxp3 Expression and Treg Activity

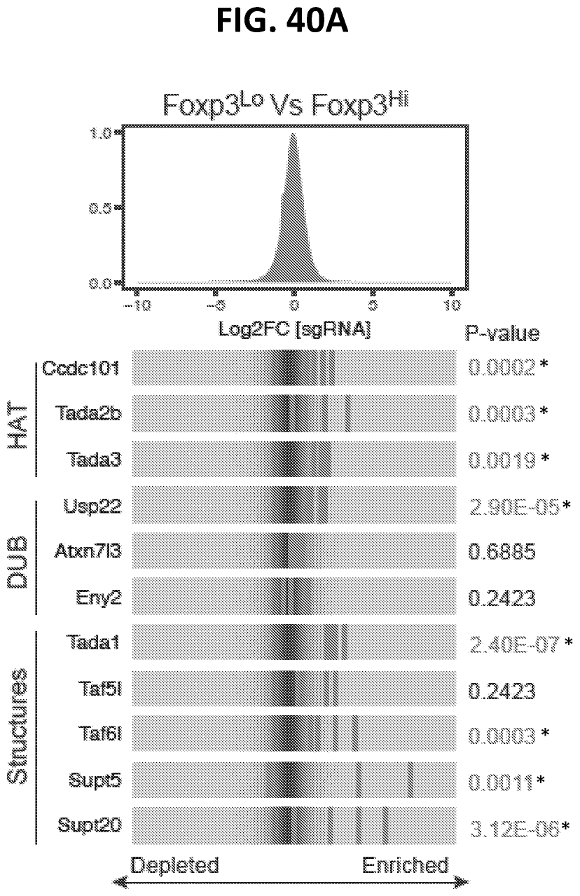

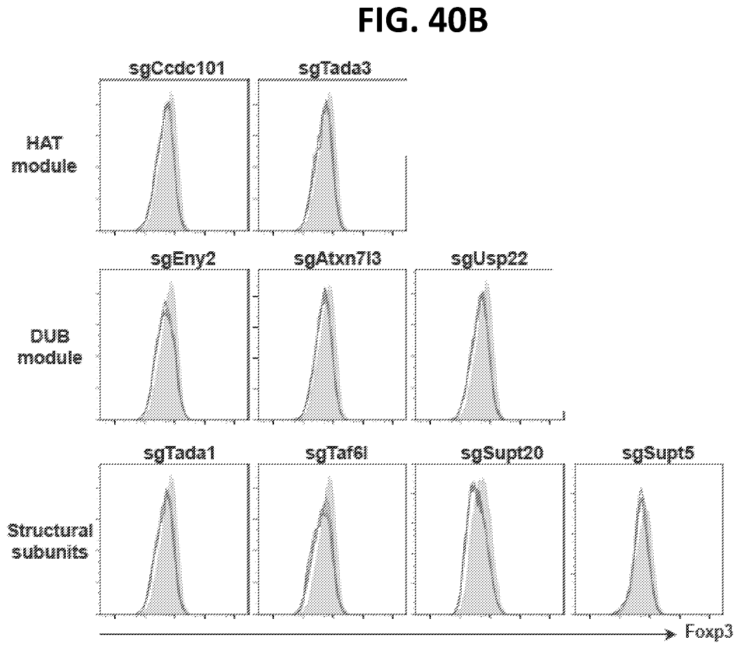

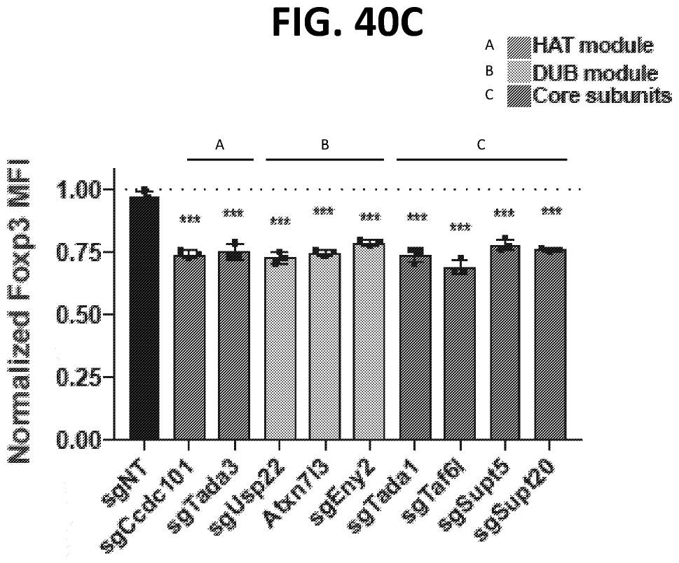

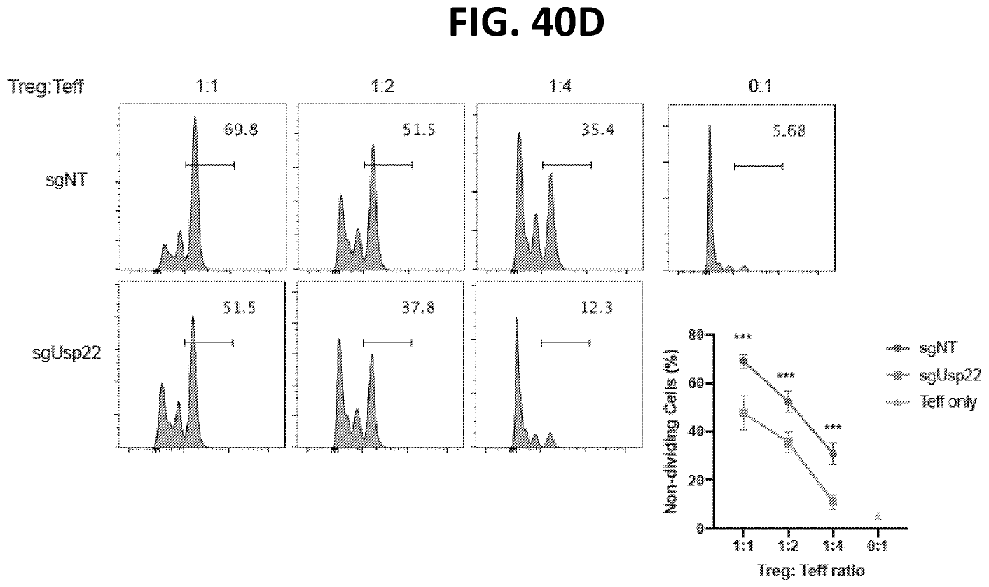

The SAGA complex possesses histone acetyltransferase (HAT) and histone deubiquitinase (DUB) activity, and functions as a transcriptional co-activator through interactions with transcription factors and the general transcriptional machinery (Helmlinger and Tora, Trends Biochem Sci 42:850-861., 2017; Koutelou et al., Curr Opin Cell Biol 22:374-382, 2010). Ccdc101, Tada2b, and Tada3 were identified in the HAT module, Usp22 in the DUB module, and Tada1, Taf61, Supt5, and Supt20 from the core structural module were identified among positive Foxp3 regulators that do not affect cell expansion or contraction (FIG. 40A). The potential regulatory function of SAGA complex subunits was investigated by using sgRNAs to target individual subunits in Treg cells and measure Foxp3 expression (FIGS. 40B and 40C). Deletion of every subunit tested resulted in a significant and 19-29% reduction in Foxp3 mean fluorescence intensity (MFI).

Next, the function of SAGA subunit Usp22 was further investigated in an in vitro suppression assay, which measures the suppression of T cell proliferation when conventional T cells are co-cultured with Treg cells at increasing ratios. Treg cells transduced with sgRNAs targeting Usp22 were found to have compromised Treg suppressor activity as compared to Treg cells transduced with a non-targeting control sgRNA, with significantly more proliferation of T effector cells (Teff) at every ratio of Treg to Teff ratio tested (FIG. 40D). These results provide independent validation of our genome-wide screen analyses for this class of chromatin regulators and demonstrate that disrupting the SAGA complex by sgUsp22 reduces Foxp3 expression and Treg suppressor function.

### Example 4

### Brd9-Containing ncBAF Complex is a Regulator of Foxp3 Expression

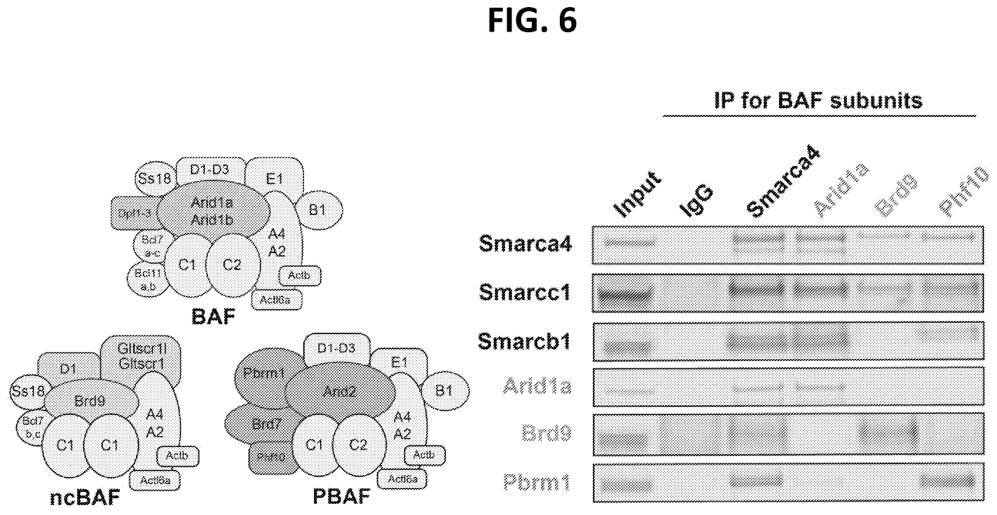

The role of SWI/SNF complex variants (BAF, ncBAF, and PBAF complexes) in Foxp3 expression was next investigated. Apart from uniquely incorporating Brd9, the ncBAF complex also contains Gltscr1 or the paralog Gltscr11 and lacks BAF- and PBAF-specific subunits Arid1a, Arid1b, Arid2, Smarce1, Smarcb1, Smarcd2, Smarcd3, Dpf1-3, Pbrm1, Brd7, and Phf10 (FIG. 6). The distinct biochemical compositions of these three SWI/SNF complex assemblies suggest functional diversity. However, it is not known which SWI/SNF complex assemblies are expressed in Treg cells and the potential roles of specific SWI/SNF variants in regulating Foxp3 expression and Treg development have not been studied. Therefore, co-immunoprecipitation assays were performed to probe the composition of SWI/SNF-related complexes in Treg cells. As expected, immunoprecipitation of Smarca4, a core component of all three SWI/SNF complexes, revealed association of common subunits Smarcc1 and Smarcb1, as well as specific subunits Arid1a, Brd9, and Pbrm1 Immunoprecipitations against Arid1a, Brd9, and Phf10 revealed the specific association of these subunits with BAF, ncBAF, and PBAF complexes, respectively (FIG. 6). These results established that all three SWI/SNF complexes are present with the expected composition in Treg cells.

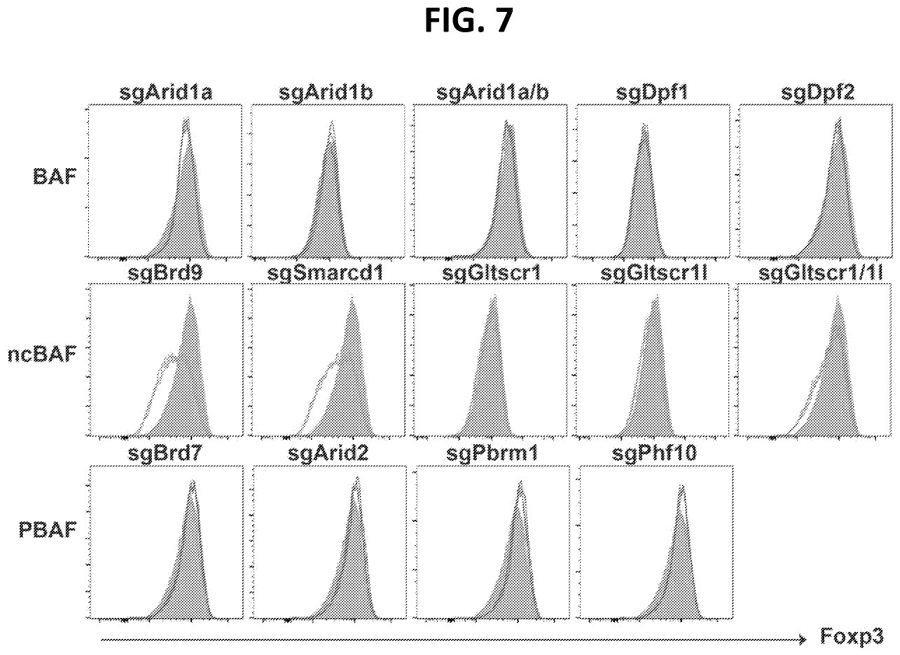

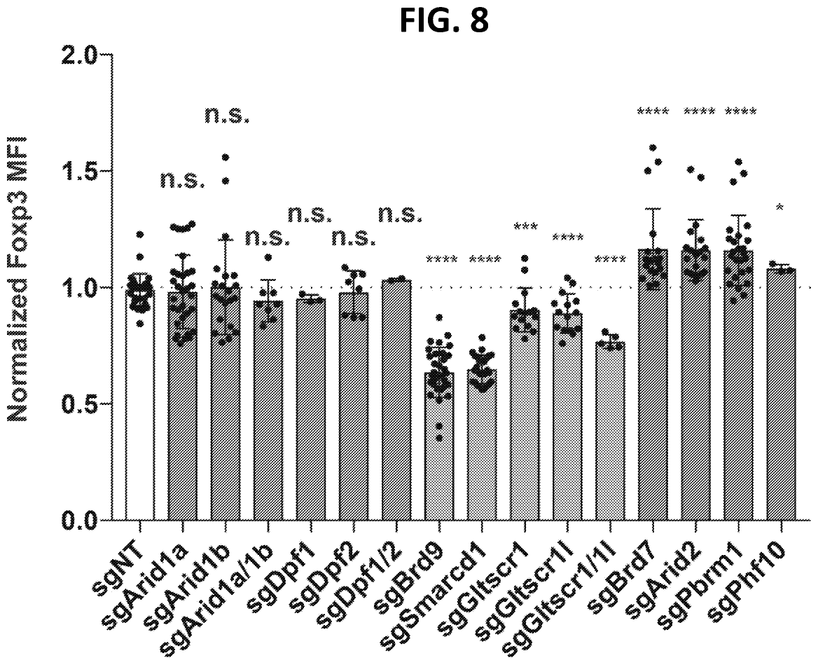

In the screen, Brd9, Smarcd1, and Arid1a were identified among positive regulators of Foxp3, whereas SWI/SNF shared subunits Smarca4, Smarcb1, Smarce1, and Actl6a were identified in cell contraction (see also Table S3 of Loo et al., Immunity 53, 143-157, 2020). This suggested a potential regulatory role for ncBAF and/or BAF complexes. To explore the specific function of BAF, ncBAF, and PBAF complexes in Foxp3 expression, independent sgRNAs were cloned to target unique subunits for each complex, and Foxp3 mean fluorescent intensity (MFI) in sgRNA transduced Treg cells was measured. A role for the ncBAF complex in Foxp3 expression was observed in Treg cells. Specifically, sgRNA targeting of ncBAF specific subunits, including Brd9 and Smarcd1, significantly diminished Foxp3 expression by nearly 40% in Treg cells (FIGS. 7 and 8). sgRNA targeting of ncBAF-specific paralogs Gltscr1 and Gltscr11 individually resulted in a slight reduction in Foxp3 expression, which was further reduced by Gltscr1/Gltscr1l double deficiency, suggesting that these two paralogs can compensate in the regulation of Foxp3 expression (FIG. 8). In contrast, sgRNA targeting of PBAF specific subunits, including Pbrm1, Arid2, Brd7, and Phf10, significantly enhanced Foxp3 expression by as much as 17% (FIG. 8). sgRNA targeting of BAF specific subunits Arid1a, Arid1b, Dpf1, or Dpf2 did not significantly affect Foxp3 expression (FIG. 8). To determine if Arid1a and Arid1b could compensate for one another, an Arid1a/Arid1 b double deletion was tested, and it was found that deletion of either or both Arid paralogs resulted in slight, but non-significant reduction in Foxp3 MFI (FIG. 8). These data suggest that ncBAF and PBAF have opposing roles in the regulation of Foxp3 expression.

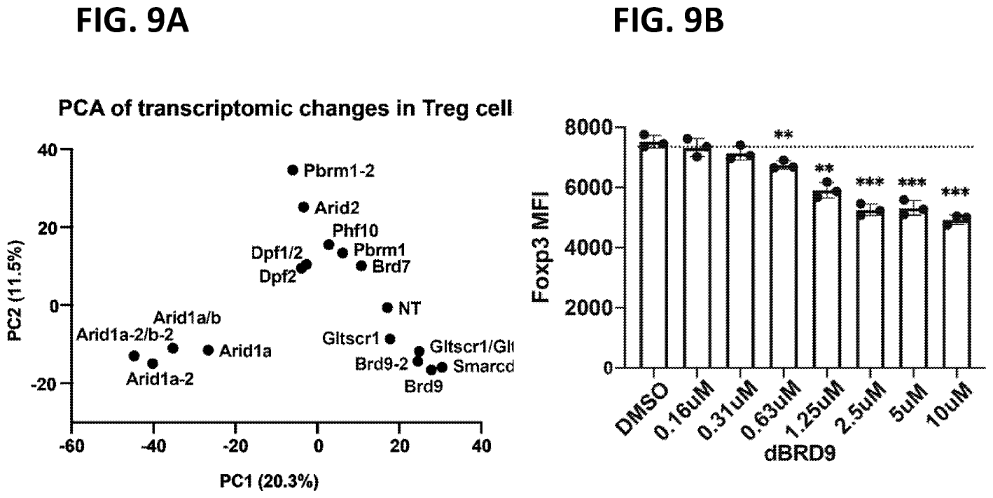

To further explore the role of different SWI/SNF complexes in Treg genome-wide transcription, RNA sequencing from Treg cells with sgRNA targeting of variant-specific subunits with one or two independent guide RNAs was performed and a principal component analysis was conducted. The results show that the ncBAF, PBAF, and BAF have distinct effects at the whole transcriptome level in Treg cells (FIG. 9A).

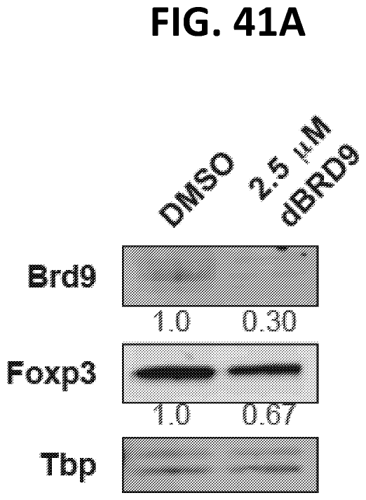

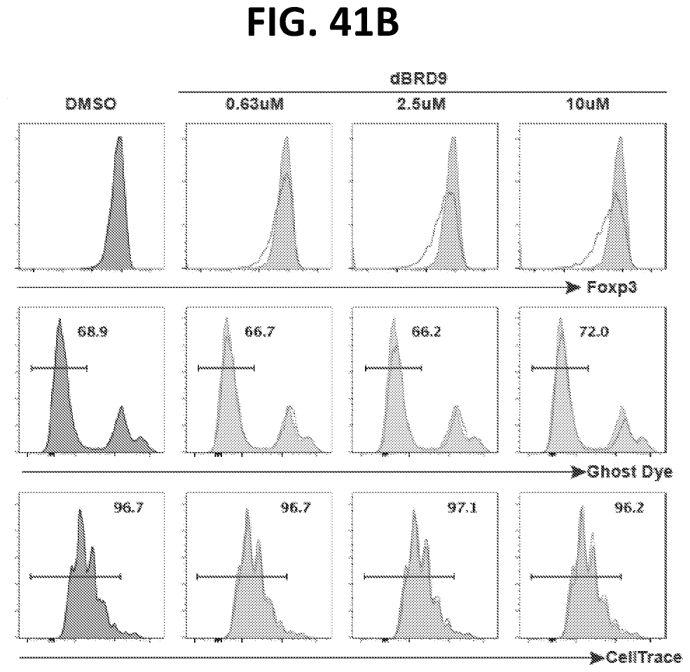

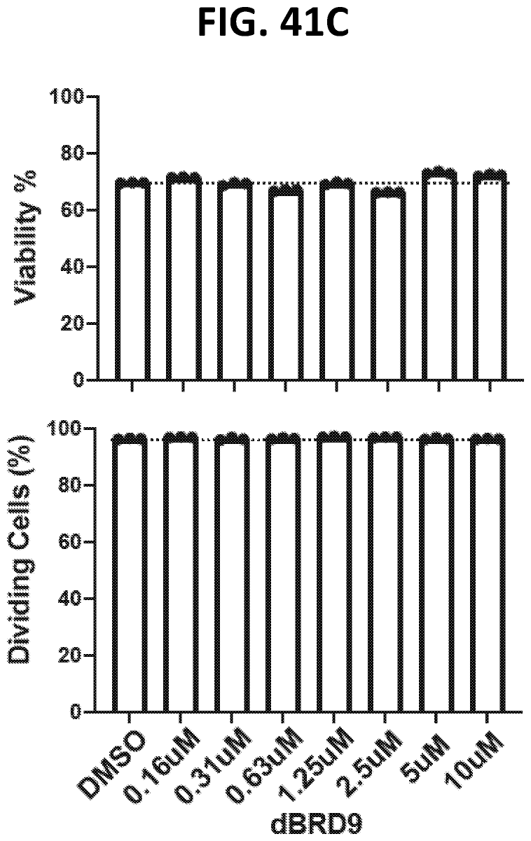

A chemical Brd9 protein degrader (dBRD9) was used as an orthogonal method to probe Brd9 function (Remillard et al., Angew Chem Int Ed Engl 56:5738-5743, 2017). dBRD9 is a bifunctional molecule that links a small molecule that specifically binds to the bromodomain of Brd9 and another ligand that recruits the cereblon E3 ubiquitin ligase. It was confirmed that treatment of Treg cells with dBRD9 resulted in reduced Brd9 protein (FIG. 41A). Similar to sgRNA depletion of Brd9, dBRD9 treatment significantly decreased Foxp3 expression in Treg cells in a concentration-dependent manner, without affecting cell viability or proliferation (FIGS. 9B and 41B). These data demonstrate the requirement for Brd9 in maintenance of Foxp3 expression using both genetic and chemically-induced proteolysis methods.

### Example 5

### Brd9 Regulates Foxp3 Binding of Target Sites

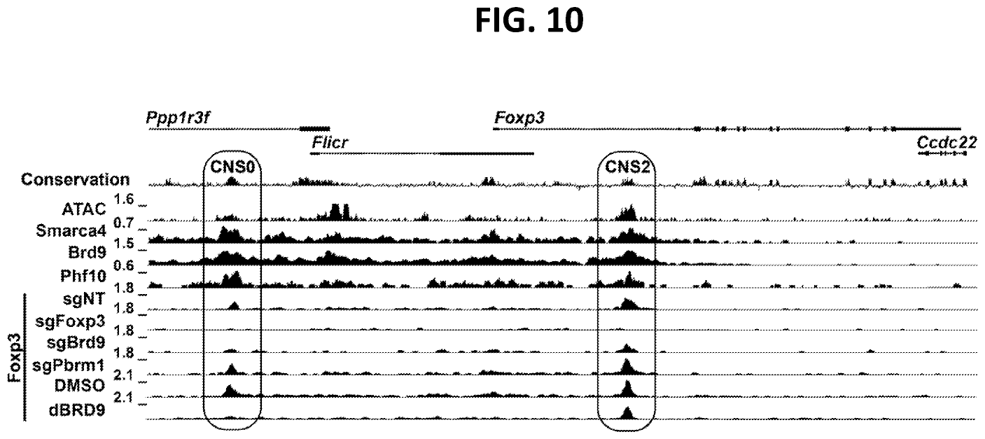

To dissect the molecular mechanism underpinning ncBAF and PBAF complex regulation of Foxp3 expression in Treg cells, chromatin immunoprecipitation was performed followed by genome-wide sequencing (ChIP-seq) in Treg cells using antibodies against the ncBAF-specific subunit Brd9, the PBAF-specific subunit Phf10 and the shared enzymatic subunit Smarca4. Data generated from these ChIP-seq experiments revealed that Brd9, Smarca4, and Phf10 co-localize at CNS2 in the Foxp3 gene locus and at CNS0 found within the Ppp1r3f gene immediately upstream of Foxp3 (FIG. 10). Since CNS2 was previously shown to regulate stable Foxp3 expression through a positive feedback loop involving Foxp3 binding (Feng et al., Cell 158:749-763, 2014; Li et al., Genome Biol 15:554, 2014), and Foxp3 is additionally bound at CNS0 in Treg cells (Kitagawa et al., Nat Immunol 18:173-183, 2017), ncBAF and/or PBAF complexes might affect Foxp3 expression by regulating Foxp3 binding at CNS2/CNS0. A Foxp3 ChIP-seq in Treg cells transduced with sgNT, sgFoxp3, sgBrd9 or sgPbrm1 was performed. A dramatic reduction in Foxp3 binding at CNS2/CNS0 in sgFoxp3 transduced cells was observed. There was also marked reduction of Foxp3 binding at CNS2/CNS0 in Brd9-depleted Treg cells (FIG. 10). In contrast, there was a subtle increase in Foxp3 binding at CNS2/CNS0 in Pbrm1 sgRNA transduced Treg cells (FIG. 10). These data suggest that Brd9 positively regulates Foxp3 expression by promoting Foxp3 binding to its own enhancers.

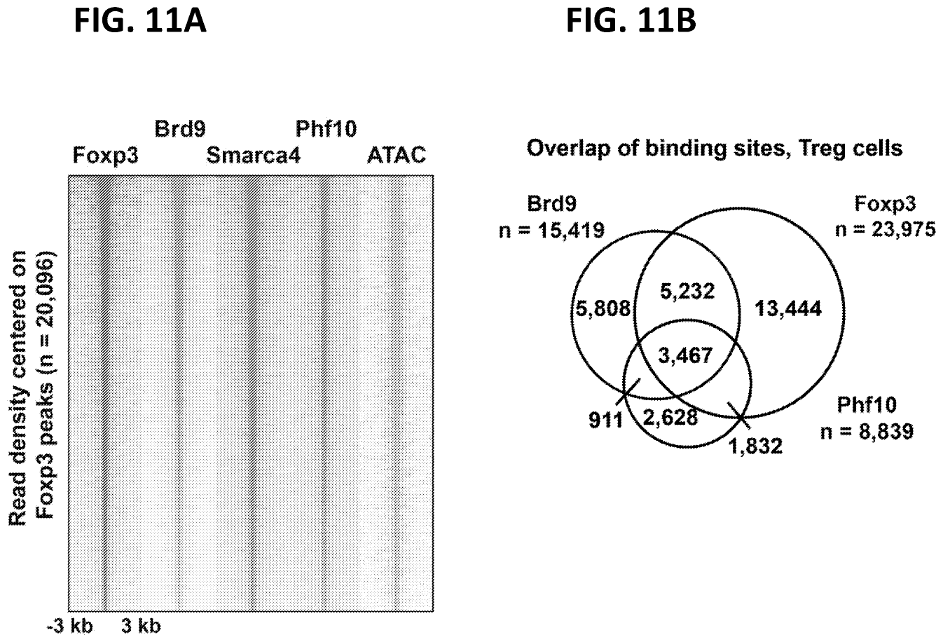

This analysis was expanded to examine the cooperation between Brd9 and Foxp3 genome-wide. Notably, co-binding of Brd9, Smarca4, and Phf10 with Foxp3 at a subset of Foxp3-bound sites was observed (FIGS. 11A and 11B). All four factors localized to promoters, intronic, and intergenic regions of the genome and their binding correlated well with chromatin accessibility as measured by assay of transposase-accessible chromatin with sequencing (ATAC-seq) (FIGS. 11A and 42A). Motif analysis of Foxp3-bound sites revealed an enrichment for motifs recognized by Ets and Runx transcription factors consistent with what has been previously shown (Samstein et al., Cell 151:153-166, 2012). Ets and Runx motifs were also among the most significant motifs at both Brd9-bound sites, along with an enrichment of the Ctcf motif (FIG. 42B). These results demonstrate that ncBAF and PBAF complexes are co-localized with Foxp3 at Foxp3 binding sites genome-wide.

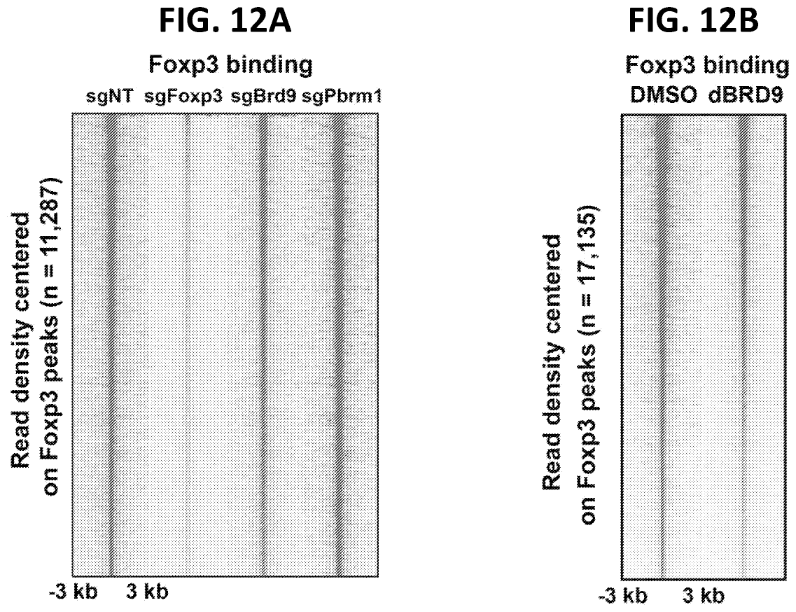

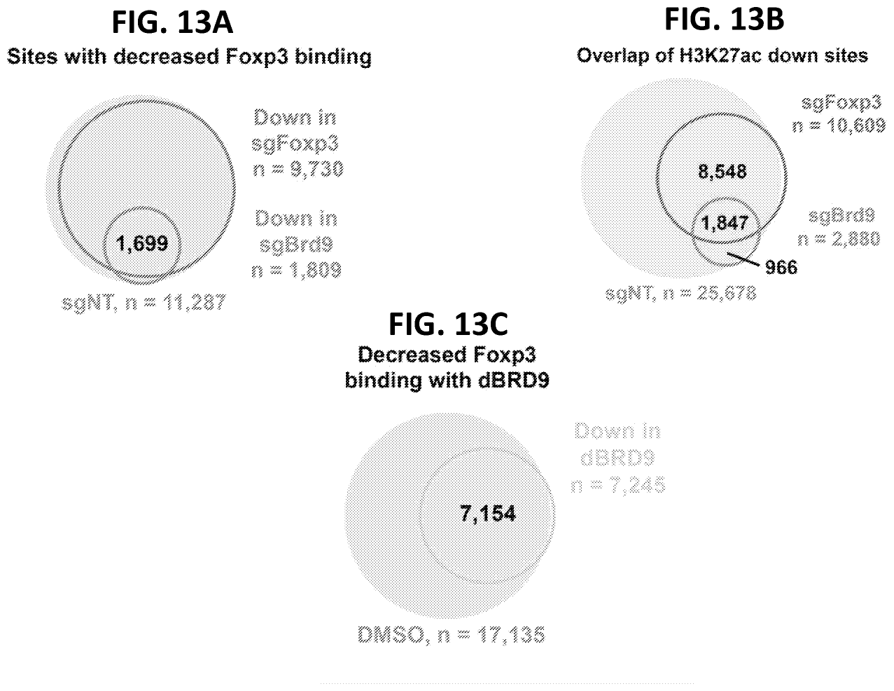

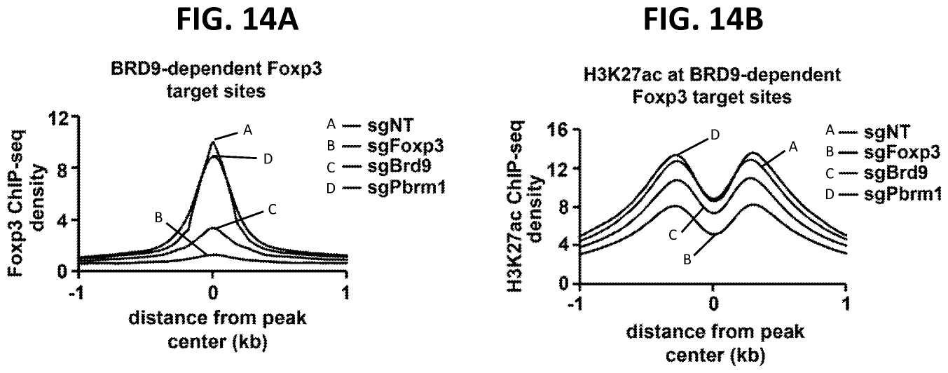

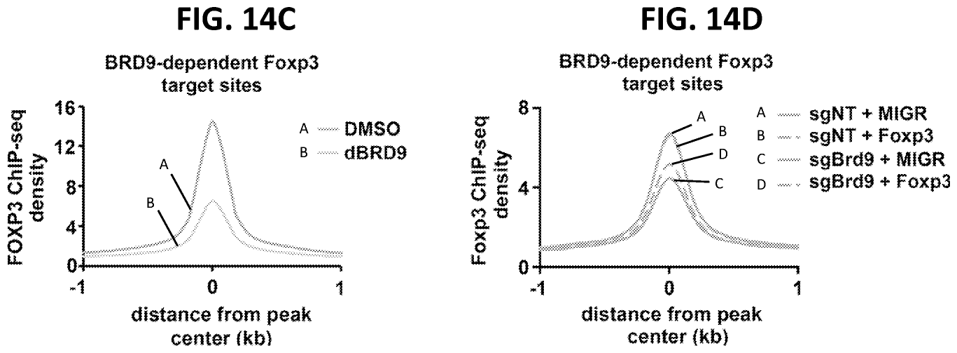

To assess the requirement for Brd9 or Pbrm1 in Foxp3 targeting genome-wide, Foxp3 binding in Treg cells transduced with sgNT, sgFoxp3, sgBrd9, or sgPbrm1 at all Foxp3 binding sites was analyzed (FIG. 12A). Foxp3 binding was lost at over 85% of its binding sites in sgFoxp3-transduced Treg cells (FIG. 13A). Foxp3 binding at a subset of these sites was also significantly reduced in sgBrd9-transduced Treg cells (FC1.5, Poisson p<0.0001) (FIGS. 13A and 42C-F). This was a specific function of Brd9, as Foxp3 binding did not change in Pbrm1-depleted Treg cells at these Brd9-dependent sites (FIGS. 14A, 42C). ChIP-seq for the active histone mark H3 lysine27 acetylation (H3K27ac) revealed that Brd9 and Foxp3 cooperate to maintain H3K27ac at over 1,800 shared sites (FIG. 13B). At Brd9-dependent Foxp3 sites, for example, a reduction in H3K27ac in sgFoxp3 and sgBrd9-transduced Treg cells, but not in sgPbrm1-transduced Treg cells was observed (FIG. 14B). Using dBRD9, we further recapitulated our observation that Brd9 loss resulted in diminished Foxp3 binding to chromatin at a subset of Foxp3 target sites (FIGS. 12B, 13C, 14C, and 42C), including at CNS2 and CNS0 (FIG. 10). To determine if ncBAF complexes maintain chromatin accessibility for Foxp3 binding, ATAC-seq was performed on sgBrd9 and sgNT Treg cells (FIG. 42G). Only 61/1699 (3.5%) of Brd9-dependent Foxp3 binding sites had a significant reduction in chromatin accessibility in sgBrd9 Treg cells, suggesting that chromatin remodeling may only minimally contribute to ncBAF-dependent maintenance of Foxp3 binding.

Since Brd9 deficiency leads to reduced Foxp3 expression, next it was investigated whether reduced Foxp3 binding to its target regions in sgBrd9 Treg cells is due to reduced Foxp3 protein, or if Brd9 plays an additional role in facilitating Foxp3 binding to a subset of its targets. To this end, Foxp3 or MIGR vector control was ectopically expressed in sgNT and sgBrd9 transduced Treg cells, and Foxp3 ChIP-seq was performed. Analysis of the Foxp3 ChIP-seq result showed that ectopic Foxp3 expression partially restored Foxp3 binding in sgBrd9 Treg cells, but not to the level of sgNT alone or sgNT with ectopic Foxp3 expression (FIG. 14D). Further analysis revealed that while ectopic Foxp3 expression restored Foxp3 binding to a portion of Brd9-dependent Foxp3 binding sites (e.g., CD44 intergenic, Tigit intergenic, and Ctla2a promoter), binding to the majority of Brd9-dependent sites (˜71%) (e.g., Icos intergenic, Ctla4 intergenic, and Ctla4 promoter) was not rescued by simply restoring Foxp3 expression (FIGS. 42H and 421). These data demonstrate that Brd9 co-binds with Foxp3 at the Foxp3 locus to positively reinforce its expression. Brd9 additionally promotes Foxp3 binding and H3K27ac at a subset of Foxp3 target sites both by potentiating Foxp3 expression and through epigenetic regulation at Brd9/Foxp3 co-bound sites.

### Example 6

### Brd9 Co-Regulates the Expression of Foxp3 and a Subset of Foxp3 Target Genes

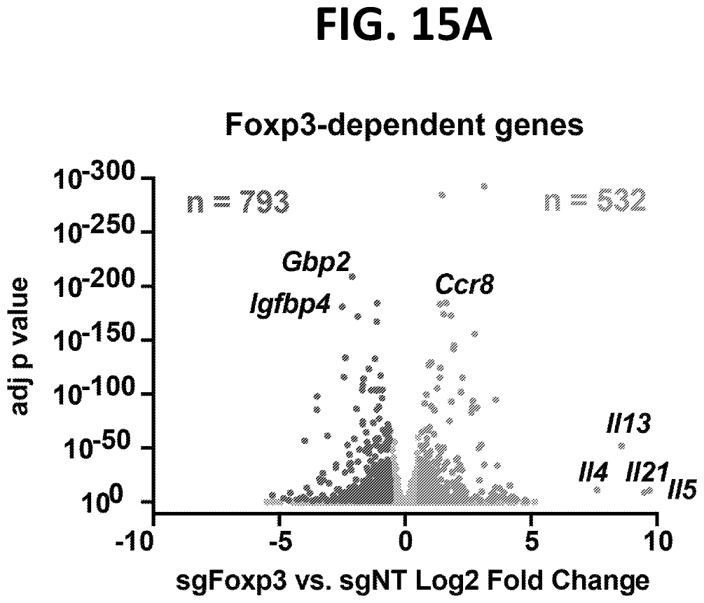

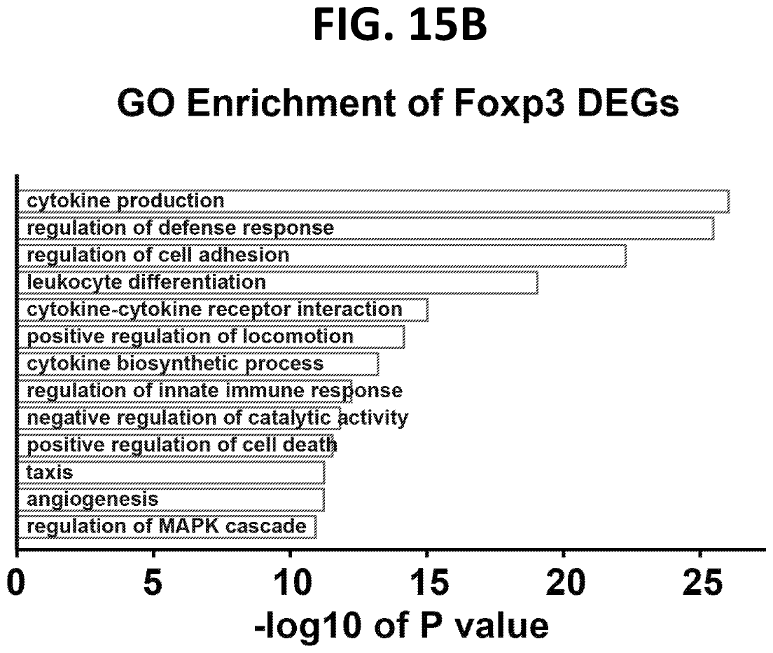

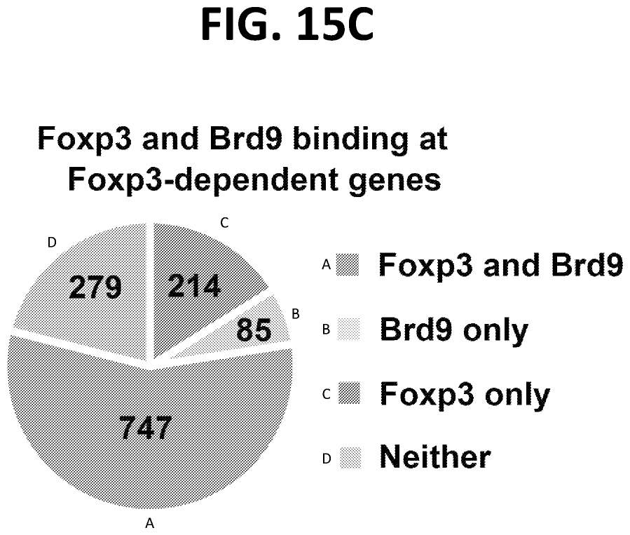

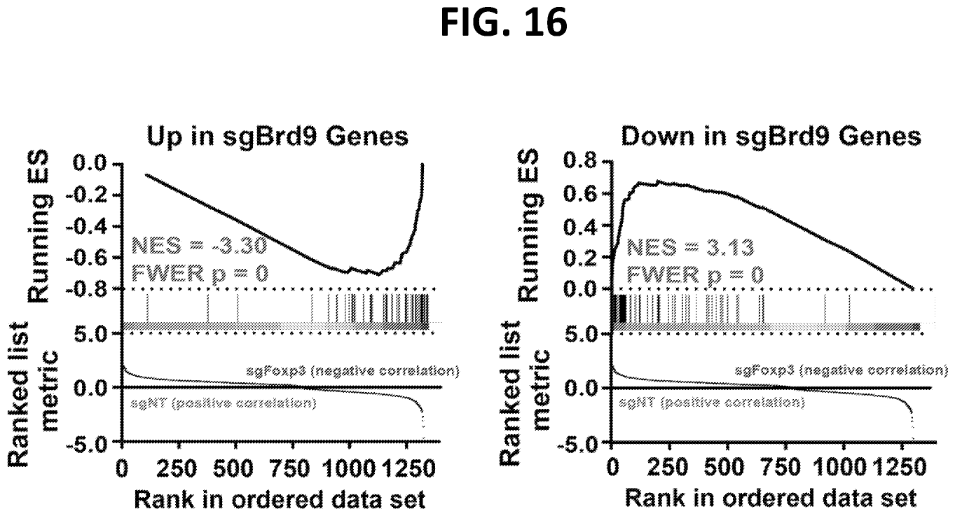

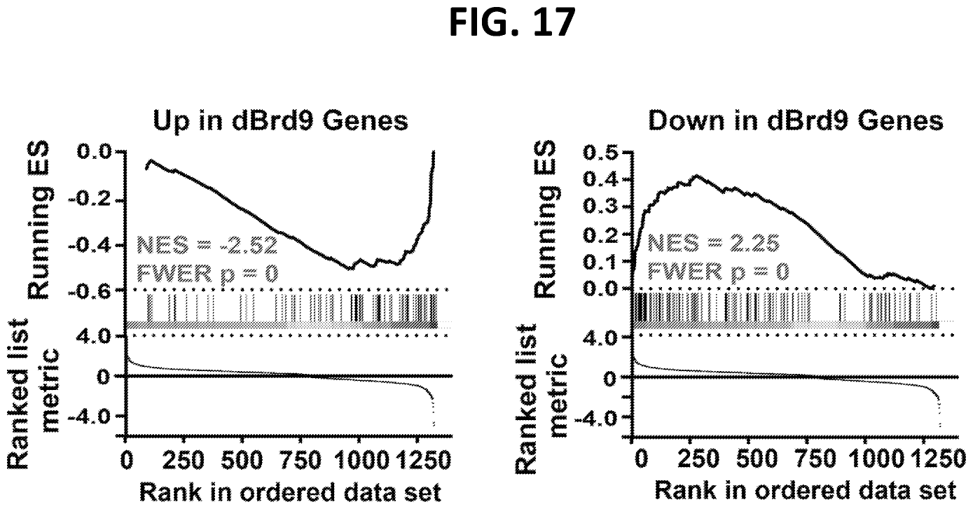

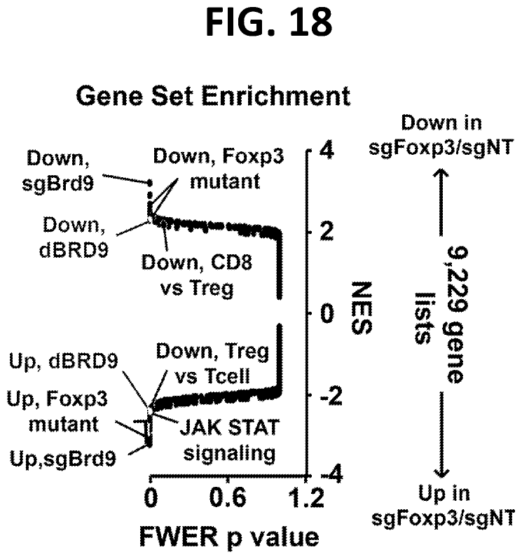

Based on co-binding of Brd9 and Foxp3 at Foxp3 target sites, the effects of Brd9 ablation on the transcription of Foxp3 target genes was further investigated. RNA-seq in Treg cells transduced with sgFoxp3, sgBrd9, or sgNT was performed. Consistent with Foxp3's role as both transcriptional activator and repressor, 793 genes with reduced expression and 532 genes with increased expression in Foxp3 sgRNA transduced Treg cells, which are enriched in ‘cytokine production’, ‘regulation of defense response’, and ‘regulation of cell adhesion,’ was observed (FIGS. 15A and 15B). Of these, 72% were directly bound by Foxp3 in our ChIP-seq dataset and 56% were co-bound by Foxp3 and Brd9 (FIG. 15C). Based on this co-binding, whether Brd9 regulates Foxp3 target gene expression through positively affecting Foxp3 binding to its targets was examined next. Gene set enrichment analysis (GSEA) demonstrated that the sgBrd9 increased genes are significantly enriched among genes that increase upon sgFoxp3 targeting, while the sgBrd9 decreased genes are enriched among genes that decrease in sgFoxp3 Treg cells (FIG. 16). RNA-seq for Treg cells treated with either vehicle or the dBRD9 degrader was also performed and similar significant enrichment for dBRD9 affected genes among the Foxp3 regulated genes was observed (FIG. 17). To determine how Brd9 control of Foxp3 binding affects gene expression, Foxp3 binding sites were divided into quartiles based on most affected (Brd9-dependent) to least affected (Brd9-independent) by sgBrd9 transduction and compared fold changes in gene expression in sgBrd9 versus sgNT Treg cells. Indeed, gene expression of Brd9-dependent Foxp3 target genes was significantly more affected upon sgBrd9 targeting than expression of Brd9-independent Foxp3 target genes (FIG. 42I). Furthermore, gene expression was significantly more affected in sgSmarcd1 transduced (an ncBAF subunit) Treg cells, but not in sgPbrm1 transduced (a PBAF subunit) Treg cells, at Brd9-dependent Foxp3 target genes (FIG. 42I). Thus, ncBAF complexes regulate Foxp3 target genes through potentiation of Foxp3 binding at its target sites. Notably, the Brd9-dependent target gene sets generated from our RNA-seq data were among the most significantly enriched dataset of 9,229 immunological, gene ontology and curated gene sets when analyzed against the sgFoxp3 transduced Treg expression data (FIG. 18). In addition, both datasets were significantly enriched for genes that are differentially expressed between Treg and conventional T cell (Feuerer et al., PNAS, 107:5919-5924, 2010), and between Foxp3 mutant Treg from scurfy mice and wild-type Treg (Hill et al., Immunity 27:786-800, 2007). These data define a role for Brd9 in Treg through specifically regulating the expression of Foxp3 itself and a subset of Foxp3 target genes.

### Example 7

### ncBAF Complex is Required for Normal Treg Suppressor Activity

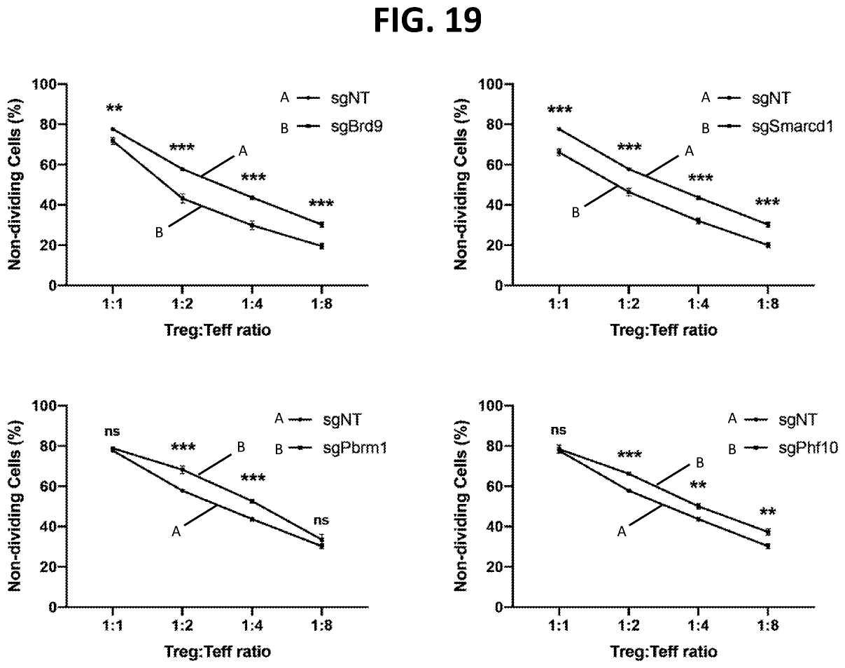

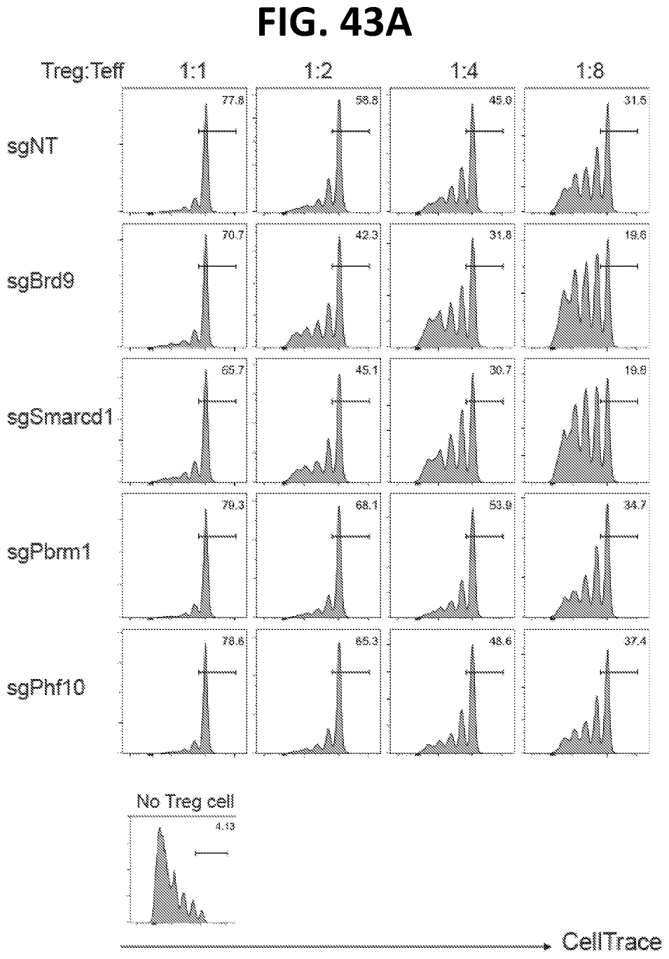

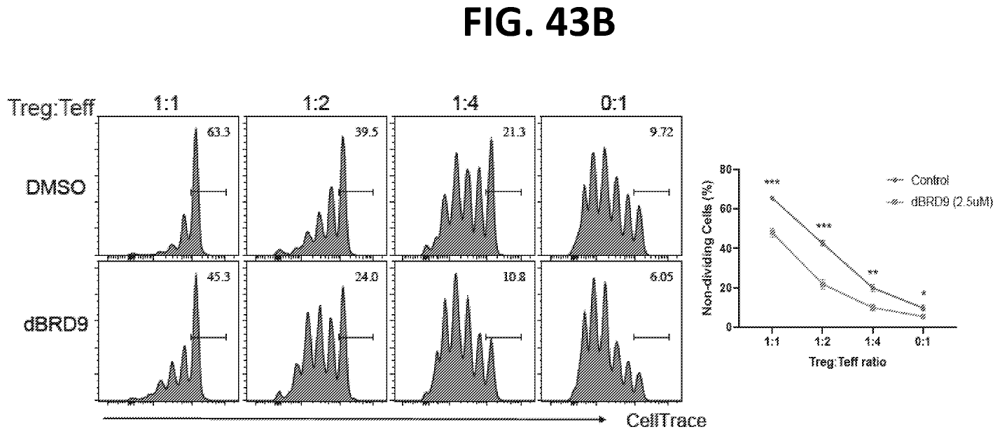

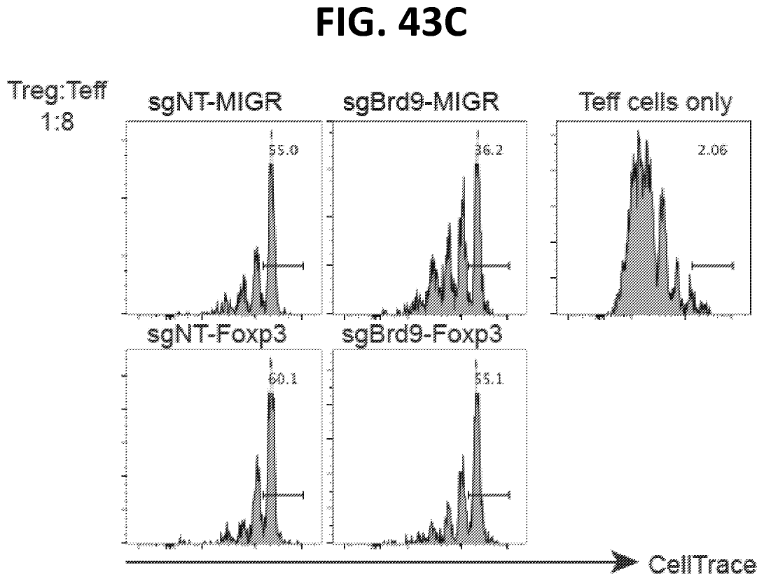

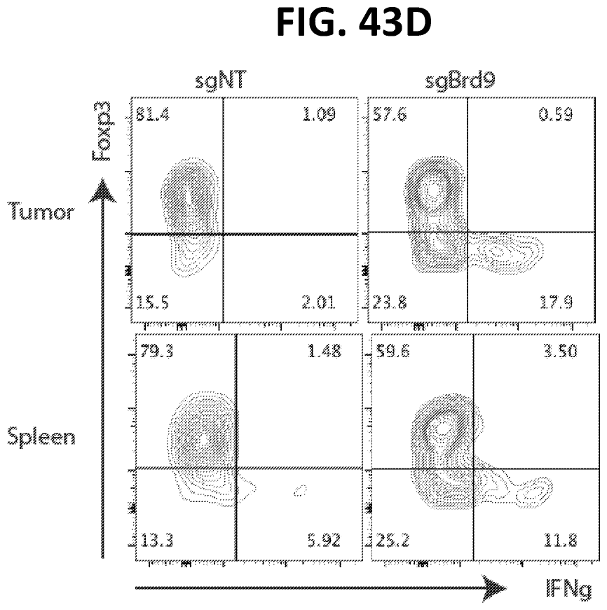

The divergent roles of ncBAF and PBAF complexes in regulating Foxp3 expression suggested that these complexes might also differentially affect Treg suppressor function. sgRNA targeting of ncBAF-specific Brd9 and Smarcd1 or PBAF-specific Pbrm1 and Phf10 was performed in Treg cells with function measured by conducting an in vitro suppression assay. Treg cells depleted of Brd9 or Smarcd1 exhibited significantly reduced suppressor function, whereas depletion of Pbrm1 or Phf10 resulted in significantly enhanced suppressor function (FIGS. 19 and 43A). These data demonstrate that the opposing regulation of Foxp3 expression by ncBAF and PBAF complexes results in decreased/increased Treg suppressor activity upon ncBAF or PBAF subunit deletion, respectively. Similar to sgRNA depletion of Brd9, Treg cells treated with dBRD9 also showed significantly and specifically compromised Treg suppressor function in vitro (FIG. 43B).

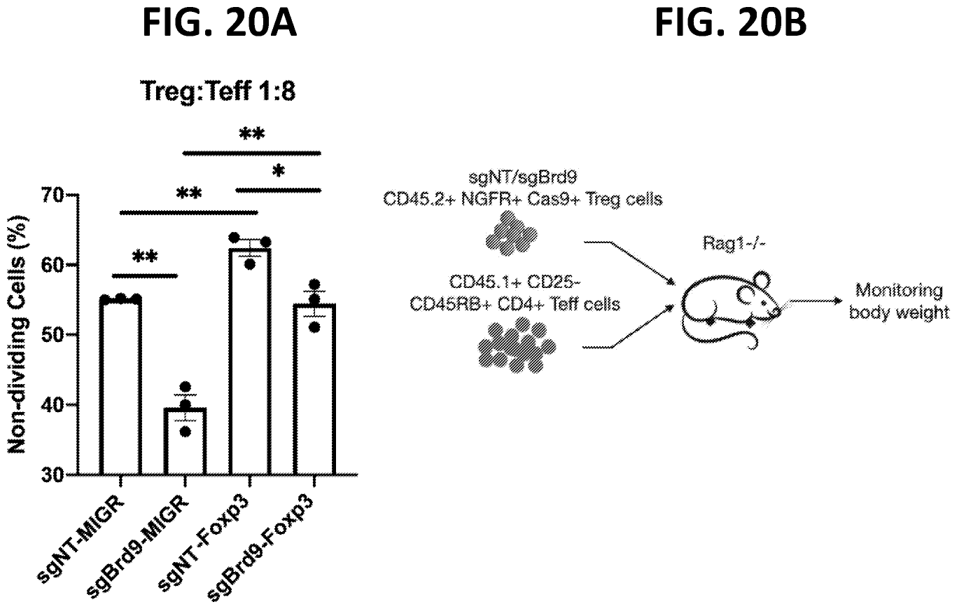

Next, it was determined if the reduced suppressor activity in sgBrd9 Treg cells could be rescued by overexpression of Foxp3. Ectopic expression of Foxp3 in sgBrd9 Treg cells partially restored Treg suppressor activity to a level comparable to sgNT controls, but still lower compared to sgNT Treg cells with ectopic Foxp3 expression (FIGS. 20A and 43C). These results underscore the role of Brd9 in Foxp3 expression maintenance and optimal Treg suppressor activity, and further demonstrate that dBRD9 reduces Treg suppressor activity without impairing T effector responses in vitro.

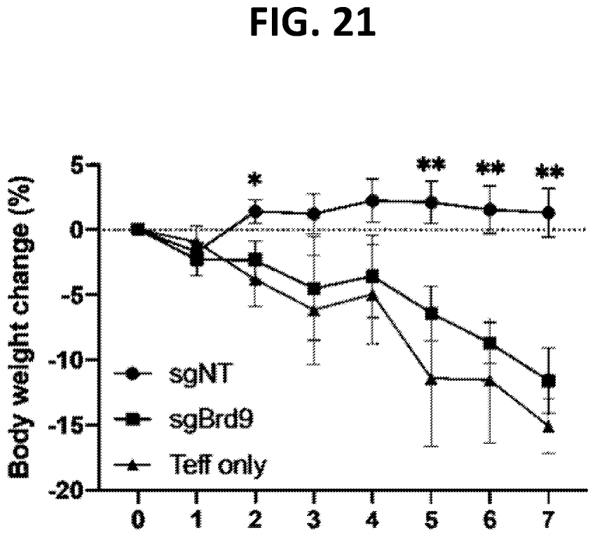

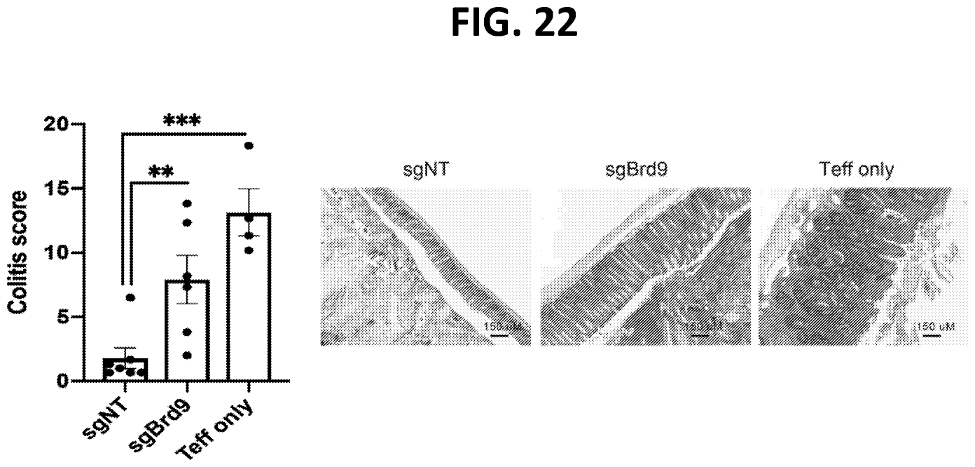

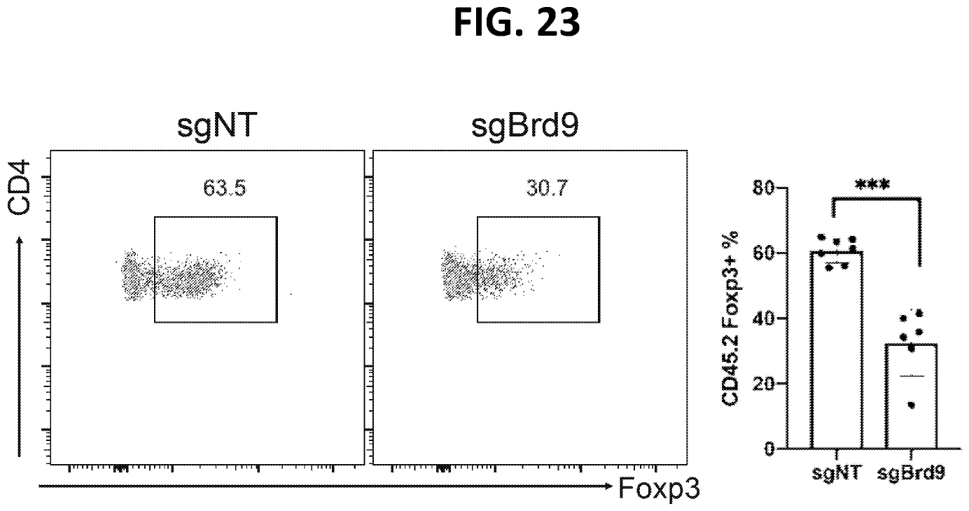

To demonstrate Brd9 also affects Treg function in vivo, a T cell transfer-induced colitis model was used. In this model, Rag1^{-/-} mice were either transferred with CD45.1⁺CD4⁺CD25⁻CD45RB^{hi} effector T cell (Teff) only, or co-transferred with Teff along with CD45.2⁺ Treg cells transduced with sgBrd9 or control sgNT (FIG. 20B). Mice transferred with Teff cells alone lost body weight progressively due to development of colitis. Co-transfer of Treg cells transduced with sgNT protected recipient mice from weight loss, whereas co-transfer of sgBrd9 transduced Treg cells failed to protect recipients from losing weight (FIG. 21). The mice transferred with Brd9-depleted Treg cells showed significant colitis pathology at seven weeks compared to mice that received control Treg cells (FIG. 22). Furthermore, Brd9 depletion also led to compromised Treg stability after transfer, manifested by reduced Foxp3⁺ cell frequencies within the CD45.2⁺CD4⁺ transferred Treg population (FIG. 23). These results demonstrate that Brd9 is a regulator of normal Foxp3 expression and Treg function in inflammatory bowel disease in vivo.

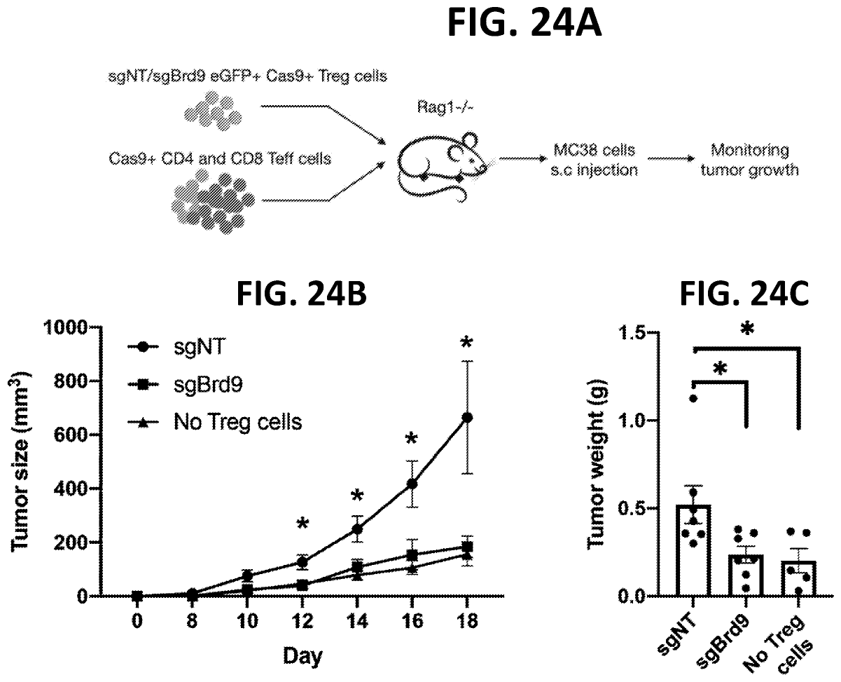

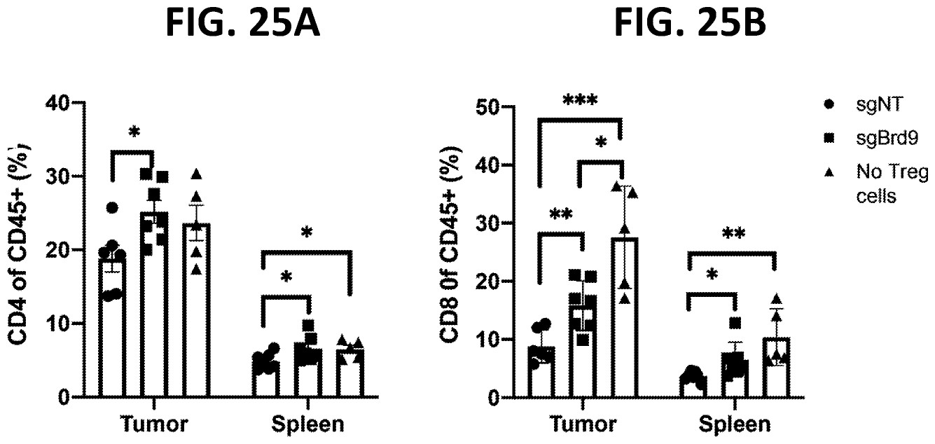

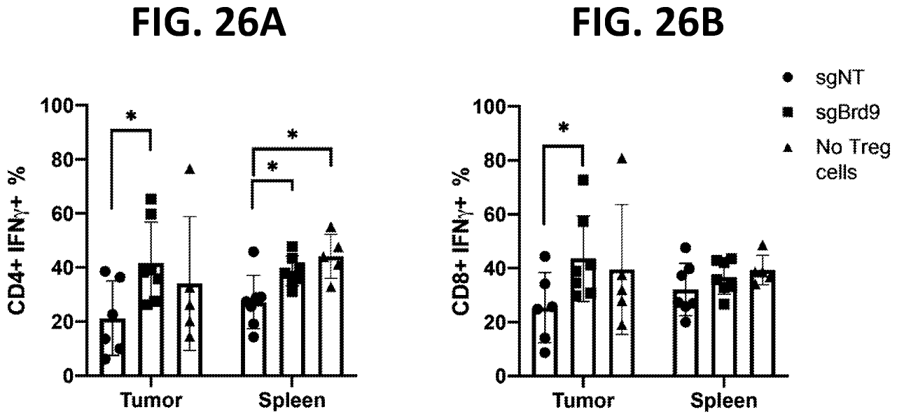

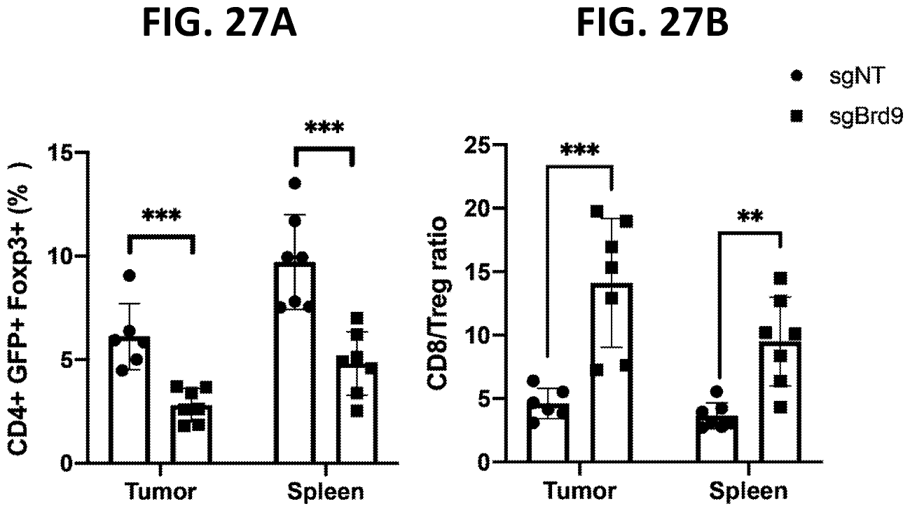

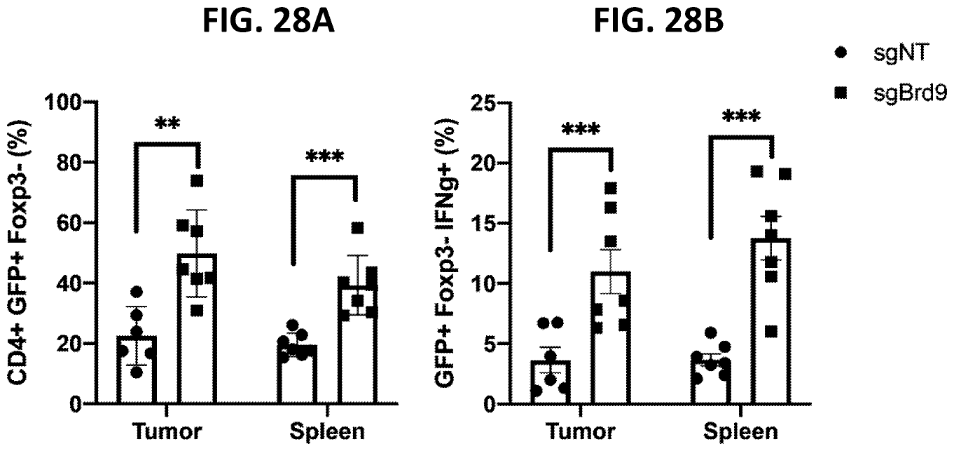

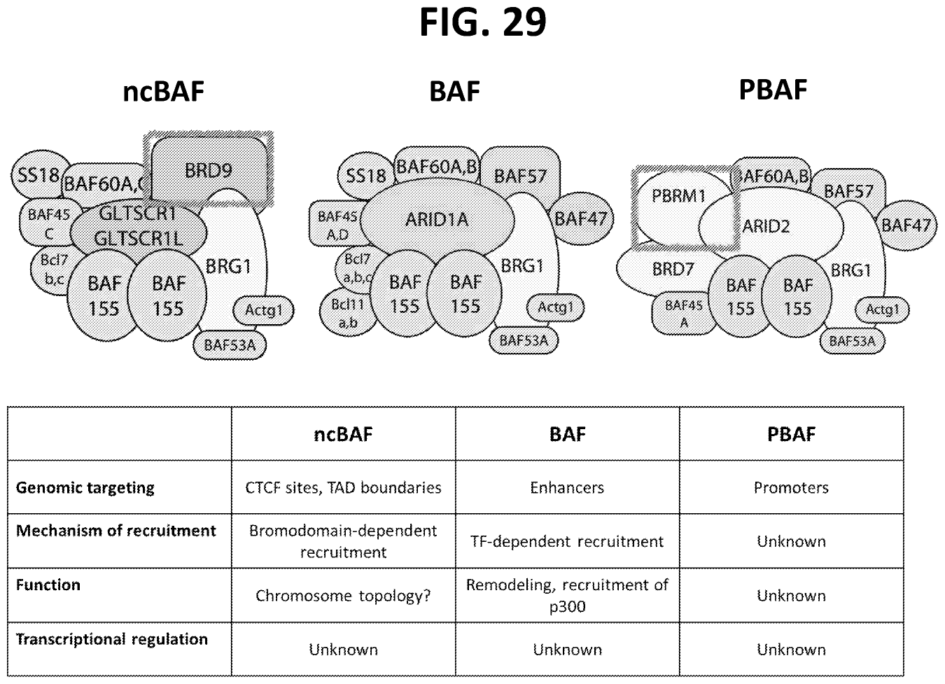

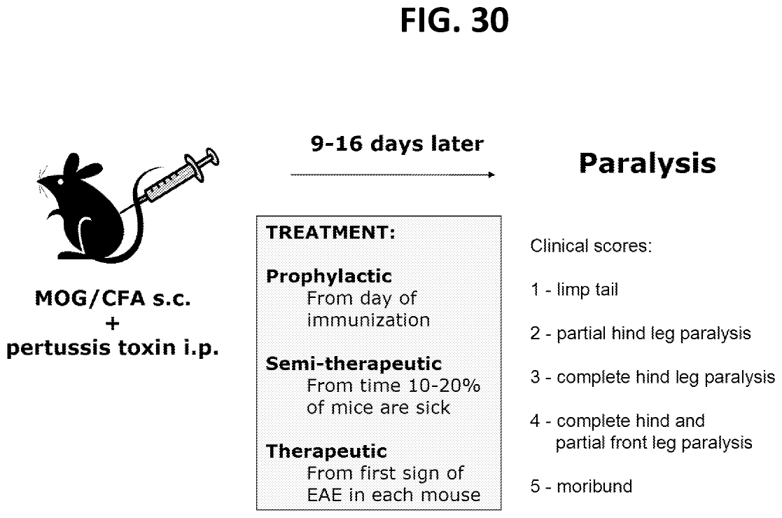

In addition to their beneficial role in preventing autoimmune diseases, Treg cells also function as a barrier to anti-tumor immunity. Thus, it was determined the compromised suppressor function shown in Brd9 deficient Treg could be exploited to disrupt Treg-mediated immune suppression in tumors. The MC38 colorectal tumor cell line was used to induce cancer due to a prominent role Treg play in this cancer model (Delgoffe et al., Nature 501(7466):252-256, 2013). Rag1^{-/-} mice were used as recipients for adoptive transfer of Treg depleted-CD4 and CD8 T cells (Teff) only, or co-transfer of Teff with Treg cells transduced with either sgBrd9 or sgNT. MC38 tumor cells were implanted subcutaneously on the following day (FIG. 24A). Transfer of sgNT Treg cells allowed for significantly faster tumor growth compared to mice that received Teff cells only (“No Treg”) due to suppression of the anti-tumor immune response by Treg cells (FIG. 24B, 24C). Furthermore, tumor growth in mice that received sgBrd9 transduced Treg cells was significantly slower than in mice that received sgNT Treg cells, consistent with our findings that Brd9 deficiency reduced Treg suppressor activity (FIG. 24B, 24C). Both CD4 and CD8 T cell tumor infiltration significantly increased in mice that received sgBrd9 transduced Treg cells compared to sgNT Treg cells (FIGS. 25A and 25B). Additionally, the percentage of IFN-γ producing intra-tumor CD4 and CD8 T cells in mice that received sgBrd9 transduced Treg cells was significantly greater than the sgNT Treg condition, and comparable to the transfer of Teff alone (“No Treg”) (FIGS. 26A and 26B). Consistent with observations that Brd9 is required for Treg persistence in vivo (FIG. 22), the percentage of transferred Treg cells was reduced in mice that received sgBrd9 transduced Treg cells relative to sgNT Treg cells (FIG. 27A). Overall, a 2-3 fold increase in the ratio of CD8 T cells to Treg cells in tumor and spleen was observed in the sgBrd9 versus the sgNT condition, consistent with the enhanced anti-tumor immune response in mice that received sgBrd9 transduced Treg cells (FIG. 27B). To examine if Brd9 deficiency promotes generation of inflammatory ex-Treg cells, Foxp3 and IFN-γ expression was measured within the transferred sgBrd9 or sgNT Treg population marked with a GFP reporter. Ablation of Brd9 led to an increase in the GFP⁺Foxp3⁻ ex-Treg population compared to sgNT Treg cells (FIGS. 28A, 43D). More importantly, a higher percentage of sgBrd9 ex-Treg cells produced IFN-γ compared to sgNT ex-Treg cells (FIGS. 28B and 43D), contributing to slower tumor growth in mice that received sgBrd9 Treg cells. These results demonstrate that Brd9 promotes Treg lineage stability and suppressive function in MC38 tumors and Brd9 deficiency in Treg improves anti-tumor immunity.

### Example 8

### Treg-Specific Pbrm1 Deletion Confers Resistance to Encephalomyelitis (EAE) In Vivo

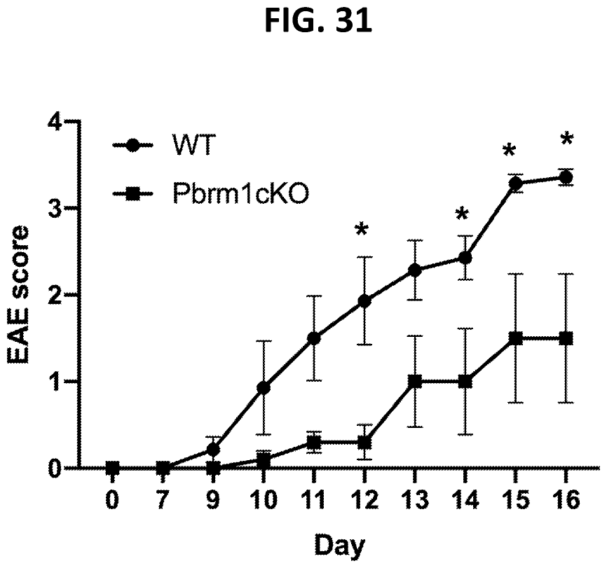

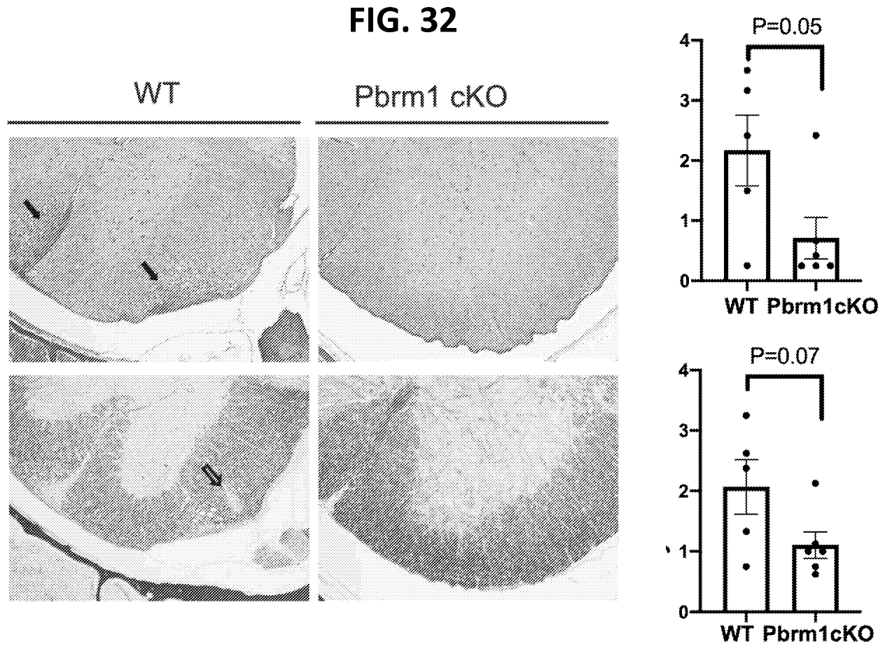

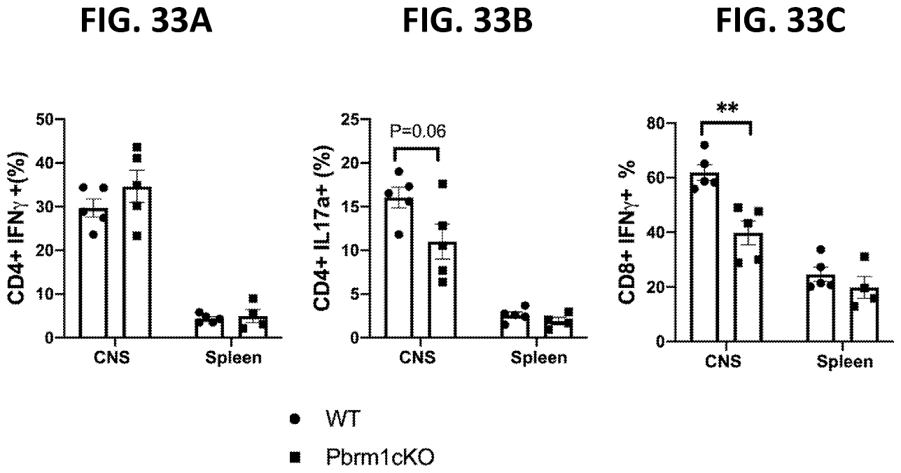

A Treg-specific Pbrm1 conditional knockout (cKO) mouse strain was generated by breeding a Pbrm1 floxed mouse (Pbrm1^{fl}) with a Foxp3^{Cre} knock-in mouse. To show the function of Pbrm1 deficient Tregs in vivo, Pbrm1 cKO mice and WT control mice were challenged with encephalomyelitis (EAE), a classic autoimmune disease model of central nervous system inflammation, by immunizing them with MOG peptide in complete Freund's adjuvant. The Pbrm1 cKO mice were significantly more resistant to disease development as shown by clinical scores (FIG. 31). Histopathology analysis showed more immune cell infiltration in the CNS tissue of wildtype mice compared to Pbrm1 cKO mice (FIG. 32). Both inflammation and demyelination of the CNS tissue were reduced in Pbrm1 cKO mice as compared to wild-type mice (FIG. 32). FACS analysis additionally showed less CD4+IL-17A+ and CD8⁺ IFN-γ+ T cells infiltrating the CNS tissue of the Pbrm1 cKO mice at the end point on day 16 (FIGS. 33B and 33C). This provides in vivo evidence that the PBAF complex restricts Treg suppressor activity in the context of autoimmune disease.

### Example 9

### Treg-Specific Deletion of Brd9 Promotes Tumor Immunity and Prolong Survival in a Glioblastoma Model

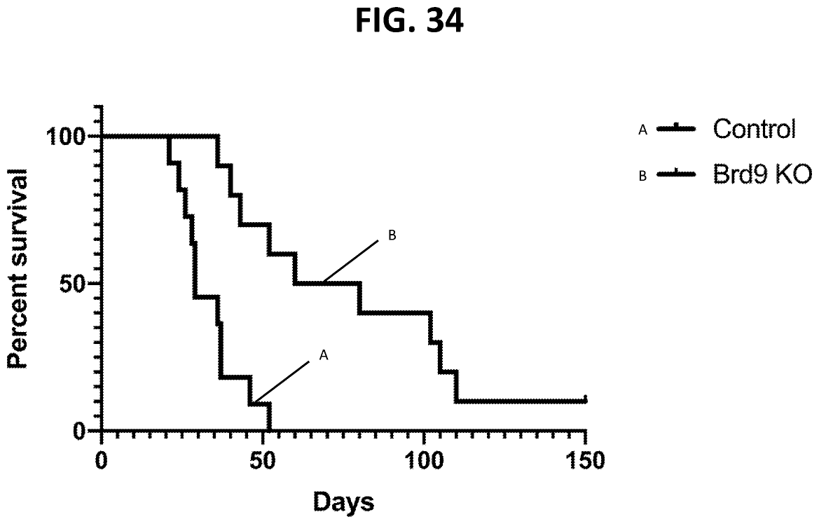

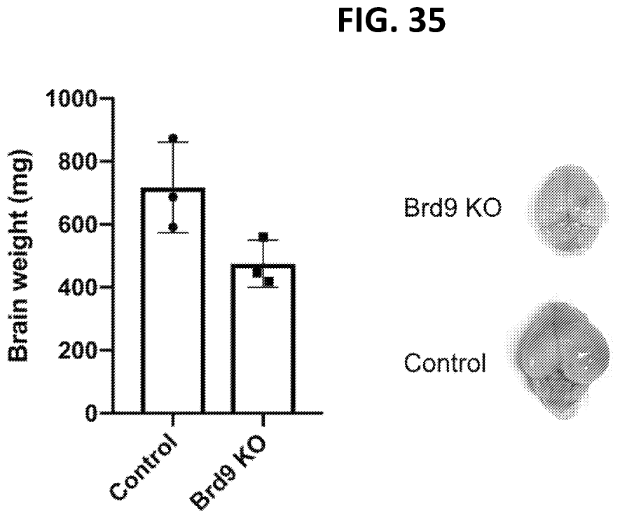

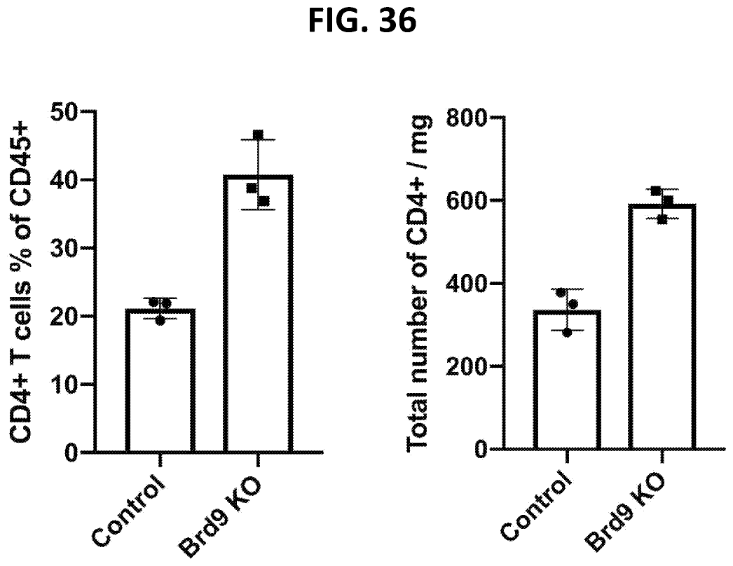

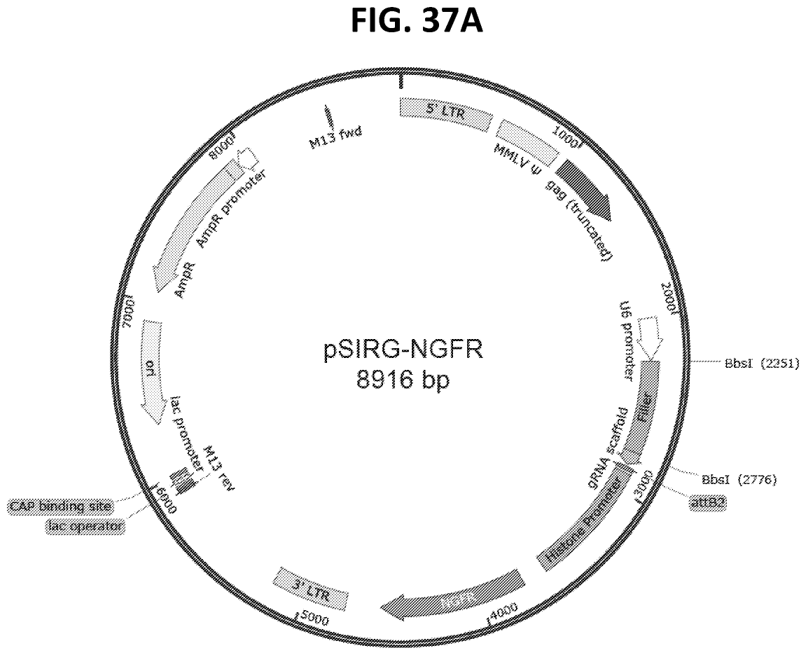

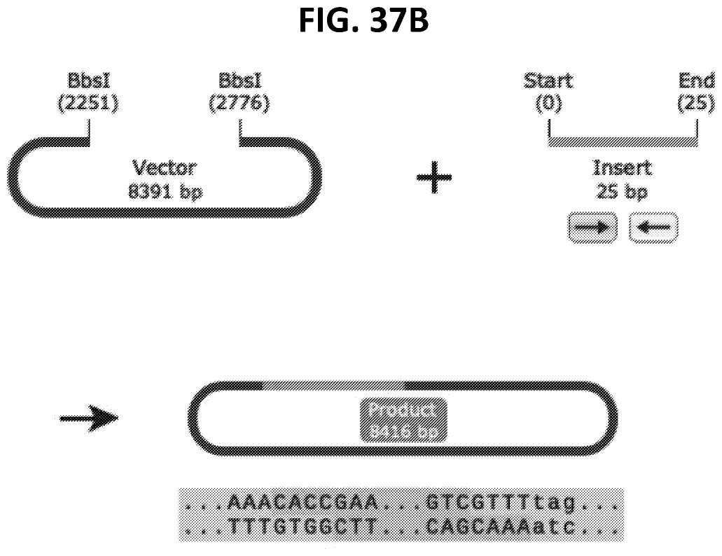

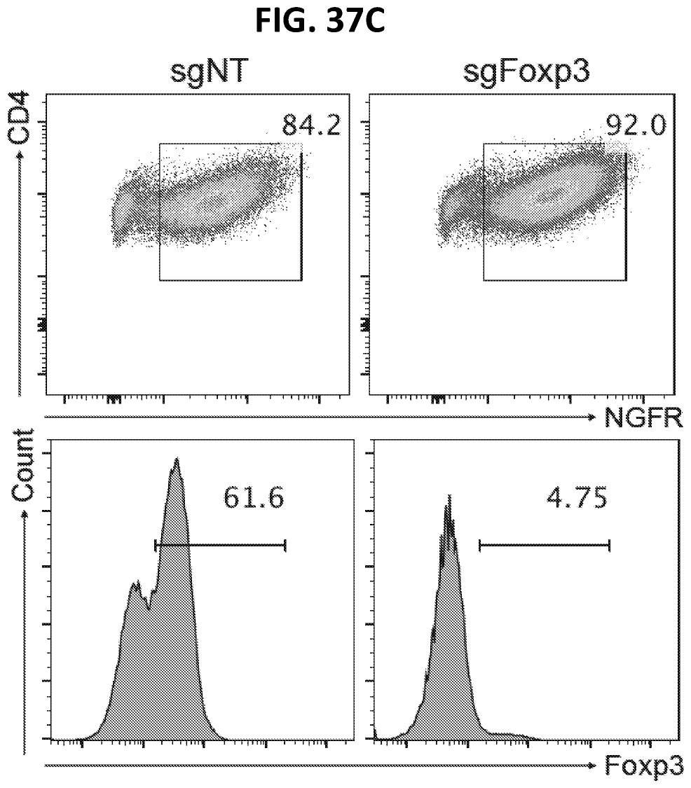

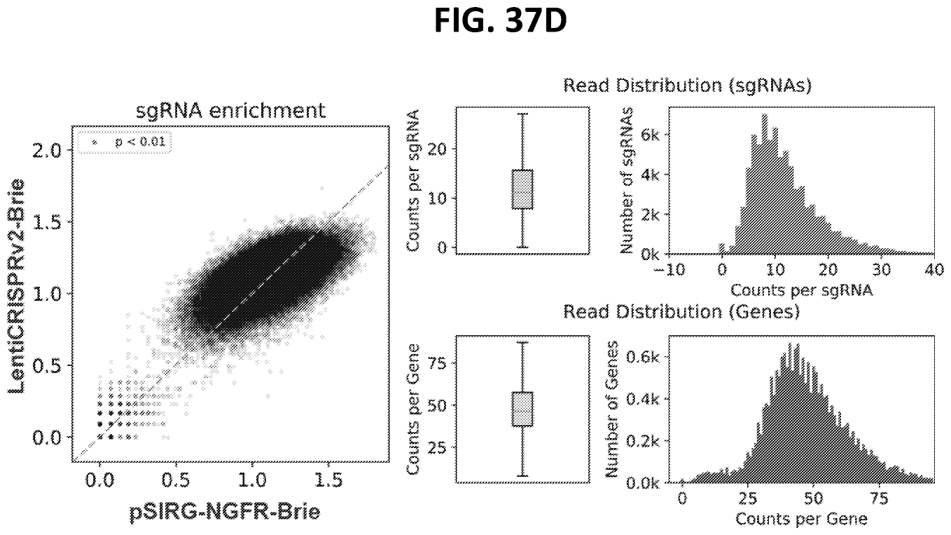

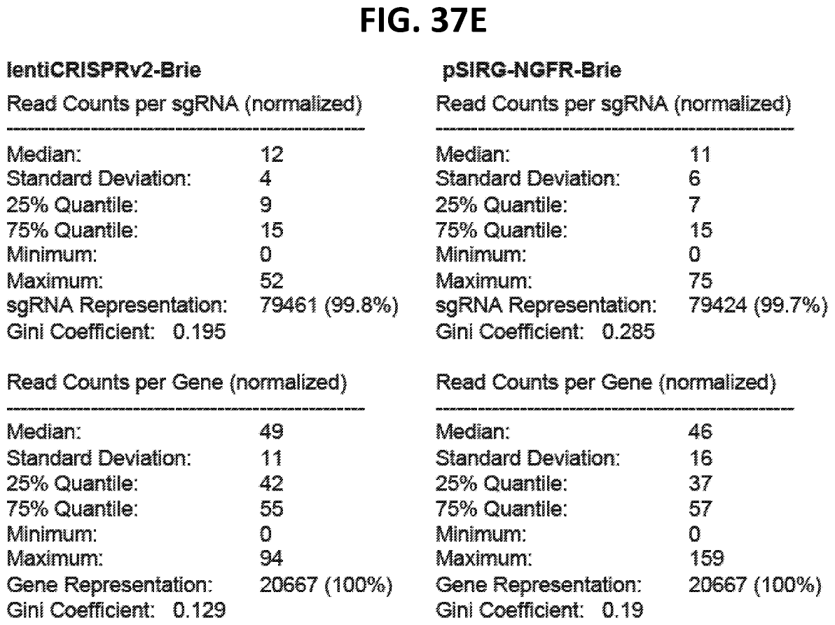

A Treg-specific Brd9 conditional knockout mouse strain was generated by breeding the Brd9 floxed mouse (Brd9^{fl}) with the Foxp3^{Cre} knock-in mouse. To test the function of Brd9 deficient Tregs in vivo, Brd9 cKO mice and WT control mice were challenged with a glioblastoma model by injecting GL261 glioblastoma cells into the brain of these mice. Tumor growth in the Brd9 cKO mice was slower than in the WT mice. Brd9 cKO mice also survived much longer than WT mice (FIG. 34). FACS analysis showed that the frequencies of intra-tumor CD4⁺ T cells were significantly higher in the Brd9 cKO mice compared to WT control mice (FIG. 36), indicating stronger anti-tumor immunity in the Brd9 cKO mice. This experiment indicates that abolishing ncBAF in Tregs can weaken Treg immune suppression and boost immune response against tumor.

In view of the many possible embodiments to which the principles of the disclosure may be applied, it should be recognized that the illustrated embodiments are only examples of the invention and should not be taken as limiting the scope of the invention. Rather, the scope of the invention is defined by the following claims. We therefore claim as our invention all that comes within the scope and spirit of these claims.

1. A method of treating an autoimmune disease or disorder in a subject, comprising administering to the subject:
a therapeutically effective amount of an agent that reduces expression or activity of bromodomain-containing 7 (Brd7), a therapeutically effective amount of an agent that reduces expression or activity of polybromo 1 (Pbrm1), or both;
a therapeutically effective amount of an agent that increases expression or activity of bromodomain-containing 9 (Brd9), or
combinations thereof.

2. The method of claim 1, wherein the method comprises administering a therapeutically effective amount of the agent that reduces expression or activity of Brd7, a therapeutically effective amount of the agent that reduces expression or activity of Pbrm1, or both to the subject.

3. The method of claim 1, wherein the method comprises administering a therapeutically effective amount of the agent that increases expression or activity of Brd9 to the subject.

4. The method of claim 1, wherein the autoimmune disease or disorder is rheumatoid arthritis, systemic lupus erythematosus, type 1 diabetes, multiple sclerosis, Sjögren's syndrome, Graves' disease, myasthenia gravis, ulcerative colitis, Hashimoto's thyroiditis, celiac disease, Crohn's disease, arthritis, inflammatory bowel disease, or scleroderma.

5. The method of claim 4, wherein the autoimmune disease or disorder is multiple sclerosis.

6. The method of claim 1, wherein
Brd9 expression or activity is increased in a Treg cell in the subject;
Brd7 expression or activity is reduced in a Treg cell in the subject;
Pbrm1 expression or activity is reduced in a Treg cell in the subject; or
combinations thereof.

7. A method of treating cancer in a subject, comprising administering to the subject:
a therapeutically effective amount of an agent that reduces expression or activity of a Brd9;
a therapeutically effective amount of an agent that increases expression or activity of a Brd7, a therapeutically effective amount of an agent that increases expression or activity of a Pbrm1, or both; or
combinations thereof.

8. The method of claim 7, wherein the method comprises administering a therapeutically effective amount of the agent that reduces expression or activity of the Brd9.

9. The method of claim 7, wherein the method comprises administering a therapeutically effective amount of the agent that increases expression or activity of the Brd7, the agent that increases expression or activity of the Pbrm1, or both.

10. The method of claim 7, further comprising treating the subject with one or more of surgery, radiation, chemotherapy, or an additional immunotherapy.

11. The method of claim 10, wherein the immunotherapy comprises administering to the subject a monoclonal antibody, a chimeric antigen receptor (CAR)-expressing T cell, an immunotoxin, or an anti-tumor vaccine.

12. The method of claim 10, wherein treating the subject with the agent enhances the additional immunotherapy.

13. The method of claim 7, wherein the cancer is glioblastoma or colorectal cancer.

14. The method of claim 7, wherein
Brd9 expression or activity is reduced in a Treg in the subject;
Brd7 expression or activity is increased in a Treg cell in the subject;
Pbrm1 expression or activity is increased in a Treg cell in the subject; or
combinations thereof.

15. A method of increasing Treg suppressor activity, comprising
reducing expression or activity of Brd7, reducing expression or activity of Pbrm1, or both;
increasing expression or activity of Brd9; or
combinations thereof in a Treg cell.

16. The method of claim 6, wherein reducing expression or activity of Brd7 and/or Pbrm1 comprises deleting all or a portion of a Brd7 and/or Pbrm1 gene in the Treg cell, respectively.

17. The method of claim 6, wherein reducing expression or activity of Brd7 and/or Pbrm1 comprises silencing expression of Brd7 and/or Pbrm1 in the Treg cell, respectively.

18. The method of claim 6, wherein reducing expression or activity of Brd7 and/or Pbrm1 comprises contacting the Treg cell with an siRNA targeting Brd7 or Pbrm1, respectively.

19. The method of claim 18, wherein the siRNA has at least 95% identity to SEQ ID NO: 43 or SEQ ID NO: 44, or comprises or consists of SEQ ID NO: 43 or SEQ ID NO: 44.

20. The method of claim 6, wherein reducing expression or activity of Brd7 and/or Pbrm1 comprises contacting the Treg cell with a gRNA targeting Brd7 or Pbrm1, respectively.

21. The method of claim 20, wherein the method further comprises expressing a Cas nuclease in the Treg cell or contacting the Treg cell with a Cas nuclease.

22. The method of claim 20, wherein the gRNA has at least 95% identity to SEQ ID NO: 10, SEQ ID NO: 8, SEQ ID NO: 46, or SEQ ID NO: 47, or comprises or consists of SEQ ID NO: 10, SEQ ID NO: 8, SEQ ID NO: 46, or SEQ ID NO: 47.

23. The method of claim 6, wherein reducing expression or activity of Brd7 and/or Pbrm1 comprises contacting the Treg cell with a small molecule inhibitor of Brd7 and/or Pbrm1, respectively.

24. The method of claim 6, wherein increasing expression or activity of Brd9 comprises contacting the Treg cell with an activator of Brd9.

25. The method of claim 6, wherein increasing expression or activity of Brd9 comprises introducing into the Treg cell an expression vector encoding Brd9 protein.

26. A method of reducing Treg suppressor activity, comprising
reducing expression or activity of Brd9;
increasing expression or activity of Brd7, increasing expression or activity of Pbrm1, or both; or combinations thereof in a Treg cell.

27. The method of claim 14, wherein
reducing expression or activity of Brd9 comprises deleting all or a portion of a Brd9 gene in the Treg cell.

28. The method of claim 14, wherein reducing expression or activity of Brd9 comprises silencing expression of Brd9 in the Treg cell.

29. The method of claim 14, wherein reducing expression or activity of Brd9 comprises contacting the Treg cell with an siRNA targeting Brd9.

30. The method of claim 29, wherein the siRNA has at least 95% identity to SEQ ID NO: 42, or comprises or consists of SEQ ID NO: 42.

31. The method of claim 14, wherein reducing expression or activity of Brd9 comprises contacting the Treg cell with an gRNA targeting Brd9.

32. The method of claim 31, wherein the method further comprises expressing a Cas nuclease in the Treg cell or contacting the Treg cell with a Cas nuclease.

33. The method of claim 31, wherein the gRNA has at least 95% identity to SEQ ID NO: 12 or SEQ ID NO: 45, or comprises or consists of SEQ ID NO: 12 or SEQ ID NO: 45.

34. The method of claim 14, wherein reducing expression or activity of Brd9 comprises contacting the Treg cell with a small molecule inhibitor of Brd9.

35. The method of claim 14, wherein increasing expression or activity of Brd7 and/or Pbrm1 comprises contacting the Treg cell with an activator of Brd7 and/or Pbrm1, respectively.

36. The method of claim 14, wherein increasing expression or activity of Brd7 and/or Pbrm1 comprises introducing into the Treg cell an expression vector encoding Brd7 and/or Pbrm1 protein, respectively.

37. The method of claim 15, wherein the Treg cell is in a subject, the method is performed in vivo, and the method comprises administering to the subject a siRNA, gRNA, small molecule inhibitor, activator, or expression vector.

38. A modified Treg cell comprising:
a) a heterologous nucleic acid molecule encoding:
i) Brd9, Brd7, or Pbrm1, or combinations thereof; or
ii) an RNAi or gRNA targeting Brd9, Brd7, or Pbrm1, or combinations thereof;
b) a small molecule inhibitor or activator targeting Brd9, Brd7, Pbrm1, or combinations thereof.

39. The modified Treg cell of claim 38, wherein the heterologous nucleic acid molecule encodes Brd9 Brd7, or Pbrm1.

40. The modified Treg cell of claim 39, wherein the heterologous nucleic acid molecule encodes an amino acid sequence having at least 90%, or at least 95% sequence identity to SEQ ID NO: 30, SEQ ID NO: 32, or SEQ ID NO: 34, or the heterologous nucleic acid molecule encodes an amino acid sequence comprising or consisting of SEQ H) NO: 30, SEQ ID NO: 32, or SEQ ID NO: 34.

41. The modified Treg cell of claim 38, comprising the heterologous nucleic acid molecule encoding the gRNA, wherein the gRNA comprises a nucleic acid sequence having at least 95% identity to SEQ ID NO: 8, SEQ ID NO: 10, SEQ ID NO: 12, SEQ ID NO: 45, SEQ ID NO: 46, or SEQ ID NO: 47, or the gRNA comprises or consists of SEQ ID NO: 8, SEQ ID NO: 10, SEQ ID NO: 12, SEQ 45, SEQ ID NC): 46, or SEQ II) NC): 47.

42. The modified Treg cell of claim 38, comprising the heterologous nucleic acid molecule encoding the siRNA, wherein the siRNA comprises a nucleic acid sequence having at least 95% identity to SEQ ID NO: 42, SEQ ID NO: 43, or SEQ ID NO: 44, or the siRNA comprises or consists of SEQ ID NO: 42, SEQ ID NO: 43, or SEQ ID NO: 44.

43. The modified Treg cell of claim 38, wherein the heterologous nucleic acid molecule is operably linked to a promoter.

44. (canceled)

45. The modified Treg cell of claim 38, wherein the small molecule inhibitor is one or more of: ACBI1, AU-15330, PFI-3, LP99, BI-7273, VZ-185, I-BRD9, BI-9564, dBRD9, and dBRD9-A.
